(19) United States
(12) Patent Application Publication
(10) Pub. No.: US 2017/0247393 A1
(43) Pub. Date: Aug. 31, 2017

(54) Aminoisoxazoline Compounds as Agonists of Alpha7-Nicotinic Acetylcholine Receptors

(71) Applicant: Forum Pharmaceuticals, Inc., Waltham, MA (US)

(72) Inventors: Raksha Acharya, Bedford, MA (US); Duane A. Burnett, Wayland, MA (US); Matthew Gregory Bursavich, Needham, MA (US); Andrew Simon Cook, Stow, MA (US); Bryce Alden Harrison, Framingham, MA (US); Andrew J. McRiner, Melrose, MA (US)

(21) Appl. No.: 15/291,740

(22) Filed: Oct. 12, 2016

Related U.S. Application Data
(60) Provisional application No. 62/243,947, filed on Oct. 20, 2015.

(51) Int. Cl. C07D 498/20; C07D 519/00

(52) U.S. Cl. CPC C07D 498/20; C07D 519/00

(57) ABSTRACT

The present invention relates to novel aminoisoxazoline compounds, and pharmaceutical compositions of the same, that are suitable as agonists or partial agonists of α7-nAChR, and methods of preparing these compounds and compositions, and the use of these compounds and compositions in methods of maintaining, treating and/or improving cognitive function. In particular, methods of administering the compound or composition to a patient in need thereof, for example a patient with a cognitive deficiency and/or a desire to enhance cognitive function, that may derive a benefit therefrom.

### CROSS REFERENCE TO RELATED APPLICATION

This application claims the benefit of priority from U.S. Provisional Application No. 62/243,947, filed Oct. 20, 2015. The foregoing related application, in its entirety, is incorporated herein by reference.

### FIELD OF THE INVENTION

The present invention relates to novel aminoisoxazoline compounds, and pharmaceutical compositions of the same, that are suitable as agonists or partial agonists of the α7- nicotinic acetylcholine receptor, and methods of preparing these compounds and compositions, and the use of these compounds and compositions in methods of maintaining, treating and/or improving cognitive function. In particular, methods of administering the compound or composition to a patient in need thereof, for example a patient with a cognitive deficiency and/or a desire to enhance cognitive function, that may derive a benefit therefrom.

### BACKGROUND OF THE INVENTION

Many forms of cognitive disease represent a steadily growing medical and social problem of our aging societies around the world. The prevalence of cognitive disease, for example dementia in North America, is approximately 6 to 10% of the population, with Alzheimer's disease accounting for a substantial portion of these cases. It is also recognized that many other neurological and psychiatric disorders may display symptoms of cognitive impairment. Some believe the main pathological features of Alzheimer's disease may relate to intraneuronal neurofibrillary tangles, formation of amyloid beta plaques and/or neurodegeneration of mainly cholinergic and, in later stages, also serotonergic, noradrenergic, and other neurons, resulting in deficiencies of acetylcholine and other neurotransmitters. Some theories suggest that the gradual develoμment of an acetylcholine signaling deficiency may be responsible for the early clinical manifestations of cognitive disease. Consequently, some believe that compounds that improve cholinergic functioning, such as acetylcholine esterase inhibitors may ameliorate the cognitive deficits in patients with cognitive disease. The most widely used acetylcholine esterase inhibitor is donepezil hydrochloride (Aricept®). In addition to Alzheimer's disease, cholinergic deficits (reductions in neurotransmitter and/or receptor levels) are observed in other disorders where there are cognitive deficits, such as schizophrenia, major depressive disorder, and Parkinson's disease.

Nicotinic acetylcholine receptors (nAChR) form a large family of ion channels which are activated by the neurotransmitter acetylcholine which is produced in the body (Galzi and Changeux, Neuropharmacol. 1995, 34, 563-582). A functional nAChR consists of five subunits which may be different (certain combinations of α1-9 and β1-4,γ,δ,ε subunits) or identical (α7-9). This leads to the formation of a diversity of subtypes which differ in the distribution in the muscles, the nervous system and other organs (McGehee and Role, Annu. Rev. Physiol. 1995, 57, 521-546). Activation of nAChR leads to influx of cations into the cell and to stimulation of nerve cells or muscle cells. Selective activation of individual nAChR subtypes restricts this stimulation to the cell types which have a corresponding nAChR subtype and is thus able to avoid unwanted side effects such as, for example, stimulation of nAChRs in the muscles. Clinical experiments with nicotine and experiments in various animal models indicate that central nicotinic acetylcholine receptors are involved in learning and memory processes (e.g. Rezvani and Levin, Biol. Psychiatry 2001, 49, 258-267). Nicotinic acetylcholine receptors of the alpha7 subtype (α7 nAChR) have a particularly high concentration in regions of the brain which are important for learning and memory, such as the hippocampus and the cerebral cortex (Seguela et al., J. Neurosci. 1993, 13, 596-604). The α7 nAChR has a particularly high permeability for calcium ions, modulates neurotransmission, influences the growth of axons and, in this way, modulates neuronal plasticity (Broide and Leslie, Mol. Neurobiol. 1999, 20, 1-16).

WO 2003/055878 describes a variety of agonists of the α7 nAChR said to be useful for improving cognition. WO 2003/055878 suggests that certain agonists of the α7 nAChR are useful for improving perception, concentration, learning or memory, especially after cognitive impairments like those occurring for example in situations/diseases/syndromes such as mild cognitive impairment, age-associated learning and memory impairments, age-associated memory loss, Alzheimer's disease, schizophrenia and certain other cognitive disorders.

### BRIEF SUMMARY OF THE INVENTION

An aspect of the invention provides an aminoisoxazoline compound represented by Formula (I):

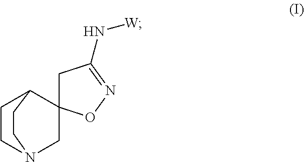

wherein:
W represents a moiety represented by ring system M-I, M-II, M-III, or M-IV:

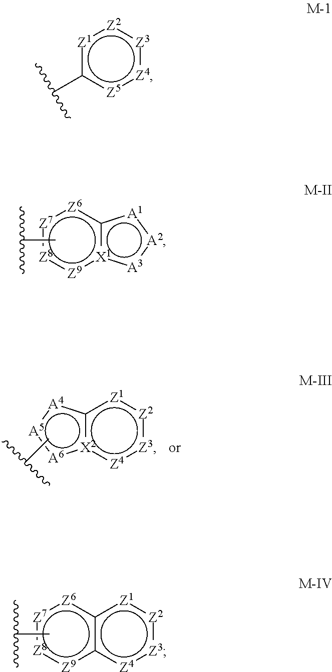

wherein:
Z¹, Z², Z³, Z⁴, and Z⁵ independently represent N or CR¹; with the proviso that no more than two of Z¹, Z², Z³, Z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2=O; wherein:
   i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, ——OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁵)(R⁶), —(CO)C₁-C₄-alkyl, —(CO)N(R⁵)(R⁶), —NR⁵(CO)(R⁶), —SO₂C₁-C₄-alkyl, —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, an aryl radical, a heteroaryl radical, or the bond directly attaching the W moiety with the aminoisoxazoline moiety; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁵)SO₂C₁-C₄-alkyl, may be independently substituted with up to 5 radical substituents comprising: —D, halogen radical, ═O, —OR⁵, —(CH₂)ₘOR⁵, —N(R⁵)(R⁶), —NR⁵(CO)(R⁶), —(CH₂)ₘN(R⁵)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR⁵, —(CH₂)ₘOR⁵, —N(R⁵)(R⁶), —NR⁵(CO)(R⁶), —(CH₂)ₘN(R⁵)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched radical, a branched C₃-C₄-alkyl radical, a C₃-C₄-cycloalkyl radical, a C₁-C₄-hydroxyalkyl radical, a C₁-C₂haloalkyl radical, or —OC₁-C₂-haloalkyl radical;
R⁵ and R⁶ independently represent —H, —D, an unbranched C₁-C₄-alkyl radical, a branched C₃-C₄-alkyl radical, a C₃-C₄-cycloalkyl radical, or the N(R⁵)(R⁶) moiety forms a cycle, wherein R⁵ and R⁶ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X¹ independently represents N or C;
A¹, A², and A³ independently represent N, NR⁷, N(CH₂)ₘR⁷, O, S, or CR⁸; with the proviso that only one A¹, A², and A³ is NR⁷, N(CH₂)ₘR⁷, O, or S; with the further proviso that when X¹ is N, then A¹, A², and A³ independently represent N or CR⁸;
R⁷ independently represents —H, —D, —SO₂(CH₂)ₘR⁹, —(CO)(CH₂)ₘR⁹, ——(CO)N(R⁹)(R¹⁰), an unbranched C₁-C₄-alkyl radical, a branched C₃-C₄-alkyl radical, a C₃-C₆-cycloalkyl radical, a (3-6 membered)-heterocycloalkyl radical, an aryl radical, or a heteroaryl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl), —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR⁹, —(CH₂)ₘOR⁹), —N(R⁹)(R¹⁰), —NR⁹(CO)(R¹⁰), —(CH₂)ₘN(R⁹)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl), —N(R⁹)(R¹⁰), —(CO)C₁-C₄-alkyl, —(CO)N(R⁹)(R¹⁰), —NR⁹(CO)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, an aryl radical, or a heteroaryl radical; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁹)SO₂C₁-C₄-alkyl, may be independently substituted with up to 5 radical substituents comprising: —D, halogen radical, ═O, —OR⁹, —(CH₂)ₘOR⁹, —N(R⁹)(R¹⁰), —NR⁶(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹), —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁹ and R¹⁰ independently represent —H, —D, an unbranched C₁-C₄-alkyl radical, a branched C₃-C₄-alkyl radical, a C₃-C₄-cycloalkyl radical, or the —N(R⁹)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, NR¹¹, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²; independently represents —H, —D, —SO₂(CH₂)ₘR¹³, —(CO)(CH₂)ₘR¹³, ——(CO)N(R¹³)(R¹⁴), an unbranched C₁-C₄-alkyl radical, a branched C₃-C₄-alkyl radical, a C₃-C₆-cycloalkyl radical, a (3-6 membered)-heterocycloalkyl radical, an aryl radical, a heteroaryl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹² independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R¹³)(R¹⁴), —(CO)C₁-C₄-alkyl, —(CO)N(R¹³)(R¹⁴), —NR¹³(CO)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, an aryl radical, a heteroaryl radical, or the bond directly attaching the W moiety with the aminoisoxazoline moiety; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R¹³)SO₂C₁-C₄-alkyl, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³(R¹⁴), —N(R¹³)(R¹⁴), —N(R¹³)SO₂C₁-C₄-alkyl, —(CO)(CH₂)ₘR¹³, —(CO)N(R¹³)(R¹⁴) an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical;
R¹³ and R^{H} independently represent —H, —D, an unbranched C₁-C₄-alkyl radical, a branched C₃-C₄-alkyl radical, a C₃-C₄-cycloalkyl radical, or the —N(R¹³)(R¹⁴) moiety forms a cycle, wherein le and R^{H} taken together represent a C₂-C₆-alkyl di-radical or a (3-6 membered)-heteroalkyl di-radical; wherein the (3- 6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
   or a pharmaceutically acceptable salt thereof

An aspect of the invention provides an aminoisoxazoline compound represented by Formula (I):

wherein:

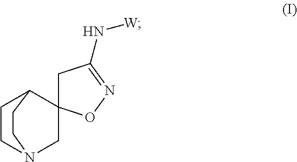

W represents a moiety represented by ring system M-I, M-II, M-III, or M-IV:

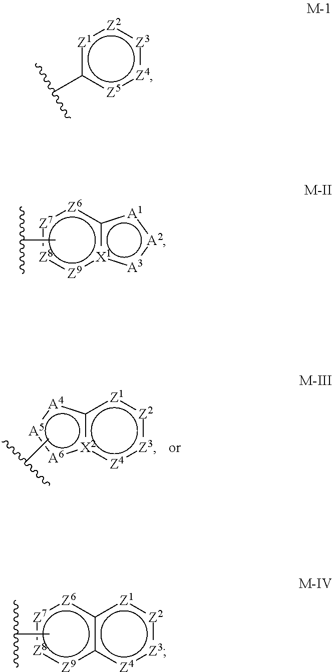

wherein:
Z¹, Z², Z³, Z⁴, and Z⁵ independently represent N or CR¹; with the proviso that no more than two of Z¹, Z², Z³, Z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein:
   i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical; and
   ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₄-alkyl radical, a branched C₃-C₄-alkyl radical, a C₃-C₆-cycloalkyl radical, a (3-6 membered)-heterocycloalkyl radical, an unbranched a branched or cyclic —OC₃-C₄-alkyl, —N(R⁵)(R⁶), —(CO)C₁-C₄-alkyl, —(CO)N(R⁵)(R⁶), —NR⁵(CO)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, an aryl radical, a heteroaryl radical, or the bond directly attaching the W moiety with the aminoisoxazoline moiety; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁵)SO₂C₁-C₄-alkyl, may be independently substituted with up to 5 radical substituents comprising: —D, halogen radical, ═O, —OR⁵, —(CH₂)ₘOR⁵, —N(R⁵)(R⁶), —NR⁵(CO)(R⁶), —(CH₂)ₘN(R⁵)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂haloalkyl radical; and
   ii) the aryl radical or the heteroaryl radical may be independently substituted with up to 5 radical substituents comprising: —D, halogen radical, —CN, —OR⁵, —(CH₂)ₘOR⁵, —N(R⁵)(R⁶), —NR⁵(CO)(R⁶), —(CH₂)ₘN(R⁵)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁵ and R⁶ independently represent —H, —D, an unbranched C₁-C₄-alkyl radical, a branched C₃-C₄-alkyl radical, a C₃-C₄-cycloalkyl radical, or the —N(R⁵)(R⁶) moiety forms a cycle, wherein R⁵ and R⁶ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X¹ independently represents N or C;
A¹, A², and A³ independently represent N, —NR⁷, N(CH₂)ₘR⁷, O, S, or CR⁸; with the proviso that only one A¹, A², and A³ is NR⁷, N(CH₂)ₘR⁷, O, or S; with the further proviso that when X¹ is N, then A¹, A², and A³ independently represent N or CR⁸;
R⁷ independently represents —H, —D, SO₂(CH₂)ₘR⁹, —(CO)(CH₂)ₘR⁹, ——(CO)N(R⁹)(R¹⁰), an unbranched C₁-C₄-alkyl radical, a branched C₃-C₄-alkyl radical, a C₃-C₆-cycloalkyl radical, a (3-6 membered)-heterocycloalkyl radical, an aryl radical, or a heteroaryl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl), —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁸ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁹)(R¹⁰), —(CO)C₁-C₄-alkyl, —(CO)N(R⁹)(R¹⁰), —NR⁹(CO)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, an aryl radical, or a heteroaryl radical; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁹)SO₂C₁-C₄-alkyl, may be independently substituted with up to 5 radical substituents comprising: —D, halogen radical, ═O, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl,) —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁹ and R¹⁰ independently represent —H, —D, an unbranched C₁-C₄-alkyl radical, a branched C₃-C₄-alkyl radical, a C₃-C₄-cycloalkyl radical, or the)N(R⁹)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, —NR¹¹, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²;
R¹¹ independently represents —H, —D, —SO₂(CH₂)ₘR¹³, —(CO)(CH₂)ₘR¹³, —(CO)N(R¹³)(R¹⁴), an unbranched C₁-C₄-alkyl radical, a branched C₃-C₄-alkyl radical, a C₃-C₆-cycloalkyl radical, a (3-6 membered)-heterocycloalkyl radical, an aryl radical, a heteroaryl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹² independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R¹³)(R¹⁴), —(CO)C₁-C₄-alkyl, —(CO)N(R¹³)(R¹⁴), —NR¹³(CO)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, an aryl radical, a heteroaryl radical, or the bond directly attaching the W moiety with the aminoisoxazoline moiety; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R¹³)SO₂C₁-C₄-alkyl, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴) an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹³ and R¹⁴ independently represent —H, —D, an unbranched C₁-C₄-alkyl radical, a branched C₃-C₄-alkyl radical, a C₃-C₄-cycloalkyl radical, or the —N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof;
with the proviso that the aminoisoxazoline compound represented by Formula (I) is exclusive of:

N-phenyl-4H-1′-azaspiro[isoxazole-5,3′-bicyclo[2.2.2]octan]-3-amine;

N-(isoquinolin-3-yl)-4H-1′-azaspiro[isoxazole-5,3′-bicyclo[2.2.2]octan]-3-amine;

N-(benzo[d]thiazol-2-yl)-4H-1′-azaspiro[isoxazole-5,3′-bicyclo[2.2.2]octan]-3-amine;

N-(6-methoxybenzo[d]thiazol-2-yl)-4H-1′-azaspiro[isoxazole-5,3′-bicyclo[2.2.2]octan]-3-amine;

N-(1H-indazol-3-yl)-4 ¹H-4-azaspiro[bicyclo[2.2.2]octane-2,5 ′-isoxazol]-3 ′-amine;

N-(7-bromopyrrolo[2,1-f][1,2,4]triazin-4 -yl)-4H-1′-azaspiro[isoxazole5,3′-bicyclo[2.2.2]octan]-3-amine;

N-(4-chlorobenzo[d]thiazol-2 -yl)-4H-1′-azaspiro[isoxazole5,3′-bicyclo[2.2.2]octan]-3-amine;

N-(5 -chlorobenzo[d]thiazol-2 -yl)-4H-1′-azaspiro[isoxazole5,3′-bicyclo[2.2.2]octan]-3-amine;

N-(6-chlorobenzo[d]thiazol-2 -yl)-4H-1′-azaspiro[isoxazole5,3′-bicyclo[2.2.2]octan]-3-amine;

N-(7-chlorobenzo[d]thiazol-2 -yl)-4H-1′-azaspiro[isoxazole5,3′-bicyclo[2.2.2]octan]-3-amine;

N-(benzo[b]thiophen-2 -yl)-4H-1′-azaspiro[isoxazole-5,3′-bicyclo[2.2.2.]octan]-3-amine;

N-(benzo[d]oxazol-2-yl)-4H-1′-azaspiro[isoxazole -5,3′-bicyclo[2.2.2]octan]-3-amine;

N-(5,6,7, 8 -tetrahydroisoquinolin-3-yl)-4H-1′-azaspiro[isoxazole-5,3′-bicyclo[2.2.2]octan]-3-amine;

N-(5 -methoxypyrimidin-2-yl)-4H-1′-azaspiro[isoxazole-5,3′-bicyclo[2.2.2]octan]-3-amine;

N-(furo[3,2-b]pyridin-5-yl)-4H-1′-azaspiro[isoxazole-5,3′-bicyclo[2.2.2]octan]-3-amine; and

N-(furo [2,3-c] pyridin-5 -yl)-4H-1′-azaspiro[isoxazole -5,3′-bicyclo[2.2.2]octan]-3-amine.

An aspect of the invention relates to the aminoisoxazoline compound of Formula (I), wherein the compound is represented by Formula (Ia):

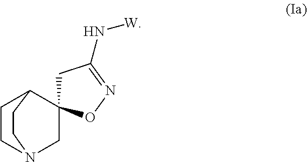

An aspect of the invention relates to the aminoisoxazoline compound of Formula (I), wherein the compound is represented by Formula (Ib):

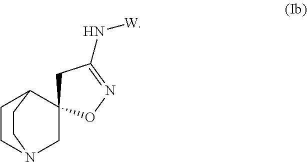

An aspect of the invention relates to a single stereoisomer of the aminoisoxazoline compound represented by Formula (I), (Ia), or (Ib), or a pharmaceutically acceptable salt thereof

An aspect of the invention relates to a single enantiomer or a single diastereomer of the aminoisoxazoline compound represented by Formula (I), (Ia), or (Ib), or a pharmaceutically acceptable salt thereof.

An aspect of the invention relates to a pharmaceutical composition comprising the aminoisoxazoline compound represented by Formula (I), (Ia), or (Ib), or a pharmaceutically acceptable salt thereof; and at least one pharmaceutically acceptable carrier, excipient or diluent.

An aspect of the invention relates to a method comprising administering to a patient in need thereof an effective dose of an aminoisoxazoline compound represented by Formula (I), (Ia), or (Ib), or a pharmaceutically acceptable salt thereof, or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating a patient in need thereof, comprising: administering to the patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of maintaining, treating, curing and/or improving at least one cognitive function in a patient in need thereof, comprising: administering to the patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of maintaining, treating, curing and/or improving at least one cognitive function in a patient in need thereof, comprising: administering to the patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating a patient diagnosed as having a cognitive impairment, comprising: administering to the an effective dose of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating a patient in need thereof, comprising: administering to the patient, for example, a patient diagnosed with having a cognitive impairment, Limited Cognitive Impairment, Mild Cognitive Impairment, Alzheimer's disease, and/or schizophrenia, an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent; such that the patient may derive a benefit therefrom.

Another aspect of the invention provides a method of treating one or more symptoms associated with a cognitive impairment, comprising administering to a patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent; wherein the patient suffers from, or has been diagnosed as having, a cognitive impairment.

Another aspect of the invention provides a method of improving cognition of a patient in need thereof, comprising: administering to the patient an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of improving cognition in a patient suffering from a cognitive impairment, such as a cognitive impairment associated with either schizophrenia or Alzheimer's disease, for example mild Alzheimer's disease, moderate Alzheimer's disease, severe Alzheimer's disease, or mild-to-moderate Alzheimer's disease, comprising administering an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating a patient suffering from, diagnosed with having, or suffers from one or more symptoms associated with, a cognitive impairment, for example, Alzheimer's disease, dementia of an Alzheimer's type, MCI, LCI, or schizophrenia, comprising: administering to the patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent. For example, the method of treating a patient suffering from, diagnosed with having, or suffers from one or more symptoms associated with, a cognitive impairment, may provide said patient at least one of the following: (i) treats, minimizes progression of, prevents the deterioration of, or reduces the rate of detioraration of, one or more symptoms associated with the cognitive impairment; (ii) treats the cognitive impairment; (iii) improves cognition in said cognitively impaired patient; (iv) improves one or more behavioral symptoms associated with the cognitive impairment; (v) provides a pro-cognitive effect; (vi) provides a pro-cognitive effect in at least one of the following: visual motor, learning, delayed memory, or executive function, or (vii) provides a positive effect on clinical function in said cognitively impaired patient.

Another aspect of the invention provides a method of treating a patient previously treated, or currently being treated, with an AChEI, that is suffering from, or has been diagnosed with having, a cognitive impairment, for example, Alzheimer's disease, dementia of an Alzheimer's type, MCI, LCI, or schizophrenia, comprising: administering to the patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluents; wherein the method improves one or more symptoms associated with the cognitive impairment in the previously, or currently, AChEI treated patient.

Another aspect of the invention provides a method of treating a patient suffering from, or diagnosed with having a cognitive impairment, comprising: administering to the patient an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent; wherein the method provides a positive effect on cognition or a positive effect on clinical function in said cognitively impaired patient, and wherein said patient has been previously treated or is currently being treated with an AChEI.

Another aspect of the invention provides a method of improving cognition in a patient diagnosed as having a probable cognitive disease, comprising: administering to the patient an effective dose of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of improving or substantially improving one or more symptoms in a cognitve disease patient, comprising: administering to the patient an effective dose of an aminoisoxazoline compound represented by Formula (I), (Ia), or (Ib), or a pharmaceutically acceptable salt thereof; or administering to the patient an effective dose of a pharmaceutical composition comprising the effective dose of the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of slowing the rate of deterioration of at least one symptom in a cognitve disease patient, comprising: administering to the patient an effective dose of an aminoisoxazoline compound represented by Formula (I), (Ia), or (Ib), or a pharmaceutically acceptable salt thereof; or administering to the patient the pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating one or more symptoms associated with a cognitive disease in a patient suffering therefrom, comprising: administering to the patient an effective dose of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent

Another aspect provides a method of minimizing or substantially halting the rate of progression of one or more cognitive diseases in a patient suffering from a cognitive disease, comprising: administering to the patient an effective dose of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of substantially stopping or reversing progression of one or more cognitive diseases, in a patient suffering therefrom, comprising: administering to the patient an effective dose of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating dementia, comprising: administering to a patient in need thereof an effective amount of an aminoisoxazoline compound represented by Formula (I), (Ia), or (Ib), or a pharmaceutically acceptable salt thereof; or administering to the patient an effective dose of a pharmaceutical composition comprising the effective amount of the aminoisoxazoline compound represented by Formula (I), (Ia), or (Ib), or a pharmaceutically acceptable salt thereof, and at least one pharmaceutically acceptable carrier, excipient or diluent; wherein said effective amount is administered in an effective dose.

Another aspect of the invention provides a method of treating dementia, comprising: administering to a patient in need thereof an effective amount of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

Another aspect of the invention provides a method of treating dementia, comprising: administering to a patient in need thereof an effective amount of an aminoisoxazoline compound represented by Formula (I), (Ia), or (Ib), or a pharmaceutically acceptable salt thereof, wherein the aminoisoxazoline compound represented by Formula (I), (Ia), or (Ib), or a pharmaceutically acceptable salt thereof, is administered in the form of a pharmaceutical composition comprising at least one pharmaceutically acceptable carrier, excipient or diluent.

Another aspect of the invention provides a method of treating dementia, comprising: administering to a patient in need thereof an effective amount of an aminoisoxazoline compound represented by Formula (I), (Ia), or (Ib), or a pharmaceutically acceptable salt thereof; or administering to the patient an effective dose of a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent; wherein the pharmaceutical composition is in the form of a tablet.

Another aspect of the invention provides a method of treating a patient having a cognitive disease and being administered an acetylcholine esterase inhibitor, comprising: administering to a patient in need thereof an effective amount of an aminoisoxazoline compound represented by Formula (I), (Ia), or (Ib), or a pharmaceutically acceptable salt thereof; or administering to the patient an effective dose of a pharmaceutical composition comprising an aminoisoxazoline compound represented by Formula (I), (Ia), or (Ib), or a pharmaceutically acceptable salt thereof, and at least one pharmaceutically acceptable carrier, excipient or diluent; wherein the treatment comprises halting the administration of the acetylcholine esterase inhibitor prior to treating with the aminoisoxazoline compound represented by Formula (I), (Ia), or (Ib), or a pharmaceutically acceptable salt thereof.

### BRIEF DESCRIPTION OF THE DRAWINGS

FIG. 1: Illustrates a 3-D representation of the formed crystal of (S)-4H-1′-azaspiro[isoxazole-5,3′-bicyclo[2.2.2]octan]-3-amine (2R,3R)-2,3-dihydroxysuccinate ((S)-A-2 L tartaric acid salt).

### DETAILED DESCRIPTION OF THE INVENTION

An embodiment of the present invention provides an aminoisoxazoline compound represented by Formula (I):

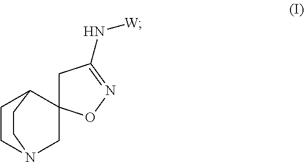

wherein:
W represents a moiety represented by ring system M-I, M-II, M-III, or M-IV:

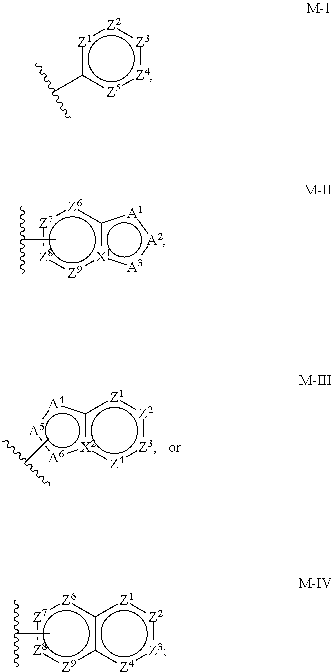

wherein:
Z¹, Z², Z³, Z⁴, and Z⁵ independently represent N or CR¹; with the proviso that no more than two of Z¹, Z², Z³, z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹ Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein:
   i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂- haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched a branched or cyclic —OC₃-C₄-alkyl, —N(R⁵)(R⁶), —(CO)C₁-C₄-alkyl, —(CO)N(R⁵)(R⁶), —NR⁵(CO)(R⁶), —SO₂C₁-C₄-alkyl, —SO₂N(R⁵)(R⁶), —(CH₂)ₘSO₂C₁-C₄-alkyl, —(CH₂)ᵢₙSO₂N(R⁵)(R⁶), —N(R⁵)SO₂C₁-C₄-alkyl, an aryl radical, a heteroaryl radical, or the bond directly attaching the W moiety with the aminoisoxazoline moiety; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁵)SO₂C₁-C₄-alkyl, may be independently substituted with up to 5 radical substituents comprising: —D, halogen radical, ═O, —OR⁵, —(CH₂)ₘOR⁵, —N(R⁵)(R⁶), —NR⁵(CO)(R⁶), —(CH₂)ₘN(R⁵)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR⁵, —(CH₂)ₘOR⁵, —N(R⁵)(R⁶), —NR⁵(CO)(R⁶), —(CH₂)ₘN(R⁵)(R⁶), —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁵ and R⁶ independently represent —H, —D, an unbranched C₁-C₄-alkyl radical, a branched C₃-C₄-alkyl radical, a C₃-C₄-cycloalkyl radical, or the —N(R⁵)(R⁶) moiety forms a cycle, wherein R⁵ and R⁶ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X¹ independently represents N or C;
A¹, A², and A³ independently represent N, —NR⁷, N(CH₂)ₘR⁷, O, S, or CR⁸; with the proviso that only one A¹, A², and A³ is NR⁷, N(CH₂)ₘR⁷, O, or S; with the further proviso that when X¹ is N, then A¹, A², and A³ independently represent N or CR⁸;
R⁷ independently represents —H, —D, —SO₂(CH₂)ₘR⁹, —(CO)(CH₂)ₘR⁹, —(CO)N(R⁹)(R¹⁰), an unbranched C₁-C₄-alkyl radical, a branched C₃-C₄-alkyl radical, a C₃-C₆-cycloalkyl radical, a (3-6 membered)-heterocycloalkyl radical, an aryl radical, or a heteroaryl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl), —SO₂N(R⁹)(R¹⁰), —(CH₂) ᵢₙS C₂ C₁-C₄-alkyl,) —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁸ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁹)(R¹⁰), —(CO)C₁-C₄-alkyl, —(CO)N(R⁹)(R¹⁰), —NR⁹(CO)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, an aryl radical, or a heteroaryl radical; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁹)SO₂C₁-C₄-alkyl, may be independently substituted with up to 5 radical substituents comprising: —D, halogen radical, ═O, —OR⁹, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl), —SO₂N(R⁹)(R¹⁰), —(CH₂)ₘSO₂C₁-C₄-alkyl,) —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂haloalkyl radical; and
   ii) the aryl radical or the heteroaryl radical may be independently substituted with up to 5 radical substituents comprising: —D, halogen radical, —CN, —OR⁹, —(CH₂)ₘOR⁹, —N(R⁹)(R¹⁰), —NR⁹(CO)(R¹⁰), —(CH₂)ₘN(R⁹)(R¹⁰), —SO₂N(R⁹)(R¹⁰), —(CH₂)ₘSO₂C₁-C₄-alkyl,)(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁹ and R¹⁰ independently represent —H, —D, an unbranched C₁-C₄-alkyl radical, a branched C₃-C₄-alkyl radical, a C₃-C₄-cycloalkyl radical, or the)N(R⁹)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, —NR¹¹, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²;
R¹¹ independently represents —H, —D, SO₂(CH₂)ₘR¹³, —(CO)(CH₂)ₘR¹³, —(CO)N(R¹³)(R¹⁴), an unbranched C₁-C₄-alkyl radical, a branched C₃-C₄-alkyl radical, a C₃-C₆-cycloalkyl radical, a (3-6 membered)-heterocycloalkyl radical, an aryl radical, a heteroaryl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —O¹³, —(CH₂)ₘOR¹³, —N(R¹³)(R¹⁴), —NR¹³(CO)(R¹⁴), —SO₂C₁-C₄-alkyl, —SO₂N(R¹³)(R¹⁴),)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹² independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R¹³)(R¹⁴), —(CO)C₁-C₄-alkyl, —(CO)N(R¹³)(R¹⁴), —NR¹³(CO)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, an aryl radical, a heteroaryl radical, or the bond directly attaching the W moiety with the aminoisoxazoline moiety; wherein:
   i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R¹³)SO₂C₁-C₄-alkyl, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH₂)ₘN(R¹³)(R¹⁴), —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical; and
   ii) the aryl radical or the heteroaryl radical may be independently substituted with up to 5 radical substituents comprising: —D, halogen radical, —CN, —(CH₂)ₘOR¹³, —N(R¹³)(R¹⁴)_{, —NR}¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ᵢₙSO₂N(R¹³)(R¹⁴), —N(R¹³)SO₂C₁-C₄-alkyl, —(CO)(CH₂)ₘR¹³, —(CO)N(R¹³)(R-¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹³ and R¹⁴ independently represent —H, —D, an unbranched C₁-C₄-alkyl radical, a branched C₃-C₄-alkyl radical, a C₃-C₄-cycloalkyl radical, or the —N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof;
with the proviso that the aminoisoxazoline compound represented by Formula (I) is exclusive of:

N-phenyl-4H-1′-azaspiro[isoxazole-5,3′-bicyclo[2.2.2]octan]-3-amine;

N-(isoquinolin-3-yl)-4H-1′-azaspiro[isoxazole -5,3′-bicyclo[2.2.2.]octan]-3-amine;

N-(benzo[d]thiazol-2-yl)-4H-1′-azaspiro[isoxazole -5 ,3′-bicyclo[2.2.2]octan]-3-amine;

N-(6-methoxybenzo[d]thiazol-2-yl)-4H-1′-azaspiro[isoxazole-5,3′-bicyclo[2.2.2]octan]-3-amine;

N-(1H-indazol-3-yl)-4 ¹H-4-azaspiro[bicyclo[2.2.2]octane-2,5 soxazol]-3 ′-amine;

N-(7-bromopyrrolo[2,1-f][1,2,4]triazin-4 -yl)-4H-1′-azaspiro[isoxazole-5,3 ′-bicyclo[2.2.2]octan]-3-amine;

N-(4-chlorobenzo[d]thiazol-2 -yl)-4H-1′-azaspiro[isoxazole-5,3 ′-bicyclo[2.2.2]octan]-3-amine;

N-(5 -chlorobenzo[d]thiazol-2 -yl)-4H-1′-azaspiro[isoxazole-5,3 ′-bicyclo[2.2.2]octan]-3-amine;

N-(6-chlorobenzo[d]thiazol-2 -yl)-4H-1′-azaspiro[isoxazole-5,3 ′-bicyclo[2.2.2]octan]-3-amine;

N-(7-chlorobenzo[d]thiazol-2 -yl)-4H-1′-azaspiro[isoxazole-5,3 ′-bicyclo[2.2.2]octan]-3-amine;

N-(benzo[b]thiophen-2 -yl)-4H-1′-azaspiro[isoxazole-5,3′-bicyclo[2.2 .2] octan]-3-amine;

N-(benzo[d]oxazol-2-yl)-4H-1′-azaspiro[isoxazole-5,3′-bicyclo[2.2.2]octan]-3-amine;

N-(5 ,6,7,8 -tetrahydroisoquinolin-3-yl)-4H-1′-azaspiro[isoxazole-5,3′-bicyclo[2.2.2]octan]-3- amine;

N-(5-methoxypyrimidin-2-yl)-4H-1′-azaspiro[isoxazole-5,3′-bicyclo[2.2.2]octan]-3-amine;

N-(furo[3,2-b]pyridin-5-yl)-4H-1′-azaspiro[isoxazole-5,3′-bicyclo[2.2.2]octan]-3-amine; and

N-(furo [2,3-c] pyridin-5 -yl)-4H-1′-azaspiro[isoxazole-5,3′-bicyclo[2.2.2]octan]-3-amine; or a pharmaceutically acceptable salt thereof.

In certain embodiments, the aminoisoxazoline compound represented by Formula (I) may be represented by Formula (Ia):

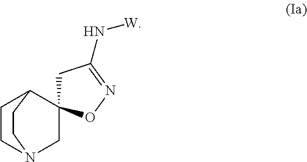

In certain embodiments, the aminoisoxazoline compound represented by Formula (I) may be represented by Formula (Ib):

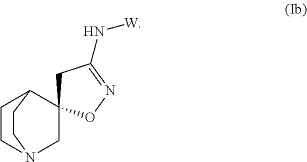

In certain embodiments, the aminoisoxazoline compound represented by Formula (I), (Ia), or (Ib), may comprise the W representing the moiety represented by the ring system M-I:

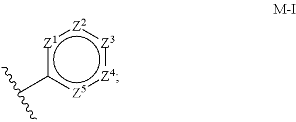

wherein:
Z¹, Z², Z³, Z⁴, and Z⁵ independently represent N or CR¹; with the proviso that no more than two of Z¹, Z², Z³, z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein:
   i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂haloalkyl radical, or OC₁-C₂- haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof;
with the proviso that the aminoisoxazoline compound represented by Formula (I) is exclusive of:

N-phenyl-4H-1′-azaspiro[isoxazole -5,3′-bicycl o [2.2.2]octan]-3-amine;

N-(5 , 6,7,8 -tetrahydroisoquinolin-3-yl)-4H-1′-azaspiro[isoxazole-5 ,3 ′ -bicyclo[2.2.2]octan]-3- amine; and

N-(5 -methoxypyrimidin-2-y0-4H-1′-azaspiro[isoxazole-5,3′-bicyclo[2.2.2]octan]-3-amine.

In certain embodiments, the aminoisoxazoline compound represented by Formula (I), (Ia), or (Ib), may comprise the W representing the moiety represented by the ring system

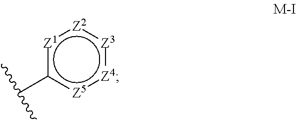

wherein:
Z¹, Z², Z³, Z⁴, and Z⁵ independently represent N or CR¹; with the proviso that no more than two of Z¹, Z², Z³, Z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂).$C₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein:
   i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
   ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; with the proviso that:
   i) when each member of Z¹, Z², Z³, Z⁴, and Z⁵ independently represent CR¹, then no more than four of the R¹ is —H;
   ii) when both of Z¹ and Z⁵ are N and Z², Z³, and Z⁴ independently represent CR¹ with the R¹ of Z³ representing OCH₃, then no more than one of the R¹ of the Z² and Z⁴ is —H; or
   iii) when Z¹ is N, each of Z², Z³, Z⁴, and Z⁵ independently represent CR¹, and adjacent members Z³ and Z⁴ form a cycle such that the adjacent R¹ substitutents taken together represents a C₄-alkyl di-radical; then:
      a) no more than one of the R¹ of the Z² and Z⁵ is —H; or
      b) the C₄-alkyl di-radical is independently substituted with at least one radical substituent;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof.

In certain embodiments, the aminoisoxazoline compound represented by Formula (I), (Ia), or (Ib), may comprise the W representing the moiety represented by the ring system M-II:

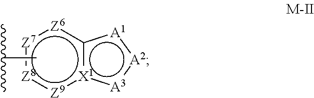

wherein:
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, or ═O;
X¹ independently represents N or C;
A¹, A², and A³ independently represent N, —NR⁷, N(CH₂)ₘR⁷, O, S, or CR⁸; with the proviso that only one A¹, A², and A³ is NR⁷, N(CH₂)ₘR⁷, O, or S; with the further proviso that when X¹ is N, then A¹, A², and A³ independently represent N or CR⁸;
R⁷ independently represents —H, —D, —SO₂(CH₂)ₘR⁹, —(CO)(CH₂)ₘR⁹, —(CO)N(R⁹)(R¹⁰), an unbranched C₁-C₄-alkyl radical, a branched C₃-C₄-alkyl radical, a C₃-C₆-cycloalkyl radical, or a (3-6 membered)-heterocycloalkyl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, or ═O;
R⁸ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁹)(R¹⁰), —(CO)C₁-C₄-alkyl,) —(CO)N(R⁹)(R¹⁰), —NR⁹(CO)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), or —N(R⁹)SO₂C₁-C₄-alkyl; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁹)SO₂C₁-C₄-alkyl, may be independently substituted with up to 5 radical substituents comprising: —D, halogen radical, or ═O;
R⁹ and R¹⁰ independently represent —H, —D, an unbranched C₁-C₄-alkyl radical, a branched C₃-C₄-alkyl radical, a C₃-C₄-cycloalkyl radical, or the MR9)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof;
with the proviso that the aminoisoxazoline compound represented by Formula (I) is exclusive of:

N-(7-bromopyrrolo[2,1-f][1,2,4]triazin-4-yl)-4H-1′-azaspiro[isoxazole-5,3′- bicyclo[2.2.2]octan]-3-amine;

N-(furo[3,2-b]pyridin-5-yl)-4H-1′-azaspiro[isoxazole-5,3′-bicyclo[2.2.2]octan]-3-amine; and

N-(furo [2,3-c] pyridin-5 -yl)-4H-1′-azaspiro[isoxazole-5,3′-bicyclo[2.2.2]octan]-3-amine.

In certain embodiments, the aminoisoxazoline compound represented by Formula (I), (Ia), or (Ib), may comprise the W representing the moiety represented by the ring system M-II:

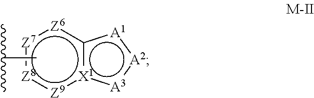

wherein:
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, or ═O;
X¹ independently represents N or C;
A¹, A², and A³ independently represent N, —NR⁷, N(CH₂)ₘR⁷, O, S, or CR⁸; with the proviso that only one A¹, A², and A³ is NR⁷, N(CH₂)ₘR⁷, O, or S; with the further proviso that when X¹ is N, then A¹, A², and A³ independently represent N or CR⁸;
R⁷ independently represents —H, —D, —SO₂(CH₂)ₘR⁹, —(CO)(CH₂)ₘR⁹, —(CO)N(R⁹)(R¹⁰), an unbranched C₁-C₄-alkyl radical, a branched C₃-C₄-alkyl radical, a C₃-C₆-cycloalkyl radical, or a (3-6 membered)-heterocycloalkyl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, or ═O;
R⁸ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁹)(R¹⁰), —(CO)C₁-C₄-alkyl,) —(CO)N(R⁹)(R¹⁰), —NR⁹(CO)(R¹⁰), —SO₂C₁-C₄-alkyl), —SO₂N(R⁹)(R¹⁰), —(CH₂)ₘSO₂C₁-C₄-alkyl, —(CH₂)ₘSO₂N(R⁹)(R¹⁰), or —N(R⁹)SO₂C₁-C₄-alkyl; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁹)SO₂C₁-C₄-alkyl, may be independently substituted with up to 5 radical substituents comprising: —D, halogen radical, or ═O; with the proviso that:
   a) when Z⁶ represents CR⁴ and the R⁴ is the bond directly attaching the W moiety with the aminoisoxazoline moiety, each of X¹, Z⁷, and Z⁹ represent N, Z⁸ represents CR⁴, both A¹ and A² independently represent CR⁸, A³ represents CR⁸ and the R⁸ of the A³ represents Br, then no more than two of the following is H: the R⁴ of the Z⁸, the R⁸ of the A¹, and the R⁸ of the A²;
   b) when Z⁷ represents CR⁴ and the R⁴ is the bond directly attaching the W moiety with the aminoisoxazoline moiety, X¹ represents C, both Z⁸ and Z⁹ independently represent CR⁴, Z⁶ represents N, A³ represents O, and both A¹ and A² independently represent CR⁸, then no more than three of the following is —H: the R⁴ of the Z⁸, the R⁴ of the Z⁹, the R⁸ of the A¹, and the R⁸ of the A²; or
   c) when Z⁷ represents CR⁴ and the R⁴ is the bond directly attaching the W moiety with the aminoisoxazoline moiety, X¹ represents C, both Z⁶ and Z⁹ independently represent CR⁴, Z⁸ represents N, A³ represents O, and both A¹ and A² independently represent CR⁸, then no more than three of the following is —H: the R⁴ of the Z⁶, the R⁴ of the Z⁹, the R⁸ of the A¹, and the R⁸ of the A²;
R⁹ and R¹⁰ independently represent —H, —D, an unbranched C₁-C₄-alkyl radical, a branched C₃-C₄-alkyl radical, a C₃-C₄-cycloalkyl radical, or the —N(R⁹)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
   or a pharmaceutically acceptable salt thereof

In certain embodiments, the aminoisoxazoline compound represented by Formula (I), (Ia), or (Ib), may comprise the W representing the moiety represented by the ring system

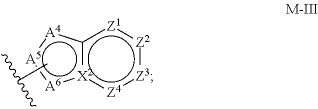

wherein:
Z¹, Z², Z³, and Z⁴ independently represent N or CR¹; with the proviso that no more than two of Z¹, Z², Z³, and Z⁴ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂N(R²)(R³), —(CF1₂)ₘSO₂C₁-C₄-alkyl, —(CH₂)ₘSO₂N(R²)(R³), or —N(R²)SO₂C₁-C₄-alkyl; wherein the alkyl portion of the unbranched Cₗ-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, or the —N(R²)SO₂C₁-C₄-alkyl, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²;
R¹¹ independently represents —H, —D, SO₂(CH₂)ₘR¹³, —(CO)(CH₂)ₘR¹³, —(CO)N(R¹³)(R-¹⁴), an unbranched C₁-C₄-alkyl radical, a branched C₃-C₄- alkyl radical, a C₃-C₆-cycloalkyl radical, or a (3-6 membered)-heterocycloalkyl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, or ═O,
R¹² independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, or a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, ═O, an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹³ and R¹⁴ independently represent —H, —D, an unbranched C₁-C₄-alkyl radical, a branched C₃-C₄-alkyl radical, a C₃-C₄-cycloalkyl radical, or the —N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof;
with the proviso that the aminoisoxazoline compound represented by Formula (I) is exclusive of:

N-(benzo[d]thiazol-2-yl)-4H-1′-azaspiro[isoxazole-5,3′-bicyclo[2.2]octan]-3-amine;

N-(6-methoxybenzo[d]thiazol-2-yl)-4H-1′-azaspiro[isoxazole-5,3′-bicyclo[2.2.2]octan]-3-amine;

N-(1H-indazol -3-yl)-4′H-4-azaspiro[bicyclo[2.2.2]octane-2,5 ‘-isoxazol]-3 ’-amine;

N-(4-chlorobenzo[d]thiazol-2 -yl)-4H-1′-azaspiro[isoxazole-5,3 ′-bicyclo[2.2.2]octan]-3-amine;

N-(5 -chlorobenzo[d]thiazol-2 -yl)-4H-1′-azaspiro[isoxazole-5,3 ′-bicyclo[2.2.2]octan]-3-amine;

N-(6-chlorobenzo[d]thiazol-2-yl)-4H-1′-azaspiro[isoxazole-5,3′-bicyclo[2.2.2]octan]-3-amine;

N-(7-chlorobenzo[d]thiazol-2-yl)-4H-1′-azaspiro[isoxazole-5,3′-bicyclo[2.2.2]octan]-3-amine;

N-(benzo[b]thiophen-2-yl)-4H-1′-azaspiro[isoxazole-5,3′-bicyclo[2.2.2]octan]-3-amine; and

N-(benzo[d]oxazol-2-yl)-4H-1′-azaspiro[isoxazole-5,3′-bicyclo[2.2.2]octan]-3-amine.

In certain embodiments, the aminoisoxazoline compound represented by Formula (I), (Ia), or (Ib), may comprise the W representing the moiety represented by the ring system M-III:

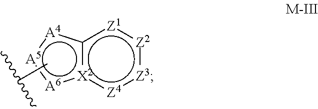

wherein:
Z¹, Z², Z³, and Z⁴ independently represent N or CR¹; with the proviso that no more than two of Z¹, Z², Z³ and Z⁴ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched OC₁-C₄-alkyl, a branched or cyclic —OC₃-C₄-alkyl, —N(R²)(R³), —(CO)N(R²)(R³), —NR²(CO)(R³), —SO₂N(R²)(R³), —(CH₂)ₘSO₂C₁-C₄-alkyl, —(CH₂)ₘSO₂N(R²)(R³), or —N(R²)SO₂C₁-C₄-alkyl; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, or the —N(R²)SO₂C₁-C₄-alkyl, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, —NR¹¹, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²;
R¹¹ independently represents —D, —SO₂(CH₂)ₘR¹³, —(CO)(CH₂)ₘR¹³, —(CO)N(R¹³)(R¹⁴), an unbranched C₁-C₄-alkyl radical, a branched C₃-C₄-alkyl radical, a C₃-C₆-cycloalkyl radical, or a (3-6 membered)-heterocycloalkyl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, or ═O, independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched OC₁-C₄-alkyl, or a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched OC₁-C₄-alkyl, or the branched or cyclic —OC₃-C₄-alkyl, may be independently substituted with up to 5 radical substituents comprising: D, halogen radical, ═O, an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; with the proviso that:
   a) when A⁵ represents CR¹² and the R¹² is the bond directly attaching the W moiety with the aminoisoxazoline moiety, X² represents C, A⁴ represents N, and A⁶ represents S, and each of Z¹, Z², Z³, and Z⁴ independently represent CR¹, then:
      i) no more than three of the R¹ is —H;
      ii) if one R¹ is —Cl, then no more than two of the R¹ is —H; or
      iii) if one of the R¹ of either Z² or Z³ is —OMe, then no more than two of the R¹ is —H;
   b) when A⁴ represents CR¹² and the R¹² is the bond directly attaching the W moiety with the aminoisoxazoline moiety, X² represents C, A⁵ represents N, and A⁶ represents NR¹¹, and each of Z¹, Z², Z³, and Z⁴ independently represent CR¹, then no more than four of the following is —H: the R¹¹ of the A⁶ and the R¹ of the Z¹ to Z⁴;
   c) when A⁵ represents CR¹² and the R¹² is the bond directly attaching the W moiety with the aminoisoxazoline moiety, X² represents C, A⁴ represents CR₁₂, A⁶ represents S, and each of Z¹, Z², Z³, and Z⁴ independently represent CR¹, then no more than four of the following is —H: the R¹² of the A⁴ and the R¹ of the Z¹ to Z⁴; or
   d) when A⁵ represents CR¹² and the R¹² is the bond directly attaching the W moiety with the aminoisoxazoline moiety, X² represents C, A⁴ represents N, A⁶ represents O, and each of Z¹, Z², Z³, and Z⁴ independently represent CR¹, then no more than three of the R¹ is —H;
R¹³ and R¹⁴ independently represent —H, —D, an unbranched C₁-C₄-alkyl radical, a branched C₃-C₄-alkyl radical, a C₃-C₄-cycloalkyl radical, or the —N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
   or a pharmaceutically acceptable salt thereof.

In certain embodiments, the aminoisoxazoline compound represented by Formula (I), (Ia), or (Ib), may comprise the W representing the moiety represented by the ring system M-IV:

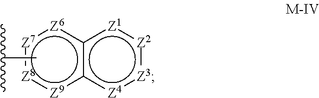

wherein:
Z¹, Z², Z³, and Z⁴ independently represent N or CR¹; with the proviso that no more than two of Z₁, Z₂, Z₃, and Z⁴ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), or N(R²)SO₂C₁-C₄-alkyl; or when adjacent members of Z¹, Z², Z³, and Z⁴, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched OC₁-C₄-alkyl, or a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: D, halogen radical, or ═O; and
m independently represents an integer from 1 to 6;
or a pharmaceutically acceptable salt thereof;
with the proviso that the aminoisoxazoline compound represented by Formula (I) is exclusive of:

N-(isoquinolin-3-yl)-4H-1′-azaspiro[isoxazole-5,3′-bicyclo[2.2.2]octan]-3-amine.

In certain embodiments, the aminoisoxazoline compound represented by Formula (I), (Ia), or (Ib), may comprise the W representing the moiety represented by the ring system M-IV:

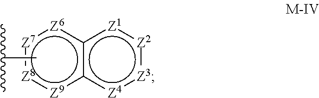

wherein:
Z¹, Z², Z³, and Z⁴ independently represent N or CR¹; with the proviso that no more than two of Z¹, Z², Z³, and Z⁴ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), or —N(R²)SO₂C₁-C₄-alkyl; or when adjacent members of Z¹, Z², Z³, and Z⁴, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the alkyl portion of the unbranched C₁-C₄-alkyl radical, the branched C₃-C₄-alkyl radical, the C₃-C₆-cycloalkyl radical, the unbranched the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, or a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, or ═O; with the proviso that when Z⁸ represents CR⁴ and the R⁴ is the bond directly attaching the W moiety with the aminoisoxazoline moiety, each of Z¹, Z², Z³, and Z⁴ independently represent CR¹, both Z⁶ and Z⁹ independently represent CR⁴, and Z⁷ represents N, then no more than five of the following is —H: the R¹ of the Z¹ to Z⁴, the R⁴ of the Z⁶, and the R⁴ of the Z⁹; and
m independently represents an integer from 1 to 6;
   or a pharmaceutically acceptable salt thereof

In certain embodiments, the aminoisoxazoline compound represented by Formula (I), (Ia), or (Ib), may comprise the W representing the moiety represented by the ring system M-I. In certain embodiments, the aminoisoxazoline compound represented by Formula (I), (Ia), or (Ib), may comprise the W representing the moiety represented by the ring system M-I, wherein, for example, each member of Z¹, Z², Z³, Z⁴, and Z⁵ represents (CR), with the proviso that no more than four of the R¹ is —H; for example, the Z¹ represents N, and Z², Z³, Z⁴, and Z⁵ each independently represent CR¹, with the proviso that if adjacent members Z³ and Z⁴ form a cycle such that the adjacent R¹ substitutents taken together represents a C₄-alkyl di-radical then a) no more than one of the R¹ of the Z² and Z⁵ is —H, or b) the C₄-alkyl di-radical is independently substituted with at least one radical substituent; for example, Z² represents N, and Z¹, Z³, Z⁴, and Z⁵ each independently represent CR¹; for example, Z³ represents N, and Z¹, Z², Z⁴, and Z⁵ each independently represent CR¹; for example, Z¹ and Z² each represent N, and Z³, Z⁴, and Z⁵ each independently represent CR¹; for example, Z¹ and Z³ each represent N, and Z², Z⁴, and Z⁵ each independently represent CR¹; for example, Z¹ and Z⁴ each represent N, and Z², Z³, and Z⁵ each independently represent CR¹; for example, Z¹ and Z⁵ each represent N, and Z², Z³, and Z⁴ each independently represent CR¹, with the proviso that if R¹ of Z³ represents —OCH₃ then then no more than one of the R¹ of the Z² and Z⁴ is —H; for example, Z² and Z³ each represent N, and Z¹, Z⁴, and Z⁵ each independently represent CR¹; or for example, Z² and Z⁴ each represent N, and Z¹, Z³, and Z⁵ each independently represent CR^{I}.

In certain embodiments, the aminoisoxazoline compound represented by Formula (I), (Ia), or (Ib), may comprise the W representing the moiety represented by the ring system M-I, wherein at least 3, 4, or each of Z¹, Z², Z³, Z⁴, and Z⁵ independently represent (CR¹), for example, at least 3, 4, or each of Z¹, Z², Z³, Z⁴, and Z⁵ independently represent (CR¹) and 2, 1, or 0 of Z¹, Z², Z³, Z⁴, and Z⁵ independently represent N, with the R¹ independently representing, such as at least one, two, or three of the R′, independently representing —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), an aryl radical, or a heteroaryl radical, or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical, or represents a (3-4 membered)-heteroalkyl di-radical with at least one ring atom of the (3-4 membered)- heteroalkyl di-radical selected from the group consisting of oxygen, nitrogen, and sulfur; wherein:
i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

In certain embodiments, the aminoisoxazoline compound represented by Formula (I), (Ia), or (Ib), may comprise the W representing the moiety represented by the ring system M-I, wherein at least 3, 4, or each of Z¹, Z², Z³, Z⁴, and Z⁵ independently represent (CR¹), for example, at least 3, 4, or each of Z¹, Z², Z³, Z⁴, and Z⁵ independently represent (CR¹) and 2, 1, or 0 of Z¹, Z², Z³, Z⁴, and Z⁵ independently represent N, with the R¹ independently representing, such as at least one, two, or three of the R′, independently representing —H, —D, —F, —Cl, —Br, —I, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, or an aryl radical, or a heteroaryl radical, or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical, such as —CH₂CH₂CH₂— or —CH₂CH₂CH₂CH₂—, or represents a (3-4 membered)-heteroalkyl di-radical with at least one ring atom of the (3-4 membered)-heteroalkyl di-radical selected from the group consisting of oxygen, nitrogen, and sulfur, such as —OCH₂CH₂CH₂—, —OCH₂CH₂N(H)—; —OCH₂CH₂N(C₁-C₄-alkyl)-, such as —OCH₂CH₂N(Me)—; —CH₂CH₂CH₂N(CO)(C₁-C₄-alkyl)-, —N(H)CH₂CH₂O—, —N(C₁-C₄-alkyl)CH₂CH₂O—, such as —N(Me)CH₂CH₂O—; —OCH₂CH₂O—; —OCH₂CH₂—; —OCH₂O—; 13 OCF₂O—; or —CH₂CH₂CH₂O—; wherein:
i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the C₃-C₄-alkyl di-radical, or the (3-4 membered)-heteroalkyl di-radical, may be independently substituted with up to 5 radical substituents comprising: —D, —F, —Cl, ═O, —OR², —(CH₂)ₘOR², —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, —F, —Cl, —Br, —I, —CN, —OR², —(CH₂)ₘOR², —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

In certain embodiments, the aminoisoxazoline compound represented by Formula (I), (Ia), or (Ib), may comprise the W representing the moiety represented by the ring system M-I, wherein at least 3, 4, or each of Z¹, Z², Z³, Z⁴, and Z⁵ independently represent (CR'), for example, at least 3, 4, or each of Z¹, Z², Z³, Z⁴, and Z⁵ independently represent (CR¹) and 2, 1, or 0 of Z¹, Z², Z³, Z⁴, and Z⁵ independently represent N, with the R¹ independently representing, such as at least one, two, or three of the R′, independently representing —H, —D, —F, —Cl, —Br, —I, —CN, an unbranched C₁-C₄-alkyl radical, a branched C₃-C₄-alkyl radical, a C₃-C₄-cycloalkyl radical, an unbranched —OC₁-C₄-alkyl, a branched or cyclic —OC₃-C₄-alkyl, or an aryl radical, or a heteroaryl radical, or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical, such as —CH₂CH₂CH₂— or —CH₂CH₂CH₂CH₂—, or represents a (3-4 membered)-heteroalkyl di-radical with at least one ring atom of the (3-4 membered)-heteroalkyl di-radical selected from the group consisting of oxygen, nitrogen, and sulfur, such as —OCH₂CH₂CH₂—, —OCH₂CH₂N(H)—; —OCH₂CH₂N(C₁-C₄-alkyl)-, such as —OCH₂CH₂N(Me)—; —CH₂CH₂CH₂N(CO)(C₁-C₄-alkyl)-, —N(H)CH₂CH₂O—, —N(C₁-C₄-alkyl)CH₂CH₂O—, such as —N(Me)CH₂CH₂O—; —OCH₂CH₂O—; —OCH₂CH₂—; —OCH₂O—; —OCF₂O—; or —CH₂CH₂CH₂O—; wherein:
i) the alkyl portion of the unbranched C₁-C₄-alkyl radical, the branched C₃-C₄-alkyl radical, the C₃-C₄-cycloalkyl radical, the unbranched —OC₁-C₄-alkyl, the branched or cyclic —OC₃-C₄-alkyl, the C₃-C₄-alkyl di-radical, or the (3-4 membered)-heteroalkyl di-radical, may be independently substituted with up to 5 radical substituents comprising: —D, —F, —Cl, ═O, —OR², —(CH₂)ₘOR², an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂- haloalkyl radical; and
ii) the aryl radical or the heteroaryl radical may be independently substituted with up to 5 radical substituents comprising: —D, —F, —Cl, —Br, —I, —CN, —OR², —(CH₂)ₘOR², —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

In certain embodiments, the aminoisoxazoline compound represented by Formula (I), (Ia), or (Ib), may comprise the W representing the moiety represented by the ring system M-I, wherein at least 3, 4, or each of Z¹, Z², Z³, Z⁴, and Z⁵ independently represent (CR¹), for example, at least 3, 4, or each of Z¹, Z², Z³, Z⁴, and Z⁵ independently represent (CR¹) and 2, 1, or 0 of Z¹, Z², Z³, Z⁴, and Z⁵ independently represent N, with the R¹ independently representing, such as at least one, two, or three of the R¹, independently representing —H, —D, —F, —Cl, —CN, —CH₃, —CH(CH₃)₂, cyclopropyl radical, cyclobutyl radical, —CH₂F, —CHF₂, —CF₃, —CH₂CF₃, —OCH₃, —OCH₂CH₃, —OCH(CH₃)₂, —O-cyclopropyl, —OCHF₂, —OCH₂F, —OCF₃, or —OCH₂CF₃. In certain embodiments, the aminoisoxazoline compound represented by Formula (I), (Ia), or (Ib), may comprise the W representing the moiety represented by the ring system M-I, wherein at least 3, 4, or each of Z¹, Z², Z³, Z⁴, and Z⁵ independently represent (CR¹), and when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical, such as —CH₂CH₂CH₂— or —CH₂CH₂CH₂CH₂—, or represents a (3-4 membered)-heteroalkyl di-radical with at least one ring atom of the (3-4 membered)-heteroalkyl di-radical selected from the group consisting of oxygen, nitrogen, and sulfur, such as —OCH₂CH₂CH₂—, —OCH₂CH₂N(H)—; —OCH₂CH₂N(C₁-C₄-alkyl)-, such as —OCH₂CH₂N(Me)—; —CH₂CH₂CH₂N(CO)(C₁-C₄-alkyl)-, —N(H)CH₂CH₂O—, —N(C₁-C₄-alkyl)CH₂CH₂O—, such as —N(Me)CH₂CH₂O—; —OCH₂CH₂O—; —OCH₂CH₂—; —OCH₂O—; —OCF₂O—; or —CH₂CH₂CH₂O—. For example, in certain embodiments, R¹ may independently represent —H, —D, —F, —Cl, —CH₃, —CH(CH₃)₂, cyclopropyl radical, cyclobutyl radical, —CH₂F, —CHF₂, —CF₃, —CH₂CF₃, —OCH₃, —OCH₂CH₃, —OCH(CH₃)₂, —O-cyclopropyl, —OCHF₂, —OCH₂F, —OCF₃, or —OCH₂CF₃, or when adjacent members of Z¹, Z², Z³, Z⁴, and Z⁵, is (CR¹)(CR¹), the (CR¹)(CR¹) may form a cycle such that the adjacent R¹ substitutents taken together represents a (3-4 membered)-heteroalkyl di-radical representing —OCF₂O—.

In certain embodiments, the aminoisoxazoline compound represented by Formula (I), (Ia), or (Ib), may comprise the W representing the moiety represented by the ring system M-II. In certain embodiments, the aminoisoxazoline compound represented by Formula (I), (Ia), or (Ib), may comprise the W representing the moiety represented by the ring system M-II, wherein X¹ represents N or C. For example, the aminoisoxazoline compound represented by Formula (I), (Ia), or (Ib), may comprise the W representing the moiety represented by the ring system M-II, wherein X¹ represents C and wherein M-II represents a moiety represented by one of the following:

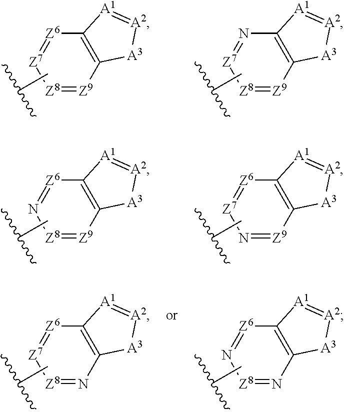

wherein Z⁶, Z⁷, Z⁸, and Z⁹ independently represent N or CR⁴, with the proviso that no more than two of Z⁶, Z⁷, Z⁸, and Z⁹ are N; and A¹ and A² independently represent N or CR⁸, and A³ independently represents NR⁷, N(CH₂)ₘR⁷, O, or S. In certain embodiments, the aminoisoxazoline compound represented by Formula (I), (Ia), or (Ib), may comprise the W representing the moiety represented by the ring system M-II, wherein Z⁶, Z⁷, Z⁸, and Z⁹ independently represent N or CR⁴, with the proviso that no more than two of Z⁶, Z⁷, Z⁸, and Z⁹ are N, for example, either Z⁶ or Z⁷ represents CR⁴ with said R⁴ representing the bond directly attaching the W moiety with the aminoisoxazoline moiety, or wherein either Z⁷ or Z⁸ represents CR⁴ with said R⁴ representing the bond directly attaching the W moiety with the aminoisoxazoline moiety, or wherein either Z⁸ or Z⁹ represents CR⁴ with said R⁴ representing the bond directly attaching the W moiety with the aminoisoxazoline moiety. For example, in certain embodiments, the aminoisoxazoline compound represented by Formula (I), (Ia), or (Ib), may comprise the W representing the moiety represented by the ring system M-II, wherein A¹ independently represents N or CR⁸, A² independently represents CR⁸, and A³ independently represents NR⁷, N(CH₂)ₘR⁷, O, or S, such as NR⁷, O, or S, or such as O or S; and wherein either Z⁶ or Z⁷ represents CR⁴ with said R⁴ representing the bond directly attaching the W moiety with the aminoisoxazoline moiety, or wherein either Z⁷ or Z⁸ represents CR⁴ with said R⁴ representing the bond directly attaching the W moiety with the aminoisoxazoline moiety, or wherein either Z⁸ or Z⁹ represents CR⁴ with said R⁴ representing the bond directly attaching the W moiety with the aminoisoxazoline moiety.

For example, in certain embodiments, the aminoisoxazoline compound represented by Formula (I), (Ia), or (Ib), may comprise the W representing the moiety represented by the ring system M-II with X¹ representing C, wherein M-II represents a moiety represented by:

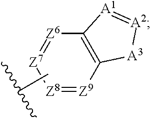

wherein A¹ and A² independently represent N or CR⁸; A³ independently represents NR⁷, N(CH₂)ₘR⁷, O, or S; and Z⁶, Z⁷, Z⁸, and Z⁹ independently represent N or CR⁴; with the proviso that no more than two of Z⁶, Z⁷, Z⁸, and Z⁹ are N, with one of said R⁴ of Z⁶, Z⁷, Z⁸, and Z⁹ representing the bond directly attaching the W moiety with the aminoisoxazoline moiety.

In certain embodiments, for example, the aminoisoxazoline compound represented by Formula (I), (Ia), or (Ib), may comprise the W representing the moiety represented by the ring system M-II-1 with X¹ representing C, said R⁴ of Z⁷ represents the bond directly attaching the W moiety with the aminoisoxazoline moiety:

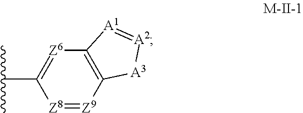

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶, Z⁸, and Z⁹ independently represent N or CR⁴; with the proviso that no more than two of Z⁶, Z⁸, and Z⁹ are N, for example each of Z⁶, Z⁸, and Z⁹, independently represent CR⁴.

In certain embodiments, for example, the aminoisoxazoline compound represented by Formula (I), (Ia), or (Ib), may comprise the W representing the moiety represented by the ring system M-II-2 with X¹ representing C, said R⁴ of Z⁸ represents the bond directly attaching the W moiety with the aminoisoxazoline moiety:

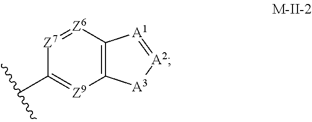

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶, Z⁷, and Z⁹ independently represent N or CR⁴; with the proviso that no more than two of Z⁶, Z⁷, and Z⁹ are N, for example each of Z⁶, Z⁷, and Z⁹, independently represent CR⁴.

For example, in certain embodiments, the aminoisoxazoline compound represented by Formula (I), (Ia), or (Ib), may comprise the W representing the moiety represented by the ring system M-II with X¹ representing C, wherein M-II represents a moiety represented by:

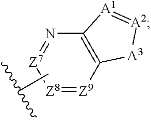

wherein A¹ and A² independently represent N or CR'; Ai independently represents NR⁷, N(CH₂)ₘR⁷, O, or S; and Z⁷, Z⁸, and Z⁹ independently represent N or CR⁴; with the proviso that no more than two of Z⁶, Z⁷, Z⁸, and Z⁹ are N, with one of said R⁴ of Z⁷, Z⁸, and Z⁹ representing the bond directly attaching the W moiety with the aminoisoxazoline moiety.

In certain embodiments, for example, the aminoisoxazoline compound represented by Formula (I), (Ia), or (Ib), may comprise the W representing the moiety represented by the ring system M-II-3 with X¹ representing C, said R⁴ of Z⁷ represents the bond directly attaching the W moiety with the aminoisoxazoline moiety:

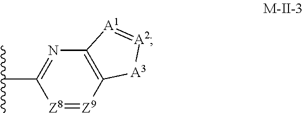

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁸ and Z⁹ independently represent N or CR⁴; with the proviso that no more than one of Z⁸ and Z⁹ are N, for example each of Z⁸ and Z⁹ independently represent CR⁴; with the proviso that when both Z⁸ and Z⁹ independently represent CR⁴, A³ represents O, and both A¹ and A² independently represent CR⁸, then no more than three of the following is —H: the R⁴ of the Z⁸, the R⁴ of the Z⁹, the R⁸ of the A′, and the R⁸ of the A².

In certain embodiments, for example, the aminoisoxazoline compound represented by Formula (I), (Ia), or (Ib), may comprise the W representing the moiety represented by the ring system M-II-4 with X¹ representing C, said R⁴ of Z⁸ represents the bond directly attaching the W moiety with the aminoisoxazoline moiety:

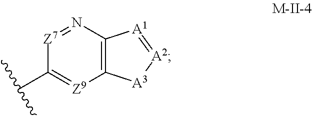

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁷ and Z⁹ independently represent N or CR⁴; with the proviso that no more than one of Z⁷ and Z⁹ are N, for example each of Z⁷ and Z⁹ independently represent CR⁴.

For example, in certain embodiments, the aminoisoxazoline compound represented by Formula (I), (Ia), or (Ib), may comprise the W representing the moiety represented by the ring system M-II with X¹ representing C, wherein M-II represents a moiety represented by:

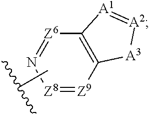

wherein A¹ and A² independently represent N or CR⁸; A³ independently represents NR⁷, N(CH₂)ₘR⁷, O, or S; and Z⁶, Z⁸, and Z⁹ independently represent N or CR⁴; with the proviso that no more than two of Z⁶, Z⁷, Z⁸, and Z⁹ are N, with one of said R⁴ of Z⁶, Z⁸, and Z⁹ representing the bond directly attaching the W moiety with the aminoisoxazoline moiety.

In certain embodiments, for example, the aminoisoxazoline compound represented by Formula (I), (Ia), or (Ib), may comprise the W representing the moiety represented by the ring system M-II-5 with X¹ representing C, said R⁴ of Z⁸ represents the bond directly attaching the W moiety with the aminoisoxazoline moiety:

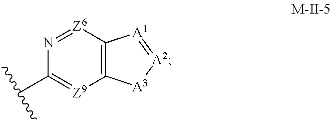

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶ and Z⁹ independently represent N or CR⁴; with the proviso that no more than one of Z⁶ and Z⁹ are N, for example each of Z⁶ and Z⁹ independently represent CR⁴.

For example, in certain embodiments, the aminoisoxazoline compound represented by Formula (I), (Ia), or (Ib), may comprise the W representing the moiety represented by the ring system M-II with X¹ representing C, wherein M-II represents a moiety represented by:

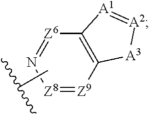

wherein A¹ and A² independently represent N or CR⁸; A³ independently represents NR⁷, N(CH₂)ₘR⁷, O, or S; and Z⁶, Z⁷, and Z⁹ independently represent N or CR⁴; with the proviso that no more than two of Z⁶, Z⁷, Z⁸, and Z⁹ are N, with one of said R⁴ of Z⁶, Z⁷, and Z⁹ representing the bond directly attaching the W moiety with the aminoisoxazoline moiety.

In certain embodiments, for example, the aminoisoxazoline compound represented by Formula (I), (Ia), or (Ib), may comprise the W representing the moiety represented by the ring system M-II-6 with X¹ representing C, said R⁴ of Z⁷ represents the bond directly attaching the W moiety with the aminoisoxazoline moiety:

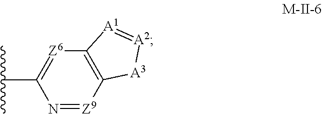

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶ and Z⁹ independently represent N or CR⁴; with the proviso that no more than one of Z⁶ and Z⁹ are N, for example each of Z⁶ and Z⁹ independently represent CR⁴; with the proviso that when both Z⁶ and Z⁹ independently represent CR⁴, A³ represents O, and both A¹ and A² independently represent CR⁸, then no more than three of the following is —H: the R^{′′} of the Z⁶, the R⁴ of the Z⁹, the R⁸ of the A′, and the R⁸ of the A².

For example, in certain embodiments, the aminoisoxazoline compound represented by Formula (I), (Ia), or (Ib), may comprise the W representing the moiety represented by the ring system M-II with X¹ representing C, wherein M-II represents a moiety represented by:

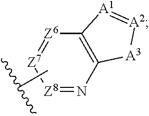

wherein A¹ and A² independently represent N or CR⁸; A³ independently represents NR⁷, N(CH₂)ₘR⁷, O, or S; and Z⁶, Z⁷, and Z⁸ independently represent N or CR⁴; with the proviso that no more than two of Z⁶, Z⁷, Z⁸, and Z⁹ are N, with one of said R⁴ of Z⁶, Z⁷, and Z⁸ representing the bond directly attaching the W moiety with the aminoisoxazoline moiety.

In certain embodiments, for example, the aminoisoxazoline compound represented by Formula (I), (Ia), or (Ib), may comprise the W representing the moiety represented by the ring system M-II-7 with X¹ representing C, said R⁴ of Z⁷ represents the bond directly attaching the W moiety with the aminoisoxazoline moiety:

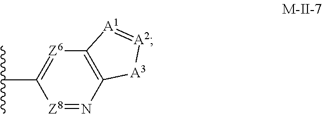

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶ and Z⁸ independently represent N or CR⁴; with the proviso that no more than one of Z⁶ and Z⁸ are N, for example each of Z⁶ and Z⁸ independently represent CR⁴.

In certain embodiments, for example, the aminoisoxazoline compound represented by Formula (I), (Ia), or (Ib), may comprise the W representing the moiety represented by the ring system M-II-8 with X¹ representing C, said R⁴ of Z⁸ represents the bond directly attaching the W moiety with the aminoisoxazoline moiety:

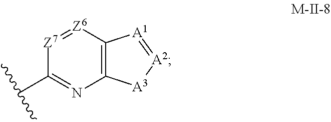

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶ and Z⁷ independently represent N or CR⁴; with the proviso that no more than one of Z⁶ and Z⁷ are N, for example each of Z⁶ and Z⁷ independently represent CR⁴.

For example, in certain embodiments, the aminoisoxazoline compound represented by Formula (I), (Ia), or (Ib), may comprise the W representing the moiety represented by the ring system M-II with X¹ representing C, wherein M-II represents a moiety represented by:

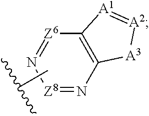

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶ and Z⁸ independently represent CR⁴, with one of said R⁴ of Z⁶ and Z⁸ representing the bond directly attaching the W moiety with the aminoisoxazoline moiety.

In certain embodiments, for example, the aminoisoxazoline compound represented by Formula (I), (Ia), or (Ib), may comprise the W representing the moiety represented by the ring system M-II-9 with X¹ representing C, said R⁴ of Z⁸ represents the bond directly attaching the W moiety with the aminoisoxazoline moiety:

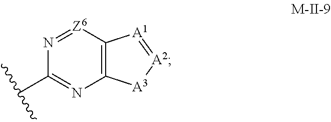

wherein A¹ and A² independently represent N or CR⁸; A³ independently represents NR⁷, N(CH₂)ₘR⁷, O, or S, such as A¹ independently represents N or CR⁸, A² independently represents CR⁸, and A³ independently represents NR⁷, N(CH₂)ₘR⁷, O, or S; and Z⁶ independently represents CR⁴.

In certain embodiments, the aminoisoxazoline compound represented by Formula (I), (Ia), or (Ib), may comprise the W representing the moiety represented by the ring system M-II, wherein X′ represents N. For example, the aminoisoxazoline compound represented by Formula (I), (Ia), or (Ib), may comprise the W representing the moiety represented by the ring system M-II, wherein M-II represents a moiety represented by:

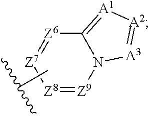

wherein A¹, A², and A³ independently represent N or CR⁸; and Z⁶, Z⁷, Z⁸ , and Z⁹ independently represent N or CR⁴; with the proviso that no more than two of Z⁶, Z⁷, Z⁸, and Z⁹ are N, with one of said R⁴ of Z⁶, Z⁷, Z⁸, and Z⁹ representing the bond directly attaching the W moiety with the aminoisoxazoline moiety; and with the proviso that when Z⁶ represents CR⁴ and the R⁴ is the bond directly attaching the W moiety with the aminoisoxazoline moiety, each of Z⁷ and Z⁹ represent N, Z⁸ represents CR⁴, both A¹ and A² independently represent CR⁸, A³ represents CR⁸ and the R⁸ of the A³ represents —Br, then no more than two of the following is —H: the R⁴ of the Z⁸, the R⁸ of the A¹, and the R⁸ of the A².

In certain embodiments, for example, the aminoisoxazoline compound represented by Formula (I), (Ia), or (Ib), may comprise the W representing the moiety represented by the ring system M-II-10 with X¹ representing N, said R⁴ of Z⁷ represents the bond directly attaching the W moiety with the aminoisoxazoline moiety:

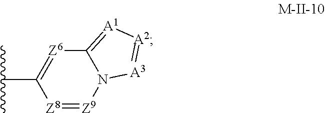

wherein A¹, A², and A³ independently represent N or CR⁸; and Z⁶, Z⁸ , and Z⁹ independently represent N or CR⁴; with the proviso that no more than two of Z⁶, Z⁸, and Z⁹ are N, for example each of Z⁶, Z⁸, and Z⁹, independently represent CR⁴.

In certain embodiments, for example, the aminoisoxazoline compound represented by Formula (I), (Ia), or (Ib), may comprise the W representing the moiety represented by the ring system M-II-11 with X¹ representing N, said R⁴ of Z⁸ represents the bond directly attaching the W moiety with the aminoisoxazoline moiety:

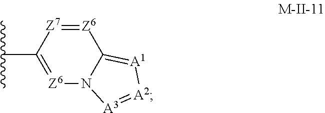

wherein A¹, A², and A³ independently represent N or CR⁸; and Z⁶, Z⁸ , and Z⁹ independently represent N or CR⁴; with the proviso that no more than two of Z⁶, Z⁷, and Z⁹ are N, for example each of Z⁶, Z⁷, and Z⁹, independently represent CR⁴.

In certain embodiments, the W represents the moiety represented by the ring system M-II, such as the ring system M-II-1, M-II-2, M-II-3, M-II-4, M-II-5, M-II-6, M-II-7, M-II-8, M-II-9, M-II-10, or M-II-11, wherein the Z⁶, Z⁷, Z⁸, and Z⁹ may independently represent CR⁴, wherein the R⁴ independently represents —H, —D, halogen radical, such as—F, —Cl, or —Br; —CN, an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₆-cycloalkyl radical, such as a cyclopropyl radical or a cyclobutyl radical; an unbranched —OC₁-C₄-alkyl, such as —OCH₃, —OCH₂CH₃, or —OCH₂CH₂CH₃; a branched or cyclic —OC₃-C₄-alkyl, such as —OCH(CH₃)₂ or —O-cyclopropyl; wherein the alkyl portion of the unbranched C₁-C₄-alkyl radical, the branched C₃-C₄-alkyl radical, the C₃-C₆-cycloalkyl radical, the unbranched —OC₁-C₄-alkyl, or the branched or cyclic —OC₃-C₄-alkyl, may be independently substituted with up to 5 radical substituents comprising: —D, halogen radical, such as —F, —Cl, or —Br, or ═O. For example, in certain embodiments, the W represents the moiety represented by the ring system M-II, such as the ring system M-II-1, M-II-2, M-II-3, M-II-4, M-II-5, M-II-6, M-II-7, M-II-8, M-II-9, M-II-10, or M-II-11, wherein the Z⁶, Z⁷, Z⁸, and Z⁹ may independently represent CR⁴, wherein the R⁴ independently represents —H, —D, —F, —Cl, —CN, —CH₃, —CH₂CH₃, —CH₂CH₂CH₃, —CH(CH₃)₂, cyclopropyl radical, cyclobutyl radical, —CH₂F, —CHF₂, —CF₃, —CH₂CF₃, —OCH₃, —OCH₂CH₃, —OCH₂CH₂CH₃, —OCH(CH₃)₂, —O-cyclopropyl, —OCHF₂, —OCH₂F, —OCF₃, or —OCH₂CF₃. For example, in certain embodiments, the W represents the moiety represented by the ring system M-II, such as the ring system M-II-1, M-II-2, M-II-3, M-II-4, M-II-5, M-II-6, M-II-7, M-II-8, M-II-9, M-II-10, or M-II-11, wherein the Z⁶, Z⁷, Z⁸, and Z⁹ may independently represent CR⁴, wherein the R⁴ independently represents —H, —D, —F, —Cl, —CN, —CH₃, —CH₂F, —CHF₂, —CF₃, —OCH₃, —OCHF₂, —OCH₂F, or —OCF₃, such as —H, —D, —F, —Cl, —CN, —CH₃, or —CF₃; such as —H, —D, —F, —Cl.

In certain embodiments, for example, the W represents the moiety represented by the ring system M-II, such as the ring system M-II-1, M-II-2, M-II-3, M-II-4, M-II-5, M-II-6, M-II-7, M-II-8, or M-II-9, wherein A¹ and A² independently represent N or CR⁸, and A³ represents NR⁷, N(CH₂)ₘR⁷, O, or S; for example, wherein A¹ and A² independently represent CR⁸, and A³ independently represents NR⁷, N(CH₂)ₘR⁷, O, or S, such as NR⁷, O, or S, for example, O or S; wherein A¹ independently represents N, A² independently represents CR⁸, and A³ independently represents NR⁷, N(CH₂)ₘR⁷, O, or S, such as NR⁷, O, or S, for example, O or S; or wherein A¹ independently represents CR⁸, A² independently represents N, and A³ independently represents NR⁷, N(CH₂)ₘR⁷, O, or S, such as NR⁷, O, or S, for example, O or S.

In certain embodiments, for example, the W represents the moiety represented by the ring system M-II, such as the ring system M-II-10 or M-II-11, wherein A¹, A², and A³ independently represent N or CR⁸, for example, wherein A¹, A², and A³ independently CR⁸; wherein A¹, A², and A³ independently represent N; wherein A¹ represents N, and A² and A³ independently represent CR⁸; wherein A² represents N, and A¹ and A³ independently represent CR⁸; wherein A³ represents N, and A¹ and A² independently represent CR⁸; or wherein A¹ and A² independently represent N, and A³ independently represent CR⁸.

In certain embodiments, the R⁷ of the NR⁷ or the —N(CH₂)ₘR⁷ of the A¹, A², and A³ of the ring system M-II, for example, the A³ of the ring system M-II-1, M-II-2, M-II-3, M-II-4, M-II-5, M-II-6, M-II-7, M-II-8, or M-II-9, may independently represent —H, —D, —SO₂(CH₂)ₘR⁹, such as —SO₂CH₃; —(CO)(CH₂)ₙR⁹, such as —(CO)CH₃; an unbranched C₁-C₄-alkyl radical, a branched C₃-C₄-alkyl radical, a C₃-C₆-cycloalkyl radical; wherein the unbranched C₁-C₄-alkyl radical, the branched C₃-C₄-alkyl radical, or the C₃-C₆-cycloalkyl radical, may be independently substituted with up to 5 radical substituents comprising: —D, halogen radical, or ═O. For example, the R⁷ of the NR⁷ or the N(CH₂)ₘR⁷ may independently represent —H, —D, —SO₂CH₃, —(CO)CH₃, an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₄-cycloalkyl radical; wherein the unbranched C₁-C₄-alkyl radical, the branched C₃-C₄-alkyl radical, or the C₃-C₄-cycloalkyl radical, may be independently substituted with up to 5 radical substituents comprising: —D, halogen radical, or ═O. In certain embodiments, the R⁷ of the NR⁷ or the —N(CH₂)ₘR⁷ may independently represent —H, —D, —SO₂CH₃, —(CO)CH₃, —CH₃, —CH₂CH₃, —CH₂CH₂CH₃, —CH(CH₃)₂, or a cyclopropyl radical. In certain embodiments, the R⁷ of the NR⁷ or the N(CH₂)ₘR⁷ may independently represent —H, —D, —SO₂CH₃, —(CO)CH₃, or —CH₃.

In certain embodiments, for example, one, two, or each of A¹, A², and A³, of the ring system M-II, may independently represent N or CR⁸, such as CR⁸; for example, one or both of the A¹ and A² of the ring system M-II-1 to M-II-9, or one, two, or each of the A¹, A², and A³, of the ring system M-II-10 or M-II-11, may independently represent CR⁸; wherein the R⁸ may independently represent —H, —D, halogen radical, —CN, an unbranched C₁-C₄-alkyl radical, a branched C₃-C₄-alkyl radical, a C₃-C₄-cycloalkyl radical, an unbranched —OC₁-C₄-alkyl, a branched or cyclic —OC₃-C₄-alkyl; wherein the alkyl portion of the unbranched C₁-C₄-alkyl radical, the branched C₃-C₄-alkyl radical, the C₃-C₄-cycloalkyl radical, the unbranched —OC₁-C₄-alkyl, or the branched or cyclic —OC₃-C₄-alkyl, may be independently substituted with up to 5 radical substituents comprising: —D, halogen radical, or ═O. For example, the R⁸ may independently represent —H, —D, halogen radical, such as —F, —Cl, or —Br; —CN, an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₄-cycloalkyl radical; an unbranched —OC₁-C₄-alkyl, such as —OCH₃, —OCH₂CH₃, or —OCH₂CH₂CH₃; a branched or cyclic —OC₃-C₄-alkyl, such as —OCH(CH₃)₂; wherein the alkyl portion of the unbranched C₁-C₄-alkyl radical, the branched C₃-C₄-alkyl radical, the C₃-C₄-cycloalkyl radical, the unbranched —OC₁-C₄-alkyl, or the branched or cyclic —OC₃-C₄-alkyl, may be independently substituted with up to 5 radical substituents comprising: —D, halogen radical, such as —F, —Cl, or ═O. In certain embodiments, the R⁸ may independently represent —H, —D, —F, —Cl, —Br, —CN, —CH₃, —CH₂CH₃, —CH₂CH₂CH₃, —CH(CH₃)₂, a cyclopropyl radical, —CH₂F, —CHF₂, —CF₃, —CH₂CF₃, —OCH₃, —OCH₂CH₃, —OCH₂CH₂CH₃, or —OCH(CH₃)₂, —O-cyclopropyl, —OCHF₂, —OCH₂F, —OCF₃, or —OCH₂CF₃. In certain embodiments, the R⁸ may independently represent —H, —D, —F, —Cl, —CH₃, —CH₂F, —CHF₂, or —CF₃.

In certain embodiments, the aminoisoxazoline compound represented by Formula (I), (Ia), or (Ib), may comprise the W representing the moiety represented by the ring system M-III, wherein X² represents C or N, preferably X² represents C; wherein A⁴, A⁵, and A⁶ independently represent N, NR¹¹, N(CH₂)ₘR¹¹, O, S, or CR¹²; and wherein Z¹, Z², Z³, and Z⁴ independently represent N or CR¹; with the proviso that no more than two of Z¹, Z², Z³, and Z⁴ are N; with the proviso that only one A⁴, A⁵, and A⁶ is NW', N(CH₂)ₘR¹¹, O, or S; with the further proviso that when X² is N, then A⁴, A⁵, and A⁶ independently represent N or CR¹², and wherein one of A⁴, A⁵, and A⁶ represents CR¹² wherein the R¹² represents the bond directly attaching the W moiety with the aminoisoxazoline moiety, for example A⁴ represents CR¹² wherein the R¹² represents the bond directly attaching the W moiety with the aminoisoxazoline moiety, and A⁵ and A⁶ independently represent N, N(CH₂)ₘR¹¹, O, S, or CR¹²; or A⁶ represents CR¹² wherein the R¹² represents the bond directly attaching the W moiety with the aminoisoxazoline moiety, and A⁴ and A⁵ independently represent N, N(CH₂)ₘR¹¹, O, S, or CR¹².

In certain embodiments, for example, the aminoisoxazoline compound represented by Formula (I), (Ia), or (Ib), may comprise the W representing the moiety represented by the ring system M-III, wherein X² represents C. For example, the aminoisoxazoline compound represented by Formula (I), (Ia), or (Ib), comprising W representing the moiety represented by the ring system M-III, may comprise a moiety represented by:

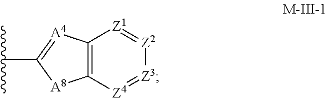

wherein A⁴ represents N or CR¹², such as A⁴ represents N, or A⁴ represents CR¹²; and A⁶ represents NR¹¹, N(CH₂)ₘR¹¹, O, or S, such as A⁶ represents NR¹¹, O, or S, such as A⁶ represents O or S; and wherein Z¹, Z², Z³, and Z⁴ independently represent N or CR¹; with the proviso that no more than two of Z¹, Z², Z³, and Z⁴ are N, such as at least 3 or each of the Z¹, Z², Z³, and Z⁴ independently represent CR¹; with the proviso that: a) when A⁴ represents N, and A⁶ represents S, and each of Z¹, Z², Z³, and Z⁴ independently represent CR¹, then: i) no more than three of the R¹ is —H; ii) if one R¹ is —Cl, then no more than two of the R¹ is —H; or iii) if one of the R¹ of either Z² or Z³ is —OMe, then no more than two of the R¹ is —H; b) when A⁴ represents CR¹², A⁶ represents S, and each of Z¹, Z², Z³, and Z⁴ independently represent CR¹, then no more than four of the following is —H: the R¹² of the A⁴ and the R¹ of the Z¹ to Z⁴; or c) when A⁴ represents N, A⁶ represents O, and each of Z¹, Z², Z³, and Z⁴ independently represent CR¹, then no more than three of the R¹ is —H.

In certain embodiments, the aminoisoxazoline compound represented by Formula (I), (Ia), or (Ib), may comprise the W representing the moiety represented by the ring system M-III, such as the ring system M-III-1, wherein at least 3, or each of Z¹, Z², Z³, and Z⁴, independently represent (CR¹), for example, at least 2, 3, or each of Z¹, Z², Z³, and Z⁴, independently represent (CR¹) and 2, 1, or 0 of Z¹, Z², Z³, and Z⁴, independently represent N, with the R¹ independently representing —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, or -SO₂C₁-C₄-alkyl; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, or the —SO₂C₁-C₄-alkyl, may be independently substituted with up to 5 radical substituents comprising: —D, halogen radical, ═O, -—OR², —(CH₂)ₘOR², —SO₂N(R²)(R³), —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

In certain embodiments, the aminoisoxazoline compound represented by Formula (I), (Ia), or (Ib), may comprise the W representing the moiety represented by the ring system M-III, such as the ring system M-III-1, wherein at least 3, or each of Z¹, Z², Z³, and Z⁴, independently represent (CR¹), for example, at least 2, 3, or each of Z¹, Z², Z³, and Z⁴, independently represent (CR¹) and 2, 1, or 0 of Z¹, Z², Z³, and Z⁴, independently represent N, with the R¹ independently representing —H, —D, —F, —Cl, —Br, —I, —CN, an unbranched C₁-C₄-alkyl radical, such as —CH₃, —CH₂CH₃, or —CH₂CH₂CH₃; a branched C₃-C₄-alkyl radical, such as —CH(CH₃)₂; a C₃-C₆-cycloalkyl radical, such as a cyclopropyl radical or a cyclobutyl radical; an unbranched —OC₁-C₄- alkyl, such as —OCH₃, —OCH₂CH₃, or —OCH₂CH₂CH₃; a branched or cyclic —OC₃-C₄-alkyl, such as —OCH(CH₃)₂; or -SO₂C₁-C₄-alkyl, such as —SO₂CH₃; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, or the —SO₂C₁-C₄-alkyl, may be independently substituted with up to 5 radical substituents comprising: —D, halogen radical, ═O, —OR², —(CH₂)ₘOR², an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

In certain embodiments, the aminoisoxazoline compound represented by Formula (I), (Ia), or (Ib), may comprise the W representing the moiety represented by the ring system M-III, such as the ring system M-III-1, wherein at least 2, 3, or each of Z¹, Z², Z³, and Z⁴, independently represent (CR¹), for example, at least 3, or each of Z¹, Z², Z³, and Z⁴, independently represent (CR¹) and 2, 1, or 0 of Z¹, Z², Z³, and Z⁴, independently represent N, with the R¹ independently representing —H, —D, —F, —Cl, —Br, —I, —CN, —CH₃, —CH₂CH₃, —CH₂CH₂CH₃, —CH(CH₃)₂, cyclopropyl radical, cyclobutyl radical, —CH₂F, —CHF₂, —CF₃, —CH₂CF₃, —OCH₃, —OCH₂CH₃, —OCH₂CH₂CH₃, —OCH(CH₃)₂, —O-cyclopropyl, —OCHF₂, —OCH₂F, —OCF₃, —OCH₂CF₃, or —SO₂CH₃. For example, in certain embodiments, Z¹, Z², Z³, and Z⁴, of the ring system M-III, may independently represent (CR'), wherein the R¹ independently representing —H, —D, —F, —Cl, —CN, —CH₃, cyclopropyl radical, —CH₂F, —CHF₂, —CF₃, —OCH₃, or —OCF₃; such as the R¹ independently representing —H, —D, —F, —Cl, —CN, —CH₃, cyclopropyl radical, —CF₃, or —OCH₃.

In certain embodiments, for example, the A⁴, A⁵, and A⁶ of the ring system M-III, such as the ring system M-III-1, may independently represent NR¹¹ or N(CH₂)ₘR¹¹, wherein the R¹¹ of the NR¹¹ or the —N(CH₂)ₘR¹¹ may independently represent —H, —D, —SO₂(CH₂)ₘR¹³, such as —SO₂CH₃; —(CO)(CH₂)ₘR¹³, such as —(CO)CH₃; —(CO)N(R¹³)(R¹⁴), such as —(CO)N(CH₃)₂;an unbranched C₁-C₄-alkyl radical, a branched C₃-C₄-alkyl radical, a C₃-C₆-cycloalkyl radical; wherein the unbranched C₁-C₄-alkyl radical, the branched C₃-C₄-alkyl radical, or the C₃-C₆-cycloalkyl radical, may be independently substituted with up to 5 radical substituents comprising: —D, halogen radical, or ═O. For example, in certain embodiments, the R¹¹ of the NR¹¹ or the —N(CH₂)ₘR¹¹ may independently represent —H, —D, —SO₂(CH₂)ₘR¹³, such as —SO₂CH₃; —(CO)(CH₂)ₘR¹³, such as —(CO)CH₃; —(CO)N(R¹³)(R¹⁴), such as —(CO)N(CH₃)₂;an unbranched C₁-C₄-alkyl radical, a branched C₃-C₄-alkyl radical, a C₃-C₆-cycloalkyl radical; wherein the unbranched C₁-C₄-alkyl radical, the branched C₃-C₄-alkyl radical, or the C₃-C₆-cycloalkyl radical, may be independently substituted with up to 5 radical substituents comprising: —D, halogen radical, or ═O. For example, the R¹¹ of the NR¹¹ or the N(CH₂)ₘR¹¹ may independently represent —H, —D, —SO₂CH₃, —(CO)CH₃, an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₄-cycloalkyl radical; wherein the unbranched C₁-C₄-alkyl radical, the branched C₃-C₄-alkyl radical, or the C₃-C₄-cycloalkyl radical, may be independently substituted with up to 5 radical substituents comprising: —D, halogen radical, or ═O. In certain embodiments, the R¹¹ of the NR^{I}″ or the —N(CH₂)ₘR¹¹ may independently represent —H, —D, —SO₂CH₃, —(CO)CH₃, —CH₃, —CH₂CH₃, —CH₂CH₂CH₃, —CH(CH₃)₂, or a cyclopropyl radical; for example, in certain embodiments, the R¹¹ of the NR¹¹ or the N(CH₂)ₘR¹¹ may independently represent —H, —D, —SO₂CH₃, —(CO)CH₃, or —CH₃; such as the R¹¹ of the NR¹ or the N(CH₂)ₘR¹¹ may independently represent —H, —D, or —CH₃.

In certain embodiments, A⁴, A⁵, and A⁶ may independently represent CR¹², such as, one or two of the A⁴, A⁵, and A⁶ may independently represent CR¹², wherein the R¹² of the CR¹² may independently represent —H, —D, halogen radical, —CN, an unbranched C₁-C₄-alkyl radical, such as —CH₃, —CH₂CH₃, or —CH₂CH₂CH₃, a branched C₃-C₄-alkyl radical, such as —CH(CH₃)₂; C₃-C₆-cycloalkyl radical, such as a cyclopropyl radical or a cyclobutylbutyl radical; a (3-6 membered)-heterocycloalkyl radical, an unbranched —OC₁-C₄-alkyl, such as —OCH₃, —OCH₂CH₃, —OCH₂CH₂CH₃; or a branched or cyclic —OC₃-C₄-alkyl, such as —OCH(CH₃)₂, —O-cyclopropyl; or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, ═O, an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical. For example, in certain embodiments, the R¹² of the CR¹² may represent —H, —D, halogen radical, —CN, an unbranched C₁-C₄-alkyl radical, such as —CH₃, —CH₂CH₃, or —CH₂CH₂CH₃, a branched C₃-C₄-alkyl radical, such as —CH(CH₃)₂; C₃-C₆-cycloalkyl radical, such as a cyclopropyl radical or a cyclobutylbutyl radical; a (3-6 membered)-heterocycloalkyl radical, an unbranched —OC₁-C₄-alkyl, such as —OCH₃, —OCH₂CH₃, —OCH₂CH₂CH₃; or a branched or cyclic —OC₃-C₄-alkyl, such as —OCH(CH₃)₂, —O-cyclopropyl; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, ═O, an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical. For example, in certain embodiments, the R¹² of the CR¹² may represent —H, —D, —F, —Cl, —Br, —CN, —CH₃, —CH₂CH₃, —CH₂CH₂CH₃, —CH(CH₃)₂, cyclopropyl radical, cyclobutyl radical, —CH₂F, —CHF₂, —CF₃, —CH₂CF₃, —OCH₃, —OCH₂CH₃, —OCH₂CH₂CH₃, —OCH(CH₃)₂, —O-cyclopropyl, —OCHF₂, —OCH₂F, —OCF₃, or —OCH₂CF₃; for example, the R¹² of the CR¹² may represent —H, —D, —F, —Cl, —CN, —CH₃, —CH₂F, —CHF₂, —CF₃, —OCH₃, —OCHF₂, —OCH₂F, or —OCF₃; such as the R¹² of the CR¹² may represent —H, —D, —F, —Cl, —CN, —CH₃, —CH₂F, —CHF₂, or —CF₃; such as the ^{R′2} of the CR^{L2} may represent —H, —D, —F, or —CH₃.

In certain embodiments, the aminoisoxazoline compound represented by Formula (I), (Ia), or (Ib), may comprise the W representing the moiety represented by the ring system M-IV. For example, in certain embodiments, the aminoisoxazoline compound represented by Formula (I), (Ia), or (Ib), may comprise the W representing the moiety represented by the ring system M-IV, wherein M-IV represents a moiety represented by one of the following:

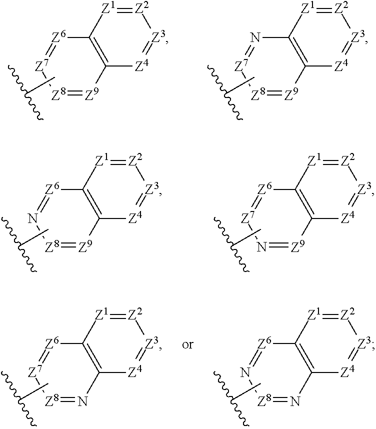

wherein Z¹, Z², Z³, and Z⁴ independently represent N or CR¹, with the proviso that no more than two of Z¹, Z², Z³, and Z⁴ are N; and wherein Z⁶, Z⁷, Z⁸, and Z⁹ independently represent N or CR⁴, with the proviso that no more than two of Z⁶, Z⁷, Z⁸, and Z⁹ are N. In certain embodiments, the aminoisoxazoline compound represented by Formula (I), (Ia), or (Ib), may comprise the W representing the moiety represented by the ring system M-IV, wherein Z⁶, Z⁷, Z⁸, and Z⁹ independently represent N or CR⁴, with the proviso that no more than two of Z⁶, Z⁷, Z⁸, and Z⁹ are N, for example, either Z⁶ or Z⁷ represents CR⁴ with said R⁴ representing the bond directly attaching the W moiety with the aminoisoxazoline moiety, or wherein either Z⁷ or Z⁸ represents CR⁴ with said R⁴ representing the bond directly attaching the W moiety with the aminoisoxazoline moiety, or wherein either Z⁸ or Z⁹ represents CR⁴ with said R⁴ representing the bond directly attaching the W moiety with the aminoisoxazoline moiety.

For example, in certain embodiments, the aminoisoxazoline compound represented by Formula (I), (Ia), or (Ib), may comprise the W representing the moiety represented by the ring system M-IV, wherein M-IV represents a moiety represented by one of the following:

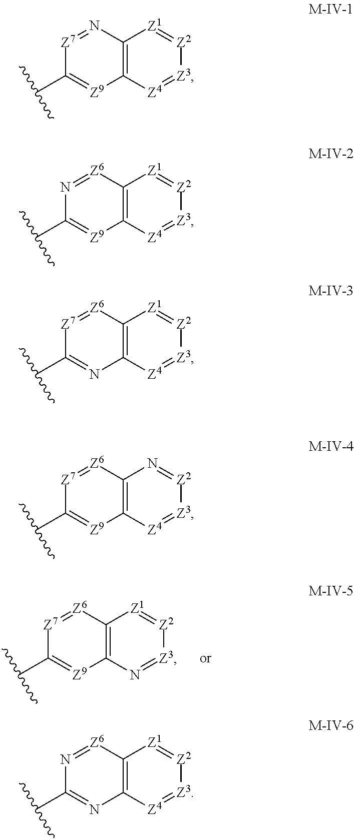

In certain embodiments, for example, the aminoisoxazoline compound represented by Formula (I), (Ia), or (Ib), may comprise the W representing the moiety represented by the ring system M-IV-1 with the R⁴ of Z⁸ represents the bond directly attaching the W moiety with the aminoisoxazoline moiety:

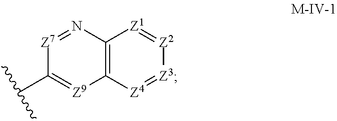

wherein Z¹, Z², Z³, and Z⁴ may independently represent N or CR¹; with the proviso that no more than two of Z¹, Z², Z³, and Z⁴ are N, and wherein Z⁷ and Z⁹ may independently represent N or CR⁴; with the proviso that no more than one of Z⁷ and Z⁹ are N.

In certain embodiments, for example, the aminoisoxazoline compound represented by Formula (I), (Ia), or (Ib), may comprise the W representing the moiety represented by the ring system M-IV-2 with the R⁴ of Z⁸ represents the bond directly attaching the W moiety with the aminoisoxazoline moiety:

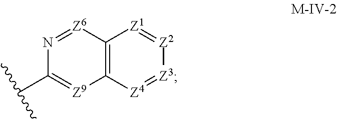

wherein Z¹, Z², Z³, and Z⁴ may independently represent N or CR¹; with the proviso that no more than two of Z¹, Z², Z³, and Z⁴ are N, and wherein Z⁶ and Z⁹ may independently represent N or CR⁴; with the proviso that no more than one of Z⁶ and Z⁹ are N; with the further proviso that when each of Z¹, Z², Z³, and Z⁴ independently represent CR¹, and both Z⁶ and Z⁹ independently represent CR⁴, then no more than five of the following is H: the R¹ of the Z¹ to Z⁴, the R⁴ of the Z⁶, and the R⁴ of the Z⁹.

In certain embodiments, for example, the aminoisoxazoline compound represented by Formula (I), (Ia), or (Ib), may comprise the W representing the moiety represented by the ring system M-IV-3 with the R⁴ of Z⁸ represents the bond directly attaching the W moiety with the aminoisoxazoline moiety:

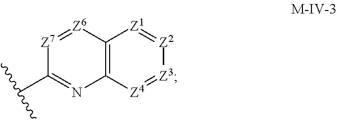

wherein Z¹, Z², Z³, and Z⁴ may independently represent N or CR¹; with the proviso that no more than two of Z¹, Z², Z³, and Z⁴ are N, and wherein Z⁶ and Z⁷ may independently represent N or CR⁴; with the proviso that no more than one of Z⁶ and Z⁷ are N.

In certain embodiments, for example, the aminoisoxazoline compound represented by Formula (I), (Ia), or (Ib), may comprise the W representing the moiety represented by the ring system M-IV-4 with the R⁴ of Z⁸ represents the bond directly attaching the W moiety with the aminoisoxazoline moiety:

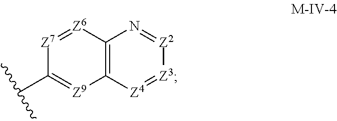

wherein Z², Z³, and Z⁴ may independently represent N or CR¹; with the proviso that no more than one of Z², Z³, and Z⁴ are N, and wherein Z⁶, Z⁷, and Z⁹ may independently represent N or CR⁴; with the proviso that no more than two of Z⁶, Z⁷, and Z⁹ are N.

In certain embodiments, for example, the aminoisoxazoline compound represented by Formula (I), (Ia), or (Ib), may comprise the W representing the moiety represented by the ring system M-IV-5 with the R⁴ of Z⁸ represents the bond directly attaching the W moiety with the aminoisoxazoline moiety:

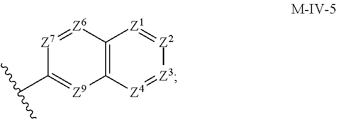

wherein Z¹, Z², and Z³ may independently represent N or CR¹; with the proviso that no more than one of Z¹, Z², and Z³ are N, and wherein Z⁶, Z⁷, and Z⁹ may independently represent N or CR⁴; with the proviso that no more than two of Z⁶, Z⁷, and Z⁹ are N.

In certain embodiments, for example, the aminoisoxazoline compound represented by Formula (I), (Ia), or (Ib), may comprise the W representing the moiety represented by the ring system M-IV-6 with the R⁴ of Z⁸ represents the bond directly attaching the W moiety with the aminoisoxazoline moiety:

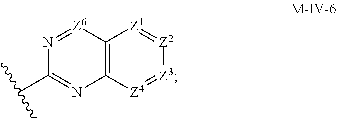

wherein Z¹, Z², Z³, and Z⁴ may independently represent N or CR¹; with the proviso that no more than two of Z¹, Z², Z³, and Z⁴ are N, and wherein Z⁶ independently represents CR⁴.

In certain embodiments, the aminoisoxazoline compound represented by Formula (I), (Ia), or (Ib), may comprise the W representing the moiety represented by the ring system M-IV, such as the ring system M-IV-1, M-IV-2, M-IV-3, M-IV-4, M-IV-5, or M-IV-6, wherein at least 2, 3, or each of Z¹, Z², Z³, and Z⁴, independently represent (CO, for example, at least 2, 3, or each of Z¹, Z², Z³, and Z⁴, independently represent (CR¹) and 2, 1, or 0 of Z¹, Z², Z³, and Z⁴, independently represent N, with the R¹ independently representing —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CF1₂)ₘSO₂C₁-C₄-alkyl, —(CH₂)ₘSO₂N(R²)(R³), or —N(R²)SO₂C₁-C₄-alkyl; or when adjacent members of Z¹, Z², Z³, and Z⁴, is (CR¹)(CR¹), the (CR¹)(CR¹) may form a cycle such that the adjacent R¹ substitutents taken together represents a C₃-C₄-alkyl di-radical or a (3-4 membered)- heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂N(R²)(R³), —(CF1₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

For example, in certain embodiments, Z¹, Z², Z³, or Z⁴, of the ring system M-IV, such as any one of the ring systems M-IV-1 to M-IV-6, may independently represent (CR¹), wherein the R¹ independently represents —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —(CO)N(R²)(R³), —SO₂C₁-C₄-alkyl, or -SO₂N(R²)(R³); wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, or the —SO₂C₁-C₄-alkyl, may be independently substituted with up to 5 radical substituents comprising: —D, halogen radical, ═O, —OR², —(CH₂)ₘOR², —SO₂N(R²)(R³), —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical.

For example, in certain embodiments, Z¹, Z², Z³, or Z⁴, of the ring system M-IV, such as any one of the ring systems M-IV-1 to M-IV-6, may independently represent (CR¹), wherein the R¹ independently represents —H, —D, halogen radical, such as —F, —Cl, or —Br; —CN, an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₆-cycloalkyl radical, such as a cyclopropyl radical or a cyclobutyl radical; an unbranched —OC₁-C₄-alkyl, such as —OCH₃, —OCH₂CH₃, or —OCH₂CH₂CH₃; a branched or cyclic —OC₃-C₄-alkyl, such as —OCH(CH₃)₂ or —O-cyclopropyl; or —SO₂CH₃; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, or the —SO₂CH₃, may be independently substituted with up to 5 radical substituents comprising: —D, halogen radical, such as —F, —Cl, or —Br, ═O, —OR², —(CH₂)ₘOR², an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical. For example, in certain embodiments, Z¹, Z², Z³, or Z⁴, of the ring system M-IV, such as any one of the ring systems M-IV-1 to M-IV-6, may independently represent (CR¹), wherein the R¹ independently represents —H, —D, —F, —Cl, —Br, —CN, —CH₃, —CH₂CH₃, —CH₂CH₂CH₃, —CH(CH₃)₂, a cyclopropyl radical, a cyclobutyl radical, —CH₂F, —CHF₂, —CF₃, —CH₂CF₃, —OCH₃, —OCH₂CH₃, —OCH₂CH₂CH₃, —OCH(CH₃)₂, —O-cyclopropyl, —OCHF₂, —OCH₂F, —OCF₃, —OCH₂CF₃, or —SO₂CH₃; such as the R¹ may independently represent —H, —D, —F, —Cl, —CH₃, —CH₂F, —CHF₂, —CF₃, —OCH₃, —OCHF₂, —OCH₂F, or —OCF₃; such as the R¹ may independently represent —H, —D, —F, —Cl, or —CH₃.

In certain embodiments, the aminoisoxazoline compound represented by Formula (I), (Ia), or (Ib), may comprise the W representing the moiety represented by the ring system M-IV, such as the ring system M-IV-1, M-IV-2, M-IV-3, M-IV-4, M-IV-5, or M-IV-6, wherein adjacent members of Z¹, Z², Z³, and Z⁴, is (CR¹)(CR¹), wherein the (CR¹)(CR¹) may form a cycle such that the adjacent R¹ substitutents taken together represents a C₃-C₄-alkyl di-radical, such as —CH₂CH₂CH₂— or —CH₂CH₂CH₂CH₂-, or represents a (3-4 membered)-heteroalkyl di-radical with at least one ring atom of the (3-4 membered)-heteroalkyl di-radical selected from the group consisting of oxygen, nitrogen, and sulfur, such as —OCH₂CH₂CH₂—, —OCH₂CH₂N(H)—; —OCH₂CH₂N(C₁-C₄-alkyl)-, such as —OCH₂CH₂N(Me)—; —CH₂CH₂CH₂N(CO)(C₁-C₄-alkyl)-, —N(H)CH₂CH₂O—, —N(C₁-C₄-alkyl)CH₂CH₂O—, such as —N(Me)CH₂CH₂O—; —OCH₂CH₂O—; —OCH₂CH₂—; —OCH₂O—; —OCF₂O—; or —CH₂CH₂CH₂O—.

In certain embodiments, the W represents the moiety represented by the ring system M-IV, such as the ring system M-IV-1, M-IV-2, M-IV-3, M-IV-4, M-IV-5, or M-IV-6, wherein the Z⁶, Z⁷, Z⁸, and Z⁹ may independently represent CR⁴, wherein the R⁴ independently represents —H, —D, halogen radical, such as —F, —Cl, or —Br; —CN, an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₆-cycloalkyl radical, such as a cyclopropyl radical or a cyclobutyl radical; an unbranched —OC₁-C₄-alkyl, such as —OCH₃, —OCH₂CH₃, or —OCH₂CH₂CH₃; a branched or cyclic —OC₃-C₄-alkyl, such as —OCH(CH₃)₂ or —O-cyclopropyl; wherein the alkyl portion of the unbranched C₁-C₄-alkyl radical, the branched C₃-C₄-alkyl radical, the C₃-C₆-cycloalkyl radical, the unbranched —OC₁-C₄-alkyl, or the branched or cyclic —OC₃-C₄-alkyl, may be independently substituted with up to 5 radical substituents comprising: —D, halogen radical, such as —F, —Cl, or —Br, or ═O. For example, in certain embodiments, Z⁶, Z⁷, Z⁸, or Z⁹, of the ring system M-IV, such as any one of the ring systems M-IV-1 to M-IV-6, may independently represent CR⁴, wherein the R⁴ independently represents —H, —D, —F, —Cl, —CN, —CH₃, —CH₂CH₃, —CH₂CH₂CH₃, —CH(CH₃)₂, cyclopropyl radical, cyclobutyl radical, —CH₂F, —CHF₂, —CF₃, —CH₂CF₃, —OCH₃, —OCH₂CH₃, —OCH₂CH₂CH₃, —OCH(CH₃)₂, —O-cyclopropyl, —OCHF₂, —OCH₂F, —OCF₃, or —OCH₂CF₃; such as the R⁴ independently represents —H, —D, —F, —Cl, —CN, —CH₃, —CH₂F, —CHF₂, —CF₃, —OCH₃, —OCHF₂, —OCH₂F, or —OCF₃; such as the R⁴ independently represents —H, —D, —F, —Cl, —CH₃, —CH₂F, —CHF₂, or —CF₃.

In certain embodiments, the aminoisoxazoline compound represented by Formula (I), (Ia), or (Ib), may comprise the W representing the moiety represented by the ring system M-I or the ring system M-IV, such as the ring system M-IV-1, M-IV-2, M-IV-3, M-IV-4, M-IV-5, or M-IV-6, wherein the compound may comprise R², R³, or both R² and R³, independently representing —H; an unbranched C₁-C₆-alkyl radical, such as —CH₃ or —CH₂CH₃, a branched C₃-C₆-alkyl radical, such as —CH(CH₃)₂; or a C₃-C₆-cycloalkyl radical, such as a cyclopropyl radical or a cyclobutyl radical. For example, R² and R³ may independently represent —H, —D, —CH₃, —CH₂CH₃, —CH(CH₃)₂, a cyclopropyl radical, or a cyclobutyl radical, such as independently represent —H, —D, —CH₃, or —CH₂CH₃.

In certain embodiments, the aminoisoxazoline compound represented by Formula (I), (Ia), or (Ib), may comprise the W representing the moiety represented by the ring system M-I or the ring system M-IV, such as the ring system M-IV-1, M-IV-2, M-IV-3, M-IV-4, M-IV-5, or M-IV-6, wherein the compound may comprise an N(R²)(R³) moiety, wherein the —N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical.

For example, in certain embodiments, the —N(R²)(R³) moiety of the ring system M-I or the ring system M-IV, such as any one of the ring systems M-IV-1 to M-IV-6, may form a cycle, wherein R² and R³ taken together represent a C₂-C₆-alkyl di-radical, such as a C₂-C₅-alkyl di-radical or C₃-C₄-alkyl di-radical; wherein the C₂-C₆-alkyl di-radical, such as a C₂-C₅-alkyl di-radical or C₃-C₄-alkyl di-radical, may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical. For example, the —N(R²)(R³) moiety may form a cycle, wherein R² and R³ taken together represent a C₂-alkyl di-radical, a C₃-alkyl di-radical, C₄-alkyl di-radical, or C₅-alkyl di-radical, such as a C₂-alkyl di-radical.

For example, in certain embodiments, the —N(R²)(R³) moiety of the ring system M-I or the ring system M-IV, such as any one of the ring systems M-IV-1 to M-IV-6, may form a cycle wherein the R² and R³ taken together represent a (3-6 membered)-heteroalkyl di-radical, such as (4-5 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H; an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₄-cycloalkyl radical; —(CO)-unbranched C₁-C₄-alkyl; —(CO)branched C₃-C₄-alkyl; —(SO₂)-unbranched C₁-C₄-alkyl; or —(SO₂)-branched C₃-C₄-alkyl; and with the further proviso that when the at least one ring atom is sulfur, the sulfur may be independently substituted with 0 to 2 ═O; wherein the alkyl portion of said (3-6 membered)-heteroalkyl di-radical may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical. For example, the —N(R²)(R³) moiety may form a cycle, wherein R² and R³ taken together represent a (4-5 membered)-heteroalkyl di-radical, wherein the (4-5 membered)-heteroalkyl di-radical comprises at least one ring atom selected from the group consisting of oxygen or nitrogen, with the proviso that when the at least one ring atom is nitrogen, the nitrogen is independently substituted with —H; —CH₃, —CH₂CH₃, —CH(CH₃)₂, a cyclopropyl radical. —(CO)CH₃, —(CO)CH₂CH₃, —(SO₂)CH₃, or —(SO₂)CH₂CH₃.

In certain embodiments, the aminoisoxazoline compound represented by Formula (I), (Ia), or (Ib), may comprise the W representing the moiety represented by the ring system M-II, such as the ring system M-II-1 to M-II-11, wherein the compound may comprise R⁹, R¹° , or both R⁹ and R¹⁰, independently representing H; an unbranched C₁-C₆-alkyl radical, such as —CH₃ or —CH₂CH₃, a branched C₃-C₆-alkyl radical, such as —CH(CH₃)₂; or a C₃-C₆-cycloalkyl radical, such as a cyclopropyl radical or a cyclobutyl radical. For example, R⁹ and R¹° may independently represent —H, —D, —CH₃, —CH₂CH₃, —CH(CH₃)₂, a cyclopropyl radical, or a cyclobutyl radical, such as independently represent —H, —D, —CH₃, or —CH₂CH₃.

In certain embodiments, the aminoisoxazoline compound represented by Formula (I), (Ia), or (Ib), may comprise the W representing the moiety represented by the ring system M-II, such as the ring system M-II-1 to M-II-11, wherein the compound may comprise an)N(R⁹)(R¹⁰) moiety, wherein the)N(R⁹)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents, for example, up to 4 radical substituents or up to 3 radical substituents, comprising: —D, halogen radical, ═O, an unbranched C₁-C₄-alkyl radical, or a branched C₃-C₄-alkyl radical.

For example, in certain embodiments, the)N(R⁹)(R¹⁰) moiety of the ring system M-II, such as any one of the ring systems M-II-1 to M-II-11, may form a cycle, wherein R⁹ and R¹⁰ taken together represent a C₂-C₆-alkyl di-radical, such as a C₂-C₅-alkyl di-radical or C₃-C₄-alkyl di-radical; wherein the C₂-C₆-alkyl di-radical, such as a C₂-C₅-alkyl di-radical or C₃-C₄-alkyl di-radical, may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical. For example, the)N(R⁹)(R¹⁰) moiety may form a cycle, wherein R⁹ and R¹⁰ taken together represent a C₂-alkyl di-radical, a C₃-alkyl di-radical, C₄-alkyl di-radical, or C₅-alkyl di-radical, such as a C₂-alkyl di-radical.

For example, in certain embodiments, the)N(R⁹)(R¹⁰) moiety of the ring system M-II, such as any one of the ring systems M-II-1 to M-II-11, may, for example, form a cycle wherein the R⁹ and R¹⁰ taken together represent a (3-6 membered)-heteroalkyl di-radical, such as (4-5 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H; an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₄-cycloalkyl radical; —(CO)-unbranched C₁-C₄-alkyl; —(CO)-branched C₃-C₄-alkyl; —(SO₂)-unbranched C₁-C₄-alkyl; or —(SO₂)-branched C₃-C₄-alkyl; and with the further proviso that when the at least one ring atom is sulfur, the sulfur may be independently substituted with 0 to 2 ═O; wherein the alkyl portion of said (3-6 membered)-heteroalkyl di-radical may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical. For example, the)N(R⁹)(R¹⁰) moiety may form a cycle, wherein R⁹ and R¹⁰ taken together represent a (4-5 membered)-heteroalkyl di-radical, wherein the (4-5 membered)-heteroalkyl di-radical comprises at least one ring atom selected from the group consisting of oxygen or nitrogen, with the proviso that when the at least one ring atom is nitrogen, the nitrogen is independently substituted with —H; —CH₃, —CH₂CH₃, —CH(CH₃)₂, a cyclopropyl radical. —(CO)CH₃, —(CO)CH₂CH₃, —(SO₂)CH₃, or —(SO₂)CH₂CH₃.

In certain embodiments, the aminoisoxazoline compound represented by Formula (I), (Ia), or (Ib), may comprise the W representing the moiety represented by the ring system M-III, such as the ring system M-III-1, wherein the compound may comprise R¹³, R¹⁴, or both R¹³ and R¹⁴, independently representing H; an unbranched C₁-C₄-alkyl radical, such as CH₃ or CH₂CH₃, a branched C₃-C₄-alkyl radical, such as —CH(CH₃)₂; or a C₃-C₄-cycloalkyl radical, such as a cyclopropyl radical or a cyclobutyl radical. For example, R¹³ and R¹⁴ may independently represent —H, —D, —CH₃, —CH₂CH₃, —CH(CH₃)₂, a cyclopropyl radical, or a cyclobutyl radical, such as independently represent —H, —D, —CH₃, or —CH₂CH₃.

In certain embodiments, the aminoisoxazoline compound represented by Formula (I), (Ia), or (Ib), may comprise the W representing the moiety represented by the ring system M-III, such as the ring system M-III-1, wherein the compound may comprise an N(R¹³)(R¹⁴) moiety, wherein the N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical.

For example, in certain embodiments, the —N(R¹³)(R¹⁴) moiety of the ring system M-III, such as the ring system M-III-1, may form a cycle, wherein R¹³ and R^{H} taken together represent a C₂-C₆-alkyl di-radical, such as a C₂-C₅-alkyl di-radical or C₃-C₄-alkyl di-radical; wherein the C₂-C₆-alkyl di-radical, such as a C₂-C₅-alkyl di-radical or C₃-C₄-alkyl di-radical, may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical. For example, the —N(R¹³)(R¹⁴) moiety may form a cycle, wherein R¹³ and R¹⁴ taken together represent a C₂-alkyl di-radical, a C₃-alkyl di-radical, C₄-alkyl di-radical, or C₅-alkyl di-radical, such as a C₂-alkyl di-radical.

For example, in certain embodiments, the —N(R¹³)(R¹⁴) moiety of the ring system M-III, such as the ring system M-III-1, may form a cycle wherein the le and le taken together represent a (3-6 membered)-heteroalkyl di-radical, such as (4-5 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H; an unbranched C₁-C₄-alkyl radical, such as —CH₃, —CH₂CH₃, or —CH₂CH₂CH₃, a branched C₃-C₄-alkyl radical, such as —CH(CH₃)₂; a C₃-C₄-cycloalkyl radical; —(CO)-unbranched C₁-C₄-alkyl; (CO)branched C₃-C₄-alkyl; —(SO₂)-unbranched C₁-C₄-alkyl; or —(SO₂)-branched C₃-C₄-alkyl; and with the further proviso that when the at least one ring atom is sulfur, the sulfur may be independently substituted with 0 to 2 ═O; wherein the alkyl portion of said (3-6 membered)-heteroalkyl di-radical may be independently substituted with up to 5 radical substituents, for example, up to 4 radical substituents or up to 3 radical substituents, comprising: —D, halogen radical, ═O, an unbranched C₁-C₆-alkyl radical, or a branched C₃-C₆-alkyl radical. For example, the —N(R¹³)(R¹⁴) moiety may form a cycle, wherein R¹³ and R¹⁴ taken together represent a (4-5 membered)-heteroalkyl di-radical, wherein the (4-5 membered)-heteroalkyl di-radical comprises at least one ring atom selected from the group consisting of oxygen or nitrogen, with the proviso that when the at least one ring atom is nitrogen, the nitrogen is independently substituted with —H; —CH₃, —CH₂CH₃, —CH(CH₃)₂, a cyclopropyl radical. —(CO)CH₃, —(CO)CH₂CH₃, —(SO₂)CH₃, or —(SO₂)CH₂CH₃.

In certain embodiments, the aminoisoxazoline compound represented by Formula (I), (Ia), or (Ib), may comprise the W representing the moiety represented by the ring system M-I, M-II, or M-IV, wherein m independently represents an integer from 1 to 6, for example, represents an integer of 1, 2, 3, 4, 5, or 6.

In certain embodiments, the aminoisoxazoline compound represented by Formula (I), (Ia), or (Ib), may comprise the W representing the moiety represented by the ring system M-I or M-IV, wherein adjacent members of Z¹, Z², Z³, Z⁴, and Z⁵, is (CR¹)(CR¹), and the (CR¹)(CR¹) forms a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di- radical selected from the group consisting of oxygen, nitrogen, and sulfur, with the proviso that when the at least one ring atom is nitrogen, the nitrogen is unsubstituted (specifically is —N(H)—) or is substituted with an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl; wherein the alkyl portion of the unbranched C₁-C₄-alkyl radical, the branched C₃-C₄-alkyl radical, the C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, may be independently substituted with up to 5, for example, up to 4 or up to 3, radical substituents comprising: —D, halogen radical, ═O, —OR², —(CH₂)ₘOR², an unbranched C₁-C₄-alkyl radical, a branched C₃-C₄-alkyl radical, a C₁-C₄-hydroxyalkyl radical, a C₁-C₂-haloalkyl radical, or —OC₁-C₂-haloalkyl radical, and with the further proviso that when the at least one ring atom is sulfur, the sulfur may substituted with O or 2 ═O; and wherein the alkyl portion of said C₃-C₄-alkyl di-radical or said (3-4 membered)-heteroalkyl di-radical may be substituted with up to 4 radical substituents comprising: —D, halogen radical, ═O, —OR², —(CH₂)ₘOR², —N(R²)(R³), an unbranched C₁-C₄-alkyl radical, a branched C₃-C₄-alkyl radical, a C₁-C₄-hydroxyalkyl radical, a C₁-C₂-haloalkyl radical, or —OC₁-C₂-haloalkyl radical. For example, in certain embodiments, the aminoisoxazoline compound represented by Formula (I), (Ia), or (Ib), may comprise the W representing the moiety represented by the ring system M-I or M-IV, wherein adjacent members of Z¹, Z², Z³, Z⁴, and Z⁵, is (CR¹)(CR¹), such as adjacent members Z¹ and Z² is (CR¹)(CR¹), adjacent members Z² and Z³ is (CR¹)(CR¹), adjacent members Z³ and Z⁴ is (CR¹)(CR¹), or adjacent members Z⁴ and Z⁵ is (CR¹)(CR¹), and the (CR¹)(CR¹) forms a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, for example, at least two ring atoms of the (3-4 membered)-heteroalkyl di-radical are independently selected from the group consisting of oxygen, nitrogen, and sulfur, with the proviso that when the at least one ring atom is, or at least two ring atoms are independently, nitrogen, then the nitrogen is unsubstituted (specifically is —N(H)—) or is substituted with an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl; wherein the alkyl portion of the unbranched C₁-C₄-alkyl radical, the branched C₃-C₄-alkyl radical, the C₃-C₄-cycloalkyl radical, —(CO)-unbranched C₁-C₄-alkyl, —(CO)-branched C₃-C₄-alkyl, —(SO₂)-unbranched C₁-C₄-alkyl, —(SO₂)-branched C₃-C₄-alkyl, the C₃-C₄-alkyl di-radical, or the (3-4 membered)-heteroalkyl di-radical, may be independently substituted with up to 5, for example, up to 4 or up to 3, radical substituents comprising: —D, halogen radical, ═O, —OR², —(CH₂)ₘOR², an unbranched C₁-C₄-alkyl radical, a branched C₃-C₄-alkyl radical, a C₁-C₄-hydroxyalkyl radical, a C₁-C₂-haloalkyl radical, or —OC₁-C₂-haloalkyl radical. For example, in certain embodiments, adjacent members of Z¹, Z², Z³, Z⁴, and Z⁵, is (CR¹)(CR¹), and the (CR¹)(CR¹) forms a cycle such that the adjacent R¹ substitutents taken together represents a C₃-C₄-alkyl di-radical or a (3-4 membered)-heteroalkyl di-radical, and the C₃-C₄-alkyl di-radical or the (3-4 membered)-heteroalkyl di-radical comprises: —CH₂CH₂CH₂—; —CH₂CH₂CH₂CH₂—; —OCH₂CH₂CH₂—, —OCH₂CH₂N(H)—; —OCH₂CH₂N(C₁-C₄-alkyl), such as —OCH₂CH₂N(Me)—; —CH₂CH₂CH₂N(CO)(C₁-C₄-alkyl), —N(H)CH₂CH₂O—, —N(C₁-C₄-alkyl)CH₂CH₂O—, such as —N(Me)CH₂CH₂O—; —OCH₂CH₂O—; —OCH₂CH₂—; —OCH₂O—; —OCF₂O—; or —CH₂CH₂CH₂O—. For purposes described herein, when the C₃-C₄-alkyl di-radical or the (3-4 membered)-heteroalkyl di-radical is specified, it is both referenced and attached on the ring system M-I or M-IV, in order from lowest to highest of adjacent members of Z1 _{z}2 _{z}3 _{z}4 _{and} , Z⁵. For example, by way of illustration, the resulting ring system of W representing the moiety represented by the ring system M-IV-2, wherein the adjacent members Z¹ and Z² is (CR¹)(CR¹), and the (CR¹)(CR¹) forms a cycle such that the adjacent R¹ substitutents taken together represents a (3-4 membered)-heteroalkyl di-radical, and the (3-4 membered)-heteroalkyl di-radical is: (i) —OCH₂CH₂CH₂—, (ii) —OCH₂CH₂N(H)—; (iii) —N(H)CH₂CH₂O—; (iv) —OCH₂CH₂—O; (v) —OCF₂O-—; or (vi) —CH₂CH₂CH₂O—, would be represented by the following structures:

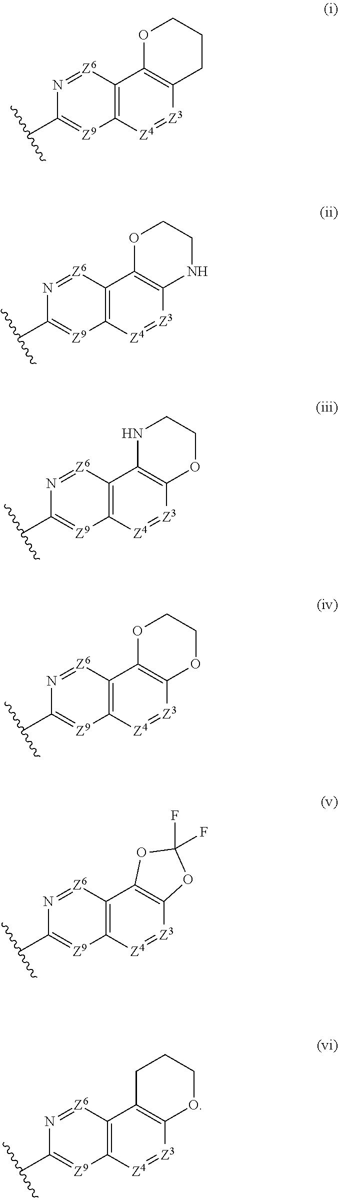

In certain embodiments, the aminoisoxazoline compound represented by Formula (I), (Ia), or (Ib), may comprise racemic mixture of enantiomers, a mixture of diastereomers, a single enantiomer, or a single diastereomer, of the compound, or a pharmaceutically acceptable salt thereof. In certain embodiments, the aminoisoxazoline compound represented by Formula (I), (Ia), or (Ib), may comprise a mixture of tautomers, substantially a single tautomer form, or a single tautomer form, such as a tautomer contained within the aminoisoxazoline ring system or a tautomer resulting from one or more substitutents substituted on the aminoisoxazoline ring system, for example, a tautomer may be contained within the aminoisoxazoline ring system or one or more substitutents substituted on the aminoisoxazoline ring system containing a heteroaryl ring nitrogen adjacent to a heteroaryl ring carbon substituted with a hydroxyl group.

The chemical names and structure diagrams used herein to describe the compounds of the present invention, supra and infra, were created with the use of ChemBioDraw Ultra® Version 12.0 (available from CambridgeSoft Corp., Cambridge, Mass.).

In certain embodiments, specific examples of the aminoisoxazoline compound represented by Formula (I), (Ia), or (Ib) may include, collectively or individually, the compounds listed below, and single (R)- or (S)- enantiomers and pharmaceutically acceptable salts thereof:

N-phenyl-4H-1′-azaspiro[isoxazole-5,3′-bicyclo[2.2.2]octan]-3-amine;

N-(isoquinolin-3-yl)-4H-1′-azaspiro[isoxazole -5,3′-bicyclo[2.2.2]octan]-3-amine;

N-(benzo[d]thiazol-2-yl)-4H-1′-azaspiro[isoxazole-5,3′-bicyclo[2.2.2]octan]-3-amine;

N-(6-methoxybenzo[d]thiazol-2-yl)-4H-1′-azaspiro[isoxazole-5,3′-bicyclo[2.2.2]octan]-3-amine;

N-(1H-indazol-3-yl)-4′H-4-azaspiro[bicyclo[2.2.2]octane-2,5¹-isoxazo1]-3′-amine;

N-(pyrrolo[2,1-f][1,2,4]triazin-4-yl)-4H-1′-azaspiro[isoxazole-5,3′-bicyclo[2.2.2]octan]-3-amine;

N-(7-bromopyrrolo[2,1-f][1,2,4]triazin-4-yl)-4H-1′-azaspiro[isoxazole-5,3′-bicyclo[2.2.2]octan]-3-amine;

N-(4-chlorobenzo[d]thiazol-2-yl)-4H-1′-azaspiro[isoxazole-5,3′-bicyclo[2.2.2]octan]-3-amine;

N-(5 -chlorobenzo[d]thiazol-2-yl)-4H-1′-azaspiro[isoxazole-5,3′-bicyclo[2.2.2]octan]-3-amine;

N-(6-chlorobenzo[d]thiazol-2-yl)-4H-1′-azaspiro[isoxazole-5,3′-bicyclo[2.2.2]octan]-3-amine;

N-(7-chlorobenzo[d]thiazol-2-yl)-4H-1′-azaspiro[isoxazole-5,3′-bicyclo[2.2.2]octan]-3-amine;

N-(benzo[b]thiophen-2-yl)-4H-1′-azaspiro[isoxazole-5,3′-bicyclo[2.2.2]octan]-3-amine;

N-(benzo[d]oxazol-2-yl)-4H-1′-azaspiro[isoxazole-5,3′-bicyclo[2.2.2]octan]-3-amine;

N-(6-methylbenzo[d]thiazol-2-yl)-4H-1′-azaspiro[isoxazole-5,3′-bicyclo[2.2.2]octan]-3-amine;

N-(6-cyclopropylbenzo[d]thiazol-2-yl)-4H-1′-azaspiro[isoxazole-5,3′-bicyclo[2.2.2]octan]-3-amine;

N-(6-fluorobenzo[d]thiazol-2-yl)-4H-1′-azaspiro[isoxazole-5,3′-bicyclo[2.2.2]octan]-3-amine;

N-(5 -chloro-6-fluorobenzo[d]thiazol-2-yl)-4H-1′-azaspiro[isoxazole-5,3′-bicyclo[2.2.2]octan]-3-amine;

N-(6-chloroisoquinolin-3-yl)-4H-1′-azaspiro[isoxazole-5,3′-bicyclo[2.2.2]octan]-3-amine;

N-(7-chloroisoquinolin-3-yl)-4H-1′-azaspiro[isoxazole-5,3′-bicyclo[2.2.2]octan]-3-amine;

N-(4-chlorophenyl)-4H-1′-azaspiro[isoxazole-5,3′-bicyclo[2.2.2]octan]-3-amine;

N-(3,4-dichlorophenyl)-4H-1′-azaspiro[isoxazole-5,3′-bicyclo[2.2.2]octan]-3-amine;

N-(quinazolin-2-yl)-4H-1′-azaspiro[isoxazole-5,3′-bicyclo[2.2.2]octan]-3-amine;

N-(8-chloroisoquinolin-3-yl)-4H-1′-azaspiro[isoxazole-5,3′-bicyclo[2.2.2]octan]-3-amine;

N-(5 -chloroisoquinolin-3-yl)-4H-1′-azaspiro[isoxazole-5,3′-bicyclo[2.2.2]octan]-3-amine;

N-(5 -methylbenzo[d]thiazol-2-yl)-4H-1′-azaspiro[isoxazole-5,3′-bicyclo[2.2.2]octan]-3-amine;

N-(5 -cyclopropylbenzo[d]thiazol-2-yl)-4H-1′-azaspiro[isoxazole-5,3′-bicyclo[2.2.2]octan]-3-amine;

N-(5 -fluorobenzo[d]thiazol-2-yl)-4H-1′-azaspiro[isoxazole-5,3′-bicyclo[2.2.2]octan]-3-amine;

N-(5 -(trifluoromethyl)benzo[d]thiazol-2-yl)-4H-1′-azaspiro[isoxazole-5,3′-bicyclo[2.2.2]octan]-3-amine;

2-((4H-1′-azaspiro[isoxazole-5,3′-bicyclo[2.2.2]octan]-3-yl)amino)benzo[d]thiazole-5-carbonitrile;

N-(5 ,6-dichlorobenzo[d]thiazol-2-yl)-4H-1′-azaspiro[isoxazole-5,3′-bicyclo[2.2.2]octan]-3-amine;

N-(6,7-dichlorobenzo[d]thiazol-2-yl)-4H-1′-azaspiro[isoxazole-5,3′-bicyclo[2.2.2]octan]-3-amine;

N-(5 ,7-dichlorobenzo[d]thiazol-2-yl)-4H-1′-azaspiro[isoxazole-5,3′-bicyclo[2.2.2]octan]-3-amine;

N-(6-chloro-5,7-difluorobenzo[d]thiazol-2-yl)-4H-1′-azaspiro[isoxazole-5,3′-bicyclo[2.2.2]octan]-3-amine;

N-(3-chlorophenyl)-4H-1′-azaspiro[isoxazole-5,3′-bicyclo[2.2.2]octan]-3-amine;

N-(3-chloro-4-(trifluoromethyl)phenyl)-4H-1′-azaspiro[isoxazole-5,3′-bicyclo[2.2.2]octan]-3-amine;

N-(3-chloro-4 -cyclopropylphenyl)-4H-1′-azaspiro[isoxazole -5,3′-bicyclo[2.2.2]octan]-3-amine;

N-(4-chloro-3-(trifluorome thyl)phenyl)-4H-1′-azaspiro[isoxazole-5,3′-bicyclo[2.2.2]octan]-3-amine;

N-(4-chloro-3-cyclopropylphenyl)-4H-1′-azaspiro[isoxazole -5,3′-bicyclo[2.2.2]octan]-3-amine;

N-(4-chloro-3-methoxyphenyl)-4H-1′-azaspiro[isoxazole-5,3′-bicyclo[2.2.2]octan]-3-amine;

N-(2-methylbenzofuran-6 -yl)-4H-1′-azaspiro[isoxazole-5,3′-bicyclo[2.2.2]octan]-3-amine;

N-(2-methylbenzofuran-5 -yl)-4H-1′-azaspiro[isoxazole-5,3′-bicyclo[2.2.2]octan]-3-amine;

N-(2-methylbenzo[b]thiophen-6 -yl)-4H-1′-azaspiro[isoxazole-5,3 ′-bicyclo[2.2.2]octan]-3-amine;

N-(2-methylbenzo[b]thiophen-5 -yl)-4H-1′-azaspiro[isoxazole-5,3 ′-bicyclo[2.2.2]octan]-3-amine;

N-(7-fluoroisoquinolin-3-yl)-4H-1′-azaspiro[isoxazole-5,3′-bicyclo[2.2.2]octan]-3-amine;

N-(6-fluoroisoquinolin-3-yl)-4H-1′-azaspiro[isoxazole-5,3′-bicyclo[2.2.2]octan]-3-amine;

N-(7-methoxyisoquinolin-3-yl)-4H-1′-azaspiro[isoxazole-5,3 ′-bicyclo[2.2.2]octan]-3-amine;

N-(6-methoxyisoquinolin-3-yl)-4H-1′-azaspiro[isoxazole-5,3 ′-bicyclo[2.2.2]octan]-3-amine;

N-(6-chloroquinazolin-2-yl)-4H-1′ -azaspiro[isoxazole-5,3′-bicyclo[2.2.2]octan]-3-amine;

N-(7-chloroquinazolin-2-yl)-4H-1′ -azaspiro[isoxazole-5,3′-bicyclo[2.2.2]octan]-3-amine;

N-(2-methylbenzo[d]thiazol-6-yl)-4H-1′-azaspiro[isoxazole -5,3′-bicyclo[2.2.2]octan]-3-amine;

N-(2 -methylbenzo[d]oxazol-6 -yl)-4H-1′-azaspiro[isoxazole-5 ,3 ′ -bicyclo[2.2.2]octan]-3-amine;

N-(6 -methylthieno[2,3-d] pyrimidin-2 -yl) -4H-1′-azaspiro[isoxazole-5 ,3 ′ -bicyclo[2.2.2]octan]-3-amine;

N-(2-methylthieno[2,3-b] pyridin-6 -yl)-4H-1′-azaspiro[isoxazole-5,3′-bicyclo[2.2.2]octan]-3-amine;

N-(2-chlorobenzo[b]thiophen-6 -yl)-4H-1′-azaspiro[isoxazole5,3′-bicyclo[2.2.2]octan]-3-amine;

N-(2,3-difluorobenzo[b]thiophen-6 -yl)-3H-1′-azaspiro[furan-2,3 ′-bicyclo[2.2.2]octan]-4-amine;

N-(3-methylbenzo[b]thiophen-6 -yl)-4H-1′-azaspiro[isoxazole5,3′-bicyclo[2.2.2]octan]-3-amine;

N-(1 -methyl - 1H-benzo[d] imidazol -6 -yl)-4H-1′-azaspiro[isoxazole5,3′-bicyclo[2.2.2]octan]-3-amine;

N-(1 -methyl - 1H-benzo[d] imidazol -5 -yl)-4H-1′-azaspiro[isoxazole5,3′-bicyclo[2.2.2]octan]-3-amine;

N-(7-methylisoquinolin-3-yl)-4H-1′-azaspiro[isoxazole-5,3′-bicyclo[2.2.2]octan]-3-amine;

N-(6-methylisoquinolin-3-yl)-4H-1′-azaspiro[isoxazole-5,3′-bicyclo[2.2.2]octan]-3-amine;

N-(6,7-difluoroisoquinolin-3-yl)-4H-1′-azaspiro[isoxazole-5,3′-bicyclo[2.2.2]octan]-3-amine;

N-(6,7-dichloroisoquinolin-3-yl)-4H-1′-azaspiro[isoxazole-5,3′-bicyclo[2.2.2]octan]-3-amine;

N-(2-chlorobenzofuran-6-yl)-4H-1′-azaspiro[isoxazole-5,3′-bicyclo[2.2.2]octan]-3-amine;

N-(1-methyl-1H-indazol-6-yl)-4H-1′-azaspiro[isoxazole-5,3′-bicyclo[2.2.2]octan]-3-amine;

N-(1-methyl-1H-indazol-5-yl)-4H-1′-azaspiro[isoxazole-5,3′-bicyclo[2.2.2]octan]-3-amine;

N-(3-methylbenzo[d]isoxazol-6-yl)-4H-1′-azaspiro[isoxazole-5,3′-bicyclo[2.2.2]octan]-3-amine;

N-(2-(trifluoromethyl)benzo[b]thiophen-6-yl)-4H-1′-azaspiro[isoxazole-5,3′-bicyclo[2.2.2]octan]-3-amine;

N-(1,2-dimethyl-1H-benzo[d]imidazol-6-yl)-4H-1′-azaspiro[isoxazole-5,3′-bicyclo[2.2.2]octan]-3-amine;

N-(1-methyl-1H-benzo[d]imidazol-2-yl)-4H-1′-azaspiro[isoxazole-5,3′-bicyclo[2.2.2]octan]-3-amine;

N-(6-methylquinazolin-2-yl)spiro[4H-isoxazole-5,3¹-quinuclidine]-3-amine;

N-(7-methylquinazolin-2-yl)-4H-1′-azaspiro[isoxazole-5,3′-bicyclo[2.2.2]octan]-3-amine;

N-(3-methylbenzo[d]isothiazol-6-yl)-4H-1′-azaspiro[isoxazole-5,3′-bicyclo[2.2.2]octan]-3- amine;

N-(3-chlorobenzofuran-6-yl)-4H-1′-azaspiro[isoxazole-5,3′-bicyclo[2.2.2]octan]-3-amine;

N-(3,5-dichlorobenzofuran-6-yl)-4H-1′-azaspiro[isoxazole-5,3′-bicyclo[2.2.2]octan]-3-amine;

N-(3-methylbenzofuran-6-yl)-4H-1′-azaspiro[isoxazole-5,3′-bicyclo[2.2.2]octan]-3-amine;

N-(6-methylfuro[2,3-d]pyrimidin-2-yl)spiro[4H-isoxazole-5,3¹-quinuclidine]-3-amine;

N-(3-chlorobenzo[b]thiophen-6-yl)-4H-1′-azaspiro[isoxazole-5,3′-bicyclo[2.2.2]octan]-3-amine;

N-(3-(trifluoromethyl)benzo[b]thiophen-6-yl)-4H-1′-azaspiro[isoxazole-5,3′-bicyclo[2.2.2]octan]-3-amine;

N-(2-chloro-3-fluorobenzo[b]thiophen-6-yl)-4H-1′-azaspiro[isoxazole-5,3′-bicyclo[2.2.2]octan]-3-amine;

N-(quinolin-7-yl)-4H-1′-azaspiro[isoxazole-5,3′-bicyclo[2.2.2]octan]-3-amine;

N-(quinolin-6-yl)-4H-1′-azaspiro[isoxazole-5,3′-bicyclo[2.2.2]octan]-3-amine;

N-(6-chloro-7-fluoroisoquinolin-3-yl)-4H-1′-azaspiro[isoxazole-5,3′-bicyclo[2.2.2]octan]-3-amine;

N-(7-chloro-6-fluoroisoquinolin-3-yl)-4H-1′-azaspiro[isoxazole-5,3′-bicyclo[2.2.2]octan]-3-amine;

N-(6-(trifluoromethyl)isoquinolin-3-yl)-4H-1′-azaspiro[isoxazole-5,3′-bicyclo[2.2.2]octan]-3-amine;

N-(2,2-difluorobenzo[d][1,3]dioxo1-5 -yl)-4H-1′-azaspiro[isoxazole-5,3′-bicyclo[2.2.2]octan]-3-amine;

N-(5 ,6,7,8-tetrahydroisoquinolin-3-yl)-4H-1′-azaspiro[isoxazole-5,3′-bicyclo[2.2.2]octan]-3-amine;

N-(5 -methoxypyrimidin-2-yl)-4H-1′-azaspiro[isoxazole-5,3′-bicyclo[2.2.2]octan]-3-amine;

N-(furo[3,2-b]pyridin-5-yl)-4H-1′-azaspiro[isoxazole-5,3′-bicyclo[2.2.2]octan]-3-amine;

2-((4H-1′-azaspiro[isoxazole-5,3′-bicyclo[2.2.2]octan]-3-yllamino)benzo[d]thiazole-6-carbonitrile;

N-(6-(trifluoromethyl)benzo[d]thiazol-2-yl)-4H-1′-azaspiro[isoxazole-5,3′-bicyclo[2.2.2]octan]-3-amine;

N-(quinolin-2-yl)-4H-1′-azaspiro[isoxazole-5,3′-bicyclo[2.2.2]octan]-3-amine;

N-(pyridin-2-yl)-4H-1′-azaspiro[isoxazole-5,3′-bicyclo[2.2.2]octan]-3-amine;

N-(4,5,6-trifluorobenzo[d]thiazol-2-yl)-4H-1′-azaspiro[isoxazole-5,3′-bicyclo[2.2.2]octan]-3-amine;

N-(3,4,5-trichlorophenyl)-4H-1′-azaspiro[isoxazole-5,3′-bicyclo[2.2.2]octan]-3-amine;

N-(4-cyclopropyl-3-methoxyphenyl)-4H-1′-azaspiro[isoxazole-5,3′-bicyclo[2.2.2]octan]-3-amine;

N-(quinolin-3-yl)-4H-1′-azaspiro[isoxazole-5,3′-bicyclo[2.2.2]octan]-3-amine;

N-(2-methylimidazo[1,2-a]pyridin-7-yl)-4H-1′-azaspiro[isoxazole-5,3′-bicyclo[2.2.2]octan]-3-amine;

N-(1,2-dimethyl - 1H-benzo[d] imidazol-5 -yl)-4H-1′-azaspiro[isoxazole-5,3 ′-bicyclo[2.2.2]octan]-3-amine;

N-(furo[3,2-c]pyridin-5-yl)-4H-1′-azaspiro[isoxazole-5,3′-bicyclo[2.2.2]octan]-3-amine;

N-(2-methylthieno[3,2-c]pyridin-6-yl)spiro[4H-isoxazole-5,3¹-quinuclidine]-3-amine; and

N-(2-methylfuro[3,2-c]pyridin-6-yl)spiro[4H-isoxazole-5,3¹-quinuclidine]-3-amine.

In certain embodiments, the specified examples of the aminoisoxazoline compound represented by Formula (I), (Ia), or (Ib), as listed above, for example, may be collectively or individually, the single (R)- enantiomer, or the pharmaceutically acceptable salts thereof, or for example, may be collectively or individually, the single (S)- enantiomer, or the pharmaceutically acceptable salts thereof.

In certain embodiments, specific examples of the aminoisoxazoline compound represented by Formula (I), (Ia), or (Ib) may include, collectively or individually, the compounds listed below, and single (R)- or (S)- enantiomers and pharmaceutically acceptable salts thereof:

N-(2-fluorobenzofuran-6-yl)-4H-1′-azaspiro[isoxazole-5,3′-bicyclo[2.2.2]octan]-3-amine;

N-(6,7-dichloroquinazolin-2-yl)-4H-1′-azaspiro[isoxazole-5,3′-bicyclo[2.2.2]octan]-3-amine;

N-(6-chloro-7-fluoroquinazolin-2-yl)-4H-1′-azaspiro[isoxazole-5,3′-bicyclo[2.2.2]octan]-3-amine;

N-(6 -chlorothieno [2,3-d]pyrimidin-2 -yl)-4H-1′-azaspiro[isoxazole-5,3′-bicyclo[2.2.2]octan]-3-amine;

N-(5 -methylthieno[2,3-d] pyrimidin-2 -yl) -4H-1′-azaspiro[isoxazole-5 ,3 ′ -bicyclo[2.2.2]octan]-3-amine;

N-(6-(trifluoromethyl)thieno [2,3-d]pyrimidin-2-yl)-4H-1′-azaspiro[isoxazole-5 ,3′-bicyclo[2.2.2]octan]-3-amine;

N-(7-(trifluoromethyl)isoquinolin-3-yl)-4H-1′-azaspiro[isoxazole-5,3′-bicyclo[2.2.2]octan]-3-amine;

N-(6-(trifluoromethyl)quinazolin-2-yl)-4H-1′-azaspiro[isoxazole-5,3′-bicyclo[2.2.2]octan]-3-amine;

N-(7-(trifluoromethyl)quinazolin-2-yl)-4H-1′-azaspiro[isoxazole-5,3′-bicyclo[2.2.2]octan]-3-amine;

N-(6,7-bis(trifluoromethyl)quinazolin-2-yl)-4H-1′-azaspiro[isoxazole5,3′-bicyclo[2.2.2]octan]-3-amine;

N-(2-methylfuro [2,3-b] pyridin-6 -yl)-4H-1′-azaspiro[isoxazole5,3′-bicyclo[2.2.2]octan]-3-amine;

N-(2-chlorofuro[2,3-b] pyridin-6-yl)-4H-1′-azaspiro[isoxazole-5,3′-bicyclo[2.2.2]octan]-3-amine;

N-(3-methylthieno[2,3-b] pyridin-6 -yl)-4H-1′-azaspiro[isoxazole-5,3′-bicyclo[2.2.2]octan]-3-amine;

N-(3-methylthieno[3 ,2-c]pyridin-6-yl)-4H-1′-azaspiro[isoxazole-5,3′-bicyclo[2.2.2]octan]-3-amine;

N-(3-(difluoromethyl)benzo[b]thiophen-6-yl)-4H-1′-azaspiro[isoxazole -5 ,3′-bicyclo[2.2.2]octan]-3-amine;

N-(2-chlorothieno [2,3-b] pyridin-6 -yl)-4H-1′-azaspiro[isoxazole-5,3′-bicyclo[2.2.2]octan]-3-amine;

N-(3-methylisothiazolo [5 ,4-b]pyridin-6 -yl)-4H-1′-azaspiro[isoxazole -5 ,3′-bicyclo[2.2.2]octan]-3-amine;

N-(3-methylisothiazolo [4,5 -c] pyridin-6 -yl)-4H-1′-azaspiro[isoxazole5,3′-bicyclo[2.2.2]octan]-3-amine;

N-(2 -cyclopropylbenzo[b]thiophen-6-yl)-4H-1′-azaspiro[isoxazole-5,3′-bicyclo[2.2.2]octan]-3-amine;

2,2-difluoro-N-(4H-1′-azaspiro[isoxazole5,3′-bicyclo[2.2.2]octan]-3-yl)-[ 1,3]dioxolo [4,5-g]isoquinolin-7 -amine;

N-(5 -chlorothieno [2,3-d] pyrimidin-2-yl)-4H-1′-azaspiro[isoxazole-5,3′-bicyclo[2.2.2]octan]-3-amine;

N -(5 -fluoro-6-methylthieno[2,3-d] pyrimidin-2 -yl)-4H-1′-azaspiro[isoxazole-5 ,3′-bicyclo[2.2.2]octan]-3-amine;

N-(6-chloro-5 -fluorothieno [2,3-d] pyrimidin-2-yl)-4H-1′-azaspiro[isoxazole-5 ,3′-bicyclo[2.2.2]octan]-3-amine;

N-(6-cyclopropylquinazolin-2-yl)-4H-1′-azaspiro[isoxazole-5,3′-bicyclo[2.2.2]octan]-3-amine;

N-(7-cyclopropylquinazolin-2-yl)-4H-1′-azaspiro[isoxazole-5,3′-bicyclo[2.2.2]octan]-3-amine;

N-(2-chloro-3-fluorothieno [2,3-b] pyridin-6 -yl)-4H-1′-azaspiro[isoxazole-5,3′-bicyclo[2.2.2]octan]-3-amine;

N-(3-fluoro-2-methylthieno[2,3-b] pyridin-6-yl)-4H-1 ′ -azaspiro[isoxazole-5 ,3′-bicyclo[2.2.2]octan]-3-amine;

N-(3-chlorofuro[2,3-b] pyridin-6-yl)-4H-1′-azaspiro[isoxazole-5,3′-bicyclo[2.2.2]octan]-3-amine;

N-(3-chlorothieno [2,3-b] pyridin-6 -yl)-4H-1′-azaspiro[isoxazole-5,3′-bicyclo[2.2.2.]octan]-3-amine; and

N-(6-(1H-imidazol- 1 -yl)pyrimidin-4-yl)-4H-1′-azaspiro[isoxazole-5,3′-bicyclo[2.2.2]octan]-3-amine.

In certain embodiments, the specified examples of the aminoisoxazoline compound represented by Formula (I), (Ia), or (Ib), as listed above, for example, may be collectively or individually, the single (R)- enantiomer, or the pharmaceutically acceptable salts thereof, or for example, may be collectively or individually, the single (S)- enantiomer, or the pharmaceutically acceptable salts thereof

In certain embodiments, specific examples of the aminoisoxazoline compound represented by Formula (I), (Ia), or (Ib) may include, collectively or individually, the single enantiomers listed below, and pharmaceutically acceptable salts thereof:

(R)—N-(6-chloroisoquinolin-3-yl)-4H-1′-azaspiro[isoxazole-5,3′-bicyclo[2.2.2]octan]-3-amine;

(R)—N-(7-chloroisoquinolin-3-yl)-4H-1′-azaspiro[isoxazole-5,3′-bicyclo[2.2.2]octan]-3-amine;

(R)—N-(3,4-dichlorophenyl)-4H-1′-azaspiro[isoxazole-5,3′-bicyclo[2.2.2]octan]-3-amine;

(R)—N-(quinazolin-2-yl)-4H-1′-azaspiro[isoxazole-5,3′-bicyclo[2.2.2]octan]-3-amine;

(R)—N-(2-methylbenzofuran-6-yl)-4H-1′-azaspiro[isoxazole-5,3′-bicyclo[2.2.2]octan]-3-amine;

(R)—N-(7-fluoroisoquinolin-3-yl)-4H-1′-azaspiro[isoxazole-5,3′-bicyclo[2.2.2]octan]-3-amine;

(R)—N-(6-fluoroisoquinolin-3-yl)-4H-1′-azaspiro[isoxazole-5,3′-bicyclo[2.2.2]octan]-3-amine;

(R)—N-(6-chloroquinazolin-2-yl)-4H-1′-azaspiro[isoxazole-5,3′-bicyclo[2.2.2]octan]-3-amine;

(R)—N-(7-chloroquinazolin-2-yl)-4H-1′-azaspiro[isoxazole-5,3′-bicyclo[2.2.2]octan]-3-amine

(R)—N-(6-methylthieno[2,3-d]pyrimidin-2-yl)-4H-1′-azaspiro[isoxazole-5,3′-bicyclo[2.2.2]octan]-3-amine;

(R)—N-(2-methylthieno[2,3-b]pyridin-6-yl)-4H-1′-azaspiro[isoxazole-5,3′-bicyclo[2.2.2]octan]-3-amine;

(R)—N-(2-chlorobenzo[b]thiophen-6-yl)-4H-1′-azaspiro[isoxazole-5,3′-bicyclo[2.2.2]octan]-3-amine;

(R)—N-(7-methylisoquinolin-3-yl)-4H-1′-azaspiro[isoxazole-5,3′-bicyclo[2.2.2]octan]-3-amine;

(R)—N-(6-methylisoquinolin-3-yl)-4H-1′-azaspiro[isoxazole-5,3′-bicyclo[2.2.2]octan]-3-amine;

(R)—N-(2-chlorobenzofuran-6-yl)-4H-1′-azaspiro[isoxazole-5,3′-bicyclo[2.2.2]octan]-3-amine;

(R)—N -(3-chlorobenzofuran-6-yl)-4H-1′-azaspiro[isoxazole -5,3′-bicyclo[2.2.2]octan]-3-amine;

(R)—N-(3-methylbenzofuran-6-yl)-4H-1′-azaspiro[isoxazole-5,3′-bicyclo[2.2.2]octan]-3-amine;

(R)—N-(6-chloro-7-fluoroisoquinolin-3-yl)-4H-1′-azaspiro[isoxazole5,3′-bicyclo[2.2.2]octan]-3-amine;

(R)—N-(7-chloro-6-fluoroisoquinolin-3-yl)-4H-1′-azaspiro[isoxazole5,3′-bicyclo[2.2.2]octan]-3-amine; and

(R)—N-(6-(trifluoromethyl)isoquinolin-3-yl)-4H-1′-azaspiro[isoxazole -5 ,3′-bicyclo[2.2.2]octan]-3-amine.

In certain embodiments, specific examples of the aminoisoxazoline compound represented by Formula (I), (Ia), or (Ib) may include, collectively or individually, the single enantiomers listed below, and pharmaceutically acceptable salts thereof:

(R)—N-(7-fluoroisoquinolin-3-yl)-4H-1′-azaspiro[isoxazole -5,3′-bicyclo[2.2.2]octan]-3-amine;

(R)—N-(6-fluoroisoquinolin-3-yl)-4H-1′-azaspiro[isoxazole -5 ,3′-bicyclo[2.2.2]octan]-3-amine;

(R)—N-(2 -methylthieno[2,3-b]pyridin-6 -yl)-4H-1′-azaspiro[isoxazole-5 ,3′-bicyclo[2.2.2]octan]-3-amine;

(R)—N-(2-chlorobenzo[b]thiophen-6-yl)-4H-1 ′ -azaspiro[isoxazole-5 ,3 ′ -bicyclo[2.2 .2] octan]-3-amine;

(R)—N-(7-methylisoquinolin-3-yl)-4H-1′-azaspiro[isoxazole-5,3′-bicyclo[2.2.2]octan]-3-amine;

(R)—N-(6-methylisoquinolin-3-yl)-4H-1′-azaspiro[isoxazole-5,3′-bicyclo[2.2.2]octan]-3-amine;

(R)—N-(6-chloro-7-fluoroisoquinolin-3-yl)-4H-1′-azaspiro[isoxazole-5,3′-bicyclo[2.2.2]octan]-3-amine; and

(R)—N-(7-chloro-6-fluoroisoquinolin-3-yl)-4H-1′-azaspiro[isoxazole-5,3′-bicyclo[2.2.2]octan]-3-amine.

In certain embodiments, specific examples of the aminoisoxazoline compound represented by Formula (I), (Ia), or (Ib), wherein the W is represented by the moiety represented by the ring system M-I, may include, collectively or individually, the single enantiomers listed below, and pharmaceutically acceptable salts thereof:

(R)—N -(3,4-dichlorophenyl)-4H-1¹-azaspiro[isoxazole-5,3′-bicyclo[2.2.2]octan]-3-amine.

In certain embodiments, specific examples of the aminoisoxazoline compound represented by Formula (I), (Ia), or (Ib), wherein the W is represented by the moiety represented by the ring system M-II, may include, collectively or individually, the single enantiomers listed below, and pharmaceutically acceptable salts thereof:

(R)—N-(2-methylbenzofuran-6-yl)-4H-1′-azaspiro[isoxazole-5,3′-bicyclo[2.2.2]octan]-3-amine;

(R)—N-(6-methylthieno[2,3-d]pyrimidin-2-yl)-4H-1′-azaspiro[isoxazole-5,3′-bicyclo[2.2.2]octan]-3-amine;

(R)—N-(2-methylthieno[2,3-b]pyridin-6-yl)-4H-1′-azaspiro[isoxazole-5,3′-bicyclo[2.2.2]octan]-3-amine;

(R)—N-(2-chlorobenzo[b]thiophen-6-yl)-4H-1′-azaspiro[isoxazole-5,3′-bicyclo[2.2.2]octan]-3-amine;

(R)—N-(2-chlorobenzofuran-6-yl)-4H-1′-azaspiro[isoxazole-5,3′-bicyclo[2.2.2]octan]-3-amine;

(R)—N -(3-chlorobenzofuran-6-yl)-4H-1′-azaspiro[isoxazole-5,3′-bicyclo[2.2.2]octan]-3-amine;

and

(R)—N-(3-methylbenzofuran-6-yl)-4H-1′-azaspiro[isoxazole-5,3′-bicyclo[2.2.2]octan]-3-amine.

In certain embodiments, specific examples of the aminoisoxazoline compound represented by Formula (I), (Ia), or (Ib), wherein the W is represented by the moiety represented by the ring system M-II, may include, collectively or individually, the single enantiomers listed below, and pharmaceutically acceptable salts thereof:

(R)—N-(2 -methylthieno[2,3-b]pyridin-6 -yl)-4H-1′-azaspiro[isoxazole-5,3′-bicyclo[2.2.2]octan]-3-amine; and

(R)—N-(2 -chlorobenzo[b]thiophen-6 -yl)-4H-1′-azaspiro[isoxazole-5,3′-bicyclo[2.2 .2] octan]-3-amine.

In certain embodiments, specific examples of the aminoisoxazoline compound represented by Formula (I), (Ia), or (Ib), wherein the W is represented by the moiety represented by the ring system M-IV, may include, collectively or individually, the single enantiomers listed below, and pharmaceutically acceptable salts thereof:

(R)—N-(6-chloroisoquinolin-3-yl)-4H-1′-azaspiro[isoxazole-5,3′-bicyclo[2.2.2]octan]-3-amine;

(R)—N-(7-chloroisoquinolin-3-yl)-4H-1′-azaspiro[isoxazole-5,3′-bicyclo[2.2.2]octan]-3-amine;

(R)—N-(quinazolin-2-yl)-4H-1′-azaspiro[isoxazole-5,3′-bicyclo[2.2.2]octan]-3-amine;

(R)—N-(7-fluoroisoquinolin-3-yl)-4H-1′-azaspiro[isoxazole-5,3′-bicyclo[2.2.2]octan]-3-amine;

(R)—N-(6-fluoroisoquinolin-3-yl)-4H-1′-azaspiro[isoxazole-5,3′-bicyclo[2.2.2]octan]-3-amine;

(R)—N-(6-chloroquinazolin-2-yl)-4H-1′-azaspiro[isoxazole-5,3′-bicyclo[2.2.2]octan]-3-amine;

(R)—N-(7-chloroquinazolin-2-yl)-4H-1′-azaspiro[isoxazole-5,3′-bicyclo[2.2.2]octan]-3-amine;

(R)—N-(7-methylisoquinolin-3-yl)-4H-1′-azaspiro[isoxazole-5,3′-bicyclo[2.2.2]octan]-3-amine;

(R)—N-(6-methylisoquinolin-3-yl)-4H-1′-azaspiro[isoxazole-5,3′-bicyclo[2.2.2]octan]-3-amine;

(R)—N-(6-chloro-7-fluoroisoquinolin-3-yl)-4H-1′-azaspiro[isoxazole-5,3′-bicyclo[2.2.2]octan]-3-amine;

(R)—N-(7-chloro-6-fluoroisoquinolin-3-yl)-4H-1′-azaspiro[isoxazole-5,3′-bicyclo[2.2.2]octan]-3-amine; and

(R)—N-(6-(trifluoromethypisoquinolin-3-yl)-4H-1′-azaspiro[isoxazole-5,3′-bicyclo[2.2.2]octan]-3-amine.

In certain embodiments, specific examples of the aminoisoxazoline compound represented by Formula (I), (Ia), or (Ib), wherein the W is represented by the moiety represented by the ring system M-IV, may include, collectively or individually, the single enantiomers listed below, and pharmaceutically acceptable salts thereof:

(R)—N-(7-fluoroisoquinolin-3-yl)-4H-1′-azaspiro[isoxazole-5,3′-bicyclo[2.2.2]octan]-3-amine;

(R)—N-(6-fluoroisoquinolin-3-yl)-4H-1′-azaspiro[isoxazole-5,3′-bicyclo[2.2.2]octan]-3-amine;

(R)—N-(7-methylisoquinolin-3-yl)-4H-1′-azaspiro[isoxazole 5,3′-bicyclo[2.2.2]octan]-3-amine;

(R)—N-(6-methylisoquinolin-3-yl)-4H-1′-azaspiro[isoxazole 5,3′-bicyclo[2.2.2]octan]-3-amine;

(R)—N-(6-chloro-7-fluoroisoquinolin-3-yl)-4H-1′-azaspiro[isoxazole-5,3′-bicyclo[2.2.2]octan]-3-amine; and

(R)—N-(7-chloro-6-fluoroisoquinolin-3-yl)-4H-1′-azaspiro[isoxazole-5,3′-bicyclo[2.2.2]octan]-3-amine.

In certain embodiments, the aminoisoxazoline compounds of the present invention represented by Formula (I), (Ia), or (Ib), or a pharmaceutically acceptable salt thereof, may be more potent against α7 nAChR (according to the α7 nAChR Binding Assay (Ki)) than against a 5-HT₃ serotonin receptor (according to the [³H]BRL 43694 competition binding (Ki)). For example, the aminoisoxazoline compounds of the present invention represented by Formula (I), (Ia), or (Ib), or a pharmaceutically acceptable salt thereof, may be at least 1.5 times more potent against α7 nAChR than against a 5-HT₃ serotonin receptor, as determined by the α7 nAChR Binding Assay and the [³H]BRL 43694 competition binding assay, respectively, such as at least 2 times more potent, at least 3 times more potent, at least 4 times more potent, at least 5 times more potent, at least 6 times more potent, at least 7 times more potent, at least 8 times more potent, at least 9 times more potent, at least 10 times more potent, at least 15 times more potent, at least 20 times more potent, or at least 25 times more potent against α7 nAChR than against a 5-HT₃ serotonin receptor, as determined by the α7 nAChR Binding Assay and the [³H]BRL 43694 competition binding assay, respectively.

As used herein, the term “treating” (or “treat” or “treatment”), unless otherwise specified, includes the generally accepted meaning which encompasses improving, modifying, decreasing, prohibiting, preventing, restraining, minimizing, slowing, halting, stopping, curing, and/or reversing a symptom associated with a disease and/or a disease. Treatment may include both therapeutic and prophylactic administration. For example, treatment of a cognitive impairment, in a patient diagnosed as having a cognitive impairment, may include, but is not limited to, curing the cognitive impairment, preventing the deterioration of one or more symptoms associated with the cognitive impairment; improving cognition in a patient suffering from the cognitive impairment, slowing the progression of the cognitive impairment and/or modifying the cognitive impairment.

As used herein, the term “effective dose” (or “dose”), unless otherwise specified, is understood to include a therapeutically acceptable dose, a thereapeutically acceptable amount, a thereapeutically effective dose, a thereapeutically effective amount, a pharmaceutically acceptable dose, a pharmaceutically acceptable amount, a pharmaceutically effective dose, or a pharmaceutically effective amount.

As used herein, the term “cognitive impairment,” unless otherwise specified, includes at least one of the following: Limited Cognitive Impairment (LCI), Mild Cognitive Impairment (MCI), Alzheimer's disease (or dementia of an Alzheimer's-type) or a particular stage of Alzheimer's disease, inclusive of pre-Alzheimer's disease, early Alzheimer's disease, mild Alzheimer's disease, moderate Alzheimer's disease, severe Alzheimer's disease, pre-Alzheimer's-to-mild Alzheimer's disease, mild-to-moderate Alzheimer's disease, moderate-to-severe Alzheimer's disease, schizophrenia (for example, paranoid type schizophrenia, disorganized type schizophrenia, catatonic type schizophrenia, undifferentiated type schizophrenia), cognitive impairment associated with schizophrenia, schizophreniform disorder, schizoaffective disorder, delusional disorder, positive symptoms of schizophrenia, negative symptoms of schizophrenia, or schizophrenia with dementia.

Alzheimer's disease may include, unless otherwise specified, any of the sub-diagnostic categories used to characterize the type or degree of cognitive impairment in a patient for treatment purposes. A commonly referenced diagnostic scale for characterizing the degree of cognitive impairment for a patient with Alzheimer's disease includes the 3-stage Alzheimer Disease Model. The 3-stages consist of: mild stage (also referred to as “early Alzheimer's disease” or “mild Alzheimer's disease” or “early stage Alzheimer's disease” or “mild dementia of an Alzheimer's- type”), moderate stage (also referred to as “middle Alzheimer's disease” or “moderate Alzheimer's disease” or “middle stage Alzheimer's disease” or “moderate dementia of an Alzheimer's-type”), and severe stage (also referred to as “late Alzheimer's disease” or “severe Alzheimer's disease” or “late stage Alzheimer's disease” or “severe dementia of an Alzheimer's-type”). For patients with a condition that has not progressed to the point of mild stage Alzheimer's disease, they may be diagnosed as having pre-Alzheimer's disease. It is also not uncommon for treatment purposes to characterize stages together, such as pre-Alzheimer's disease-to-mild stage Alzheimer's disease, mild-to-moderate Alzheimer's disease, or moderate-to-severe Alzheimer's disease. Another useful diagnostic scale that is used in characterizing the degree of cognitive impairment for a patient having Alzheimer's disease is the Seven Stage Alzheimer's Disease Model (sometimes known as the “Seven Stage Global Deterioration Scale” or the “Reisberg Scale”). This diagnostic scale divides the progression of the cognitive disorder associated with Alzheimer's disease as follows: Stage 1-no Alzheimer's disease (generally characterized by absence of impairment, no impairment, or normal function), Stage 2-pre-Alzheimer's disease (generally characterized by minimal impairment, normal forgetfulness, or very mild cognitive decline), Stage 3-early-stage Alzheimer's disease (generally characterized by a noticeable cognitive decline, early confusional/mild cognitive impairment, or mild cognitive decline), Stage 4-early-stage/mild Alzheimer's disease (also referred to as late confusional/mild Alzheimer's, and generally characterized by moderate cognitive decline), Stage 5-middle-stage/moderate Alzheimer's (also referred to as early dementia/moderate Alzheimer's disease and generally characterized by moderately severe cognitive decline), Stage 6-middle dementia/moderately severe Alzheimer's disease (also referred to as middle-stage/moderate to late-stage/severe Alzheimer's disease and generally characterized by severe cognitive decline), and Stage 7-late-stage/severe Alzheimer's disease (also referred to as severe dementia or failure-to-thrive, and generally characterized by very severe cognitive decline). It is also not uncommon for treatment purposes to characterize stages together, such as pre-Alzheimer's disease-to-mild stage Alzheimer's disease, mild-to-moderate Alzheimer's disease, or moderate-to-severe Alzheimer's disease. As used herein, unless otherwise specified, Alzheimer's disease includes all of the above named diagnostic catagories or disease characterizations. It is also not uncommon for a physician to categorize any one or more of the above noted states of Alzheimer's disease as being probable, for example, probable mild-to-moderate Alzheimer's disease or probable severe Alzheimer's disease, when their diagnosis does not include, for example a physical biopsy or other definitive analysis.

Mild Cognitive Impairment (MCI) is considered by some to be an intermediate stage between normal aging and the onset of Alzheimer's disease. For example, MCI may be characterized by persistent forgetfulness, but may lack some or many of the more debilitating symptoms of Alzheimer's disease. Another set of criteria that may characterize a patient as having mild cognitive impairment suitable for treatment includes a patient that meets the following: 1) memory complaints corroborated by an informant, 2) objective memory impairment for age and education, 3) normal general cognitive function, 4) intact activities of daily living, and 5) the patient does not meet criteria for dementia. In general, a patient characterized as having mild cognitive impairment may not yet have a clinical cognitive deficit. Mild cognitive impairment may also be distinguished from senile dementia in that mild cognitive impairment involves a more persistent and troublesome problem of memory loss for the age of the patient. On the clinical diagnostic scale, mild cognitive impairment is followed, in increased severity, by Alzheimer's disease.

Limited Cognitive Impairment (LCI) describes a cognitive impairment (i.e., symptoms or conditions), which precedes mild cognitive impairment on a clinical diagnostic scale, and includes any chronic or temporary impairment in cognition, learning or memory that prevents or reduces the ability of a patient from achieving their individual potential in these areas. For example, LCIs may include minor impairments to memory associated with focus and concentration (e.g., accuracy and speed of learning and recalling information), working memory (e.g., used in decision making and problem solving), cognition, focus, mental quickness, and mental clarity.

The term “stereoisomer” refers to a molecule capable of existing in more than one spatial atomic arrangement for a given atomic connectivity (e.g., enantiomers, meso compounds, and diastereomers). As used herein, the term “stereoisomer” means either or both enantiomers and diastereomers.

The aminoisoxazoline compounds of the present invention represented by Formula (I), (Ia), or (Ib), or a pharmaceutically acceptable salt thereof, may contain one or more stereogenic centers. Accordingly, compounds of this invention can exist as either individual stereoisomers or mixtures of two or more stereoisomers. A compound of the present invention will include both mixtures (e.g., racemic mixtures) and also individual respective stereoisomers that are substantially free from another possible stereoisomer. The term “substantially free of other stereoisomers” as used herein means less than 25% of other stereoisomers, less than 10% of other stereoisomers, less than 5% of other stereoisomers, less than 2% of other stereoisomers, or less than “X”% of other stereoisomers (wherein X is a number between 0 and 100, inclusive) are present.

The aminoisoxazoline compounds of the present invention represented by Formula (I), (Ia), or (Ib), or a pharmaceutically acceptable salt thereof, may contain one or more tautomeric forms. Accordingly, compounds of this invention can exist as either individual tautomers or mixtures of tautomeric forms. A compound of the present invention will include both mixtures (e.g., mixtures of tautomeric forms) and also individual respective tautomers that are substantially free from another possible tautomer.

The aminoisoxazoline compounds of the present invention represented by Formula (I), (Ia), or (Ib), or a pharmaceutically acceptable salt thereof, may contain one or more geometric isomers. Accordingly, compounds of this invention can exist as either geometric isomers or mixtures of geometric isomers. A compound of the present invention will include both mixtures (e.g., mixtures of geometric isomers) and also individual respective geometric isomers that are substantially free from another possible geometric isomer.

The term “haloalkyl” refers to an alkyl group having from 1 to 5 halogen substituents independently selected from —F, —Cl, —Br, and —I. For example, a haloalkyl may represent a —CF₃ group, a —CCl₃ group, a —CH₂CF₃ group, or a —CF₂CF₃ group.

The term “heteroaryl” refers to an aromatic ring system comprising at least one or more hetero- ring atoms, such as two, three, four, or five hetero- ring atoms, independently selected from N, O, and S. Suitable heteroaryl groups may include a single ring, for example, thienyl, pyridyl, thiazolyl, pyrazinyl, pyrimidyl, imidazolyl, furanyl, isothiazolyl, pyrazolyl, triazolyl, tetrazolyl, isoxazolyl, oxazolyl, pyrrolyl, pydridazinyl, triazinyl, oxadiazolyl, and furazanyl. Sutiable heteroaryl groups may include a fused ring system, for example, a six-six fused ring system, a six-five fused ring system, or a five-six fused ring system, such as benzothienyl, quinolyl, benzofuranyl, benzothiazolyl, benzisothiazolyl, benzisoxazolyl, benzimidazolyl, indolyl, benzoxazolyl, isoquinolinyl, cinnolinyl, indazolyl, indolizinyl, phthalazinyl, isoindolyl, purinyl, benzofurazanyl, benzothiophenyl, benzothiazolyl, quinazolinyl, quinoxalinyl, naphthridinyl, and furopyridinyl.

Suitable “heterocycloalkyl” groups include those having at least one or more hetero- ring atoms, such as two or three hetero- ring atoms, independently selected from at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 =0. Suitable heterocycloalkyl groups may include, for example, tetrahydrofurano, tetrahydropyrano, morpholino, pyrrolidino, piperidino, piperazino, azetidino, azetidinono, oxindolo, oxetano, dihydroimidazolo, and pyrrolidinono.

The pharmaceutically acceptable salt of the aminoisoxazoline compounds represented by Formula (I), (Ia), or (Ib), according to the present invention may be acid addition salts with inorganic or organic acids. Specific examples of these salts include acid addition salts with, for instance, mineral acids such as hydrochloric acid, hydrobromic acid, hydriodic acid, sulfuric acid, nitric acid or phosphoric acid; organic acids, for example carboxylic acids or sulfonic acids, such as formic acid, acetic acid, propionic acid, oxalic acid, malonic acid, succinic acid, fumaric acid, maleic acid, lactic acid, malic acid, tartaric acid, citric acid, benzoic acid, p-toluenesulfonic acid, benzenesulfonic acid, naphthalenedisulfonic acid, isethionic acid, glucuronic acid, gluconic acid, methanesulfonic acid or ethanesulfonic acid; or acidic amino acids such as aspartic acid or glutamic acid.

In certain embodiments, a pharmaceutical composition may comprise an aminoisoxazoline compound represented by Formula (I), (Ia), or (Ib), or a pharmaceutically acceptable salt thereof, and at least one pharmaceutically acceptable carrier, excipient or diluent.

In certain embodiments, the aminoisoxazoline compounds represented by Formula (I), (Ia), or (Ib), or a pharmaceutically acceptable salt thereof, are suitable for use as medicaments for the treatment and/or prophylaxis of diseases in humans and/or animals.

In certain embodiments, the invention relates to a method comprising administering to a patient in need thereof an effective dose of an aminoisoxazoline compound represented by Formula (I), (Ia), or (Ib), or a pharmaceutically acceptable salt thereof; or administering to the patient a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

In certain embodiments, the aminoisoxazoline compound represented by Formula (I), (Ia), or (Ib), or a pharmaceutically acceptable salt thereof, act as ligands, in particular as α7-nAChR agonists.

In certain embodiments, a method of treating a patient in need thereof, comprising administering an aminoisoxazoline compound represented by Formula (I), (Ia), or (Ib), or a pharmaceutically acceptable salt thereof. In certain embodiments, a method of treating a patient in need thereof, comprising administering a pharmaceutical composition comprising an aminoisoxazoline compound represented by Formula (I), (Ia), or (Ib), or a pharmaceutically acceptable salt thereof. For example, the patient may suffer from a cognitive impairment or suffers from one or more symptoms associated with a cognitive impairment, such as Limited Cognitive Impairment (LCI), Mild Cognitive Impairment (MCI), Alzheimer's disease, dementia of an Alzheimer's-type, schizophrenia, schizophreniform disorder, schizoaffective disorder, delusional disorder, positive symptoms of schizophrenia, negative symptoms of schizophrenia, schizophrenia with dementia, or major depressive disorder.

In certain embodiments, a method of treating Alzheimer's disease, such as preventing the progression or disease modification of the Alzheimer's disease, in a patient in need thereof, comprising administering an aminoisoxazoline compound represented by Formula (I), (Ia), or (Ib), or a pharmaceutically acceptable salt thereof For example, in certain embodiments, a method of treating Alzheimer's disease, such as preventing the progression or disease modification of the Alzheimer's disease, in a patient in need thereof, comprising administering a pharmaceutical composition comprising an aminoisoxazoline compound represented by Formula (I), (Ia), or (Ib), or a pharmaceutically acceptable salt thereof. In certain embodiments, a method of treating cognitive impairment associated with Alzheimer's disease, such as preventing the progression or disease modification of the Alzheimer's disease, in a patient in need thereof, comprising administering an aminoisoxazoline compound represented by Formula (I), (Ia), or (Ib), or a pharmaceutically acceptable salt thereof. For example, in certain embodiments, a method of treating cognitive impairment associated with Alzheimer's disease, such as preventing the progression or disease modification of the Alzheimer's disease, in a patient in need thereof, comprising administering a pharmaceutical composition comprising an aminoisoxazoline compound represented by Formula (I), (Ia), or (Ib), or a pharmaceutically acceptable salt thereof.

In certain embodiments, a method of treating dementia of an Alzheimer's-type in a patient, such as preventing the progression or disease modification of the dementia of an Alzheimer's-type, in need thereof, comprising administering an aminoisoxazoline compound represented by Formula (I), (Ia), or (Ib), or a pharmaceutically acceptable salt thereof. For example, in certain embodiments, a method of treating dementia of an Alzheimer's-type in a patient, such as preventing the progression or disease modification of the dementia of an Alzheimer's-type, in need thereof, comprising administering a pharmaceutical composition comprising an aminoisoxazoline compound represented by Formula (I), (Ia), or (Ib), or a pharmaceutically acceptable salt thereof In certain embodiments, a method of treating cognitive impairment associated with dementia of an Alzheimer's-type in a patient, such as preventing the progression or disease modification of the dementia of an Alzheimer's-type, in need thereof, comprising administering an aminoisoxazoline compound represented by Formula (I), (Ia), or (Ib), or a pharmaceutically acceptable salt thereof. For example, in certain embodiments, a method of treating cognitive impairment associated with dementia of an Alzheimer's-type in a patient, such as preventing the progression or disease modification of the dementia of an Alzheimer's-type, in need thereof, comprising administering a pharmaceutical composition comprising an aminoisoxazoline compound represented by Formula (I), (Ia), or (Ib), or a pharmaceutically acceptable salt thereof.

In certain embodiments, a method of treating cognitive impairment associated with schizophrenia in a patient in need thereof, comprising administering an aminoisoxazoline compound represented by Formula (I), (Ia), or (Ib), or a pharmaceutically acceptable salt thereof In certain embodiments, a method of treating cognitive impairment associated with schizophrenia in a patient in need thereof, comprising administering a pharmaceutical composition comprising an aminoisoxazoline compound represented by Formula (I), (Ia), or (Ib), or a pharmaceutically acceptable salt thereof.

In certain embodiments, the aminoisoxazoline compounds represented by Formula (I), (Ia), or (Ib), or a pharmaceutically acceptable salt thereof, can, because of their pharmacological properties, be employed, alone or in combination with other active ingredients, for the treatment and/or prevention of cognitive impairments, for example, Alzheimer's disease, dementia of an Alzheimer's-type, or schizophrenia. Because of their selective effect as α7-nAChR agonists, the aminoisoxazoline compounds represented by Formula (I), (Ia), or (Ib), or a pharmaceutically acceptable salt thereof, are particularly suitable for improving cognition, providing procognitive effects, improving perception, improving concentration, improving learning or memory, improving one or more aspects of cognition, e.g., one or more of: executive function, memory (e.g., working memory), social cognition, visual learning, verbal learning and speed of processing, especially after or associated with cognitive impairments like those occurring for example in situations/diseases/syndromes such as mild cognitive impairment, age-associated learning and memory impairments, age-associated memory loss, vascular dementia, craniocerebral trauma, stroke, dementia occurring after strokes (post-stroke dementia), post-traumatic brain syndrome, post- traumatic stress disorder, general concentration impairments, concentration impairments in children with learning and memory problems, attention deficit hyperactivity disorder, autism spectrum disorder, Fragile X syndrome, Alzheimer's disease, Lewy body dementia, dementia with degeneration of the frontal lobes, including Pick's syndrome, frontotemporal dementia, Parkinson's disease, dyskinesias associated with dopamine agonist therapy in Parkinson's Disease, progressive nuclear palsy, dementia with corticobasal degeneration, amyotrophic lateral sclerosis (ALS), Huntington's disease, multiple sclerosis, thalamic degeneration, Creutzfeld-Jakob dementia, HIV dementia, schizophrenia (e.g., paranoid type, disorganized type, catatonic type, and undifferentiated type), schizophreniform disorder, schizoaffective disorder, delusional disorder, positive symptoms of schizophrenia, negative symptoms of schizophrenia, schizophrenia with dementia, Korsakoff s psychosis, depression, anxiety, mood and affective disorders, bipolar disorder, major depressive disorder, traumatic brain injury, chronic traumatic encephalopathy, withdrawal symptoms associated with smoking cessation and dependent drug cessation, Gilles de la Tourette's Syndrome, age-related macular degeneration, glaucoma, neurodegeneration associated with glaucoma, treatment of cognitive deficits following coronary artery bypass graft surgery, treatment (including amelioration, prevention or delay of progression) of sleep disorders (e.g., narcolepsy, excessive daytime sleepiness, nocturnal sleep disruption and/or cataplexy), cognitive deficits associated with sleep disorders, treatment (including amelioration, prevention or delay) of progression of fatigue, or use for facilitation of emergence from general anesthesia.

In certain embodiments, the aminoisoxazoline compounds represented by Formula (I), (Ia), or (Ib), or a pharmaceutically acceptable salt thereof, can be employed alone or in combination with other active ingredients for the prophylaxis and treatment of acute and/or chronic pain (for a classification, see “Classification of Chronic Pain, Descriptions of Chronic Pain Syndromes and Definitions of Pain Terms”, 2^{nd} edition, Meskey and Begduk, editors; IASP Press, Seattle, 1994), especially for the treatment of cancer-induced pain and chronic neuropathic pain like, for example, that associated with diabetic neuropathy, postherpetic neuralgia, peripheral nerve damage, central pain (for example as a consequence of cerebral ischaemia) and trigeminal neuralgia, and other chronic pain such as, for example, lumbago, backache, or rheumatic pain. In addition, these active ingredients are also suitable for the therapy of primary acute pain of any origin and of secondary states of pain resulting therefrom, and for the therapy of states of pain which were formerly acute and have become chronic.

In certain embodiments, the invention relates to a method comprising administering to a patient in need thereof, such as a patient suffering from, or diagnosed as having, a cognitive impairment or having one or more symptoms associated with a cognitive impairment, an effective dose of an aminoisoxazoline compound represented by Formula (I), (Ia), or (Ib), or a pharmaceutically acceptable salt thereof; or administering to the patient a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent. For example, the method may treat and/or improve the one or more symptoms associated with a cognitive impairment and/or the cognitive impairment. For example, in certain embodiments, the cognitive impairment is Alzheimer's disease, dementia of an Alzheimer's type, or schizophrenia.

A certain embodiment of the present invention provides a method of improving one or more cognitive symptoms, improving one or more behavioral symptoms, or both, associated with a cognitive impairment, comprising: administering to a patient in need thereof an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent. For example, in certain embodiments, the cognitive impairment is Alzheimer's disease, dementia of an Alzheimer's type, or schizophrenia.

In a certain embodiment of the present invention, the method provides a pro-cognitive effect in a patient suffering from, or diagnosed as having, a cognitive disease or dementia, comprising: administering to a patient in need thereof an effective dose of an aminoisoxazoline compound represented by Formula (I), (Ia), or (Ib), or a pharmaceutically acceptable salt thereof; or administering to the patient a pharmaceutical composition comprising an effective dose of the aminoisoxazoline compound represented by Formula (I), (Ia), or (Ib), or a pharmaceutically acceptable salt thereof, and at least one pharmaceutically acceptable carrier, excipient or diluent; wherein the method provides at least one of the following: visual motor, learning, delayed memory, or executive function; for example provides a pro-cognitive effect, exclusive of attention, in said patient; for example provides a pro-cognitive effect in at least one of the following: visual motor, learning, delayed memory, or executive function.

A certain embodiment of the present invention provides a method of treating a patient with a cognitive disease, comprising: administering to the patient a daily dose of a pharmaceutical composition comprising an aminoisoxazoline compound represented by Formula (I), (Ia), or (Ib), or a pharmaceutically acceptable salt thereof, and at least one pharmaceutically acceptable carrier, excipient or diluent.

In a certain embodiment of the present invention, the method provides a pro-cognitive effect in a patient suffering from, or diagnosed as having, schizophrenia, for example, paranoid type schizophrenia, disorganized type schizophrenia, catatonic type schizophrenia, undifferentiated type schizophrenia, schizophreniform disorder, schizoaffective disorder, delusional disorder, positive symptoms of schizophrenia, negative symptoms of schizophrenia, or schizophrenia with dementia, comprising: administering to a patient in need thereof an effective dose of an aminoisoxazoline compound represented by Formula (I), (Ia), or (Ib), or a pharmaceutically acceptable salt thereof; or administering to a patient in need thereof, a pharmaceutical composition comprising an effective dose of an aminoisoxazoline compound represented by Formula (I), (Ia), or (Ib), or a pharmaceutically acceptable salt thereof, and at least one pharmaceutically acceptable carrier, excipient or diluents; wherein the method provides at least one of the following: visual motor, learning, delayed memory, or executive function; for example provides a pro-cognitive effect, exclusive of attention, in said patient; for example provides a pro-cognitive effect in at least one of the following: visual motor, learning, delayed memory, or executive function.

In an embodiment of the present invention, any one of the above-noted embodiments, includes wherein the daily dose is an initial daily dose.

In a certain embodiment of the present invention provides a method of improving cognition of a patient in need thereof, comprising: administering to the patient an aminoisoxazoline compound represented by Formula (I), (Ia), or (Ib), or a pharmaceutically acceptable salt thereof; or administering to the patient a pharmaceutical composition comprising an effective dose of an aminoisoxazoline compound represented by Formula (I), (Ia), or (Ib), or a pharmaceutically acceptable salt thereof, and at least one pharmaceutically acceptable carrier, excipient or diluents. For example, in certain embodiments, the present invention provides a method of improving cognition in a patient suffering from Alzheimer's disease, dementia of an Alzheimer's type, or schizophrenia, comprises: administering to the patient an aminoisoxazoline compound represented by Formula (I), (Ia), or (Ib), or a pharmaceutically acceptable salt thereof; or administering to the patient a pharmaceutical composition comprising an effective dose of an aminoisoxazoline compound represented by Formula (I), (Ia), or (Ib), or a pharmaceutically acceptable salt thereof, and at least one pharmaceutically acceptable carrier, excipient or diluents.

In a certain embodiment of the present invention provides a method of treating or improving one or more symptoms associated with a cognitive disease and/or a cognitive impairment in a patient in need thereof, comprising: administering to the patient an effective dose of an aminoisoxazoline compound represented by Formula (I), (Ia), or (Ib), or a pharmaceutically acceptable salt thereof; or administering to the patient a pharmaceutical composition comprising the aminoisoxazoline compound represented by Formula (I), (Ia), or (Ib), or a pharmaceutically acceptable salt thereof, and at least one pharmaceutically acceptable carrier, excipient or diluent.

In an embodiment of the present invention, any one of the above-noted embodiments, wherein the method specifically includes treating a symptom associated with a cognitive disease.

In an embodiment of the present invention, any one of the above-noted embodiments, wherein the method specifically includes improving a symptom associated with a cognitive disease.

In an embodiment of the present invention, any one of the above-noted embodiments, wherein the method specifically includes preventing progression of a cognitive disease.

In an embodiment of the present invention, any one of the above-noted embodiments, wherein the patient has been diagnosed as having a cognitive disease.

In an embodiment of the present invention, any one of the above-noted embodiments, wherein the patient has been diagnosed as having Alzheimer's disease.

In an embodiment of the present invention, any one of the above-noted embodiments, wherein the method specifically includes treating a symptom associated with Alzheimer's disease.

In an embodiment of the present invention, any one of the above-noted embodiments, wherein the method specifically includes improving a symptom associated with Alzheimer's disease.

In an embodiment of the present invention, any one of the above-noted embodiments, wherein the method specifically includes preventing progression of Alzheimer's disease.

In an embodiment of the present invention, any one of the above-noted embodiments, wherein the method specifically includes disease modification of Alzheimer's disease.

In an embodiment of the present invention, any one of the above-noted embodiments, wherein the patient has been diagnosed as having mild-to-moderate Alzheimer's disease.

In an embodiment of the present invention, any one of the above-noted embodiments, wherein the patient has been diagnosed as having dementia of an Alzheimer's type.

In an embodiment of the present invention, any one of the above-noted embodiments, wherein the method specifically includes treating a symptom associated with schizophrenia.

In an embodiment of the present invention, any one of the above-noted embodiments, wherein the method specifically includes improving a symptom associated with schizophrenia.

In an embodiment of the present invention, any one of the above-noted embodiments, wherein the method specifically includes preventing progression of schizophrenia.

In an embodiment of the present invention, any one of the above-noted embodiments, wherein the patient has been diagnosed as having schizophrenia.

In an embodiment of the present invention, any one of the above-noted embodiments, wherein the method specifically includes treating a symptom associated with positive symptoms of schizophrenia.

In an embodiment of the present invention, any one of the above-noted embodiments, wherein the method specifically includes improving a symptom associated with positive symptoms of schizophrenia.

In an embodiment of the present invention, any one of the above-noted embodiments, wherein the method specifically includes preventing progression of positive symptoms of schizophrenia.

In an embodiment of the present invention, any one of the above-noted embodiments, wherein the method specifically includes the patient has been diagnosed as having positive symptoms of schizophrenia.

In an embodiment of the present invention, any one of the above-noted embodiments, wherein the method specifically includes treating a symptom associated with negative symptoms of schizophrenia.

In an embodiment of the present invention, any one of the above-noted embodiments, wherein the method specifically includes improving a symptom associated with negative symptoms of schizophrenia.

In an embodiment of the present invention, any one of the above-noted embodiments, wherein the method specifically includes preventing progression of negative symptoms of schizophrenia.

In an embodiment of the present invention, any one of the above-noted embodiments, wherein the method specifically includes the patient has been diagnosed as having negative symptoms of schizophrenia.

In an embodiment of the present invention, any one of the above-noted embodiments, wherein the method specifically includes treating a symptom associated with schizophrenia with dementia.

In an embodiment of the present invention, any one of the above-noted embodiments, wherein the method specifically includes improving a symptom associated with schizophrenia with dementia.

In an embodiment of the present invention, any one of the above-noted embodiments, wherein the method specifically includes preventing progression of schizophrenia with dementia.

In an embodiment of the present invention, any one of the above-noted embodiments, wherein the method specifically includes the patient has been diagnosed as having schizophrenia with dementia.

In an embodiment of the present invention, any one of the above-noted embodiments, wherein the method specifically includes treating a symptom associated with major depressive disorder.

In an embodiment of the present invention, any one of the above-noted embodiments, wherein the method specifically includes improving a symptom associated with major depressive disorder.

In an embodiment of the present invention, any one of the above-noted embodiments, wherein the method specifically includes preventing progression of major depressive disorder.

In an embodiment of the present invention, any one of the above-noted embodiments, wherein the method specifically includes the patient has been diagnosed as having major depressive disorder.

In an embodiment of the present invention, any one of the above-noted embodiments, wherein the method specifically includes the patient has been diagnosed as having a disease associated with chronic inflammation, including atherosclerosis, rheumatoid arthritis and inflammatory bowel diseases.

In an embodiment of the present invention, any one of the above-noted embodiments, wherein the pharmaceutical composition is in the form of a tablet.

Pharmaceutical Compositions

In certain embodiments, the invention also includes pharmaceutical preparations which, besides inert, nontoxic, pharmaceutically suitable excipients, adjuvants and carriers, contain one or more aminoisoxazoline compounds represented by Formula (I), (Ia), or (Ib), or a pharmaceutically acceptable salt thereof, or consist of one or more aminoisoxazoline compounds represented by Formula (I), (Ia), or (Ib), or a pharmaceutically acceptable salt thereof, and processes for producing these preparations.

An aminoisoxazoline compound represented by Formula (I), (Ia), or (Ib), or a pharmaceutically acceptable salt thereof, may be formulated for administration in solid or liquid form. For example, an aminoisoxazoline compound represented by Formula (I), (Ia), or (Ib), or a pharmaceutically acceptable salt thereof, may be formulated for administration in a capsule, a tablet, or a powder form. For example, an aminoisoxazoline compound represented by Formula (I), (Ia), or (Ib), or a pharmaceutically acceptable salt thereof, may be formulated alone or as part of a pharmaceutical composition, suitable for oral administration, such as in a capsule or tablet, intravenous administration, parenteral administration, topical administration, or transdermal administration, such as in a patch, to a patient in need thereof.

An aminoisoxazoline compound represented by Formula (I), (Ia), or (Ib), or a pharmaceutically acceptable salt thereof, may be administered as a pharmaceutical composition, for example, in the presence of carriers, adjuvants, excipients, diluents, fillers, buffers, stabilizers, preservatives, lubricants, and the like, for example, administered as a pharmaceutical composition (e.g., formulation) comprising at least an aminoisoxazoline compound represented by Formula (I), (Ia), or (Ib), or a pharmaceutically acceptable salt thereof, together with one or more pharmaceutically acceptable carriers, adjuvants, excipients, diluents, or other materials well known to those skilled in the art. As used herein, the term “pharmaceutically acceptable”, unless otherwise specified, includes the generally accepted meaning which encompasses combinations, compounds, materials, compositions, and/or dosage forms which are, within the scope of sound medical judgment, suitable for consumption by humans without excessive toxicity, irritation, allergic response, or other problem or complication, commensurate with a reasonable benefit/risk ratio.

Suitable pharmaceutically acceptable carriers, adjuvants, excipients, and diluents, can include, but are not limited to, lactose, dextrose, sucrose, sorbitol, mannitol, starches, gum, acacia, calcium phosphate, alginates, tragacanth, gelatin, calcium silicate, microcrystalline cellulose, polyvinylpyrrolidone, cellulose, water syrup, methyl cellulose, methyl and propyl hydroxybenzoates, talc, magnesium stearate, and mineral oil. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene- polyoxypropylene-block polymers, polyethylene glycol and wool fat.

The formulations can additionally include, but are not limited to, pharmaceutically acceptable lubricating agents, glidants, wetting agents, emulsifying and suspending agents, preserving agents, sweetening agents, and/or flavoring agents. The pharmaceutical compositions of the present invention may be formulated so as to provide quick release, immediate release, sustained release, or delayed release of an aminoisoxazoline compound represented by Formula (I), (Ia), or (Ib), or a pharmaceutically acceptable salt thereof, after administration to the patient by employing procedures well-known in the art.

Another embodiment of the invention further comprises methods of making Pharmaceutical Composition, comprising admixing at least an aminoisoxazoline compound represented by Formula (I), (Ia), or (Ib), or a pharmaceutically acceptable salt thereof, together with one or more pharmaceutically acceptable carriers, excipients, buffers, adjuvants, stabilizers, or other materials.

In certain embodiments, the aminoisoxazoline compounds represented by Formula (I), (Ia), or (Ib), or a pharmaceutically acceptable salt thereof, are to be present in these preparations in a concentration of from 0.1 to 99.5% by weight, preferably from 0.5 to 95% by weight, of the complete mixture. Besides the aminoisoxazoline compounds represented by Formula (I), (Ia), or (Ib), or a pharmaceutically acceptable salt thereof, the pharmaceutical preparations may also contain other active pharmaceutical ingredients.

In certain embodiments, the novel active ingredients can be converted in a known manner into conventional formulations such as tablets, coated tablets, pills, granules, aerosols, syrups, emulsions, suspensions and solutions, using inert, nontoxic, pharmaceutically suitable excipients or solvents. In these cases, the therapeutically active compound should in each case be present in a concentration of about 0.5 to 90% by weight of the entire mixture, i.e., in amounts which are sufficient to reach the stated dose range.

In certain embodiments, the formulations are produced, for example, by extending the active ingredients with solvents and/or excipients, where appropriate with use of emulsifiers and/or dispersants, it being possible for example when water is used as diluent where appropriate to use organic solvents as auxiliary solvents.

In certain embodiments, administration may take place in a conventional way, for example, orally, transdermally or parenterally, especially perlingually or intravenously. In certain embodiments, administration may also take place by inhalation through the mouth or nose, for example, with the aid of a spray, or topically via the skin.

In certain embodiments, the aminoisoxazoline compounds represented by Formula (I), (Ia), or (Ib), or a pharmaceutically acceptable salt thereof, may be administered in amounts of about 0.01 to 10 mg/kg, on oral administration, for example, about 0.05 to 5 mg/kg, of body weight to achieve effective results.

### EXAMPLES

Analytical Instrument Model:

TABLE 1

| | |
|---|---|
| LCMS | Shimadzu UFLC MS: LCMS-2020 |
| | Agilent Technologies 1200 series MS: |
| | Agilent Technologies 6110 |
| | Agilent Technologies 1200 series MS: LC/MSD VL |
| NMR | BRUKER ADVANCE III/400 (400 MHz) |
| Prep-HPLC | Gilson GX-281 systems: instruments GX-A, GX-B, |
| | GX-C, GX-D, GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

LCMS:

LCMS Conditions A (“LCMS (A)”): Instrument: Shimadzu LCMS 2020; Mobile phase A: 4L H2O\1.5 mL TFA, Mobile phase B: 4L ACN\0.75 mL TFA; Method name: 10-80AB_4MIN_2W; Flow Rate: 0.8 mL/min.; Gradient: 10%-80%; Column: Boston Green ODS 2.1×30 mm, 3 μm; Column temperature: 50° C.; Wavelength: 220 nm & 254 nm.

LCMS Conditions B (“LCMS (B)”): Instrument: Agilent 1200 Series; Mobile phase A: 4L H2O\1.5 ml TFA, Mobile phase B: 4L ACN\0.75 mL TFA; Method name: 5-95AB_R_2W; Flow Rate: 1.5 mL/min.; Gradient: 5%-95%; Column: Chromolith@Flash RP-18e 25×2 mm; Column temperature: 50° C.; Wavelength: 220 nm & 254 nm.

LCMS Conditions C (“LCMS (C)”): Instrument: Agilent 1200 Series; Mobile phase A: 4L H2O\2 mL NH3H2O; Mobile phase B: Acetonitrile; Method name: 5-95CD_4.5MIN_2W; Flow Rate: 0.8 mL/min.; Gradient: 5%-95%; Column: Chromolith@Flash RP-18e 25×2 mm; Column temperature 50° C.; Wavelength: 220 nm & 254 nm.

LCMS Conditions D (“LCMS (D)”): Instrument: Agilent 1200 Series; Mobile phase A: 4L H2O\1.5 mL TFA, Mobile phase B: 4L ACN\0.75 mL TFA; Method name: 5-95AB_R_4MIN_2W; Flow Rate: 0.8 mL/min.; Gradient: 5%-95%; Column: Chromolith@Flash RP-18e 25×2 mm; Column temperature: 50° C.; Wavelength: 220 nm & 254 nm.

LCMS Conditions E (“LCMS (E)”): Instrument: Agilent 1200 Series; Mobile phase A: 4L H2O\1.5 ml TFA, Mobile phase B: 4L ACN\0.75 mL TFA; Method name: 5-95AB_R; Flow Rate: 1.5 mL/min.; Gradient: 5%-95%; Column: Chromolith@Flash RP-18e 25×2 mm; Column temperature: 50° C.; Wavelength: 220 nm & 254 nm.

LCMS Conditions F (“LCMS (F)”): Instrument: Agilent 1200 Series; Mobile phase A: 4L H2O\2 ml NH3H2O, Mobile phase B: Acetonitrile; Method name: 5-95CD_2MIN_2W; Flow Rate: 1.2 mL/min.; Gradient: 5%-95%; Column: XBrige Shield RP-18 2.1×50 mm, 5 μm; Column temperature: 30° C.; Wavelength: 220 nm & 254 nm.

LCMS Conditions G (“LCMS (G)”): Instrument: Agilent 1200 Series; Mobile phase A: 4L H2O\2 mL NH3H2O, Mobile phase B: Acetonitrile; Method name: 10-80CD_4MIN_2W; Flow Rate: 0.8 mL/min.; Gradient: 10%-80%; Column: XBridge C-18 2.1×50 mm, 5 μm; Column temperature: 40° C.; Wavelength: 220 nm & 254 nm.

LCMS Conditions H (“LCMS (H)”): Instrument: Agilent 1200 Series; Mobile phase A: 4L H2O\1.5 mL TFA, Mobile phase B: 4L ACN\0.75 mL TFA; Method name: 10-80AB_4MIN_2W; Flow Rate: 0.8 mL/min.; Gradient: 10%-80%; Column: Xtimate C-18, 2.1×30 mm, 3 μm; Column temperature: 50° C.; Wavelength: 220 nm & 254 nm.

LCMS Conditions I (“LCMS (I)”): Instrument: Agilent 1200 Series; Mobile phase A: 4L H2O\2 mL NH3H2O, Mobile phase B:Acetonitrile; Method name:0-60CD_4.5MIN_2W; Flow Rate: 0.8 ml/min.; Gradient: 0%−60%; Column: XBrige Shield RP-18 2.1×50 mm, 5 μm; Column temperature 50° C.; Wavelength: 220 nm & 254 nm.

LCMS Conditions J (“LCMS (J)”): Instrument: Agilent 1200 Series; Mobile phase A: 4L H2O\2 mL NH3H2O, Mobile phase B: Acetonitrile; Method name: 10-80CD_2MIN_POS_2W; Flow Rate: 1.2m1/min.; Gradient: 10%-80%; Column: Xbridge C-18 2.1×50 mm, 5 μm; Column temperature: 40° C.; Wavelength: 220 nm & 254 nm.

LCMS Conditions K (“LCMS (K)”): Instrument: Shimadzu LCMS 2020; Mobile phase A: 4L H2O\1.5 mL TFA, Mobile phase B: 4L ACN\0.75 mL TFA; Method name: 0-30AB_2MIN_2W; Flow Rate: 1.2 mL/min.; Gradient: 0%−30%; Column: Chromolith@Flash RP-18E 25×2 mm; Column temperature: 50° C.; Wavelength: 220 nm & 254 nm.

LCMS Conditions L (“LCMS (L)”): Instrument: Shimadzu LCMS 2020; Mobile phase A: 4L H2O\1.5 mL TFA, Mobile phase B: 4L ACN\0.75 mL TFA;Method name: 0-30AB_4MIN_2W; Flow Rate: 0.8 mL/min.; Gradient: 0%−30%;Column: Chromolith@Flash RP-18E 25×2 mm; Column temperature: 50° C.; Wavelength: 220 nm & 254 nm.

LCMS Conditions M (“LCMS (M)”): Instrument: Shimadzu LCMS 2020; Mobile phase A: 4L H2O\1.5 mL TFA, Mobile phase B: 4L ACN\0.75 mL TFA; Method name: 0- 60AB_2MIN_2W; Flow Rate: 1.2 mL/min.; Gradient: 0%−60%; Column: Chromolith@Flash RP-18E 25×2 mm; Column temperature: 50° C.; Wavelength: 220 nm & 254 nm.

LCMS Conditions N (“LCMS (N)”): Instrument: Shimadzu LCMS 2020; Mobile phase A: 4L H2O\1.5 mL TFA, Mobile phase B: 4L ACN\0.75 mL TFA; Method name: 0- 60AB_4MIN_2W; Flow Rate: 0.8 mL/min.; Gradient: 0%−60%; Column: Chromolith@Flash RP-18E 25×2 mm; Column temperature: 50° C.; Wavelength: 220 nm & 254 nm.

LCMS Conditions 0 (“LCMS (0)”): Instrument: Agilent 1200 Series LCMS; Mobile phase A: 4L H2O\2 mL NH3H2O, Mobile phase B: CAN; Method name: 0-30CD_2MIN_POS_2W; Flow Rate: 1.0 mL/min.; Gradient: 0%−30%; Column: Xbridge C18 2.1×50 mm, 5 um; Column temperature: 40° C.; Wavelength: 220 nm & 254 nm.

LCMS Conditions P (“LCMS (P)”): Instrument: Agilent 1200 Series LCMS; Mobile phase A: 4L H2O\2 mL NH3H2O, Mobile phase B: CAN; Method name: 0-60CD_2MIN_POS_2W; Flow Rate: 1.0 mL/min.; Gradient: 0%−60%; Column: Xbridge C18 2.1×50 mm, 5 um; Column temperature: 40° C.; Wavelength: 220 nm & 254 nm.

LCMS Conditions Q (“LCMS (Q)”): Instrument: Agilent 1200 Series LCMS; Mobile phase A: 4L H2O\2 mL NH3H2O, Mobile phase B: CAN; Method name: 0-60CD_4MIN_2W; Flow Rate: 0.8 mL/min.; Gradient: 0%−60%; Column: Xbridge C18 2.1×50 mm, 5 um; Column temperature: 40° C.; Wavelength: 220 nm & 254 nm.

LCMS Conditions R (“LCMS (R)”): Instrument: Agilent 1200 Series LCMS; Mobile phase A: 4L H2O\1.5 mL TFA, Mobile phase B: 4L ACN\0.75 mL TFA; Method name: 10-80AB_2MIN_2W; Flow Rate: 1.2 mL/min.; Gradient: 10%-80%; Column: Xtimate C18, 2.1×30 mm, 3 um; Column temperature: 50° C.; Wavelength: 220 nm & 254 nm.

LCMS Conditions S (“LCMS (S)”): Instrument: Agilent 1200 Series LCMS; Mobile phase A: 4L H2O\2 mL NH3H2O, Mobile phase B: CAN; Method name: 30-90CD_4MIN_POS_2W; Flow Rate: 0.8 mL/min.; Gradient: 30%-90%; Column: Xbridge C18 2.1×50 mm, 5 um; Column temperature: 40° C.; Wavelength: 220 nm & 254 nm.

LCMS Conditions T (“LCMS (T)”): Instrument: Agilent 1200 Series LCMS; Mobile phase A: 4L H2O\1.5 mL TFA, Mobile phase B: 4L ACN\0.75 mL TFA; Method name: 5-95AB 15MIN YMC; Flow Rate: 1.0 mL/min.; Gradient: 5%-95%; Column: YMC-Pack ODS-A 5 μm 150×4.6 mm; Column temperature: 50° C.; Wavelength: 220 nm & 254 nm.

LCMS Conditions U (“LCMS (U)”): Instrument: Agilent 1200 Series LCMS; Mobile phase A: 4L H2O\1.5 mL TFA, Mobile phase B: 4L ACN\0.75 mL TFA; Method name: 0-30AB_2MIN_2W; Flow Rate: 1.2 mL/min.; Gradient: 0%−30%;Column: Chromolith@Flash RP-18E 25×2 mm; Column temperature: 50° C.; Wavelength: 220 nm & 254 nm.

LCMS Conditions V (“LCMS (V)”): Instrument: Agilent 1200 Series LCMS;Mobile phase A: 4L H2O\1.5 mL TFA, Mobile phase B: 4L ACN\0.75 mL TFA;Method name: 0-30AB_4MIN_2W; Flow Rate: 0.8 mL/min.; Gradient: 0%−30%;Column: Chromolith@Flash RP-18E 25×2 mm; Column temperature: 50° C.; Wavelength: 220 nm & 254 nm.

LCMS Conditions W (“LCMS (W)”): Instrument: Agilent 1200 Series LCMS; Mobile phase A: 4L H2O\1.5 mL TFA, Mobile phase B: 4L ACN\0.75 mL TFA; Method name: 0-60AB_2MIN_2W; Flow Rate: 1.2 mL/min.; Gradient: 0%−60%; Column: Chromolith@Flash RP-18E 25×2 mm; Column temperature: 50° C.; Wavelength: 220 nm & 254 nm.

LCMS Conditions X (“LCMS (X)”): Instrument: Agilent 1200 Series LCMS; Mobile phase A: 4L H2O\1.5 mL TFA, Mobile phase B: 4L ACN\0.75 mL TFA; Method name: 0-60AB_4MIN_2W; Flow Rate: 0.8 mL/min.; Gradient: 0%−60%; Column: Chromolith@Flash RP-18E 25×2 mm; Column temperature: 50° C.; Wavelength: 220 nm & 254 nm.

LCMS Conditions Y (“LCMS (Y)”): Instrument: Shimadzu LCMS 2020; Mobile phase A: 4L H2O\1.5 ml TFA, Mobile phase B: 4L ACN\0.75 mL TFA; Method name: 5-95AB_R_2W; Flow Rate: 1.5 mL/min.; Gradient: 5%-95%; Column: Chromolith@Flash RP-18e 25×2 mm; Column temperature: 50° C.; Wavelength: 220 nm & 254 nm.

LCMS Conditions Z (“LCMS (Z)”): Instrument: Shimadzu LCMS 2020; Mobile phase A: 4L H2O\1.5 mL TFA, Mobile phase B: 4L ACN\0.75 mL TFA; Method name: 5-95AB_R_4MIN_2W; Flow Rate: 0.8 mL/min.; Gradient: 5%-95%; Column: Chromolith@Flash RP-18e 25×2 mm; Column temperature: 50° C.; Wavelength: 220 nm & 254 nm.

LCMS Conditions AA (“LCMS (AA)”): Instrument: Agilent 1200 Series LCMS; Mobile phase A: 4L H2O\2 mL NH₃·H₂O, Mobile phase B: ACN; Method name: 10-80CD_2MIN_NEG; Flow Rate: 1.2 mL/min.; Gradient: 10%-80%; Column: Xbridge C18 2.1×50 mm, 5 μm; Column temperature: 40° C.; Wavelength: 220 nm & 254 nm.

LCMS Conditions BB (“LCMS (BB)”): Instrument: Agilent 1200 Series LCMS; Mobile phase A: 4L H2O\1.5 mL TFA, Mobile phase B: 4L ACN\0.75 mL TFA;Method name: 0-60AB_R_2W; Flow Rate: 1.5 mL/min.; Gradient: 0%−60%;Column: Chromolith@Flash RP-18E 25×2 mm; Column temperature: 50° C.; Wavelength: 220 nm & 254 nm.

LCMS Conditions CC (“LCMS (CC)”): Instrument: Agilent 1200 Series LCMS; Mobile phase A: 4L H2O\1.5 mL TFA, Mobile phase B: 4L ACN\0.75 mL TFA;Method name: 0-30AB_R_2W; Flow Rate: 1.5 mL/min.; Gradient: 0%−30%;Column: Chromolith@Flash RP-18E 25×2 mm; Column temperature: 50° C.; Wavelength: 220 nm & 254 nm.

LCMS Conditions DD (“LCMS (DD)”): Instrument: Agilent 1200 Series LCMS; Mobile phase A: 4L H2O\1.5 mL TFA, Mobile phase B: 4L ACN\0.75 mL TFA;Method name: 10-80AB_R_2W; Flow Rate: 1.5 mL/min.; Gradient: 10%-80%;Column: Chromolith@Flash RP-18E 25×2 mm; Column temperature: 50° C.; Wavelength: 220 nm & 254 nm.

LCMS Conditions EE (“LCMS (EE)”): Instrument: Agilent 1200 Series; Mobile phase A: 1L H2O\0.375 mL TFA, Mobile phase B: 4L ACN\0.75 mL TFA; Method name: WUXIAB00; Flow Rate: 0.6 -1.0 mL/min; Gradient: 0%-80%-100%; Column: Agilent 5 TC-C18 50×2.1 mm; Column temperature: 50° C.; Wavelength: 220 nm & 254 nm.

LCMS Conditions FF (“LCMS (FF)”): Instrument: Agilent 1200 Series; Mobile phase A: 1L H2O\0.375 mL TFA, Mobile phase B: 4L ACN\0.75 mL TFA; Method name: WUXIAB01; Flow Rate: 0.8 -1.0 mL/min; Gradient: 1%-90%-100%; Column: Agilent 5 TC-C18 50×2.1 mm; Column temperature: 50° C.; Wavelength: 220 nm & 254 nm.

LCMS Conditions GG (“LCMS (GG)”): Instrument: Agilent 1200 Series; Mobile phase A: 1L H2O\0.375 mL TFA, Mobile phase B: 4L ACN\0.75 mL TFA; Method name: WUXIAB10; Flow Rate: 0.8 -1.0 mL/min; Gradient: 10%-100%; Column: Agilent 5 TC-C18 50×2.1 mm; Column temperature: 50° C.; Wavelength: 220 nm & 254 nm.

LCMS Conditions HH (“LCMS (HH)”): Instrument: Agilent 1200 Series; Mobile phase A: 4L H2O\2 mL NH₃·H₂O, Mobile phase B: ACN; Method name: 0-60CD_2MIN_NEG; Flow Rate: 1.0 mL/min.; Gradient: 0%−60%; Column: Xbridge C18 2.1×50 mm, 5 μm; Column temperature: 40° C.; Wavelength: 220 nm & 254 nm.

Instrument: Agilent 1100 Bin. Pump: G1312A, degasser; autosampler, ColCom, DAD: Agilent G1315B, 210 nm, MSD: Agilent LC/MSD G1956B ESI, pos/neg 100-800; MS parameters: Source: ESI, Capillary voltage: 3000V, Drying gas flow: 12 L/min., Nebulizer Pressure 60 psig, Drying Gas Temperature: 350° C., Fragmentor 70, MS scan: MS range 100-800 (positive and negative mode), Flow into MS 0.4 mL/min.; Mobile phase A: 95% acetonitrile +5% 10 mM ammonium bicarbonate in water; Mobile phase B: 10 mM ammonium bicarbonate in water pH=9.0; Flow Rate: 0.8 mL/min; Linear Gradient: t=0 min 5% A, t=3.5 min 98% A, t=6 min 98% A; Column: Phenomenex Gemini NX (C18, 50×2.0 mm, particle size: 3 μm); Column temperature: 25° C.; Detection DAD: Wavelength 220-320 nm.

Preparative HPLC (1): MS instrument type: Agilent Technologies G1956B Quadrupole LC-MS; HPLC instrument type: Agilent Technologies 1200 preparative LC; column: Phenomenex Gemini-NX(C18, 100×21.2 mm, 10 μ); flow: 25 ml/min; column temp: RT; eluent A: 99% acetonitrile +1% 10 mM ammonium bicarbonate in water pH=9.0, eluent B: 10mM ammonium bicarbonate in water pH=9.0; detection: DAD (220-320 nm); detection: MSD (ESI pos/neg) mass range: 100-800; fraction collection based on MS and DAD.

Chiral preparative HPLC instrument (“cHPLC1”): HPLC instrument modules: Shimadzu LC8-A preparative pumps, Shimadzu SCL-10Avp system controller, Shimadzu SPD-10Avp UV-VIS detector; Fraction Collector: Gilson 215 Liquid Handler.

cHPLC Analytical Conditions 1 (“cHPLC analytical (1)”): Instrument: cHPLC 2, Flow: 1 mL/min, isocratic, time: 30 min., Column temp: 25° C. Column: Chiralpak IC, 250×4.6 mm, 5 μ; Eluent: heptane+0.1% diethylamine/ethanol=7/3.

cHPLC Analytical Conditions 2 (“cHPLC analytical (2)”): Instrument: cHPLC 2, Flow: 1 mL/min, isocratic, time: 30 min., Column temp: 25° C. Column: Chiralpak IC, 250×4.6 mm, 5 μ; Eluent: heptane+0.1% diethylamine/ethanol=8/2.

cHPLC Analytical Conditions 3 (“cHPLC analytical (3)”): Instrument: cHPLC 2, Flow: 1 mL/min, isocratic, time: 30 min., Column temp: 25° C. Column: Chiralpak IC, 250×4.6 mm, 5 μ; Eluent: heptane+0.1% diethylamine/ethanol=9/1.

cHPLC Analytical Conditions 4 (“cHPLC analytical (4)”): Instrument: cHPLC 2, Flow: 1 mL/min, isocratic, time: 30 min., Column temp: 25° C. Column: Chiralpak IA, 250×4.6 mm, 5 μ; Eluent: heptane+0.1% diethylamine/ethanol=7/3.

GCMS:

GCMS Conditions Instrument: SHIMADZU GCMS-QP2010 Ultra; Carrier gas: He; Column Flow: 1.5 mL/min; Injector: 250° C.; Split Ratio:100:1; Column: HP-5MS 15m×0.25 mm×0.25 μm; FILM From: 40° C. (holding 3min) to 250° C. (holding 3min) at the rate of 25 ° C./min.

cSFC Analytical:

cSFC Analytical Conditions: Flow rate: 3 mL/min; Wavelength: 220 nm; and Column temperature: 35° C., were used for each of the specified conditions below:

cSFC Analytical Conditions A (“cSFC analytical (A)”): Column: Chiralcel OD-3 100×4.6 mm I.D., 3 um ; Mobile phase: ethanol (0.05% diethylamine (“DEA”) in CO₂ from 5% to 40%.

cSFC Analytical Conditions B (“cSFC analytical (B)”): Column: Chiralcel OD-3 100×4.6 mm I.D., 3 um; Mobile phase: methanol (0.05% DEA) in CO₂ from 5% to 40%.

cSFC Analytical Conditions C (“cSFC analytical (C)”): Column: Chiralcel OD-3 100×4.6 mm I.D., 3 um; Mobile phase: 40% ethanol (0.05% DEA) in CO₂.

cSFC Analytical Conditions D (“cSFC analytical (D)”): Column: Chiralpak AY-3 100×4.6 mm I.D., 3 um; Mobile phase: ethanol (0.05% DEA) in CO₂ from 5% to 40%.

cSFC Analytical Conditions E (“cSFC analytical (E)”): Column: Chiralcel OJ-3 100×4.6 mm I.D., 3 um; Mobile phase: ethanol (0.05% DEA) in CO₂ from 5% to 40%.

cSFC Analytical Conditions F (“cSFC analytical (F)”): Column: Chiralcel OJ-3 100×4.6 mm I.D., 3 um; Mobile phase: methanol (0.05% DEA) in CO₂ from 5% to 40%.

cSFC Analytical Conditions G (“cSFC analytical (G)”): Column: Chiralpak AD-3 100×4.6 mm I.D., 3 um; Mobile phase: ethanol (0.05% DEA) in CO₂ from 5% to 40%.

cSFC Analytical Conditions H (“cSFC analytical (H)”): Column: Chiralpak AD-3 100×4.6 mm I.D., 3 um; Mobile phase: methanol (0.05% DEA) in CO₂ from 5% to 40%.

cSFC Analytical Conditions I (“cSFC analytical (I)”): Column: Chiralpak AS-3 100×4.6 mm I.D., 3 um; Mobile phase: ethanol (0.05% DEA) in CO₂ from 5% to 40%.

cSFC Analytical Conditions J (“cSFC analytical (J)”): Column: Chiralcel AD-3 100×4.6 mm I.D., 3 um; Mobile phase: 15% iso-propanol (0.05% DEA) in CO₂.

cSFC Analytical Conditions K (“cSFC analytical (K)”): Column: Chiralcel AD-3 100×4.6 mm I.D., 3 um; Mobile phase: 40% iso-propanol (0.05% DEA) in CO₂

cSFC Analytical Conditions L (“cSFC analytical (L)”): Column: Chiralcel AD-3 100×4.6 mm I.D., 3 um; Mobile phase: 60% ethanol (0.05% DEA) in CO₂.

cSFC Analytical Conditions M (“cSFC analytical (M)”): Column: Chiralpak IC-3 100×4.6 mm I.D., 3 um; Mobile phase: 40% methanol (0.05% DEA) in CO₂.

cSFC Analytical Conditions N (“cSFC analytical (N)”): Column: Chiralpak IC-3 100×4.6 mm I.D., 3 um; Mobile phase: methanol (0.05% DEA) in CO₂ from 5% to 40%.

cSFC Analytical Conditions 0 (“cSFC analytical (0)”): Column: Chiralpak AS-3 100×4.6 mm I.D., 3 um; Mobile phase: 40% ethanol (0.05% DEA) in CO₂.

cSFC Analytical Conditions P (“cSFC analytical (P)”): Column: Chiralpak AS-3 150×4.6 mm I.D., 3 um; Mobile phase: ethanol (0.05% DEA) in CO₂ from 5% to 40%.

cSFC Analytical Conditions Q (“cSFC analytical (Q)”): Column: Chiralpak AS-3 100×4.6 mm I.D., 3 um; Mobile phase: methanol (0.05% DEA) in CO₂ from 5% to 40%.

cSFC Analytical Conditions R (“cSFC analytical (R)”): Column: Chiralpak AS-3 100×4.6 mm I.D., 3 um; Mobile phase: 40% methanol (0.05% DEA) in CO₂.

cSFC Analytical Conditions S (“cSFC analytical (S)”): Column: Chiralpak AS-3 100×4.6 mm I.D., 3 um; Mobile phase: 30% ethanol (0.05% DEA) in CO₂.

For each final compound prepared below that indicates the presence of a salt associated with the final compound (i.e., a salt complex), the specific molar equivalence of salt included in the final compound, unless specified, was not determined.

### Example 1A

### 2-(quinuclidin-3-ylidene)acetonitrile (A-1)

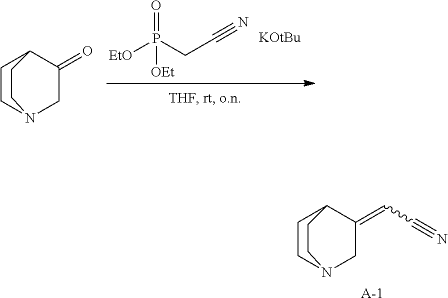

A solution of diethyl cyanomethylphosphonate (8.3 g, 46.9 mmol) in anhydrous tetrahydrofuran (100 mL) was degassed with nitrogen. Subsequently, potassium tert-butoxide (5.3 g, 46.9 mmol) was added at room temperature, and the reaction mixture was degassed with nitrogen and stirred for 20 minutes. Then, quinuclidin-3-one (5.9 g, 46.9 mmol) in anhydrous tetrahydrofuran (10 mL) was added. After stirring for 20 hours, the reaction mixture was concentrated, affording a brown oil. The residue was suspended in chloroform (500 mL), washed with saturated NaHCO₃ (3×250 mL), dried over Na₂SO₄ and concentrated in vacuo to afford compound A-1 (6.8 g, 95% yield) as a sticky brown solid. LCMS (1): tR=2.157/2.244 min. (major/minor isomer), (ES⁺) m/z (M+H)⁺=149.2. ¹H NMR (300 MHz, Chloroform-d, major isomer) δ 5.11 (t, J=2.1 Hz, 1H), 3.59-3.51 (m, 2H), 3.15-3.04 (m, 1H), 3.04-2.74 (m, 4H), 1.95-1.78 (m, 2H), 1.80-1.62 (m, 2H).

### Example 2A

### 4H-1′-azaspiro[isoxazole-5,3′-bicyclo[2.2.2]octan]-3-amine (rac-A-2)

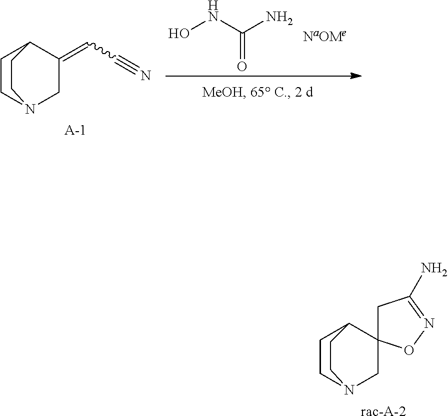

To a solution of sodium methoxide in methanol (12.5 wt%, 15.4 mL, 67.1 mmol) was added 1-hydroxyurea (4.7 g, 61.5 mmol) and compound A-1 (8.3 g, 55.9 mmol) in methanol (15 mL). The reaction mixture was stirred at 65° C. for two days, cooled to room temperature, filtered, concentrated onto silica (8 g) and purified by silica gel column chromatography [chloroform: 7M NH₃ in methanol=1:0 to 9:1] to afford compound rac-A-2 (7.5 g, 49% yield) as an off-white solid. LCMS (1): tR=0.607 min., (ES⁺) m/z (M+H)⁺=182.0.

Chiral Separation:

rac-A-2 (2.0 g, 11.0 mmol) in 40 mL of methanol/chloroform (1:1) was separated by preparative chiral HPLC [Instrument: cHPLC1, Flow: 18 mL/min, isocratic, time: 30 min., Column temp: 25° C., Column: Chiralpak IC, 20×250 mm, 5 μ, Eluent: heptane+0.2% diethylamine/ethanol=7/3)] to give:

(S)-4H-1′-azaspiro[isoxazole-5,3¹-bicyclo[2.2.2]octan]-3-amine (compound A-2-P1 (0.76 g, 38% yield) as an off-white solid: cHPLC analytical (1): tR=15.89 min., purity: 100%; LCMS (1): tR=0.600 min., (ES⁺) m/z (M+H)⁺=182.2; 1H NMR (300 MHz, Chloroform-d) 6 3.93 (s, 2H), 3.24 (dd, J=14.5, 2.0 Hz, 1H), 3.05 (d, J=15.8 Hz, 1H), 2.99-2.58 (m, 6H), 2.21-2.04 (m, 1H), 2.04-1.95 (m, 1H), 1.74-1.33 (m, 3H); Absolute configuration of compound A-2-P1 determined to be S by X-ray analysis in Example 86.

(R)-4H-1′-azaspiro[isoxazole-5,3¹-bicyclo[2.2.2]octan]-3-amine (compound A-2-P2) (0.60 g, 30% yield) as an off-white solid: cHPLC analytical (1): tR=19.44 min., purity: 100%; LCMS (1): tR=0.506 min., (ES⁺) m/z (M+H)⁺=182.2; 1H NMR (300 MHz, Chloroform-d) 6 3.98 (s, 1H), 3.23 (dd, J=14.5, 2.0 Hz, 1H), 3.05 (d, J=15.8 Hz, 1H), 2.99-2.63 (m, 6H), 2.21-2.04 (m, 1H), 2.04-1.95 (m, 1H), 1.80-1.31 (m, 3H); Absolute configuration of compound A-2-P2 determined to be R in view of the X-ray analysis of compound A-2-P1 in Example 86.

### Example 1B

### 2-bromobenzo[b]thiophene (B-1)

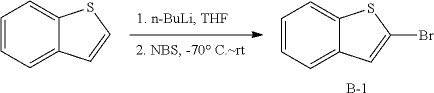

To a solution of benzo[b]thiophene (2.0 g, 15 mmol) in tetrahydrofuran (20 mL) at −70° C. under nitrogen was added dropwise n-butyllithium (2.5 M in hexane, 12 mL, 30 mmol). The reaction mixture was stirred at this temperature for 30 min. Then N-bromosuccinimide (5.3 g, 30 mmol) was added, and the resulting solution was allowed to warm from −70° C. to room temperature over 1 hour. On completion, the mixture was quenched with saturated aqueous ammonium chloride (20 mL) and extracted with ethyl acetate (3×30 mL). The combined organic phases were concentrated in vacuo, and the residue was purified by silica gel chromatography [petroleum ether: ethyl acetate=100:1] to give compound B-1 (0.50 g, 16% yield) as a white solid.

### Example 2B

### 2-bromobenzo[d]oxazole (B-2)

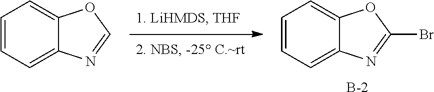

To a solution of benzo[d]oxazole (1.0 g, 8.4 mmol) in tetrahydrofuran (10 mL) at −25° C. under nitrogen was added dropwise lithium bis(trimethylsilyl)amide (1.0 M in tetrahydrofuran, 10 mL, 10 mmol). The reaction mixture was stirred at this temperature for 1 hour. Then N-bromosuccinimide (2.2 g, 13 mmol) was added, and the resulting solution was allowed to warm from −25° C. to room temperature over 1 hour. On completion, the mixture was quenched with saturated aqueous ammonium chloride (20 mL) and extracted with ethyl acetate (3×30 mL). The combined organic phases were concentrated in vacuo, and the residue was purified by silica gel chromatography [petroleum ether: ethyl acetate=100:1] to give compound B-2 (1.2 g, 72% yield) as a white oil. ¹H-NMR (CD₃OD, 400 MHz): δ 7.70-7.68 (m, 1H), 7.65-7.63 (m, 1H), 7.45-7.39 (m, 2H).

### Example 3B

### 6-cyclopropylbenzo[d]thiazole (B-3)

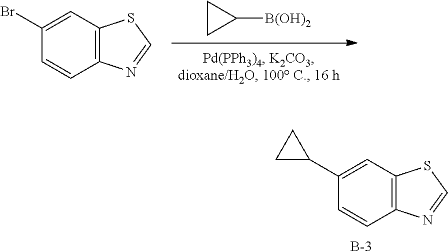

To a solution of 6-bromobenzo[d]thiazole (3.5 g, 16 mmol), cyclopropylboronic acid (2.1 g, 25 mmol) and potassium carbonate (6.8 g, 49 mmol) in dioxane (30 mL) and water (6 mL) was added tetrakis(triphenylphosphine)palladium(0) (1.9 g, 1.6 mmol). The solution was degassed and purged with nitrogen 3 times. The mixture was stirred at 100° C. for 16 hours, then diluted with water (10 mL) and extracted with ethyl acetate (3×30 mL). The combined organic layers were concentrated and purified by silica gel chromatography [petroleum ether: ethyl acetate=50:1] to give compound B-3 (1.0 g, 35% yield) as a yellow oil. ¹H-NMR (CDCl₃, 400 MHz): δ 8.92 (s, 1H), 8.03 (d, J=8.4 Hz, 1H), 7.68 (s, 1H), 7.29-7.24 (m, 1H), 2.10-2.05 (m, 1H), 1.09-1.05 (m, 2H), 0.82-0.78 (m, 2H).

### Example 4B

### 2-bromo-6-cyclopropylbenzo[a]thiazole (B-4)

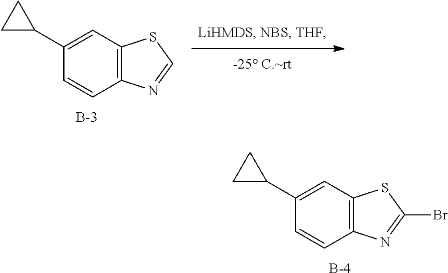

To a solution of compound B-3 (1.0 g, 5.7 mmol) in tetrahydrofuran (10 mL) at −25° C. was added dropwise lithium bis(trimethylsilyl)amide (lmol/L in tetrahydrofuran, 11 mL). The solution was stirred at −25° C. for 1 hour. Then N-bromosuccinimide (2.0 g, 11 mmol) was added at −25° C., and the reaction was allowed to warm to 25° C. and stirred for another 4 hours. On completion the reaction was quenched with methanol (5 mL) and concentrated in vacuo. The residue was purified by column chramotagraphy [petroleum ether: ethyl acetate=15:1] to give compound B-4 (0.80 g, 55% yield) as a yellow oil. LCMS (J): tR=0.657 min., (ES⁺) m/z (M+H)⁺=254.0

### Example 5B

### 5-chloro-6-fluorobenzo[d]thiazole-2(3H)-thione (B-5)

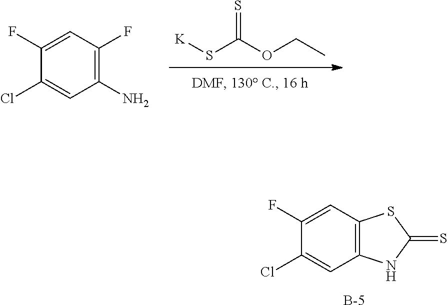

A solution of 5-chloro-2,4-difluoro-aniline (5.0 g, 31 mmol) and potassium O-ethyl carbonodithioate (12 g, 73 mmol) in N, N-dimethylformamide (50 mL) was stirred at 130° C. for 16 hours. On completion, the reaction was cooled to room temperature, and 1N hydrochloric acid (100 mL) was added. The mixture was stirred for 1 hour, resulting in formation of a solid. The solid was collected by filtration, washed with water (2x100 mL) and dried under vacuum to give compound B-5 (5.6 g, 79% yield) as a white solid. LCMS (B): tR=0.759 min., (ES⁺) m/z (M+H)⁺=220.1.

### Example 6B

### 2,5 -dichloro-6-fluorobenzo[d]thiazole (B-6)

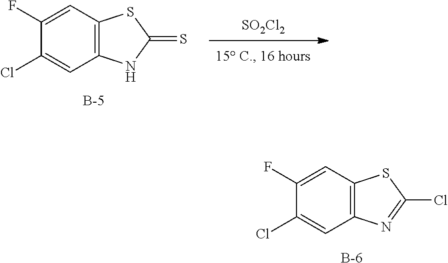

A solution of compound B-5 (2.0 g, 9.1 mmol) in sulfonyl chloride (34 g, 0.25 mol) was stirred at 15° C. for 16 hours. On completion, the solution was poured into ice-water (30 mL) and extracted with ethyl acetate (3×50 mL). The combined organic layers were concentrated in vacuo, and the residue was purified by column chramotagraphy [petroleum ether: ethyl acetate=20:1] to give compound B-6 (1.8 g, 89% yield) as a white solid. ¹H-NMR (CDCl₃, 400 MHz): δ 8.01 (d, J=6.8 Hz, 1H), 7.58 (d, J=8.0 Hz, 1H).

### Example 7B

### 2-chloroquinazoline (B-7)

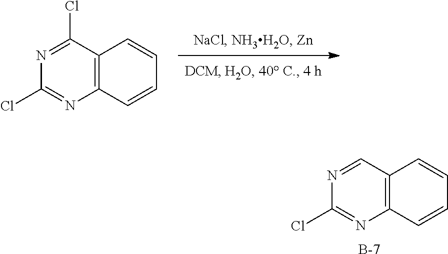

To a mixture of 2,4-dichloroquinazoline (5.0 g, 25 mmol), sodium chloride (0.7 g, 13 mmol) and ammonium hydroxide (27.3 g, 779 mmol) in dichloromethane (100 mL) and water (70 ml) was added zinc (4.93 g, 75.4 mmol). The mixture was stirred at 40° C. for 4 hour under nitrogen, then poured into water and extracted with ethyl acetate. The organic layer was dried over anhydrous sodium sulfate and concentrated in vacuo. The residue was purified by silica gel chromatography [petroleum ether: ethyl acetate=5:1] to give compound B-7 (2.0 g, 43% yield) as a white solid. ¹H-NMR (CD₃OD, 400 MHz): δ 9.46 (s, 1H), 7.16 (d, J=8.4 Hz, 1H), 8.07 (t, J=8.4 Hz, 1H), 7.95 (d, J=8.4 Hz, 1H), 7.78 (t, J=7.6 Hz, 1H).

### Example 8B

### N-(2-chlorobenzyl)-2,2-dimethoxyacetamide (B-8)

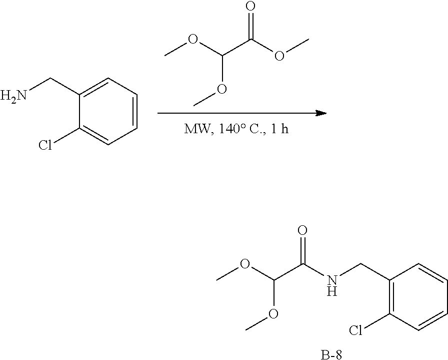

A mixture of (2-chlorophenyl)methanamine (2.0 g, 14 mmol) and methyl 2,2-dimethoxyacetate (3.8 g, 28 mmol) was stirred at 140° C. for 1 hour in the microwave. On completion, the mixture was diluted with dichloromethane (200 mL) and concentrated in vacuo to give compound B-8 (4.5 g, crude) as a yellow gum.

### Example 9B

### 8-chloroisoquinolin-3-ol (B-9)

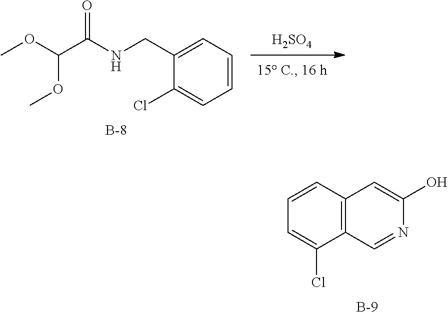

Compound B-8 (1.2 g, crude) was added to concentrated sulfuric acid (15 mL) at 15° C. The mixture was stirred at this temperature for 16 hours, then poured into ice-water (20 mL), filtered and adjusted to pH 9 with 33% ammonium hydroxide. The resulting precipitate was collected by filtration, dried in vacuo and purified by silica gel chromatography [dichloromethane: methanol=10:1] to give compound B-9 (0.75 g, crude) as a yellow solid. LCMS (M): tR=0.807 min., (ES⁺) m/z (M+H)⁺=180.0, ¹H-NMR (CD₃OD, 400 MHz): 8.95 (s, 1H), 7.56 (d, J=8.4 Hz, 1H), 7.46 (t, J=7.2 Hz, 1H), 7.32 (d, J=7.6 Hz, 1H), 6.93 (s, 1H).

### Example 10B

### 8-chloroisoquinolin-3-yltrifluoromethanesulfonate (B-10)

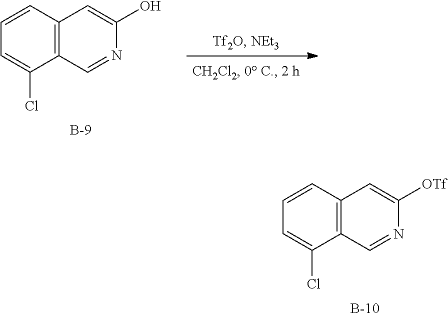

To a solution of compound B-9 (0.45 g, 2.5 mmol), triethylamine (0.51 g, 5.0 mmol) in dichloromethane (10 mL) at 0° C. was added dropwise trifluoromethanesulfonic anhydride (0.92 g, 3.3 mmol). The mixture was stirred at 0° C. for 2 hours, then diluted with dichloromethane (40 mL) and washed with water (20 mL), sodium bicarbonate solution (20 mL) and brine (20 mL). The organic layer was concentrated in vacuo and purified by silica gel chromatography [petroleum ether: ethyl acetate=10:1] to give compound B-10 (0.35 g, 45% yield) as a pale yellow solid. LCMS (B): tR=0.996 min., (ES⁺) m/z (M+H)⁺=312.0.

### Example 11B

### 3-chloro-5-nitroisoquinoline (B-11)

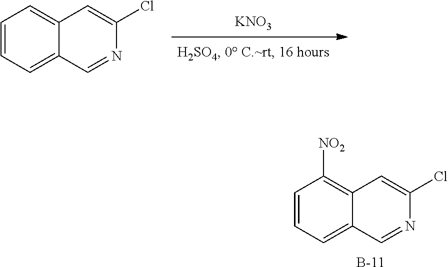

To a solution of 3-chloroisoquinoline (0.50 g, 3.1 mmol) in concentrated sulfuric acid (10 mL) at 0° C. was added a solution of potassium nitrate (0.34 g, 3.4 mmol) in concentrated sulfuric acid (5 mL). The mixture solution was stirred at 0° C. for 2 hours and at room temperature for another 14 hours. On completion, the mixture was poured into ice-water (30 mL), resulting in formation of a precipitate. The precipitate was collected by filtration and dried in vacuo to give compound B-11 (0.6 g, crude) as a yellow solid. ¹H-NMR (CDC13, 400 MHz): δ 9.25 (s, 1H), 8.70-8.66 (m, 2H), 8.35 (d, J=8 Hz, 1H), 7.76 (t, J=8 Hz, 1H).

### Example 12B

### 3-chloroisoquinolin-5-amine (B-12)

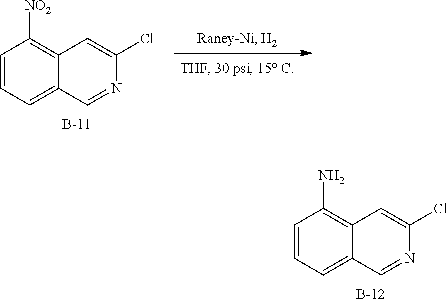

A mixture of compound B-11 (2.3 g, 11 mmol) and Raney-Ni (0.94 g, 11 mmol) in tetrahydrofuran (40 mL) was stirred at 15° C. under 30 psi hydrogen for 6 hours. On completion, the mixture was filtered and concentrated in vacuo to give compound B-12 (1.97 g, crude) as a brown solid. LCMS (J): tR=0.904 min., (ES⁺) m/z (M+H)⁺=179.0.

### Example 13B

### 3,5-dichloroisoquinoline (B-13)

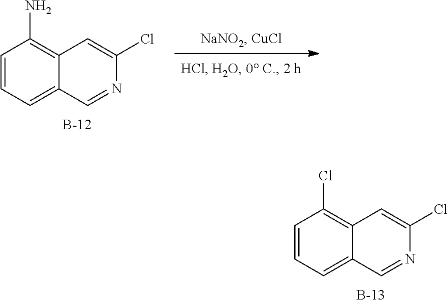

To a solution of compound B-12 (0.45 g, 2.5 mmol) in 37% hydrochloric acid (10 mL) at 0° C. was added a solution of sodium nitrite (0.26 g, 3.8 mmol) in water (7 mL). The mixture was stirred at 0° C. for 1 hour. Then a solution of cuprous chloride (0.37 g, 3.8 mmol) in 37% hydrochloric acid (5 mL) was added and stirring was continued at 0° C. for another 1 hour. On completion, the solution was adjusted to pH 12 with 1 N aqueous sodium hydroxide and extracted with ethyl acetate (3×150 mL). The combined organic layers were concentrated in vacuo, and the residue purified by silica gel chromatography [petroleum ether: ethyl acetate=20:1] to give compound B-13 (0.15 g, 30% yield) as a white solid. LCMS (J): tR=1.491 min., (ES⁺) m/z (M+H)⁺=198.0.

### Example 14B

### 5-methylbenzo[d]thiazole (B-14)

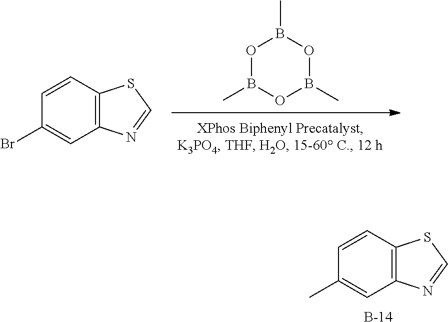

A mixture of 5-bromobenzo[d]thiazole (0.34 g, 1.6 mmol), 2,4,6-trimethyl-1,3,5,2,4,6-trioxatriborinane (0.60 g, 4.8 mmol), dicyclohexyl43-(2,4,6-triisopropylphenyl)phenyllphosphane [2-(2-aminophenyl)phenyl]-chloro-palladium; (0.06 g, 0.08 mmol) and potassium phosphate (0.67 g, 3.2 mmol) in tetrahydrofuran (12 mL) and water (3 mL) at 15° C. was degassed and purged with nitrogen 3 times. The mixture was stirred at 60° C. for 12 hours under nitrogen, then diluted with ethyl acetate (250 mL) and washed with brine (6×20 mL), dried over anhydrous sodium sulfate, filtered and concentrated in vacuo to give compound B-14 (0.27 g, 78% yield) as brown gum, which used directly without further purification. LCMS (Y): tR=0.633 min., (ES⁻) m/z (M+H)⁺=150.0.

### Example 15B

### 2-bromo-5 -methylbenzo[d]thiazole (B-15)

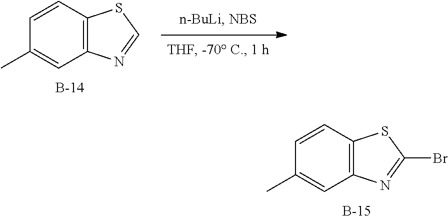

To a solution of compound B-14 (0.27 g, 1.2 mmol) in tetrahydrofuran (4 mL) at −70° C. under nitrogen was added n-butyllithium (0.6 mL, 2.5 mol/L in hexane, 1.5 mmol). The mixture was stirred at −70° C. for 0.5 hours. Then a solution of N-bromosuccinimide(0.26 g, 1.5 mmol) in tetrahydrofuran (2 mL) was added at −70° C., and stirring was continued for an additional 0.5 hours. The mixture was quenched with saturated aqueous ammonium chloride (1.2 mL) at −70° C., then diluted with ethyl acetate (200 mL) at 0° C., washed with brine (6×10 mL), dried over anhydrous sodium sulfate, filtered and concentrated in vacuo. The residue was purified by prep-TLC [silica gel, petroleum ether/ethyl acetate=15:1, R_{f}=0.52] to give compound B-15 (80 mg, 25% yield, 88% purity) as a yellow solid. ¹H-NMR (CDCl₃, 400 MHz): 7.79 (s, 1H), 7.68 (d, J=8.0 Hz, 1H), 7.26 (d, J=11.2 Hz, 1H), 2.51 (s, 3H).

### Example 16B

### 5-cyclopropylbenzo[d]thiazole (B-16)

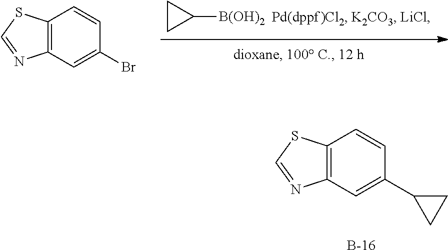

A mixture of 5-bromobenzo[d]thiazole (1.0 g, 4.7 mmol), cyclopropylboronic acid (1.2 g, 14.0 mmol), potassium carbonate (1.3 g, 9.3 mmol) and lithium chloride (19.8 mg, 0.47 mmol) in dioxane (10.0 mL) and water (1.0 mL) was degassed and purged with nitrogen 3 times. To the mixture [1,1′-bis(diphenylphosphino)ferrocene]palladium(II) dichloride (341.7 mg, 0.47 mmol) was added, and the resulting mixture was stirred at 100° C. for 12 hours. On completion, the mixture was poured into water and extracted with ethyl acetate. The organic layer was dried over anhydrous sodium sulfate and concentrated in vacuo. The residue was purified by silica gel chromatography [petroleum ether: ethyl acetate=10:1] to give compound B-16 (720 mg, 81% yield) as a white solid. ¹H-NMR (CD₃OD, 400 MHz): δ 9.18 (s, 1H), 7.90 (d, J=8.4 Hz, 1H), 7.76 (s, 1H), 7.23 (d, J=8.4 Hz, 1H), 2.09-2.05 (m, 1H), 1.05-1.03 (m, 2H), 0.78-0.76 (m, 2H).

### Example 17B

### 2-bromo-5 -cyclopropylbenzo[d]thiazole (B-17)

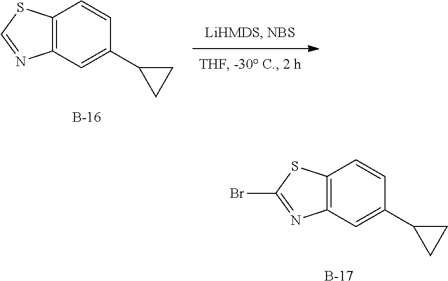

To a solution of compound B-16 (300 mg, 1.7 mmol) in tetrahydrofuran (5.0 mL) at −30° C. was added lithium bis(trimethylsilyl)amide (1 M in tetrahydrofuran, 2.6 mL, 2.6 mmol). The solution was stirred for 1 hour. Then N-bromosuccinimide(457 mg, 2.6 mmol) was added, and stirring was continued at −30° C. for another 2 hours. On completion, the mixture was poured into water and extracted with ethyl acetate. The organic layer was dried over anhydrous sodium sulfate and concentrated in vacuo. The residue was purified by silica gel chromatography [petroleum ether: ethyl acetate=10:1] to give compound B-17 (220 mg, 51% yield) as a white solid. ¹H-NMR (CD₃OD, 400 MHz): δ 7.78 (d, J=8.0 Hz, 1H), 7.63 (s, 1H), 7.21 (d, J=8.8 Hz, 1H), 2.09-2.01 (m, 1H), 1.07-1.02 (m, 2H), 0.78-0.74 (m, 2H).

### Example 18B

### 5-fluorobenzo[ ci] thiazole-2(3H)-thione (B-18)

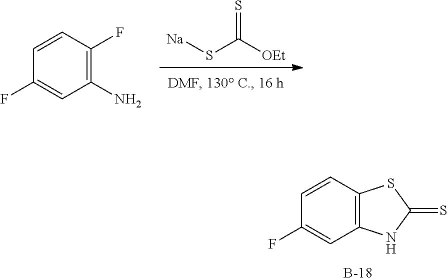

A solution of 2,5-difluoroaniline (5.0 g, 39 mmol) and sodium O-ethyl carbonodithioate (14 g, 97 mmol) in N,N-dimethyl formamide (10 mL) was stirred at 130° C. for 16 hours. One completion, the pH was adjusted to 3 with 2N hydrochloric acid, resulting in formation of a solid. The solid was collected by filtration and dried in vacuo to give compound B-18 (7.00 g, crude) as a pale yellow solid. LCMS (B): tR=0.648 min, 186.1, m/z (M+1).

### Example 19B

### 2-chloro-5-fluorobenzo[d]thiazole (B-19)

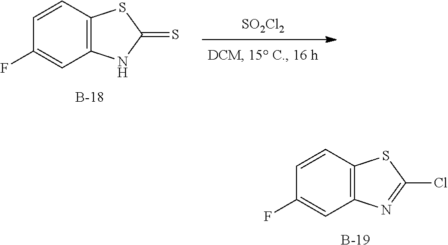

To a solution of compound B-18 (1.0 g, 5.4 mmol) in dichloromethane (10 mL) was added dropwise sulfonyl chloride (3.6 g, 27 mmol). The mixture was stirred at 15° C. for 16 hours, then quenched at 0° C. with saturated aqueous ammonium chloride (2 mL) and extracted with ethyl acetate (2×25 mL). The combined organic phase was concentrated in vacuo, and the residue was purified by silica gel silica gel chromatography [petroleum ether: ethyl acetate=1:0] to give compound B-19 (0.6 g, 3.2 mmol, 59% yield) as a white solid. GCMS: tR=7.673 min, 186.9, (EI) m/z (M)⁻.

### Example 20B

### 5 -(trifluoromethyl)benzo[d]thiazole -2 (3H)-thione (B-20)

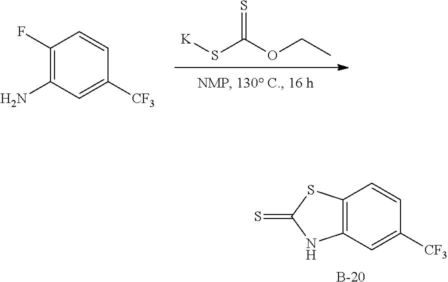

To a mixture of 2-fluoro-5-(trifluoromethyl)aniline (5.0 g, 28 mmol) in N-methyl-2-pyrrolidone (50 mL) at room temperature was added potassium O-ethyl carbonodithioate (5.4 g, 34 mmol). The mixture was stirred at 130° C. for 16 hours, then poured into ice water (100 mL) and acidified with 1 N HCl (20 mL), resulting in formation of a solid. The solid was collected by filteration to give compound B-20 (6.0 g, 88% yield) as a yellow solid. LCMS (B): tR=0.769 min., (ES⁺) m/z (M+H)⁺=236.1.

### Example 21B

### 2-chloro-5-(trifluoromethyl)benzo[d]thiazole (B-21)

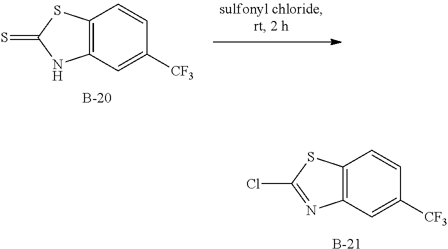

To compound B-20 (1.0 g, 4.3 mmol) at 0° C. was added sulfonyl chloride (10 mL) in portions. The mixture was stirred at room temperature for 2 hour then concentrated in vacuo. The residue was purified by silica gel chromatography [petroleum ether: ethyl acetate=20:1] to give compound B-21 (0.1 g, 9.9% yield) as a white solid.

### Example 22B

### 2-thioxo-2,3-dihydrobenzo[d]thiazole-5-carbonitrile (B-22)

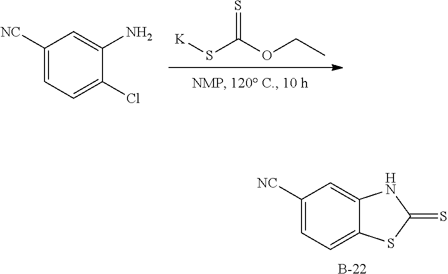

A mixture of 3-amino-4-chlorobenzonitrile (1.0 g, 6.6 mmol) and potassium O-ethyl carbonodithioate (2.1 g, 13.1 mmol) in 1-methylpyrrolidin-2-one (5 mL) at 25° C. was degassed and purged with nitrogen 3 times. The mixture was stirred at 120° C. for 10 hours, then poured into ice water (200 mL) and acidified with 1 mL concentrated hydrochloric acid, resulting in formation of a solid. The solid was collected by filtration, washed with water (3×10 mL) and dried in vacuo to give compound B-22 (1.1 g, crude) as a yellow solid, which was used directly without further purification. LCMS (B): tR=0.613 min., (ES⁺) m/z (M+H)⁺=193.1.

### Example 23B

### 2-chlorobenzo[a] thiazole-5-carbonitrile (B-23)

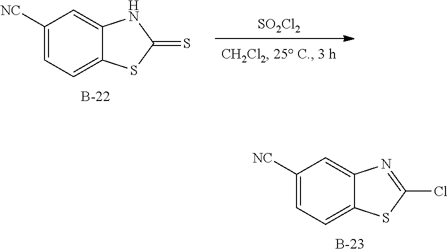

To a solution of compound B-22 (1.0 g, 5.2 mmol) in dichloromethane (10 mL) at 0° C. was added sulfuryl dichloride (13.5 g, 100 mmol). The mixture was stirred at 25° C. for 3 hours, then poured into water (200 mL), partially concentrated in vacuo to remove dichloromethane and extracted with ethyl acetate (3×90 mL). The combined organic phase was washed with brine (3×20 mL), dried over anhydrous sodium sulfate, filtered and concentrated in vacuo. The residue was purified by silica gel chromatography [petroleum ether/ethyl acetate=15:1 to 10:1] to give compound B-23 (0.4 g, 40% yield) as a yellow solid. ¹H-NMR (CDCl₃, 400 MHz): 8.26 (d, J=0.8 Hz, 1H), 7.92 (d, J=8.4 Hz, 1H), 7.68 (dd, J=8.4, 1.6 Hz, 1H).

### Example 24B

### 5,6-dichlorobenzo[d]thiazol-2-amine (B-24)

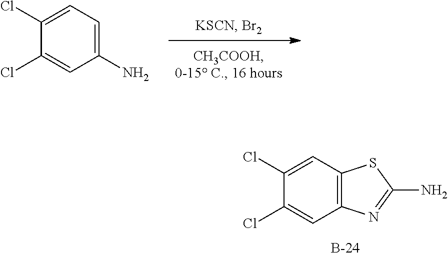

To a mixture of 3,4-dichloroaniline (10 g, 62 mmol) and potassium thiocyanate (48 g, 0.49 mol) in acetic acid (160 mL) at 0° C. was added slowly with constant stirring a solution of liquid bromine (31 g, 0.19 mol) in acetic acid (160 mL). The temperature was maintained at 0° C. throughout the addition. The solution was stirred for 2 hours at 0° C. and 14 hours at 15° C., then diluted with water (100 mL), adjusted to pH 7-8 with ammonium hydroxide and extracted with ethyl acetate (3×50 mL). The combined organic layers were washed with brine (3×300 mL) and concentrated in vacuo. The residue was purified by prep-HPLC [Instrument: GX-B; Column: GEMINI 250×50 mm, particle size: 10 μm; Mobile phase: 25-50% acetonitrile in H₂O (add 0.1% TFA, v/v)] to give compound B-24 (2.6 g, 19% yield) as a white solid. LCMS (J): tR=0.694 min. (ES⁺) m/z (M+H)⁺=219.0. ¹H-NMR (CD₃OD, 400 MHz): δ 7.89 (s, 1H), 7.55 (s, 1H).

### Example 25B

### 2-bromo-5,6-dichlorobenzo[d]thiazole (B−25)

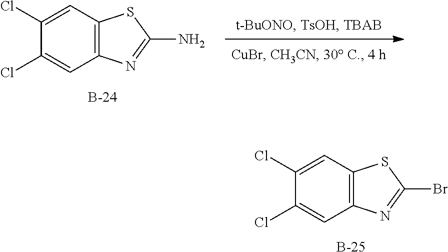

To a solution of compound B-24 (0.25 g, 1.1 mmol), tert-butyl nitrite (0.35 g, 3.4 mmol), p-toluenesulfonic acid monohydrate (0.59 g, 3.4 mmol) and tetrabutylammonium bromide (2.2 g, 6.8 mmol) in acetonitrile (25 mL) was added copper(I) bromide (16 mg, 0.11 mmol). The reaction mixture was stirred at 30° C. for 4 hours, and then concentrated. The residue was purified by column chramotagraphy [petroleum ether: ethyl acetate=20:1) to give compound B-25 (0.18 g, 56% yield) as a yellow solid.

### Example 26B

### 6,7-dichlorobenzo[d]thiazole-2(3H)-thione (B-26)

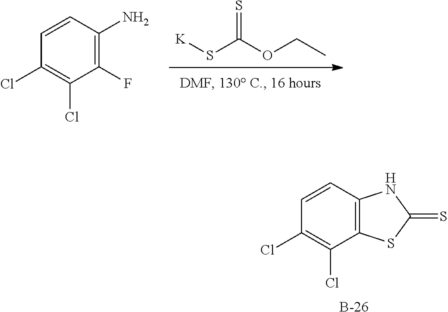

A solution of 3,4-dichloro-2-fluoroaniline (5.0 g, 31 mmol) and potassium O-ethyl carbonodithioate (11 g, 67 mmol) in N, N-dimethylformamide (50 mL) was stirred at 130° C. for 16 hours. On completion, the reaction was cooled to room temperature, and hydrochloric acid (1M, 75 mL) was added. The mixture was stirred for 0.5 hour, resulting in formation of a solid. The solid was collected by filtration, washed with water (2×100 mL) and dried in vacuo to give compound B-26 (5.2 g, 79% yield) as a white solid. LCMS (B): tR=0.847 min., (ES⁺) m/z (M+H)⁺=237.0.

### Example 27B

### 2,6,7-trichlorobenzo[d]thiazole (B-27)

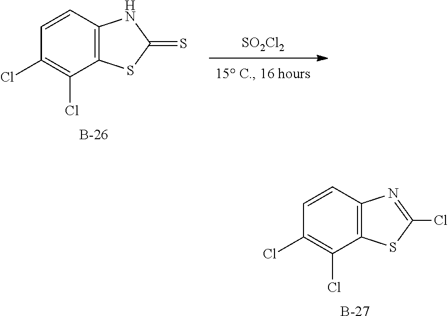

A solution or compound B-26 (2.0 g, 8.5 mmol) in sulfonyl chloride (34 g, 0.25 mol) was stirred at 15° C. for 16 hours. On completion, the solution was poured into ice-water (30 mL) and extracted with ethyl acetate (3×50 mL). The combined organic layers were concentrated in vacuo and purified by silica gel chromatography [petroleum ether: ethyl acetate=20:1) to give compound B-27 (1.8 g, 89% yield) as a white solid. ¹H-NMR (CDCl₃, 400 MHz): δ 7.80 (d, J=8.8 Hz, 1H), 7.60 (d, J=8.8 Hz, 1H).

### Example 28B

### 1-(3,5-dichlorophenyl)thiourea (B-28)

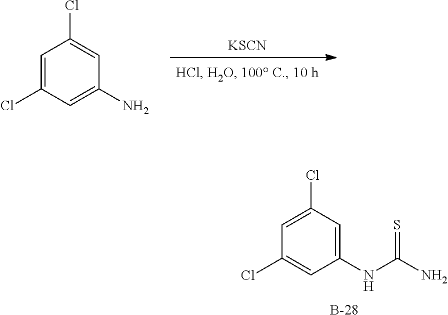

To 3,5-dichloroaniline (1.0 g, 6.2 mmol) was added hydrochloric acid (1 M, 12 mL) and potassium thiocyanate (0.72 g, 7.4 mmol). The mixture was stirred at 100° C. for 10 hours. The resulting solid was collected by filtration, washed with water (3×5 mL) and dried in vacuo. The residue was purified by silica gel chromatography [petroleum ether: ethyl acetate=10:1 to 1:2] to give compound B-28 (0.36 g, 22% yield) as a yellow solid. LCMS (J): tR=1.135 min., (ES⁺) m/z (M+H)⁺=221.0.

### Example 29B

### 5,7-dichlorobenzo[d]thiazol-2-amine (B-29)

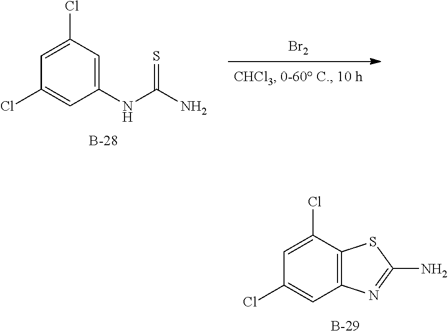

To a solution of compound B-28 (1.0 g, 3.7 mmol) in chloroform (20 mL) at 0° C. was added bromine (1.2 g, 7.4 mmol). The mixture was stirred at 60° C. for 10 hours, then cooled to room temperature and filtered to collect the solid. The solid was washed with acetone (4×5 mL), then added into water (45 mL). The mixture was stirred at 95° C. for 30 min, then adjusted to pH 9 with sodium hydroxide (0.5 M). The solid was collected by filtration and dried in vacuo to give compound B-29 (0.68 g, 81% yield) as a purple gray solid. ¹H-NMR (CDCl₃, 400 MHz): 7.43 (d, J=1.6 Hz, 1H), 7.15 (d, J=1.6 Hz, 1H), 5.32 (bs, 2H).

### Example 30B

### 2-bromo-5,7-dichlorobenzo[d]thiazole (B−30)

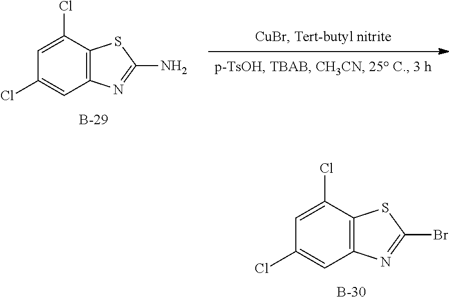

To a solution of compound B-29 (0.6 g, 2.7 mmol), tetra-n-butyl-ammonium (1.8 g, 5.5 mmol), tert-butyl nitrite (0.34 g, 3.3 mmol) and 4-methylbenzenesulfonic acid hydrate (0.63 g, 3.3 mmol) in acetonitrile (30 mL) was added copper(I) bromide (3.9 mg, 0.03 mmol). The mixture was stirred at 25° C. for 3 hours, and concentrated in vacuo. The residue was purified by silica gel chromatography [SiO₂, petroleum ether: ethyl acetate=6:1] to give compound B-30 (0.56 g, δ 9% yield) as a yellow solid. ¹H-NMR (CDCl₃, 400 MHz): 7.89 (d, J=1.2 Hz, 1H), 7.44 (d, J=1.2 Hz, 1H).

### Example 31B

### 6-chloro-5,7-difluorobenzo[d]thiazol-2-amine (B-31)

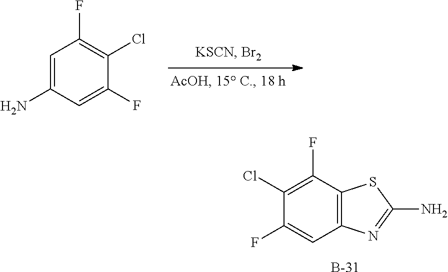

To a solution of 4-chloro-3,5-difluoroaniline (3.0 g, 18 mmol) and potassium thlocyanate (3.6 g, 37 mmol) in acetic acid (30 mL) at 15° C. was added slowly bromine (4.4 g, 28 mmol). The mixture was stirred at 15° C. for 3 hours, then adjusted to pH 8 with saturated aqueous sodium carbonate and extracted by ethyl acetate (2×20 ml). The combined organic phase was concentrated and purified by silica chromatography [petroleum ether: ethyl acetate=10/1 to 5/1) to give compound B-31 (1.4 g, 35%) as a yellow solid.

### Example 32B

### 2-bromo-6-chloro-5,7-difluorobenzo[d]thiazole (B-32)

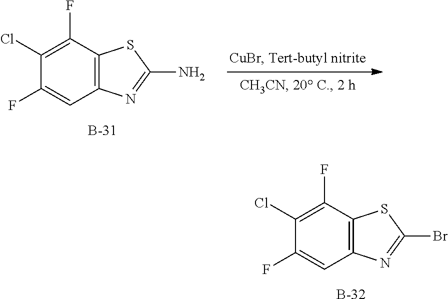

To a solution of compound B-31 (0.50 g, 2.1 mmol) in acetonitrile (2.0 mL) was added copper(I) bromide (0.33 g, 2.3 mmol) and tert-butyl nitrite (0.37 g, 3.2 mmol). The resulting mixture was stirred at 20° C. for 2 hours, and then concentrated in vacuo. The residue was purified by silica gel chromatography [petroleum ether: ethyl acetate=30:110:1] to give compound B-32 (0.4 g, 63% yield) as a white solid. ¹H-NMR (CDCl₃, 400 MHz): δ 7.66-7.64 (m, 1H).

### Example 33B

### 4-bromo-2-chloro-l-cyclopropylbenzene (B-33)

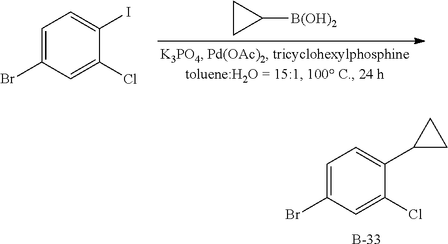

A solution of 4-bromo-2-chloro-l-iodo-benzene (1.0 g, 3.2 mmol), cyclopropylboronic acid (0.35 g, 4.1 mmol), potassium phosphate (2.3 g, 11 mmol), palladium acetate (35 mg, 0.16 mmol) and tricyclohexylphosphine (88 mg, 0.032 mmol) in toluene (30 mL) and water (1.6 mL) was stirred at 100° C. under nitrogen for 24 hours. On completion, the mixture was poured into water (20 mL) and extracted with ethyl acetate (3×20 mL). The combined organic layers were washed with brine (2×20 mL), dried over anhydrous sodium sulfate, filtered and concentrated in vacuo. The residue was purified by silica gel chromatography [petroleum ether: ethyl acetate=1:0] to give compound B-33 (0.80 g, 46% yield) as a yellow solid. GCMS (B): tR=8.562 min, (ES+) m/z=232.0.

### Example 34B

### 4-bromo-1 -chloro-2-cyclopropylbenzene (B-34)

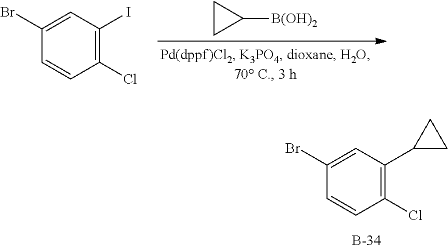

To a solution of 4-bromo-l-chloro-2-iodobenzene (0.30 g, 0.95 mmol), cyclopropylboronic acid (97 mg, 1.1 mmol), [1,1¹-bis(diphenylphosphino)ferrocene] dichloropalladium(II) (39 mg, 0.047 mmol) in dioxane (6 mL) and water (2 mL) under nitrogen at room temperature was added potassium phosphate (0.76 g, 3.6 mmol). The reaction mixture was stirred at 70° C. for 3 hours, then diluted with water (10 mL) and extracted with ethyl acetate (3×10 mL). The combined organic layers were washed with brine (2×10 mL), dried over sodium sulfate, filtered and concentrated in vacuo to give compound B-34 (0.30 g, crude) as a yellow oil.

### Example 35B

### 6-bromo-2-methylbenzofuran (B-35)

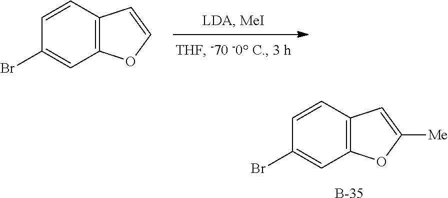

To a solution of 6-bromobenzofuran (0.5 g, 2.5 mmol) in anhydrous tetrahydrofuran (5.0 mL) at −70° C. under nitrogen was added dropwise lithium diisopropylamide (2.0 M in tetrahydrofuran/n-heptane, 0.28 mL, 0.57 mmol). The solution was stirred at this temperature for 1 hour, and then methyl iodide (0.43 g, 3.1 mmol) was added. The mixture was stirred at −70° C. for 3 hours and at 0° C. 1 hour, then poured into 0.5 N hydrochloric acid (20 mL) and extracted with methyl tert-butyl ether (2×20 mL). The combined organic layers were washed with brine, dried over anhydrous sodium sulfate, filtered and concentrated in vauo. The residue was purified by silica gel chromatography [petroleum ether] to give compound B-35 (0.5 g, 90% yield) as a yellow liquid. GCMS (B): tR=8.097 min, (ES⁺) m/z=209.9.

### Example 36B

### 5-bromo-2-methylbenzofuran (B-36)

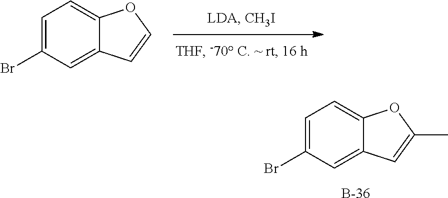

To a solution of 5-bromobenzofuran (2.0 g, 10 mmol) in tetrahydrofuran (20 mL) at −70 ° C. under nitrogen was added dropwise lithium diisopropylamide (2 M in tetrahydrofuran, 6.1 mL, 12 mmol). The solution was stirred at −70° C. for 1 hour, and then iodomethane (2.2 g, 15 mmol) was added. The mixture was stirred at −70° C. for 2 hours and at room temperature for another 13 hours, then poured into 5% hydrochloric acid (100 mL) and extracted with ethyl acetate (2×100 mL). The combined organic layers were washed with saturated aqueous sodium bicarbonate solution (2×30 mL) and concentrated in vacuo. The residue was purified by flash column chramotagraphy [100% petroleum ether] to give compound B-36 (1.9 g, crude) as oil.

### Example 37B

### 6-bromo-2-methylbenzo[b]thiophene (B-37)

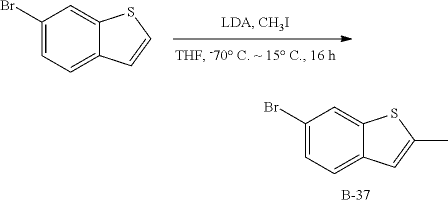

To a solution of 6-bromobenzo[b]thiophene (2.0 g, 9.4 mmol) in tetrahydrofuran (10 mL) at −70° C. under nitrogen was added dropwise lithium diisopropylamide (2 M in tetrahydrofuran, 5.6 mL, 11 mmol). The solution was stirred at −70° C. for 0.5 hour, and then iodomethane (12 g, 85 mmol) was added. The resulting solution was stirred at 15° C. for 15.5 hours, then quenched by saturated aqueous ammonium chloride solution (100 mL) and extracted with ethyl acetate (3×80 mL). The combined organic layers were concentrated in vacuo and purified by silica gel chromatography [100% petroleum ether] to give compound B-37 (1.2 g, crude) as a white solid. GCMS (B): tR=9.386 min, (ES⁺) m/z=227.9.

### Example 38B

### 5-bromo-2-methylbenzo[b]thiophene (B-38)

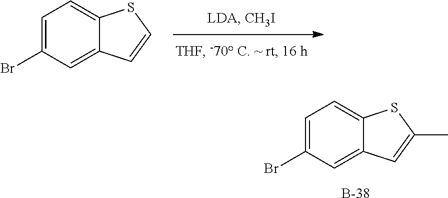

To a solution of 5-bromobenzo[b]thiophene (2.0 g, 9.4 mmol) in tetrahydrofuran (20 mL) at −70° C. under nitrogen was added dropwise lithium diisopropylamide (2 M in tetrahydrofuran, 5.6 mL, 11 mmol). The solution was stirred at −70° C. for 1 hour, and then iodomethane (2.0 g, 14 mmol) was added. The resulting solution was stirred at −70° C. for 2 hours and at room temperature for another 13 hours, then poured into 5% hydrochloric acid (100 mL) and extracted with ethyl acetate (2×100 mL). The combined organic layers were washed with aqueous sodium bicarbonate (2×30 mL) and concentrated in vacuo. The residue was purified by flash column chramotagraphy [100% petroleum ether] to give compound B-38 (1.9 g, 89% yield) as a white solid. 1H-NMR (CDC13, 400 MHz): δ 7.80 (d, J=2.0 Hz, 1H), 7.61 (d, J=8.4 Hz, 1H), 7.36 (dd, J1=8.4 Hz, J2=1.6 Hz, 1H), 6.93 (s, 1H), 2.61 (s, 3H).

### Example 39B

### N-(3-fluorobenzyl)-2,2-dimethoxyacetamide (B-39)

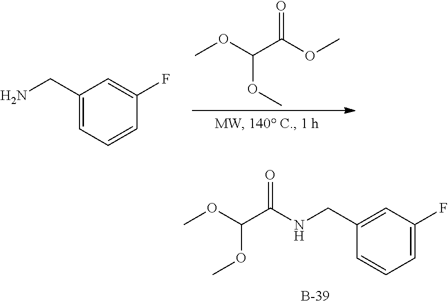

A mixture of (3-fluorophenyl)methanamine (1.0 g, 14 mmol) and methyl 2,2-dimethoxyacetate (2.1 g, 16 mmol) was stirred at 140° C. for 1 hour in the microwave. The solution was concentrated in vacuo, and the residue was purified by silica gel chromatography [petroleum ether: ethyl acetate=5:1) to give compound B-39 (1.4 g, 77% yield) as a white solid. LCMS (B): tR=0.594 min., (ES⁺) m/z (M+H)⁺=228.1.

### Example 40B

### 7-fluoroisoquinolin-3-ol (B−40)

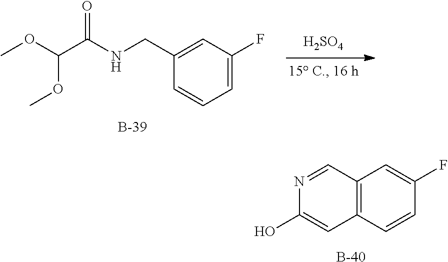

Compound B-39 (2.5 g, 11 mmol) was added to concentrated sulfuric acid (15 mL) at 15° C. The mixture was stirred at 15° C. for 16 hours, then poured into ice-water (30 mL). The resulting solid was collected by filtration, washed with water (100 mL) and dried in vacuo to give compound B-40 (1.6 g, 89% yiled) as a white solid. LCMS (M): tR=0.578 min., (ES⁺) m/z (M+H)⁺=164.1.

### Example 41B

### 7-fluoroisoquinolin-3-yl trifluoromethanesulfonate (B-41)

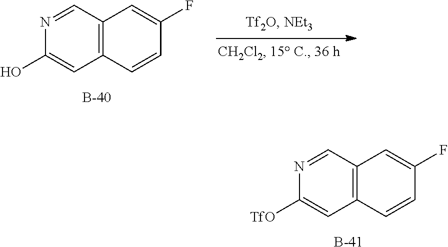

To a solution of compound B-40 (2.0 g, 12 mmol) and triethylamine (2.5 g, 24 mmol) in dichloromethane (20 mL) at 15° C. was added dropwise trifluoromethanesulfonic anhydride (6.9 g, 24 mmol). The mixture was stirred at 15° C. for 36 hours, then diluted with dichloromethane (200 mL), washed with water (100 mL), aqueous sodium bicarbonate (100 mL) and brine (100 mL) and concentrated in vacuo. The residue was purified by silica gel chromatography [petroleum ether: ethyl acetate=1:1] to give compound B-41 (1.7 g, 45% yield) as a yellow oil. LCMS (B): tR=0.932 min., (ES⁺) m/z (M+H)⁺=296.0; ¹H-NMR (CDCl₃, 400 MHz): δ 9.06 (s, 1H), 7.96 (dd, J₁=12.4 Hz, J₂ =6.8 Hz, 1H), 7.72-7.59 (m, 3H).

### Example 42B

### N-(4-fluorobenzyl)-2,2-dimethoxyacetamide (B-42)

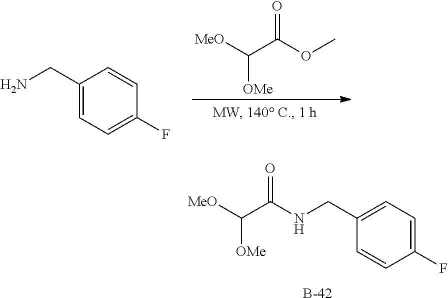

A mixture of (4-fluorophenyl)methanamine (3.0 g, 24 mmol) and methyl 2,2-dimethoxyacetate (6.4 g, 48 mmol) was heated at 140° C. for 1 hour in the microwave. On completion, the mixture was concentrated in vacuo and purified by silica gel chromatography column [petroleum ether: ethyl acetate=1:1] to give compound B-42 (4.1 g, 74% yield) as a light yellow solid. ¹H-NMR (CD₃OD, 400 MHz): δ 7.30-7.28 (m, 2H), 7.05 (t, J=8.4 Hz, J=8.8 Hz, 2H), 6.91 (br, 1H), 4.78 (s 1H), 4.78 (d, J=6.0 Hz, 2H), 3.43 (s, 6H).

### Example 43B

### 6-fluoroisoquinolin-3(2H)-one (B-43)

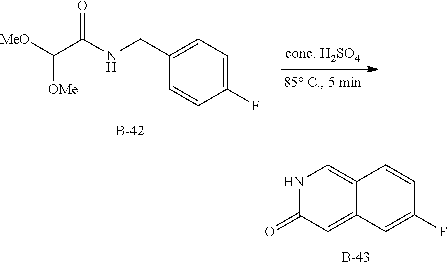

A mixture of compound B-42 (1.0 g, 6.6 mmol) and concentrated sulfuric acid (1.4 mL) was stirred at 85° C. for 5 minute. On completion, the mixture was cooled to room temperature, poured into ice water (40 mL), adjusted to pH=7-8 with saturated aqueous sodium bicarbonate, diluted with dichloromethane (100 mL) and stirred at 20° C. for 12 hours. On completion, the precipitated product was collected by filtration. The solid was further purified by silica gel chromatography column [dichloromethane: methanol=10:1] to give compound B-43 (0.9 g , 63% yield) as a yellow solid. 1H-NMR (CDCl₃, 400 MHz): δ 8.67 (s, 1H), 7.90 (q, J=8.0 Hz, 1H), 7.23 (dd, J=4.0 Hz, J=12 Hz, 1H), 7.07-7.02 (m, 1H), 6.82 (s, 1H).

### Example 44B

### 6-fluoroisoquinolin-3-yltrifluoromethanesulfonate (B-44)

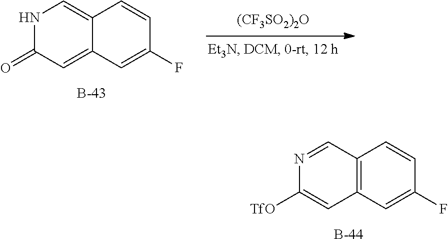

To a solution of compound B-43 (1.0 g, 6.1 mmol) and triethylamine (1.4 g, 13 mmol) in anhydrous dichloromethane (20 mL) at 0° C. was added dropwise trifluoromethanesulfonic anhydride (3.8 g, 13 mmol). The mixture was stirred at 0° C. for 1 hour and room temperature for 11 hours, then quenched with saturated aqueous sodium bicarbonate (20 mL). The layers were separated, and the organic layer was washed with brine, dried over anhydrous sodium sulfate, filtered and concentrated in vacuo. The residue was purified by silica gel chromatography column [petroleum ether: ethyl acetate=10:1] to give compound B-44 (1.0 g, 53% yield) as a yellow solid. ¹H-NMR (CDCl₃, 400 MHz): δ 9.07 (s, 1H), 8.12 (q, J=8.0 Hz, 1H), 7.53-7.44 (m, 3H).

### Example 45B

### 2-(hydroxyimino)-6-methoxy-2,3-dihydro-1H-inden-l-one (B-45)

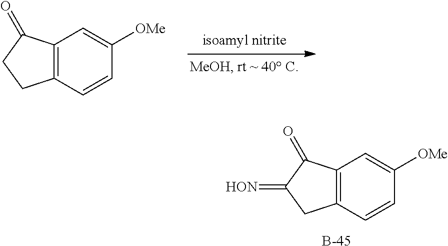

To a solution of 6-methoxyindan-l-one (1.0 g, 6.2 mmol) in methanol (20 mL) at room temperature was added hydrochloric acid (1.0 mL, 12 mmol) and isoamyl nitrite (1.6 g, 14 mmol). The solution was stirred at 40° C. for 2 hours, then filtered and concentrated in vacuo to give compound B-45 (0.80 g, 68% yield) as a yellow solid. LCMS (B): tR=0.592 min., (ES+) m/z (M+H)+=192.1.

### Example 46B

### 1,3-dichloro-7-methoxyisoquinoline (B-46)

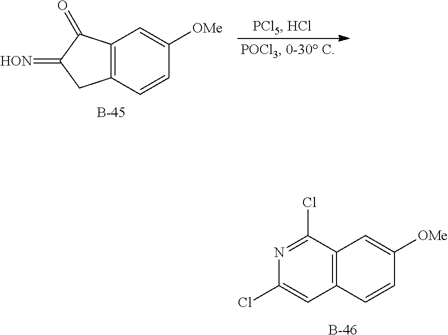

To a solution of compound B-45 (5.4 g, 28 mmol) in phosphorus oxychloride (181 mL) at 0° C. was added phosphorus pentachloride (5.9 g, 28 mmol). Then hydrogen chloride gas was bubbled through the reaction until the solution was saturated. The reaction mixture was stirred at 80° C. for 12 hours, then concentrated in vacuo and added into ice water. The resulting solid was collected by filtration, washed with water (30 mL) and dried in vacuo. The residue was purified by silica gel chromatography [petroleum ether: ethyl acetate=10:1] to give compound B-46 (6.0 g, 93% yield) as a brown solid. LCMS (M): tR=1.055 min., (ES+) m/z (M+H)+=228.0.

### Example 47B

### 3-chloro-7-methoxyisoquinoline (B-47)

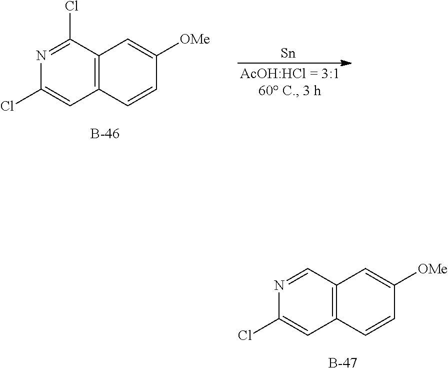

A mixture of compound B-46 (2.0 g, 8.8 mmol), tin (3.1 g, 26 mmol) and hydrochloric acid (4.0 mL) in acetic acid (12 mL) was stirred at 60° C. for 3 hours. On completion, the mixture was adjusted to pH=9 with aqueous saturated ammonia (30 mL) and extracted with ethyl acetate (3×50 mL). The combined organic layer was washed with saturated aqueous sodium bicarbonate (40 mL), dried over anhydrous sodium sulfate and concentrated in vacuo. The residue was purified by silica gel chromatography [petroleum ether: ethyl acetate=10:1] to give compound B-47 (0.70 g, 41% yield) as a brown solid. ¹H-NMR (DMSO-d6, 300 MHz): δ 9.08 (s, 1H), 7.95 (s, 1H), 7.89 (d, J=9 Hz, 1H), 7.55 (d, J=2.1 Hz, 1H), 7.48 (dd, J1=9.3 Hz, J2=2.7 Hz, 1H).

### Example 48B

### 2-bromo-6-chloroquinazoline (B-48)

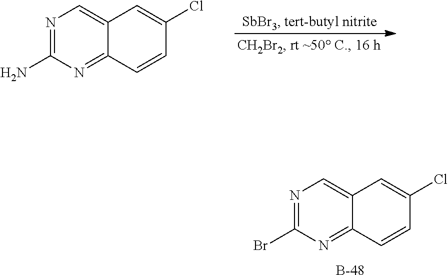

To a solution of 6-chloroquinazolin-2-amine (0.20 g, 1.1 mmol) in dibromomethane (20 mL) at 25° C. was added antimony tribromide (0.80 g, 2.2 mmol) followed by tert-butyl nitrite (0.46 g, 4.4 mmol). The mixture was stirred at 50° C. for 16 hours, then filtered, diluted with dichloromethane (20 mL), washed with water (2×20 mL) and concentrated in vacuo. The residue was purified by prep-TLC [petroleum ether: ethyl acetate=8:1) to give compound B-48 (0.10 g, 23% yield) as a brown solid. LCMS (B): tR=0.709 min., (ES⁺) m/z (M+H)⁺=242.9.

### Example 48B

### 2-bromo-7-chloroquinazoline (B-49)

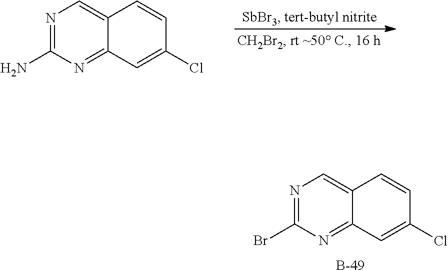

To a solution of 7-chloroquinazolin-2-amine (0.20 g, 1.1 mmol) in dibromomethane (25 mL) at 25° C. was added antimony tribromide (0.80 g, 2.2 mmol) followed by tert-butyl nitrite (0.46 g, 4.4 mmol). The mixture was stirred at 50° C. for 16 hours, then filtered, diluted with dichloromethane (20 mL), washed with water (2×20 mL) and concentrated in vacuo. The residue was purified by prep-TLC [petroleum ether: ethyl acetate=8:1) to give compound B-49 (0.15 g, 46% yield) as a brown solid. LCMS (B): tR=0.754 min., (ES⁺) m/z (M+H)⁺=243.0.

### Example 50B

### 6-methylthieno[2,3-d]pyrimidine-2,4(1H,3H)-dione (B-50)

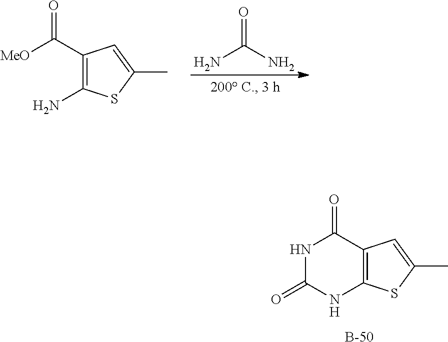

A mixture of methyl 2-amino-5-methyl-thiophene-3-carboxylate (3.0 g, 18 mmol) and urea (5.3 g, 88 mmol) was stirred at 200° C. for 3 hours. On completion, the reaction was diluted with water (20 mL) and extracted with ethyl acetate (2×20 mL). The combined organic layers were dried over anhydrous sodium sulfate and concentrated in vacuo. The residue purified by silica gel chromatography [dichloromethane: methanol=10:1] to give compound B-50 (3.0 g, 94% yield) as a black solid. LCMS (J): tR=0.122 min., (ES+) m/z (M+H)+=183.0.

### Example 51B

### 2,4-dichloro-6-methylthieno[2,3-d]pyrimidine (B-51)

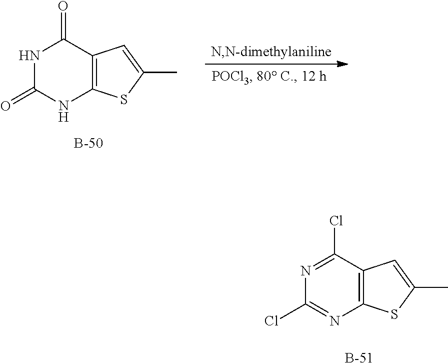

A mixture of compound B-50 (2.0 g, 11 mmol) and dimethylaniline (1.9 g, 16 mmol) in phosphorus oxychloride (272 mL) was stirred at 80° C. for 12 hours. On completion, the mixture was concentrated, poured into water (40 mL) and extracted with ethyl acetate (2×50 mL). The combined organic phase was washed with brine (30 mL), dried over anhydrous sodium sulfate, filtered and concentrated in vacuum. The residue was purified by silica gel chromatography [petroleum ether: ethyl acetate=5:1] to give compound B-51 (0.50 g, 21% yield) as a yellow solid. LCMS (J): tR=1.221 min., (ES+) m/z (M+H)+=241.2

### Example 52B

### 2-chloro-6-methylthieno[2,3-d]pyrimidine (B-52)

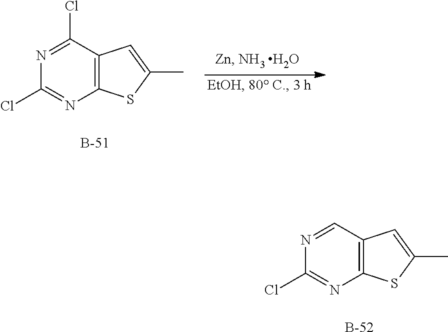

A mixture of compound B-51 (0.45 g, 2.1 mmol), zinc (1.1 g, 16 mmol) and aqueous ammonia (0.40 mL, 10 mmol, 27%) in ethanol (5.0 mL) was stirred at 80° C. for 3 hours. On completion, the mixture was filtered, concentrated in vacuo, dissolved in water (40 mL) and extracted with ethyl acetate (2×50 mL). The combined organic phase was washed with brine (30 mL), dried with anhydrous sodium sulfate, filtered and concentrated in vacuum. The residue was purified by silica gel chromatography [petroleum ether: ethyl acetate=10:1] to give compound B-52 (0.16 g, 42% yield) as a yellow solid. ¹H-NMR (CDCl₃, 400 MHz): δ 8.82 (s, 1H), 6.98 (d, J=1.2 Hz, 1H), 2.64 (s, 3H).

### Example 53B

### 1-(5-bromothiophen-2-yl)ethanone oxime (B-53)

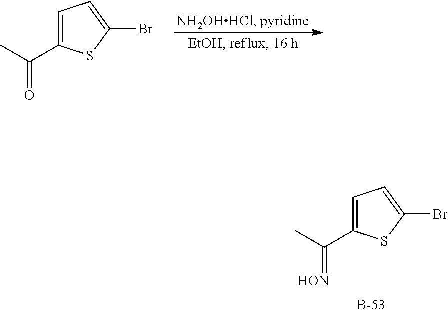

To a solution of 1-(5-bromothiophen-2-yl)ethanone (4.0 g, 20 mmol) in ethanol (50 mL) at room temperature was added hydroxylamine hydrochloride (3.5 g, 50 mmol) and pyridine (4.0 g, 50 mmol). The mixture was stirred at reflux for 16 hours, then concentrated in vacuo, diluted with water (40 mL) and extracted with dichloromethane (3×100 mL). The combined organic layers were washed with water and brine, dried over anhydrous sodium sulfate and concentrated in vacuo. Two products were observed by TLC, and the residue was purified by silica gel chromatography [petroleum ether: ethyl acetate=20:1] to isolate the lower polarity compound B-53 (2.4 g, 56% yield) as a yellow solid: ¹H-NMR (CD₃OD, 400 MHz): δ 6.92 (d, J=8.0 Hz, 2H), 2.07 (s, 3H).

### Example 54B

### N-(5-bromothiophen-2-yl)acetamide (B-54)

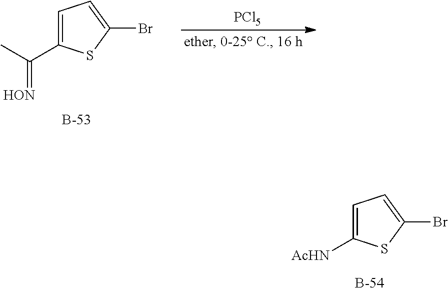

To a solution of compound B-53 (2.4 g, 11 mmol) in ether (30 mL) at 0° C. was added phosphorus pentachloride (2.7 g, 13 mmol). The mixture was stirred at 0° C. for 2 hour and at 25° C. for 14 hours, then poured into ice water (200 mL), adjusted to pH 7 with saturated aqueous sodium bicarbonate and extracted with ethyl acetate (3×100 mL). The combined organic layers were washed with water and brine, dried over anhydrous sodium sulfate and concentrated in vacuo. The residue was purified by silica gel chromatography [petroleum ether: ethyl acetate=10:1] to give compound B-54 (1.8 g, 75% yield) as a yellow solid. ¹H-NMR (DMSO-d6, 400 MHz): δ 11.4 (s, 1H), 6.95 (d, J=5.6 Hz, 1H), 6.42 (d, J=5.6 Hz, 1H), 2.06 (s, 3H).

### Example 55B

### 2-bromo-6-chlorothieno[2,3-b]pyridine (B-55)

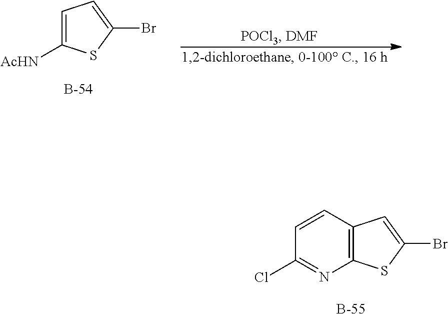

To a mixture of anhydrous N, N - dimethylformamide (0.60 g, 8.2 mmol) in anhydrous 1,2-dichloroethane (5 mL) at 0° C. was added phosphorus oxychloride (5.0 g, 33 mmol) followed by a solution of compound B-54 (1.8 g, 8.2 mmol) in anhydrous 1,2-dichloroethane (20 mL). The reaction mixture was stirred at room temperature for 10 minutes and at 100° C. for 16 hours, then poured into ice water (100 mL), adjusted to pH 9 with saturated aqueous sodium bicarbonate and extracted with dichloromethane (3×50 mL). The combined organic layers were washed with water and brine, dried over anhydrous sodium sulfate and concentrated in vacuo. The residue was purified by silica gel chromatography [petroleum ether: ethyl acetate=50:1] to give compound B-55 (0.35 g, 17% yield) as a yellow solid. LCMS (B): (ES+) m/z (M+H)+=250.0, tR=0.871.

### Example 56B

### 6-chloro-2-methylthieno[2,3-b]pyridine (B-56)

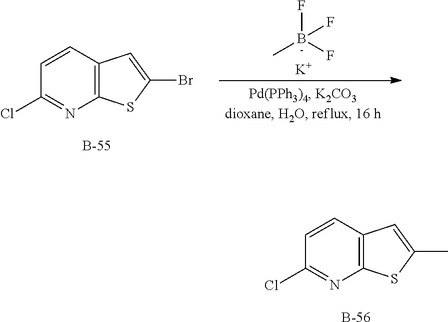

To a mixture of compound B-55 (0.20 g, 0.80 mmol), potassium methyltrifluoroborate (0.10 g, 0.80 mmol) and potassium carbonate (0.28 g, 2.0 mmol) in dioxane (20 mL) and water (2 mL) under nitrogen was added tetrakis(triphenylphosphine)palladium(0) (93 mg, 0.08 mmol). The reaction mixture was stirred at reflux for 16 hours, then diluted with water and extracted with ethyl acetate (3 x 50 mL). The combined organic layers were washed with water and brine, dried over anhydrous sodium sulfate and concentrated in vacuo. The residue was purified by prep-TLC [petroleum ether: ethyl acetate=20:1] to give compound B-56 (90 mg, 51% yield) as a yellow solid. LCMS (B): (ES+) m/z (M+H)+=184.1, tR=0.839.

### Example 57B

### 6-bromo-2-chlorobenzo[b]thiophene (B-57)

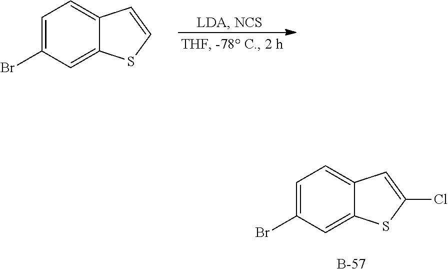

To a solution of 6-bromobenzo[b]thiophene (1.0 g, 4.7 mmol) in tetrahydrofuran (10 mL) at −78° C. was added lithium diisopropylamide (2 M in THF and n-heptane, 2.6 mL, 5.2 mmol). The mixture was stirred at −78° C. for 0.5 hour, and then N-chlorosuccinimide (0.94 g, 7.0 mmol) was added. The mixture was stirred at −78° C. for another 1.5 hours, then quenched with water (20 mL) at 0° C. and extracted with ethyl acetate (2×20 mL). The combined organic layers were washed with water (2×30 mL) and dried over anhydrous sodium sulfate, filtered and concentrated in vacuo. The residue was purified by prep-HPLC [Instrument: GX-C; Column: Daiso 250×50 mm, particle size: 10 μm; Mobile phase: 50-75% acetonitrile in H₂O (add 0.1% TFA, v/v)] and lyophilized to give compound B-57 (0.3 g, 26%) as a brown solid. ¹H-NMR (CD₃OD, 400 MHz): δ 8.00 (s, 1H), 7.64 (d, J=8.4 Hz, 1H), 7.51 (dd, J=8.4 Hz, 1H), 7.31 (s, 1H)

### Example 58B

### 6-bromo-2-fluorobenzo[b]thiophene (B-58)

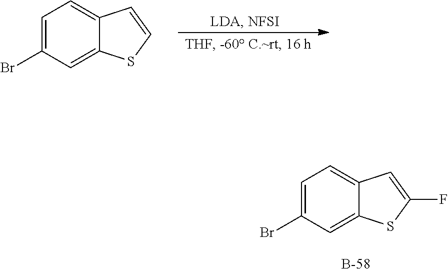

To a solution of 6-bromobenzothiophene (5.0 g, 23 mmol) in tetrahydrofuran (50 mL) at −60° C. was added slowly lithium diisopropylamide (2 M in tetrahydrofuran, 23 mL, 46 mmol). The mixture was stirred at −30° C. for 1 hour, and then N-fluorobenzenesulfonimide (22 g, 70 mmol) in tetrahydrofuran (30 mL) was added at −60° C. The mixture was stirred at room temperature for 15 hours, then quenched with water (20 mL) and extracted with ethyl acetae (2×100 mL). The combined organic layers were concentrated in vacuo and purified by silica gel chromatography [100% petroleum ether] to give compound B-58 (1.2 g, crude) as a white solid.

### Example 59B

### 6-bromo-2,3-difluorobenzo[b]thiophene (B-59)

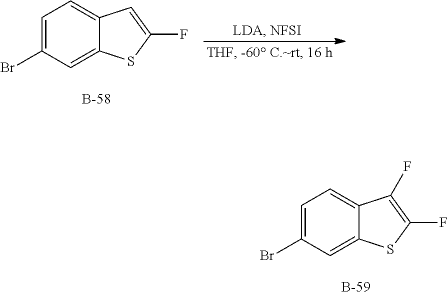

To a mixture of compound B-58 (1.0 g, crude) in tetrahydrofuran (15 mL) at −60° C. was added slowly lithium diisopropylamide (2 M in tetrahydrofuran, 4.5 mL). The mixture was stirred at - 30° C. for 1 hour, and then N-fluorobenzenesulfonimide (4.2 g, 13 mmol) in tetrahydrofuran (30 mL) was at −60° C. The solution was stirred at room temperature for 15 hours, then quenched with water (10 mL) and extracted with ethyl acetae (2×30 mL). The combined organic layers were concentrated in vacuo and purified by silica gel chromatography [100% petroleum ether] to give compound B-59 (0.80 g, 14% yield for two steps) as a white solid.

### Example 60B

### methyl 6-chloro-3-methylbenzo[b]thiophene-2-carboxylate (B−60)

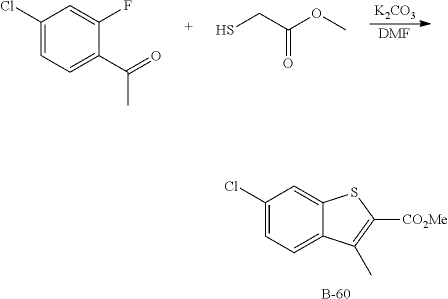

To a solution of 1-(4-chloro-2-fluoro-phenyl)ethanone (7.0 g, 41 mmol) and potassium carbonate (11 g, 81 mmol) in dimethyl formamide (50 mL) at 25° C. was added methyl thioglycolate (7.3 g, δ 9 mmol). The mixture was stirred for 5 hours, then diluted with water (50 mL) and extracted with ethyl acetate (3×40 mL). The combined organic phases were concentrated in vauo to give compound B−60(8 g, crude) as a yellow solid.

### Example 61B

### 6-chloro-3-methylbenzo[b]thiophene-2-carboxylic acid (B-61)

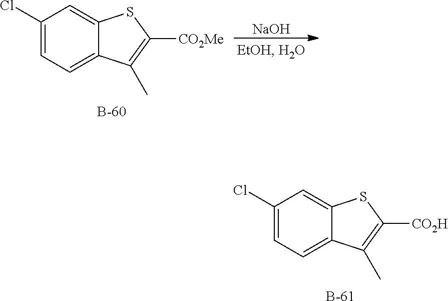

To a solution of compound B-60 (3 g, 12.5 mmol) in ethanol (15 mL) and water (3 mL) was added sodium hydroxide (1.5 g, 37.4 mmol). The mixture was stirred at 25° C. for 2 hours, then acidified to pH 3 with hydrochloric acid (1 M, 10 mL). The resulting solid was collected by filtration and dried in vacuo to give compound B-61 (1.5 g, 53% yield) as a white solid. LCMS (J): tR=0.84 min., (ES⁻) m/z (M+H)⁺=227.1.

### Example 62B

### 6-chloro-3-methylbenzo[b]thiophene(B-62)

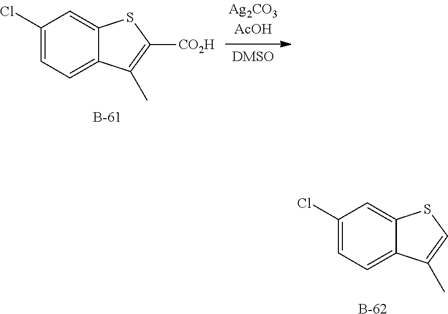

To a solution of compound B-61 (1.5 g, 6.6 mmol) in dimethylsulfoxide (15 mL) under nitrogen was added silver carbonate (0.18 g, 0.66 mmol) and acetic acid (0.019 mL, 0.33 mmol). The mixture was stirred at 150° C. for 5 hours, then diluted with hydrochloric acid (1 M, 10 mL) and extracted with ethyl acetate (3×20 mL). The combined organic phase was concentrated in vacuo. The residue was purified by silica gel chromatography [petroleum ether: ethyl acetate=3:1] to give compound B-62 (0.4 g, 33% yield) as a colourless oil. GCMS: tR=8.87 min., (EI) m/z (M) =147.1.

### Example 63B

### 3-chloro-6-methylisoquinoline (B-63)

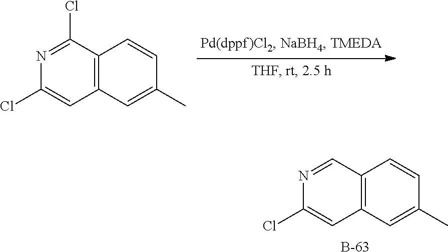

To a solution of 1,3-dichloro-6-methylisoquinoline (0.80 g, 3.8 mmol) in anhydrous tetrahydrofuran (80 mL) under nitrogen at room temperature was added [1,1′-bis(diphenylphosphino)ferrocene] dichloropalladium(ii) (0.14 g, 0.19 mmol), sodium borohydride (0.24 g, 6.4 mmol) and tetramethylethylenediamine (0.74 g, 6.4 mmol). The mixture was stirred at room temperature for 2.5 hours, then diluted with water (10 mL) and extracted with ethyl acetate (2×20 mL). The combined organic layers were dried over anhydrous sodium sulfate and concentrated in vacuo. The residue was purified by silica gel chromatography [petroleum ether: ethyl acetate=100:1] to give compound B- 63 (0.50 g, 74% yield) as a white solid. LCMS (B): tR=0.790 min., (ES⁺) m/z (M+H)⁺=178.1.

### Example 64B

### N-[(3,4-difluorophenyl)methyl]-2,2-dimethoxy-acetamide (B-64)

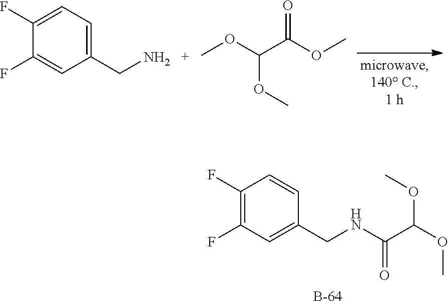

A mixture of (3,4-difluorophenyl) methanamine (2.0 g, 14 mmol) and methyl 2,2-dimethoxyacetate (3.8 g, 28 mmol) was stirred at 140° C. for 1 hour in the microwave. On completion, the reaction mixture was diluted with dichloromethane (20 mL) and concentrated in vacuo to give crude compound B-64 (4.3 g, crude) as a yellow solid. LCMS (E): tR=0.596 min., (ES⁺) m/z (M+H)⁺=246.1.

### Example 65B

### 6,7-difluoroisoquinolin-3-ol (B-65)

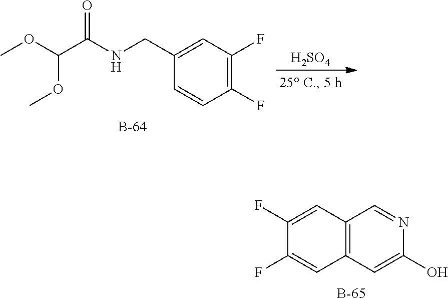

A solution of compound B-64 (13 g, 53 mmol) in concentrated sulfuric acid (100 mL) was stirred at 25° C. for 5 hours. On completion, the solution was poured slowly into ice-water (600 mL), then at 0° C. neutralized to pH 6-7 with saturated aqueous sodium bicarbonate. The resulting yellow solid was was collected by filtration to give compound B-65 (2.1 g, crude). LCMS (E): tR=0.483 min., (ES⁺) m/z (M+H)⁻=182.0.

### Example 66B

### (6,7-difluoro-3-isoquinolyl) trifluoromethanesulfonate (B-66)

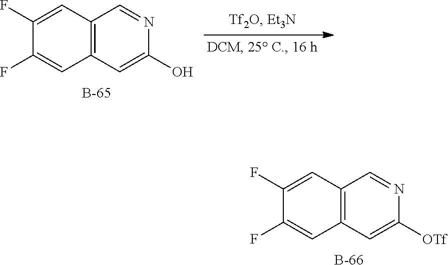

To a solution of compound B-65 (1.0 g, 5.5 mmol) and triethylamine (1.5 mL, 11 mmol) in dichloromethane (30 mL) at 25° C. was added dropwise trifluoromethanesulfonic anhydride (1.8 mL, 11 mmol). The mixture was stirred at 25° C. for 16 hours, then washed with water (2×30 mL), dried over over anhydrous sodium sulfate, filtered and concentrated under reduced pressure. The residue was purified by silica gel chromatography [petroleum ether: ethyl acetate=10:1] to give compound B-66 (1.0 g, 57.79% yield) as a white solid. LCMS (E): tR=0.856 min., (ES⁺) m/z (M+H)⁻=314.0; ¹H-NMR (CD₃OD, 400 MHz): δ 9.11 (s, 1H), 8.12 (t, J=7.6 Hz, 1H), 7.96 (t, J=7.6 Hz, 1H), 7.85 (s, 1H).

### Example 67B

### N-(3,4-dichlorobenzyl)-2,2-dimethoxyacetamide (B-67)

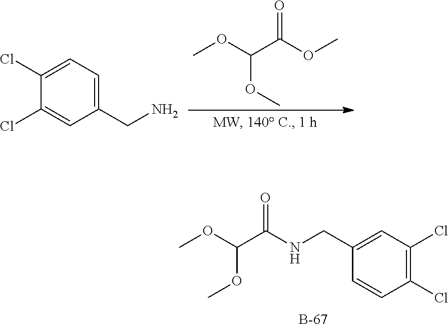

A mixture of (3,4-dichlorophenyl)methanamine (2.0 g, 11 mmol) and methyl 2,2-dimethoxyacetate (3.1 g, 23 mmol) was stirred at 140° C. for 1 hour in the microwave. The solution was diluted with dichloromethane (40 mL) and concentrated in vacuo to give compound B-67 (4.5 g, crude) as a yellow gum. LCMS (J): tR=1.290 min., (ES⁺) m/z (M+H)⁺=278.0.

### Example 68B

### 6,7-dichloroisoquinolin-3-ol (B-68)

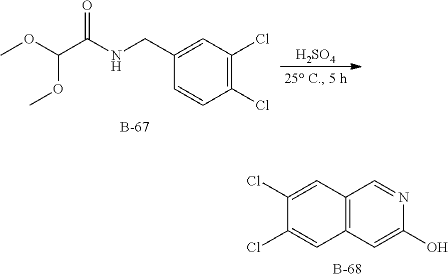

A mixture of compound B-67 (9 g, crude) in concentrated sulfuric acid (30 mL) was stirred at 25° C. for 5 hours. On completion, the mixture was poured into ice-water (500 mL), filtered and adjusted to pH 9 with aqueous sodium hydroxide (2 M). The resulting solid was collected by filtration and purified by silica gel chromatography [petroleum ether/ethyl acetate=3:1] to give compound B-68 (2.0 g, crude) as a yellow solid. LCMS (B): tR=0.648 min., (ES⁻) m/z (M+H)⁺=214.0.

### Example δ 9B

### 6,7-dichloroisoquinolin-3-yl trifluoromethanesulfonate (B-δ 9)

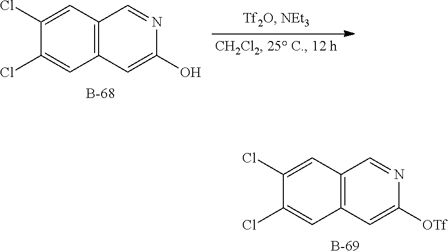

To a solution of compound B-68 (2 g, crude), triethylamine (2.4 g, 23.6 mmol) in dichloromethane (20 mL) at 25° C. was added dropwise trifluoromethanesulfonic anhydride (6.7 g, 23.6 mmol). The mixture was stirred at 25° C. for 12 hours, then concentrated in vacuo. The residue was purified by silica gel chromatography [petroleum ether/ethyl acetate=20:1] to give compound B-δ 9 (0.40 g, 5% yield over 3 steps) as a yellow solid. ¹H-NMR (CD₃OD, 400 MHz): 9.09 (s, 1H), 8.39 (s, 1H), 8.26 (s, 1H), 7.79 (s, 1H).

### Example 70B

### 6-bromo-2-chlorobenzofuran (B−70)

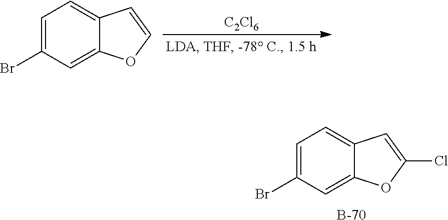

To a solution of 6-bromobenzofuran (1.0 g, 5.1 mmol) in anhydrous tetrahydrofuran (15 mL) at −78° C. under nitrogen was added dropwise lithium diisopropylamide (2.0 M in n-heptane, 3.1 mL, 6.2 mmol). The mixture was stirred at at −78° C. for 30 min, and then hexachloroethane (1.2 g, 5.1 mmol) was added dropwise. The reaction was stirred at −78° C. for another one hour, then poured into water (20 mL) and extracted with ethyl acetate (2×30 mL). The organic layer was dried over anhydrous sodium sulfate, filtered and concentrated in vacuo. The residue was purified by silica gel chromatography [petroleum ether: ethyl acetate=1:0] to give compound B-70 (0.36 g, 28% yield) as a colorless oil. ¹H-NMR (CD₃OD, 400 MHz): δ 7.69 (s, 1H), 7.46 (d, J=8.0 Hz, 1H), 7.40 (dd, J₁=8.4 Hz, J₂=1.6 Hz, 1H), 6.77 (s, 1H).

### Example 71B

### (6-bromobenzo[b]thiophen-2-yl)boronic acid (B-71)

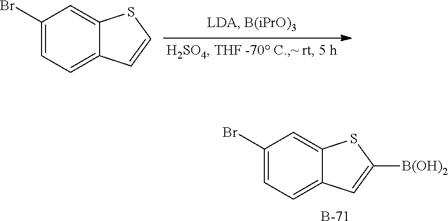

To a mixture of 6-bromobenzo[b]thiophene (2.0 g, 9.4 mmol) in anhydrous tetrahydrofuran (20 mL) at −70° C. was added dropwise lithium diisopropylamide (2 M in tetrahydrofuran, 7.1 mL, 14 mmol). The mixture was stirred at −70° C. for 1 hour. Then triisopropyl borate (3.5 g, 19 mmol) was added, and the solution was stirred at −70° C. for 2 hours. Then sulfuric acid (1.9 g, 19 mmol) was added slowly. The solution was stirred at room temperature for 2 hours, then diluted with water (10 mL) and extracted with ethyl acetate (3×20 mL). The combined organic layers was washed with brine, dried over anhydrous sodium sulfate, concentrated in vacuo to give compound B-71 (2.2 g, crude) as a white oil.

### Example 72B

### 6-bromo-2-(trifluoromethyl)benzo[b]thiophene (B-72)

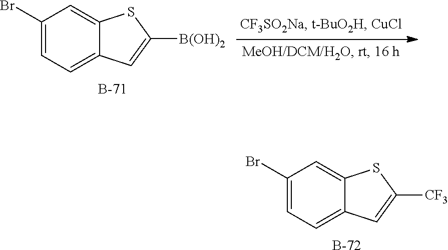

To a mixture of compound B-71 (1.0 g, 3.9 mmol), sodium trifluoromethanesulfinate (1.8 g, 12 mmol) and copper (I) chloride (0.39 g, 3.9 mmol) in methanol/dichloromethane/water (5:5:4, 28 mL) at room temperature was added slowly tert-butyl hydroperoxide (1.8 g, 19 mmol). The resulting mixture was stirred at room temperature for 16 hours, then quenched with sodium sulfite, concentrated in vacuo to remove most of organic solvent, diluted with water (50 mL) and extracted with dichloromethane (2×80 mL). The combined organic layers was washed with brine, dried over anhydrous sodium sulfate, and concentrated in vacuo. The residue was purified by silica gel chromatography [100% petroleum ether] to give compound B-72 (0.70 g, 58% yield for two steps) as a yellow oil.

### Example 73B

### 5-bromo-N-methyl-2-nitroaniline (B-73)

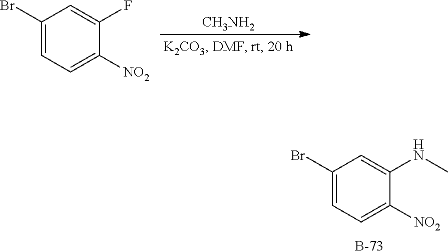

To a solution of 4-bromo-2-fluoro-l-nitrobenzene (5.0 g, 22.7 mmol) in N,N-dimethylformamide (80 mL) was added methylamine (2 M in tetrahydrofuran, 22.7 mL) and potassium carbonate (3.93 g, 28.4 mmol). The mixture was stirred at room temperature for 20 hours, then poured into water (50 mL) and extracted with ethyl acetate (3×150 mL). The combined organic layers were dried over anhydrous sodium sulfate and concentrated in vacuo to give compound B-73 (4.0 g, 77% yield) as a yellow solid. LCMS (B): tR=0.777 min., (ES⁺) m/z (M+H)⁺=231.0. ¹H-NMR (CD₃Cl, 400 MHz): δ 8.03 (d, J=9.2 Hz, 2H), 7.02 (d, J=1.6 Hz, 1H), 6.78 (dd, J₁=1.2 Hz, J₂=1.2 Hz, 1H), 3.02 (s, 3H).

### Example 74B

### 5 -bromo-N¹-methylbenzene-1,2 -diamine (B-74)

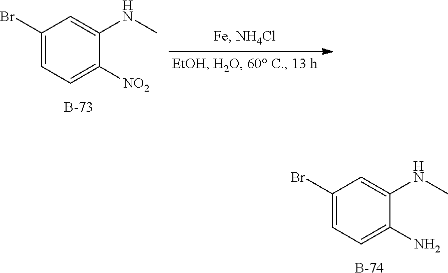

To a solution of compound B-73 (2.5 g, 10.8 mmol) in ethanol (100 mL) and water (75 mL) was added iron powder (3.6 g, 65 mmol) and ammonium chloride (3.5 g, 65 mmol). The mixture was stirred at 60° C. for 13 hours, then concentrated in vacuo to remove ethanol, diluted with water (30 mL) and extracted with ethyl acetate (3×90 mL). The combined organic layers were dried over anhydrous sodium sulfate and concentrated in vacuo to give compound B-74 (2.1 g, crude) as a brown oil. LCMS (B): tR=0.468 min., (ES⁺) m/z (M+H)⁺=201.0.

### Example 75B

### 6-bromo-1,2-dimethyl-1H-benzo[d]imidazole (B-75)

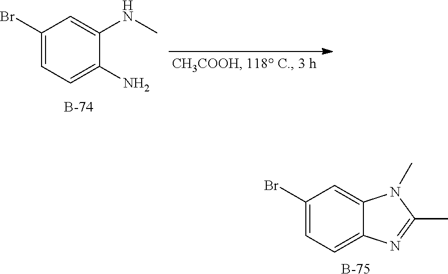

A solution of compound B-74 (1.8 g, 9.0 mmol) in acetic acid (27 mL) was stirred at 118° C. for 3 hours, then diluted with water (20 mL) and extracted with ethyl acetate (3×60 mL). The combined organic layers were concentrated and purified by silica gel chromatography [petroleum ether: ethyl acetate=1:0 to 3:1] to give compound B-75 (1.6 g, 77% yield) as a red solid. LCMS (B): tR=0.291 min., (ES⁻) m/z (M+H)⁺=225.0. ¹H-NMR (CD₃Cl, 400 MHz): δ 7.54 (d, J=8.4 Hz, 1H), 7.44 (d, J=1.2 Hz, 1H), 7.33 (dd, J₁=1.6 Hz, J₂=2.0 Hz, 1H), 3.71 (s, 3H), 2.60 (s, 3H).

### Example 76B

### 6-methyl-1H-quinazoline-2,4-dione (B-76)

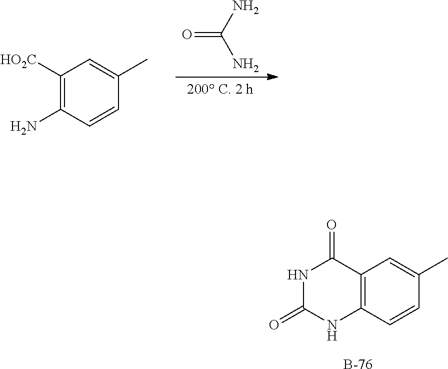

2-amino-5-methyl-benzoic acid (10 g, 66 mmol) and urea (14 g, 230 mmol) were stirred at 200° C. for 2 hours. On completion the mixture was poured into water (100 mL) and stirred for 10 hours. The resulting solid was collected by filtration and dried in vacuum to give compound B-76 (8.0 g, crude) as a yellow solid. LCMS (E): tR=0.472 min., (ES⁺) m/z (M+H)⁺=177.1.

### Example 77B

### 2,4-dichloro-6-methyl-quinazoline (B-77)

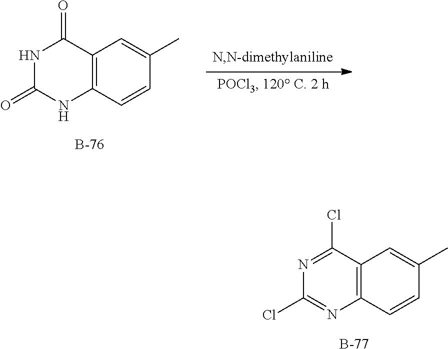

To a solution of compound B-76 (8.0 g, crude) in phosphorus oxychloride (0.24 L, 400 g, 2.6 mol) at room temperature was added dropwise N,N-dimethylaniline (5.7 mL, 5.5 g, 0.45 mol). The mixture was stirred at 120° C. for 2 hours, then poured into ice-water (500 mL). The resulting solid was collected by filtration and dried in vacuum to give compound B-77 (4 g, crude) as a yellow solid. LCMS (E): tR=0.794 min., (ES⁺) m/z (M+H)⁺=213.0

### Example 78B

### 2-chloro-6-methyl-quinazoline (B−78)

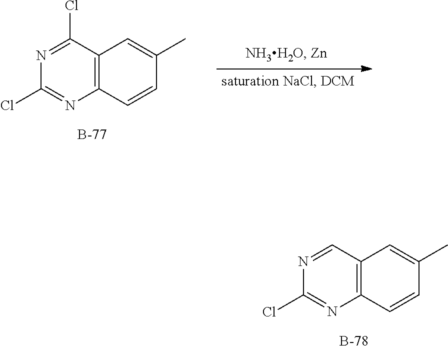

To a mixture of compound B-77 (1.9 g, 8.9 mmol) and ammonium hydroxide (9.1 g, 0.26 mol, 10 mL) in saturated aqueous sodium chloride (20 mL) and dichloromethane (30 mL) was added zinc powder (1.8 g, 27 mmol). The mixture was stirred at 50° C. for 5 hours, then poured into water (500 mL) and extracted with dichloromethane (3×300 mL). The combined organic phase was washed with brine (2×500 mL), dried over anhydrous sodium sulfate, filtered and concentrated in vacuum. The residue was purified by silica gel chromatography [petroleum ether: ethyl acetate=10:1 to 5:1] to give compound B-78 (500 mg, 31% yield) as a yellow solid. ¹H-NMR (CDCl₃, 400 MHz): 9.23 (s, 1H), 7.92 (d, J=8.8 Hz, 1H), 7.81 (d, J=8.8 Hz, 1H), 2.60 (s, 3H).

### Example 79B

### 7-methylquinazoline-2,4(1H,3H)-dione (B-79)

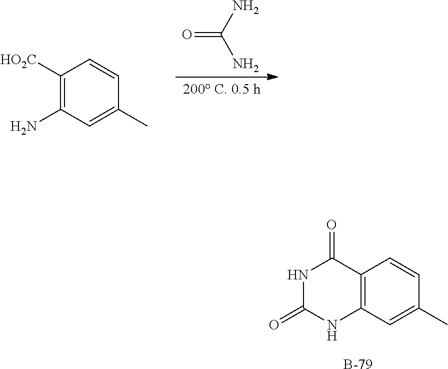

A mixture of 2-amino-4-methyl-benzoic acid (5 g, 33 mmol) and urea (8 g, 0.13 mol) was heated with stirring to 200° C. in a round-bottom flask open to the atmosphere for 0.5 hour. The mixture was cooled and added to water (50 mL). The resulting solid was collected by filtration to give compound B-79 (3.5 g, crude) as a white solid. LCMS (E): tR=0.504 min., (ES⁺) m/z (M+H)⁺=177.1. 1H-NMR (DMSO, 400 MHz): 11.17 (s, 1H), 11.09 (s, 1H), 7.77 (d, J=8.0 Hz, 1H), 7.00 (d, J=8.0 Hz, 1 H),6.95 (s, 1H), 2.36 (s, 3H).

### Example 80B

### 2,4-dichloro-7-methylquinazoline (B-80)

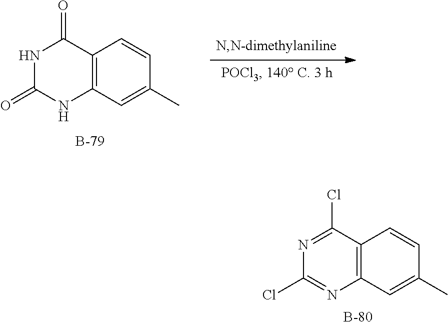

To a solution of compound B-79 (3.0 g, crude) in phosphorus oxychloride (31 g, 0.2 mmol) at room temperature was added dropwise N,N-dimethylaniline (2.1 g, 17 mmol). The mixture was heated to 140° C. for 3 hours, then poured into ice-water (100 mL). The resulting solid was collected by filtration and purified by silica gel chromatography (petroleum ether) to give compound B-80 (1.80 g, 45% yield) as a white solid. LCMS (E): tR=0.830 min., (ES+) m/z (M+H)+=213.1 1H-NMR (CDCl3, 400 MHz): 8.14 (d, J=8.8 Hz, 1H), 7.77 (s, 1H), 7.56 (d, J=8.8 Hz, 1H), 2.62 (s, 3H).

### Example 81B

### 2-chloro-7-methylquinazoline (B-81)

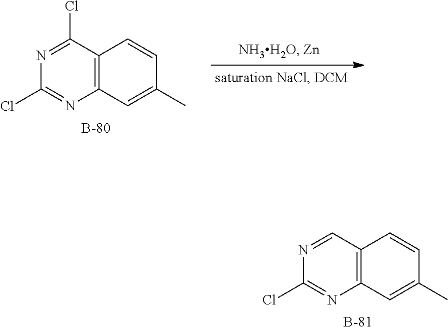

To a solution of compound B-80 (1 g, 4.7 mmol) in dichloromethane (15 mL) was added saturated aqueous sodium chloride (10 mL), ammonium hydroxide (27%, 4.6 g, 35 mmol) and zinc powder (0.92 g, 14 mmol). The mixture was stirred at 50° C. for 3 hours, then filtered and concentrated in vacuum. The residue was diluted with ethyl acetate (50 mL), washed with water (20 mL) and brine (20 mL), dried over anhydrous sodium sulfate and concentrated in vacuo. The residue was purified by silica gel chromatography [petroleum ether: ethyl acetate=100:1] to give compound B-81 (315 mg, 34% yield) as s yellow solid. 1H-NMR (DMSO-d6, 400 MHz): 9.52 (s, 1H), 8.12 (d, J=8.8 Hz, 1H), 7.77 (s, 1H), 7.66 (d, J=8.8 Hz, 1H), 2.58 (s, 3H).

### Example 82B

### 1-(2-(benzylthio)-4-bromophenyl)ethanone (B-82)

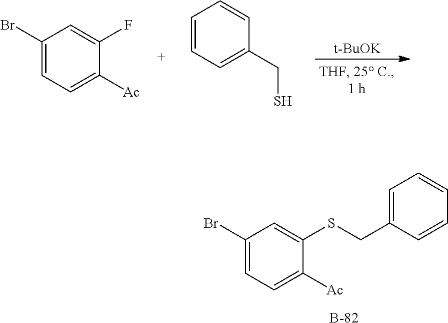

To a solution of potassium tert-butoxide (0.52 g, 4.6 mmol) in tetrahydrofuran (36 mL) at room temperature was added dropwise a solution of phenylmethanethiol (0.54 mL, 4.6 mmol) in tetrahydrofuran (6 mL). The mixture was stirred at room temperature for 5 mins, and 1-(4-bromo-2- fluoro-phenyl)ethanone (1.0 g, 4.6 mmol) was added. The mixture was stirred at room temperature for another 1 hour, then quenched with saturated aqueous ammonium chloride (40 mL) and extracted with ethyl acetate (3×40 mL). The combined organic layers were washed with brine (3×20 mL), dried over anhydrous sodium sulfate, filtered and concentrated in vacuo. The residue was purified by silica gel chromatography [petroleum ether: ethyl acetate=50:1] to give compound B-82 (1.1 g, 72% yield) as a white solid. ¹H-NMR (CD₃Cl, 400 MHz): δ 7.66 (d, J=8.4 Hz, 1H), 7.55 (d, J=1.2 Hz, 1H), 7.40 (d, J=7.2 Hz, 2H), 7.35-7.28 (m, 4H), 4.13 (s, 2H), 2.58 (s, 3H).

### Example 83B

### 6-bromo-3-methylbenzo[d]isothiazole (B-83)

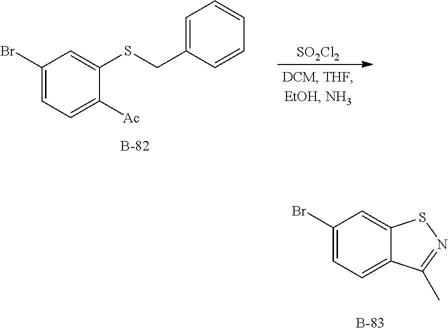

To a solunon or compounu B-82 (1.1 g, 3.3 mmoi) in dichloromethane (10 mL) at room temperature was added dropwise sulfuryl chloride (0.33 mL, 3.3 mmol). The mixture was stirred at room temperature for 0.5 hour, then concentrated in vacuo. The residue was dissolved in tetrahydrofuran (10 mL) and treated with a saturated solution of ammonia in ethanol (10 mL). The resulting mixture was stirred at room temperature for 1 hour, diluted with water (20 mL) and extracted with ethyl acetate (3×20 mL). The combined organic layers were washed with brine (3×20 mL), dried over anhydrous sodium sulfate, filtered and concentrated in vacuo. The residue was purified by silica gel chromatography [petroleum ether: ethyl acetate=30:1] to give compound B-83 (0.50 g, 66% yield) as a yellow solid. ¹H-NMR (CD₃Cl, 400 MHz): δ 8.09 (d, J=1.2 Hz, 1H), 7.79 (d, J=8.4 Hz, 1H), 7.55 (dd, J₁=1.2 Hz, J₂=1.2 Hz, 1H), 2.74 (s, 3H).

### Example 84B

### 6-bromo-3-chlorobenzofuran (B-84-1) and 6-bromo-3,5-dichlorobenzofuran (B-84-2)

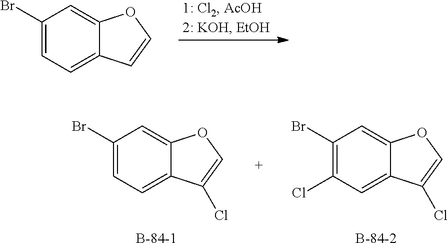

Chlorine gas was bubbled through a solution of 6-bromobenzofuran (0.50 g, 2.54 mmol) in acetic acid (6 mL) at 0° C. for 0.5 hours. The mixture was concentrated and added to a solution of potassium hydroxide (1.29 g, 23.0 mmol) in ethanol (23 mL). The mixture was stirred at 25° C. for 18 hours, then partially concentrated in vacuo, neutralized to pH 7 with hydrochloric acid (6 N) and extracted with ethyl acetate (3×20 mL). The combined organic layers were concentrated in vacuo and purified by column chromatography [petroleum ether: ethyl acetate=100:1] to give a mixture of compound B-84-1 and compound B-84-2 (0.6 g, ratio of products 1:2.3 by GCMS) as a white solid. GCMS (B): tR=4.910 min, (ES+) m/z=231.9. and GCMS (B): tR=5.752 min, (ES+) m/z=265.9.

### Example 85B

### methyl 2-(2-acetyl-5-bromophenoxy)acetate (B-85)

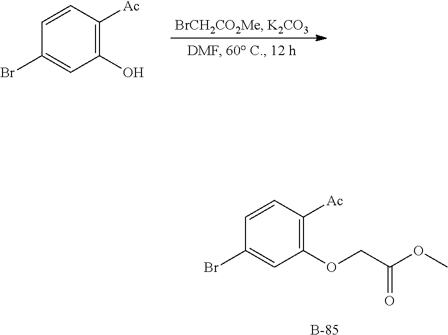

To a solution of 1-(4-bromo-2-hydroxyphenyl)ethanone (5.0 g, 23.3 mmol) in N,N-dimethylformamide (50 mL) was added potassium carbonate (6.4 g, 46.5 mmol) and methyl 2-bromoacetate (3.9 g, 25.6 mmol). The mixture was stirred at 60° C. for 12 hours, then diluted with ethyl acetate (400 mL) and washed with saturated aqueous sodium carbonate (4×50 mL) and brine (2×50 mL), dried over anhydrous sodium sulfate, filtered and concentrated in vacuo. The residue was purified by silica gel chromatography [petroleum ether: ethyl acetate=40:1 to 20:1) to give compound B-85 (0.7 g, 53% purity, 6% yield) as a yellow solid. LCMS (B): tR=0.769 min., (ES⁺) m/z (M+H)⁺=287.1.

### Example 86B

### 2-(2-acetyl-5-bromophenoxy)acetic acid (B-86)

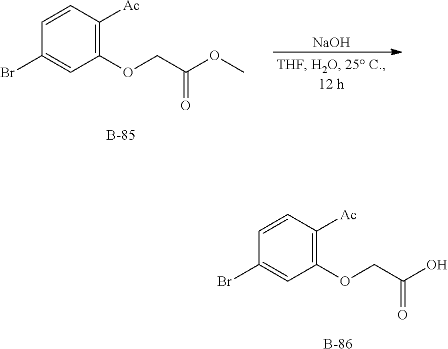

To a solution of compound B-85 (0.69 g, 2.4 mmol) in tetrahydrofuran (6 mL) and water (2 mL) at 25° C. was added sodium hydroxide (0.21g, 5.3 mmol). The mixture was stirred at 25° C. for 12 hours, then concentrated in vacuo to remove tetrahydrofuran, diluted with water (90 mL), washed with ethyl acetate (3×10 mL), adjusted to pH 2 with concentrated hydrochloric acid and extracted with ethyl acetate (3×50 mL). The combined organic layers were washed with brine (3×10 mL), dried over sodium sulfate, filtered and concentrated under in vacuo to give compound B-86 (0.69 g, crude) as a yellow solid. LCMS (Y): tR=0.739 min., (ES⁺) m/z (M+H)⁺=273.1.

### Example 87B

### 6-bromo-3-methylbenzofuran (B-87)

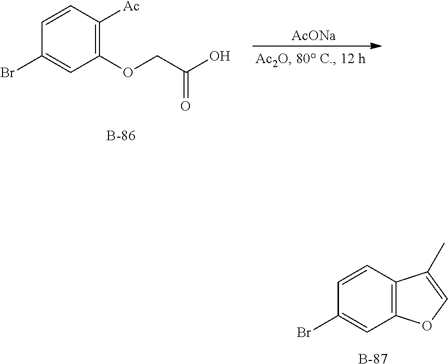

A mixture of compound B-86 (0.68 g, crude) and sodium acetate (0.51 g, 6.3 mmol) in acetic anhydride (10 mL) was stirred at 80° C. for 12 hours. On completion, the mixture was concentrated in vacuo, and the residue was purified by silica gel chromatography [petroleum ether] to give compound B-87 (0.38 g, 8% yield over 2 steps) as colorless oil. ¹H-NMR (CDCl₃, 400 MHz): 7.63 (s, 1H), 7.40-7.35 (m, 3H), 2.23 (s, 1H).

### Example 88B

### 2-chloro-5-iodopyrimidin-4(3H)-one (B-88)

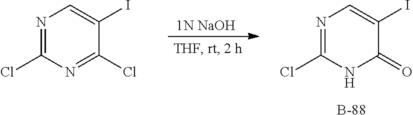

To a solution of 2,4-dichloro-5-iodo-pyrimidine (1.0 g, 3.6 mmol) in THF (20 mL) was added sodium hydroxide (1 N, 15 mL). The mixture was stirred at 25° C. for 2 hours, then poured into water (100 mL) and extracted with ethyl acetate (4×50 mL). The combined organic phase was washed with brine (2×100 mL), dried with anhydrous sodium sulfate, filtered and concentrated in vacuo to give compound B-88 (0.6 g, 64% yield) as a yellow solid. ¹H-NMR (MeOD, 400 MHz): 8.40 (s, 1H), 7.85(s, 1H).

### Example 89B

### 2-chloro-6-methyl-furo[2,3-d]pyrimidine (B-89)

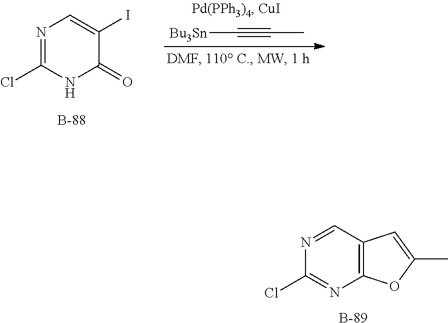

A solution of compound B-88 (600 mg, 2.3 mmol), tetrakis(triphenylphosphine) palladium(0) (135 mg, 0.12 mmol), cuprous iodide (45 mg, 0.23 mmol) and tributyl(prop-1-ynyl)stannane (1.5 g, 2.7 mmol) in N,N-dimethylformamide (6 mL) was stirred at 110° C. for 1 hour under nitrogen in the microwave. On completion, the mixture was poured into water (60 mL) and extracted with ethyl acetate (3×45 mL). The combined organic layers were concentrated in vacuo and purified by silica gel chromatography [petroleum ether: ethyl acetate=10:1] to give compound B-89 (120 mg, 30% yield) as a yellow soild. ¹H-NMR (CD₃OD, 400 MHz): δ 8.74 (s, 1H), 6.49 (s, 1H), 2.55 (s, 3H).

### Example 90B

### 6-bromo-3-chlorobenzo[b]thiophene-2-carboxylic acid (B-90)

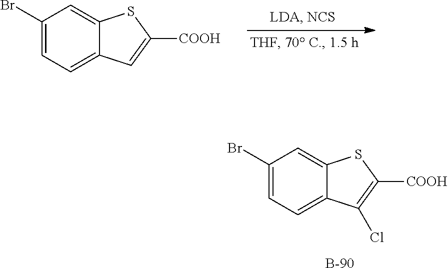

To a solution or 6-bromobenzothiophene-2-carboxylic acid (5.0 g, 20 mmoi) in tetrahydropyran (100 mL) at −70° C. under nitrogen was added lithium diisopropylamide (2 M in THF, 100 mL). The mixture was stirred for 1 hour, and then N-chlorosuccinimide (26 g, 195 mmol) was added at −70° C. The solution was stirred for 0.5 hour, then quenched with saturated aqueous ammonium chloride (100 mL) and extracted with ethyl acetate (3×60 mL). The organic layer was washed with brine (3×40 mL), dried over anhydrous sodium sulfate, filtered and concentrated in vacuo to give compound B-90 (3.5 g, 40% purity, 61% yield) as a brown solid.

### Example 91B

### 6-bromo-3-chloro-1H-indene (B-91)

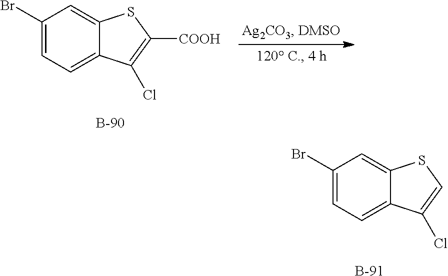

To a solution of compound B-90 (3.5 g, 4.8 mmol, 40% purity) in dimethyl sulfoxide (30 mL) was added silver carbonate (1.32 g, 4.8 mmol). The mixture was stirred at 120° C. for 4 hours, then diluted with water (20 mL), filtered, adjusted to pH 9 with saturated aqueous sodium carbonate and extracted with ethyl acetate (3×20 mL). The combined orangic phase was concentrated in vacuo and purified by silica gel chromatography [petroleum ether] to give compound B-91 (0.50 g, 33% yield) as a white solid. GCMS: tR=5.897 min., (ES⁺) m/z (M)⁺=247.9.

### Example 92B

### 1-(4-bromo-2-fluorophenyl)-2,2,2-trifluoroethanol (B-92)

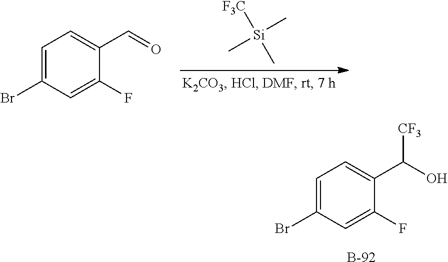

To a solution of 4-bromo-2-fluorobenzaldehyde (5.0 g, 25 mmol) in N,N-dimethylformamide (30 mL) was added trimethyl(trifluoromethyl)silane (4.2 g, 30 mmol) and potassium carbonate (0.34 g, 2.5 mmol). The mixture was stirred at room temperature for 3 hours. Then hydrochloric acid (1 M, 37 mL) was added dropwise to the solution. The mixture was stirred for another 4 hours, then extracted with ethyl acetate (2×100 mL). The combined organic layers were washed with water (5×20 mL), dried over anhydrous sodium sulfate and concentrated in vacuo to give compound B-92 (6.7 g, crude) as a yellow oil. TLC [petroleum ether: ethyl acetate=20:1]: Rf=0.10.

### Example 93B

### 1-(4-bromo-2-fluorophenyl)-2,2,2-trifluoroethanone (B-93)

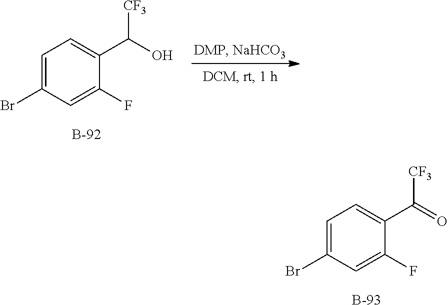

To a solution of compound B-92 (6.7 g, 25 mmol) in dichloromethane (150 mL) was added 1,1,1-triacetoxy-1,1-dihydro-1,2-benziodoxo1-3(11-1)-one (39 g, 91 mmol) and sodium bicarbonate (8.3 g, 98 mmol). The mixture was stirred at room temperature for 1 hour, then poured into saturated aqueous sodium sulfite (40 mL) and extracted with ethyl acetate (2×100 mL). The combined organic layers were concentrated and purified by silica gel chromatography [petroleum ether: ethyl acetate=20:1] to give compound B-93 (5.0 g, 75% yield for two steps) as a light yellow oil. TLC [petroleum ether: ethyl acetate=10:1]: Rf=0.22.

### Example 94B

### methyl 6-bromo-3-(trifluoromethyl)benzo[b]thiophene-2-carboxylate (B-94)

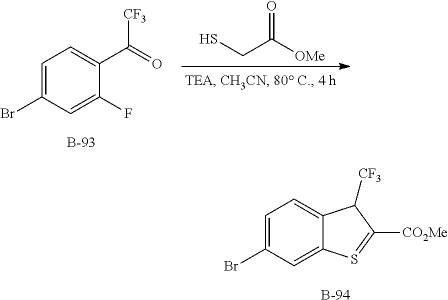

To a solution of compound B-93 (5.0 g, 12 mmol) in acetonitrile (50 mL) was added methyl 2-mercaptoacetate (1.4 g, 13 mmol) and triethylamine (1.6 g, 16 mmol). The mixture was stirred at 80° C. for 4 hours, then concentrated in vacuo to remove acetonitrile, diluted with water (50 mL) and extracted with ethyl acetate (2×100 mL). The combined organic layers were concentrated and purified by silica gel chromatography [petroleum ether: ethyl acetate=100:1] to give compound B-94 (2.7 g, 50% yield) as a yellow solid. ¹H-NMR (CDCl₃, 400 MHz): δ 8.03 (d, J=1.6 Hz, 1H), 7.99 (d, J=8.8 Hz, 1H), 7.61 (dd, J₁=8.8 Hz, J₂=1.6 Hz, 1H), 3.99 (s, 3H).

### Example 95B

### 6-bromo-3-(trifluoromethyl)benzo[b]thiophene-2-carboxylic acid (B-95)

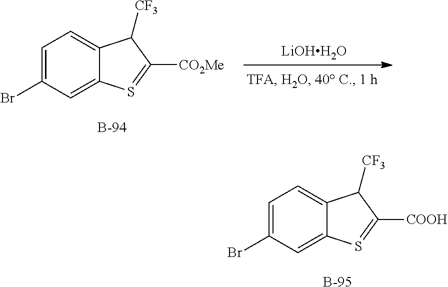

To a solution of compound B-94 (1.5 g, 4.4 mmol) in tetrahydrofuran (20 mL) and water (4 mL) was added lithium hydroxide monohydrate (0.37 g, 8.8 mmol). The mixture was stirred at 40° C. for 1 hour, then adjusted to pH 5.0 with 1 N hydrochloric acid, diluted with water (20 mL) and extracted with ethyl acetate (2×50 mL). The combined organic layers were concentrated in vacuo to give compound B-95 (1.3 g, crude) as a yellow solid. LCMS (B): tR=0.906 min., (ES⁺) m/z (M+H)⁺=326.8.

### Example 96B

### 6-bromo-3-(trifluoromethyl)benzo[b]thiophene (B-96)

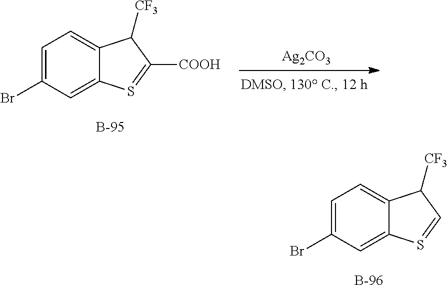

To a solution of compound B-95 (1.3 g, 4.0 mmol) in dimethylsulfoxide (10 mL) was added silver carbonate (0.11 g, 4.0 mmol). The mixture was stirred at 130° C. for 12 hours, then poured into water (20 mL) and extracted with ethyl acetate (2×50 mL). The combined organic layers were concentrated and purified by silica gel chromatography [petroleum ether: ethyl acetate=500:1] to give compound B-96 (0.7 g, 56% yield) as a colourless oil. ¹H-NMR (CD₃OD, 400 MHz): δ 8.24 (s, 1H), 8.21 (s, 1H), 7.80 (d, J=8.8 Hz, 1H), 7.63 (dd, J₁=8.8 Hz, J₂=1.6 Hz, 1H).

### Example 97B

### 6-bromo-3-fluorobenzo[b]thiophene-2-carboxylic acid (B-97)

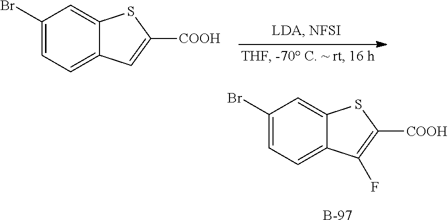

To a solution of 6-bromobenzo[b]thiophene-2-carboxylic acid (20 g, 78 mmol) in tetrahydrofuran (200 mL) at −70° C. was added dropwise lithium diisopropylamide (2 M in tetrahydrofuran, 195 mL, 0.39 mol). The mixture was stirred at −70° C. for 1 hour, and then N- fluorobenzenesulfonimide (123 g, 0.39 mol) in tetrahydrofuran (200 mL) was added. The mixture was allowed to room temperature and stirred for 15 hours, then quenched with water (300 mL), adjusted to pH 3˜4 with 2N hydrochloric acid and extracted with ethyl acetate (2×500 mL). The combined organic layers were concentrated and purified by silica gel chromatography [ethyl acetate: methanol=100:1 to 10:1) to give compound B-97 (20 g, crude) as a brown solid.

### Example 98B

### 6-bromo-3-fluorobenzo[b]thiophene (B-98)

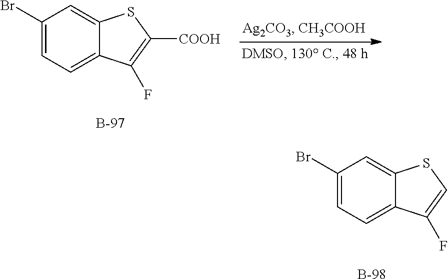

To a solution of compound B-97 (20 g, crude) in dimethyl sulfoxide (300 mL) was added silver carbonate (2.0 g, 7.3 mmol) and acetic acid (0.23 g, 3.6 mmol). The mixture was stirred at 130 ° C. for 48 hours, then diluted with water (500 mL) and extracted with ethyl acetate (2×500 mL). The combined organic layers were concentrated in vacuo and purified by silica gel chromatography [100% petroleum ether] twice to give compound B-98 (1 g, 6% yield for two steps) as an oil.

### Example 99B

### 6-bromo-2-chloro-3-fluorobenzo[b]thiophene (B-99)

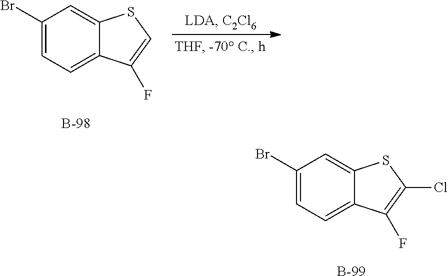

To a solution of compound B-98 (1 g, 4.3 mmol) in tetrahydrofuran (15 mL) at −70° C. was added dropwise lithium diisopropylamide (2 M in tetrahydrofuran, 3.3 mL, 6.5 mmol). The mixture was stirred at −70° C. for 1 hour, and then hexachloroethane (1.0 g, 4.3 mmol) in tetrahydrofuran (5 mL) was added. The mixture was stirred for another 1 hour, then quenched with water (20 mL) and extracted with ethyl acetate (2×50 mL). The combined organic layers were concentrated in vacuo and purified by silica gel chromatography [100% petroleum ether] to give compound B-99 (0.40 g, 35% yield) as a white solid.

### Example 100B

### N-(4-chloro-3-fluorobenzyl)-2, 2-dimethoxyacetamide (B-100)

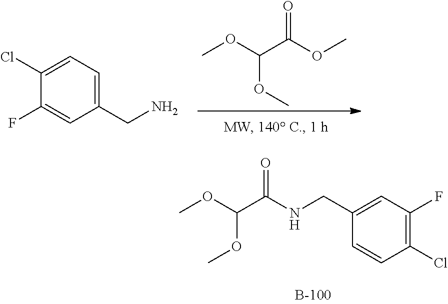

A mixture of (4-chloro-3-fluorophenyl)methanamine (2.0 g, 12.5 mmol) and methyl 2,2-dimethoxyacetate (3.4 g, 25.1 mmol) was stirred at 140° C. for 1 hour in microwave. On completion, the reaction was diluted with dichloromethane (200 mL) and concentrated under reduced pressure to give compound B-100 (4.9 g, crude) as a yellow gum. LCMS (B): tR=0.665 min., (ES⁺) m/z (M+H)⁺=262.0.

### Example 101B

### 6-chloro-7-fluoroisoquinolin-3-ol (B-101)

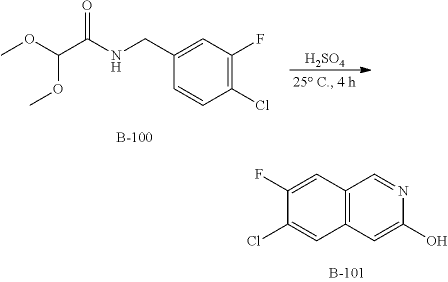

Compound B-100 (14 g, crude) was added to concentrated sulfuric acid (50 mL) at 25° C. The mixture was stirred at 25° C. for 4 hours, then poured into ice-water (1.5 L), filtered, adjusted to pH 9 with aqueous sodium hydroxide (2 M) and extracted with ethyl acetate (4×400 mL). The combined organic phase was washed with brine (2×300 mL), dried over anhydrous sodium sulfate, filtered and concentrated in vacuo to give compound B-101 (14.0 g, crude) as a yellow solid. LCMS (B): tR=0.586 min., (ES) m/z (M+H)⁺=198.0.

### Example 102B

### 6-chloro-7-fluoroisoquinolin-3-yl trifluoromethanesulfonate (B-102)

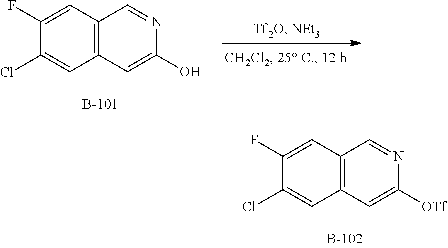

To a solution of compound B-101 (13.8 g, crude) and triethylamine (28.3 g, 279.4 mmol) in dichloromethane (100 mL) at 25° C. was added dropwise trifluoromethanesulfonic anhydride (78.8 g, 279.4 mmol). The mixture was stirred at 25° C. for 12 hours, then concentrated in vacuo. The residue was purified by silica gel chromatography (petroleum ether/ethyl acetate=20:1) to give compound B-102 (8.0 g, 76% purity, 33% yield over 3 steps) as a brown oil. LCMS (B): tR=1.073 min., (ES⁻) m/z (M+H)⁺=329.9; ¹H-NMR (CD₃OD, 400 MHz): 9.10 (s, 1H), 8.39 (d, J=6.8 Hz, 1H), 8.03 (d, J=9.2 Hz, 1H), 7.81 (s, 1H).

### Example 103B

### N-(3-chloro-4-fluorobenzyl)-2,2-dimethoxyacetamide (B-103)

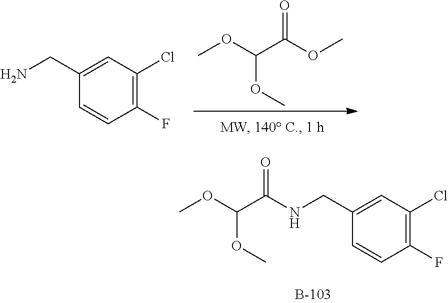

A mixture of (3-chloro-4-fluorophenyl)methanamine (2.0 g, 13 mmol) and methyl 2,2-dimethoxyacetate (1.7 g, 13 mmol) was stirred at 140° C. for 1 hour in microwave. On completion, the mixture was concentrated in vacuo to give compound B-103 (3.3 g, crude) as a yellow gum. LCMS (B): tR=0.711 min., (ES⁻) m/z (M+H) =262.2.

### Example 104B

### 7-chloro-6-fluoroisoquinolin-3-ol (B-104)

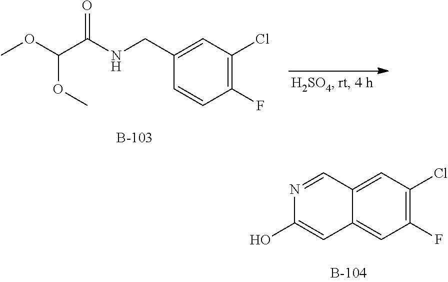 [SMILES: COC(OC)C(=O)NCc1ccc(F)c(Cl)c1 ; Oc1cc2cc(F)c(Cl)cc2cn1]

Compound B-103 (9.8 g, crude) was added to concentrated sulfuric acid (100 mL) at room temperature. The mixture was stirred at room temperature for 4 hours, then poured into ice-water (500 mL) and adjusted to pH 10 with aqueous sodium hydroxide (2 M), resulting in formation of a solid. The solid was collected by filration and dried in vacuo to give compound B-104 (5.0 g, crude) as a yellow solid. LCMS (B): tR=0.585 min., (ES⁺) m/z (M+H)⁺=198.0.

### Example 105B

### 7-chloro-6-fluoroisoquinolin-3-yl trifluoromethanesulfonate (B-105)

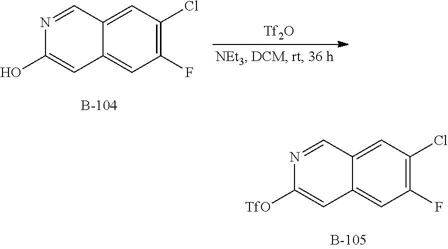 [SMILES: "Tf2O" ; Oc1cc2cc(F)c(Cl)cc2cn1 ; O=S(=O)(Oc1cc2cc(F)c(Cl)cc2cn1)C(F)(F)F]

To a solution of compound B-104 (5.0 g, 25 mmol) and triethylamine (10 g, 0.10 mol) in dichloromethane (100 mL) at room temperature was added dropwise trifluoromethanesulfonic anhydride (29 g, 0.10 mol). The mixture was stirred at room temperature for 36 hours, and then poured into water (100 mL) and extracted with dichloromethane (2×100 mL×2). The combined organic layers were concentrated and purified by silica gel chromatography [petroleum ether: ethyl acetate=50:1] to give compound B-105 (1.0 g, 8% yield over three steps) as a white solid. LCMS (B): tR=1.061 min., (ES⁻) m/z (M+H)⁺=329.9; ¹H-NMR (CD₃OD, 400 MHz): δ 9.10 (s, 1H), 8.43 (d, J=7.2 Hz, 1H), 7.92 (d, J=10.0 Hz, 1H), 7.84 (s, 1H).

### Example 106B

### 2,2-dimethoxy-N-(4-(trifluoromethyl)benzyl)acetamide (B-106)

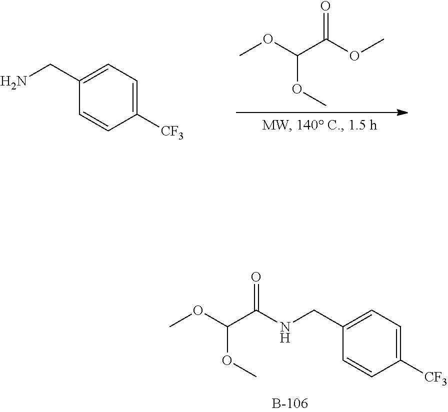

A mixture of (4-(trifluoromethyl)phenyl)methanamine (2.0 g, 11 mmol) and methyl 2,2-dimethoxyacetate (1.7 g, 13 mmol) was stirred at 140° C. for 1.5 hours in microwave. On completion, the mixture was concentrated in vacuo to give compound B-106 (3.1 g, crude) as a yellow gum. LCMS (B): tR=0.718 min., (ES⁺) m/z (M+H)⁺=278.0.

### Example 107B

### 6-(trifluoromethyl)isoquinolin-3-ol (B-107)

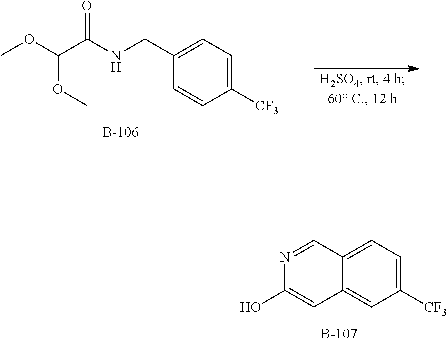 [SMILES: COC(OC)C(=O)NCc1ccc(C(F)(F)F)cc1 ; Oc1cc2cc(C(F)(F)F)ccc2cn1]

Compound B-106 (9.3 g, crude) was added to concentrated sulfuric acid (40 mL) at room temperature. The mixture was stirred at room temperature for 4 hours, then heated to 60° C. for another 12 hours. On completion, the mixture was poured into ice-water (500 mL) and adjusted to pH 8.0 with aqueous sodium hydroxide (2 M), resulting in formation of a solid. The solid was collected by filtration and dried in vacuo to give compound B-107 (3.7 g, crude) as a yellow solid. LCMS (B): tR=0.630 min., (ES⁺) m/z (M+H)⁻=214.1.

### Example 108B

### 6-(trifluoromethyl)isociuinolin-3-yltrifluoromethanesulfonate (B-108)

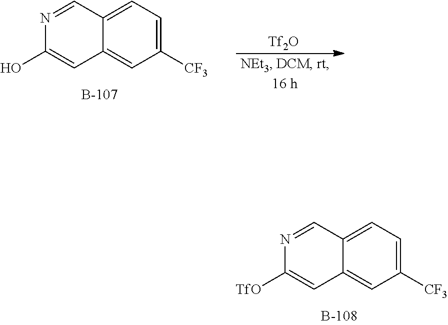 [SMILES: "Tf2O" ; Oc1cc2cc(C(F)(F)F)ccc2cn1 ; O=S(=O)(Oc1cc2cc(C(F)(F)F)ccc2cn1)C(F)(F)F]

To a solution of compound B-107 (3.7 g, 17 mmol) and triethylamine (7.0 g, δ 9 mmol) in dichloromethane (40 mL) at room temperature was added dropwise 3- trifluoromethanesulfonic anhydride (20 g, δ 9 mmol). The mixture was stirred at room temperature for 16 hours, then poured into water (100 mL) and extracted with dichloromethane (2×200 mL). The combined organic layers were concentrated and purified by silica gel chromatography [petroleum ether: ethyl acetate=30:1] to give compound B-108 (0.45 g, 4% yield over three steps) as a white solid. LCMS (B): tR=0.957 min., (ES⁻) m/z (M+H)⁺=346.0; ¹H-NMR (CD₃OD, 400 MHz): δ 9.29 (s, 1H), 8.49 (s, 1H), 8.41 (d, J=8.8 Hz, 1H), 8.03 (s, 1H), 7.96 (dd, J₁=8.8 Hz, J₂=1.6 Hz, 1H).

### Example 109B

### methyl 5-chlorofuro[2,3-c]pyridine-2-carboxylate (B-109)

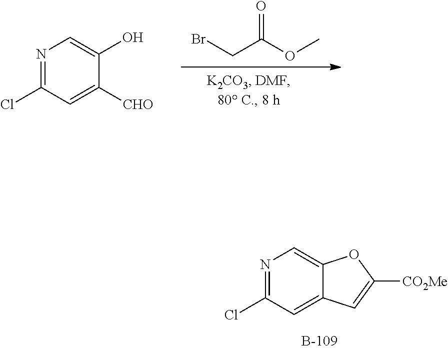

To a mixture of 2-chloro-5-hydroxyisonicotinaldehyde (4.0 g, 25 mmol) and methyl 2-bromoacetate (5.8 g, 38 mmol) in N,N-dimethylformamide (40 mL) under nitrogen at room temperature was added potassium carbonate (6.9 g, 50 mmol). The reaction mixture was stirred at 80° C. for 8 hours, then diluted with water (100 mL) and extracted with ethyl acetate (3×100 mL). The combined organic layers were washed with brine (2×100 mL), dried over anhydrous sodium sulfate and concentrated in vacuo. The residue was purified by silica gel silica gel chromatography [petroleum ether: ethyl acetate=25:1] to give compound B-109 (4.2 g, 79% yield) as a white solid. LCMS (E): tR=0.68 min., 212.6 m/z (M+1).

### Example 110B

### 5-chlorofuro[2,3-c]pyridine-2-carboxylic acid (B-110)

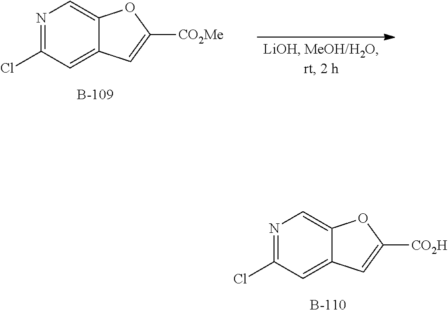 [SMILES: COC(=O)c1cc2cc(Cl)ncc2o1 ; O=C(O)c1cc2cc(Cl)ncc2o1]

To a mixture of compound B-109 (3.5 g, 16 mmol) in methanol (25 mL) and water (5 mL) under nitrogen at room temperature was added lithium hydroxide (1.4 g, 33 mmol). The reaction mixture was stirred at room temperature for 2 hours, then concentrated to remove methanol, diluted with water, and acidified to pH 4-5 with 1 M hydrochloric acid, resulting in precipitation of a solid. The solid was collected by filtration and dried in vacuo to give compound B-110 (3.0 g, 92% yield) as a white solid. ¹H-NMR (CD₃OD, 400 MHz): δ 8.79 (s, 1H), 7.85 (s, 1H), 7.62 (s, 1H).

### Example 111B

### 5-chlorofuro[2,3-c]pyridine (B-111)

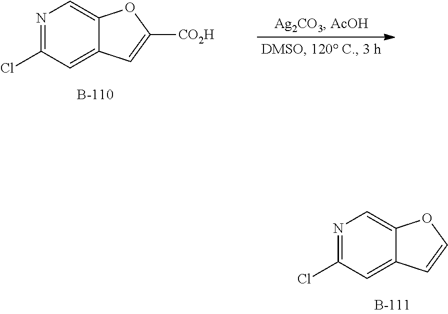 [SMILES: O=C(O)c1cc2cc(Cl)ncc2o1 ; Clc1cc2ccoc2cn1]

To a mixture of compound B-110 (1.5 g, 7.6 mmol) in dimethyl sulfoxide (15 mL) under nitrogen at room temperature was added silver carbonate (0.21 g, 0.76 mmol) and acetic acid (23 mg, 0.38 mmol). The reaction mixture was stirred at 120° C. for 3 hours, then diluted with water (20 mL) and extracted with ethyl acetate (3×30 mL). The combined organic layers were washed with brine (2×30 mL), dried over anhydrous sodium sulfate and concentrated in vacuo. The residue was purified by silica gel silica gel chromatography [petroleum ether: ethyl acetate=10:1] to give compound B-111 (0.45 g, 39% yield) as a yellow solid. ¹H-NMR (CDCl₃, 400 MHz): δ 8.67 (s, 1H), 7.82 (s, 1H), 7.59 (s, 1H), 6.81 (s, 1H). Compound B-111 was used to prepare compounds 98-P1 and 98-P2.

### Example 112B

### methyl 6-chlorothieno[3,2-c]pyridine-2-carboxylate (B-112)

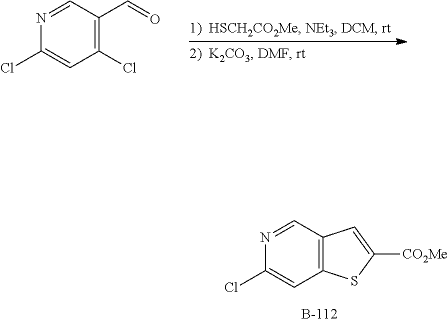 [SMILES: O=Cc1cnc(Cl)cc1Cl ; COC(=O)c1cc2cnc(Cl)cc2s1]

To a solution of 4,6-dichloropyridine-3-carbaldehyde (6.0 g, 34 mmol) in dichloromethane (10 mL) at 0° C. were added methyl thioglycolate (3.6 g, 34 mmol) and triethylamine (6.9 g, 68 mmol). The mixture was stirred at 25° C. for 1 hour, then filtered and concentrated in vacuum. The residue was dissolved in N,N-dimethylformamide (10 mL), and potassium carbonate (7.1 g, 51 mmol) was added. The mixture was stirred at 25° C. for 11 hours, then diluted with water (100 mL) and extracted with ethyl acetate (3×60 mL). The combined organic phase was washed with brine (2×30 mL), dried with anhydrous Na₂SO₄, filtered and concentrated in vacuo. The residue was purified by silica gel chromatography [petroleum ether: ethyl acetate=30:1 to 10:1] to give compound B-112 (3.5 g, 45% yield) as an off-white solid. ¹H-NMR (CDCl₃, 400 MHz): δ 8.99 (s, 1H), 8.13 (s, 1H), 7.86 (s, 1H), 4.00 (s, 3H).

### Example 113B

### 6-chlorothieno[3,2-c]pyridine-2-carboxylic acid (B-113)

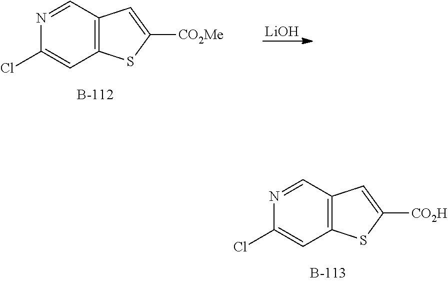 [SMILES: COC(=O)c1cc2cnc(Cl)cc2s1 ; "LiOH" ; O=C(O)c1cc2cnc(Cl)cc2s1]

To a solution of compound B-112 (3.5 g, 15 mmol) in methanol (20 mL) and water (5 mL) was added lithium hydroxide (1.9 g, 46 mmol). The mixture was stirred at 25° C. for 2 hours, then diluted with 0.2N hydrochloric acid (200 mL) and extracted with ethyl acetate (3×50 mL). The combined organic phase was washed with brine (2×200 mL), dried with anhydrous Na₂SO₄, filtered and concentrated in vacuo to give compound B-113 (3 g, crude) as a white solid. ¹H-NMR (DMSO- d6, 400 MHz): δ 9.10 (s, 1H), 8.34 (s, 1H), 8.25(s, 1H).

### Example 114B

### 6-chlorothieno[3,2-c]pyridine (B-114)

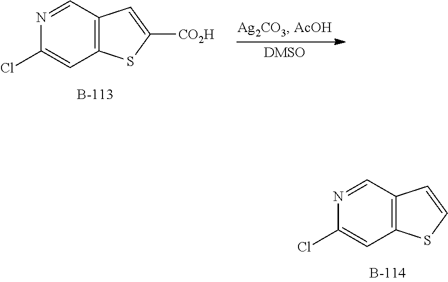

To a solution of compound B-113 (3.0 g, crude) in dimethylsulfoxide (15 mL) were added silver carbonate (387 mg, 1.4 mmol) and acetic acid (42 mg, 0.070 mmol). The mixture was stirred at 120° C. for 2 hours, then diluted with water (20 mL) and extracted with ethyl acetate (3×30 mL). The combined organic phase was washed with brine (3×20 mL) and concentrated in vacuo. The residue was purified by silica gel chromatography [petroleum ether: ethyl acetate=5:1] to give compound B-114 (2 g, 84% yield) as a white solid. ¹H-NMR (CDCl₃, 400 MHz): δ 8.91 (s, 1H), 7.86 (s, 1H), 7.52 (d, J=5.2 Hz, 1H), 7.44 (d, J=5.6 Hz, 1H).

### Example 115B

### 6-chloro-2-methyl-thieno[3,2-c]pyridine (B-115)

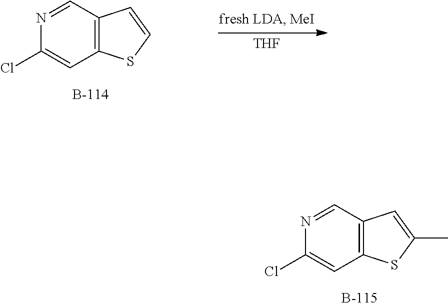

To a solution of diisopropylamine (4.3 g, 42 mmol) in tetrahydrofuran (15 mL) at −25° C. under nitrogen was added n-butyllithium (2.5 M in hexane, 19 mL). The mixture was stirred at −25° C. for 30 min and then cooled to −70° C. Compound B-114 (1.2 g, 7.1 mmol) was added, and the mixture was stirred at −70° C. for 30 min. Methyl iodide (13 g, 92 mmol) was added, and the mixture was stirred at −40° C. for 2 hours. On completion, the mixture was poured into water (150 mL) and extracted with ethyl acetate (3×100 mL). The combined organic phase was washed with brine (3×20 mL) and concentrated in vacuo. The residue was purified by pre-HPLC [Instrument: GX-E; Column: Phenomenex Synergi C18 250×21.2 mm, particle size: 4 μm; Mobile phase: 25-55% acetonitrile in H₂O (add 0.05% TFA, v/v)] and lyophilized to give compound B-115 (400 mg, 44% yield) as a white solid. ¹H-NMR (CDCl₃, 400 MHz): δ 8.72 (s, 1H), 7.72 (s, 1H), 7.06 (s, 1H), 2.63 (s, 3H). Compound B-115 was used to prepare compound (R)-99.

### Example 116B

### 2-chloro-5-iodo-4-methoxy-pyridine (B-116)

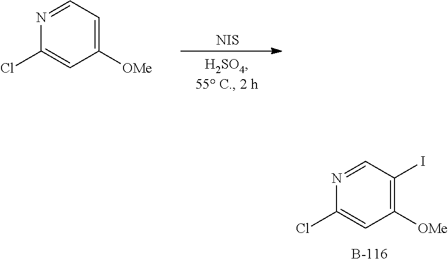 [SMILES: COc1ccnc(Cl)c1 ; "NIS" ; COc1cc(Cl)ncc1I]

To a solution of 2-chloro-4-methoxy-pyridine (10 g, 70 mmol) in concentrated sulfuric acid (50 mL) at room temperature was added a solution of N-iodosuccinimide (15.7 g, 70 mmol) in concentrated sulfuric acid (10 mL). The mixture was stirred at 55° C. for 2 hours, then diluated with water (100 mL), adjusted to pH=9-10 by slow addition of 8 N aqueous sodium hydroxide and extracted with dichloromethane (3×50 mL). The combined organic layers were washed with brine (50 mL), dried over sodium sulfate, filtered and concentrated in vacuo. The residue was purified by silica gel chromatography [petroleum ether: ethyl acetate=20:1] to give compound B-116(4 g, 21% yield) as a white solid. GC-MS: tR=6.569 min., (ES⁻) m/z (M)⁺=268.9.

### Example 117B

### 2-chloro-5-iodo-pyridin-4-ol (B-117)

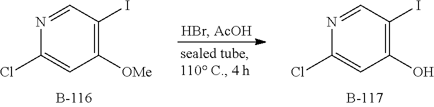

To a solution of compound B-116 (4.0 g, 7.4 mmol) in acetic acid (30 mL) was added hydrogen bromide (40% in water, 23 g, 111 mmol). The vessel was sealed and heated to 110° C. for 4 hours. On completion, the mixture was concentrated in vacuo. The residue was dissolved in methanol (20 mL) and triethylamine (20 mL), concentrated again in vacuo and purified by silica gel chromatography [dichloromethane: methanol=30:1] to give compound B-117 (3.0 g, 75% yield) as a white solid. ¹H-NMR (CD₃OD, 400 MHz): δ 8.33 (s, 1H), 6.73 (s, 1H).

### Example 118B

### 6-chloro-2-methyl-furo[3,2-c]pyridine (B-118)

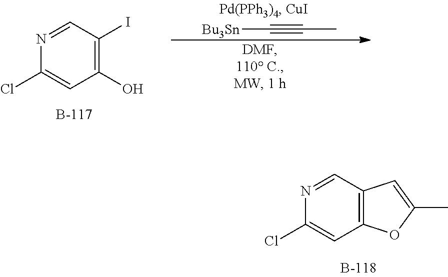

A solution of compound B-117 (0.90 g, 3.5 mmol), Pd(PPh₃)₄(68 mg, 0.059 mmol), cuprous iodide (22 mg, 0.12 mmol) and tributyl(prop-1-ynyl)stannane (773 mg, 2.4 mmol) in dimethyl formamide (12 mL) was stirred at 110° C. for 1 hour under nitrogen in the micwave. On completion, the mixture was poured into water (60 mL) and extracted with ethyl acetate (3×45 mL). The combined organic layers were concentrated and purified by silica gel chromatography [petroleum ether: ethyl acetate=50:1] to give compound B-118 (240 mg, 41% yield) as yellow soild. ¹H-NMR (CD₃OD, 400 MHz): δ 8.53 (s, 1H), 7.38 (s, 1H), 6.43 (s, 1H), 2.49 (s, 3H). Compound B-118 was used to prepare compound (R)-100.

General Procedure A: Synthesis of N-substituted 4H-1′-azaspiro[isoxazole-5,3′-bicyclo[2.2.2]octan]-3-amines.

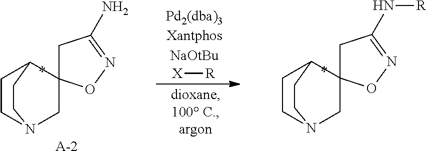

A 8 mL vial with septum was charged with compound (R)-A-2, (S)-A-2, or rac-A-2 (1.05 eq.), tris(dibenzylideneacetone)dipalladium (0.08 eq.), 9,9-dimethyl-4,5-bis(diphenylphosphino)xanthene (0.12 eq.), halide (1.00 eq.), and degassed 1,4-dioxane. Subsequently, sodium tert-butoxide (2.00 eq.) was added, and the reaction mixture was subjected to three cycles of evacuation-backfilling with argon and heated to 100° C. for the indicated number of hours.

The reaction mixtures were worked up as indicated and purified by column chromatography [chloroform: 7M NH₃ in methanol=1:0 to 9:1] to give enantiomerically pure or racemic product. Racemic products were further purified by preparative chiral HPLC.

Chiral Separation:

A solution of racemic product in a minimal amount of solvent was separated by preparative chiral HPLC using the indicated conditions. Each set of collected fractions was concentrated at room temperature, taken up in methanol and purified by SCX chromatography. The resulting products were lyophilized to give each enantiomer of the final product.

### Example 1

### N-phenyl-4H-1′-azaspiro[isoxazole-5,3′-bicyclo[2.2.2]octan]-3-amine (rac-1)

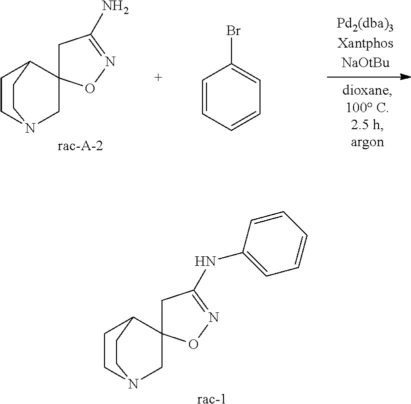

Compound rac-1 (73 mg, 54% yield) was prepared from compound rac-A-2 (100 mg, 0.60 mmol) according to general procedure A using 3 mL of 1,4-dioxane and a reaction time of 2.5 hours. The reaction mixture was diluted with THF (10 mL), filtered, concentrated and further purified according to general procedure A.

Chiral Separation:

Compound rac-1 (73 mg, 0.3 mmol) in 2.4 mL of acetonitrile was separated by preparative chiral HPLC [Instrument: cHPLC1, Flow: 18 mL/min, isocratic, time: 30 min., Column temp: 25° C., Chiralpak IA, 20×250 mm, Eluent: heptane+0.2% diethylamine/ethanol=7/3) to give:

N-phenyl-4H-1′-azaspiro[isoxazole-5,3′-bicyclo[2.2.2]octan]-3-amine enantiomer1 (compound 1-P1) (22 mg, 16% yield) as a white solid: cHPLC analytical (4): tR=9.864 min., purity: 100%; LCMS (1): tR=2.901 min., (ES⁺) m/z (M+H)⁺=258.2; ¹H NMR (300 MHz, Chloroform-d) δ 7.41-7.21 (m, 4H), 7.04-6.94 (m, 1H), 5.88 (s, 1H), 3.35-3.12 (m, 2H), 3.04-2.58 (m, 6H), 2.29-2.10 (m, 1H), 2.09-1.99 (m, 1H), 1.76-1.33 (m, 3H); and

N-phenyl-4H-1′-azaspiro[isoxazole-5,3′-bicyclo[2.2.2]octan]-3-amine enantiomer2 (compound 1-P2) (22 mg, 16% yield) as a white solid: cHPLC analytical (4): tR=23.180 min., purity: 100%; LCMS (1): tR=2.900 min., (ES⁺) m/z (M+H)⁺=258.2; ¹H NMR (300 MHz, Chloroform-d) δ 7.45-7.24 (m, 4H), 7.04-6.93 (m, 1H), 5.87 (s, 1H), 3.36-3.16 (m, 2H), 3.07-2.62 (m, 6H), 2.30-2.11 (m, 1H), 2.11-1.96 (m, 1H), 1.80-1.23 (m, 3H).

### Example 2

### N-(isoquinolin-3-yl)-4H-1′-azaspiro[isoxazole-5,3′-bicyclo[2.2.2]octan]-3-amine (rac-2)

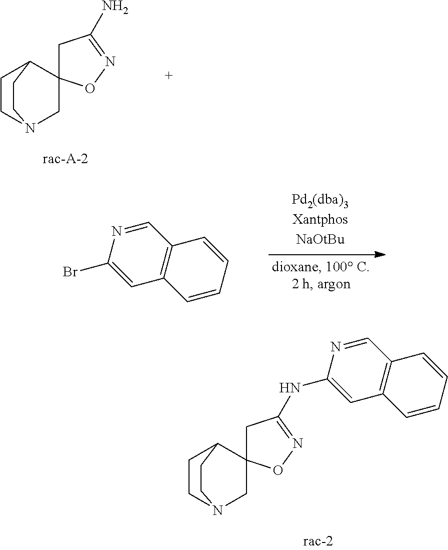 [SMILES: NC1=NOC2(C1)CN1CCC2CC1 ; Brc1cc2ccccc2cn1 ; c1ccc2cc(NC3=NOC4(C3)CN3CCC4CC3)ncc2c1]

Compound rac-2 (127 mg, 78% yield) was prepared from compound rac-A-2 (100 mg, 0.60 mmol) according to general procedure A using 3 mL of 1,4-dioxane and a reaction time of 2 hours. The reaction mixture was filtered, and the residue was further purified according to general procedure A.

Chiral Separation:

Compound rac-2 (127 mg, 0.4 mmol) in 2.0 mL of methanol/chloroform (4:1) was separated by preparative chiral HPLC [Instrument: cHPLC1, Flow: 18 mL/min, isocratic, time: 30 min., Column temp: 25° C., Column: Chiralpak IC, 20×250 mm, Eluent: heptane+0.2% diethylamine/ethanol=7/3] to give:

N-(isoquinolin-3-yl)-4H-1′-azaspiro[isoxazole-5,3′-bicyclo[2.2.2]octan]-3-amine enantiomer1 (compound 2-P1) (29 mg, 17% yield) as a white fluffy solid: cHPLC analytical (1): tR=7.649 min., purity: 100%; LCMS (1): tR=3.174 min., (ES⁻) m/z (M+H)⁺=309.2; ¹H NMR (300 MHz, Chloroform-d) δ 8.92 (s, 1H), 8.16 (s, 1H), 7.86 (d, J=8.2 Hz, 1H), 7.75 (d, J=7.8 Hz, 1H), 7.66-7.55 (m, 1H), 7.47-7.34 (m, 1H), 6.91 (s, 1H), 3.42-3.26 (m, 2H), 3.14-2.69 (m, 6H), 2.29-2.14 (m, 1H), 2.13-2.06 (m, 1H), 1.81-1.38 (m, 3H); and

N-(isoquinolin-3-yl)-4H-1′-azaspiro[isoxazole-5,3′-bicyclo[2.2.2]octan]-3-amine enantiomer2 (compound 2-P2) (32 mg, 19% yield) as a white fluffy solid: cHPLC analytical (1): tR=9.770 min., purity: 100%; LCMS (1): tR=3.177 min., (ES⁻) m/z (M+H)⁺=309.2; ¹H NMR (300 MHz, Chloroform-d) δ 8.92 (s, 1H), 8.16 (s, 1H), 7.86 (d, J=8.2 Hz, 1H), 7.75 (d, J=8.3 Hz, 1H), 7.66-7.54 (m, 1H), 7.44-7.36 (m, 1H), 6.91 (s, 1H), 3.43-3.23 (m, 2H), 3.18-2.66 (m, 6H), 2.31-2.13 (m, 1H), 2.15-2.04 (m, 1H), 1.81-1.30 (m, 3H).

### Example 3

### N-(benzo[d]thiazol-2-yl)-411-1¹-azaspiro[isoxazole-5,3′-bicyclo[2.2.2]octan]-3-amine (rac-3)

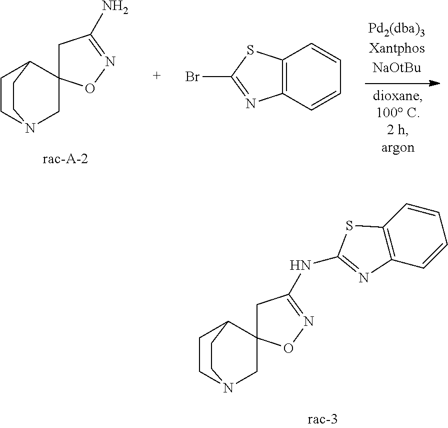 [SMILES: NC1=NOC2(C1)CN1CCC2CC1 ; Brc1nc2ccccc2s1 ; c1ccc2sc(NC3=NOC4(C3)CN3CCC4CC3)nc2c1]

Compound rac-3 (93 mg, 56% yield) was prepared from compound rac-A-2 (100 mg, 0.60 mmol) according to general procedure A using 3 mL of 1,4-dioxane and a reaction time of 2 hours. The reaction mixture was filtered, and the residue was further purified according to general procedure A.

Chiral Separation:

Compound rac-3 (93 mg, 0.3 mmol) in 3.5 mL of methanol/chloroform (4:1) was separated by preparative chiral HPLC [Instrument: cHPLC1, Flow: 18 mL/min, isocratic, time: 30 min., Column temp: 25° C., Column: Chiralpak IC, 20×250 mm, 5 μ, Eluent: heptane+0.2% diethylamine/ethanol=7/3] to give:

N-(benzo[d]thiazol-2-yl)-4H-1′-azaspiro[isoxazole-5,3′-bicyclo[2.2.2]octan]-3-amine enantiomer1 (compound 3-P1) (24 mg, 14% yield) as an off-white solid: cHPLC analytical (1): tR=6.958 min., purity: 100%; LCMS (1): tR=3.062 min., (ES⁺) m/z (M+H)⁺=315.2; ¹H NMR (300 MHz, Chloroform-d) δ 7.89-7.56 (m, 2H), 7.52-7.31 (m, 1H), 7.31-7.11 (m, 2H), 3.45-3.21 (m, 2H), 3.12-2.65 (m, 6H), 2.31-2.12 (m, 1H), 2.08 (bs, 1H), 1.78-1.34 (m, 3H); and

N-(benzo[d]thiazol-2-yl)-4H-1′-azaspiro[isoxazole-5,3′-bicyclo[2.2.2]octan]-3-amine enantiomer2 (compound 3-P2) (24 mg, 14% yield) as a white solid: cHPLC analytical (1): tR=8.376 min., purity: 100%; LCMS (1): tR=3.068 min., (ES⁺) m/z (M+H)⁺=315.2; ^{I}H NMR (300 MHz, Chloroform-d) δ 7.97-7.55 (m, 2H), 7.49-7.32 (m, 1H), 7.31-7.18 (m, 2H), 3.43-3.20 (m, 2H), 3.13-2.64 (m, 6H), 2.30-2.11 (m, 1H), 2.10-2.04 (m, 1H), 1.81-1.33 (m, 3H).

### Example 4

### N-(6-methoxybenzo[d]thiazol-2-yl)-4H-1′-azaspiro[isoxazole-5,3′-bicyclo[2.2.2]octan]-3-amine (rac-4)

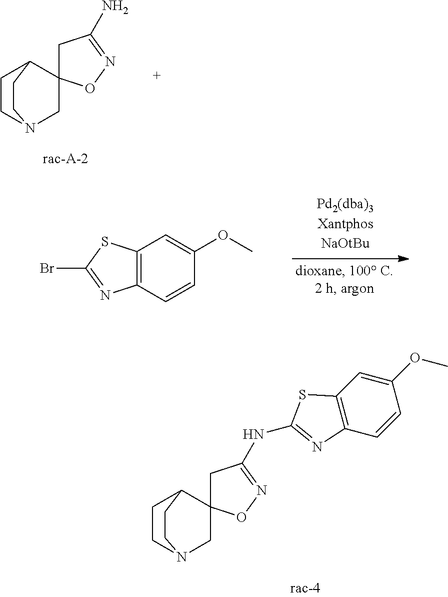 [SMILES: NC1=NOC2(C1)CN1CCC2CC1 ; COc1ccc2nc(Br)sc2c1 ; COc1ccc2nc(NC3=NOC4(C3)CN3CCC4CC3)sc2c1]

Compound rac-4 (93 mg, 51% yield) was prepared from compound rac-A-2 (100 mg, 0.60 mmol) according to general procedure A using 3 mL of 1,4-dioxane and a reaction time of 2 hours. The reaction mixture was filtered, and the residue was further purified according to general procedure A.

Chiral Separation:

Compound rac-4 (93 mg, 0.3 mmol) in 2.0 mL of methanol/chloroform (4:1) was separated by preparative chiral HPLC [Instrument: cHPLC1, Flow: 18 mL/min, isocratic, time: 30 min., Column temp: 25° C., Column: Chiralpak IC, 20×250 mm, 5 μ, Eluent: heptane+0.2% diethylamine/ethanol=7/3] to give:

N-(6-methoxybenzo[d]thiazol-2-yl)-4H-1′-azaspiro[isoxazole-5,3′-bicyclo[2.2.2]octan]-3-amine enantiomer1 (compound 4-P1) (15 mg, 8% yield) as a fluffy white solid: cHPLC analytical (1): tR=8.547 min., purity: 100%; LCMS (1): tR=3.090 min., (ES⁺) m/z (M+H)⁺=245.0; ¹H NMR (300 MHz, Chloroform-d) δ 7.56 (d, J=8.4 Hz, 1H), 7.26 (s, 1H), 7.01 (d, J=8.3 Hz, 1H), 3.86 (s, 3H), 3.47-3.21 (m, 2H), 3.10-2.69 (m, 6H), 2.27-2.11 (m, 1H), 2.07 (bs, 1H), 1.86-1.35 (m, 3H); and

N-(6-methoxybenzo[d]thiazol-2-yl)-4H-1′-azaspiro[isoxazole-5,3′-bicyclo[2.2.2]octan]-3-amine enantiomer2 (compound 4-P2) (28 mg, 15% yield) as a fluffy white solid: cHPLC analytical (1): tR=8.547 min., purity: 100%; LCMS (1): tR=3.089 min., (ES⁺) m/z (M+H)⁺=245.0; ¹H NMR (300 MHz, Chloroform-d) δ 7.56 (d, J=8.1 Hz, 1H), 7.26 (s, 1H), 7.00 (d, J=7.8 Hz, 1H), 3.86 (s, 3H), 3.41- 3.25 (m, 2H), 3.12-2.54 (m, 6H), 2.29-2.11 (m, 1H), 2.07 (bs, 1H), 1.79-1.27 (m, 3H).

### Example 5

### N-(1H-indazol-3-yl)-4′H-4-azaspiro[bicyclo[2.2.2]octane -2,5¹-isoxazol]-3 ′- amine (rac-5)

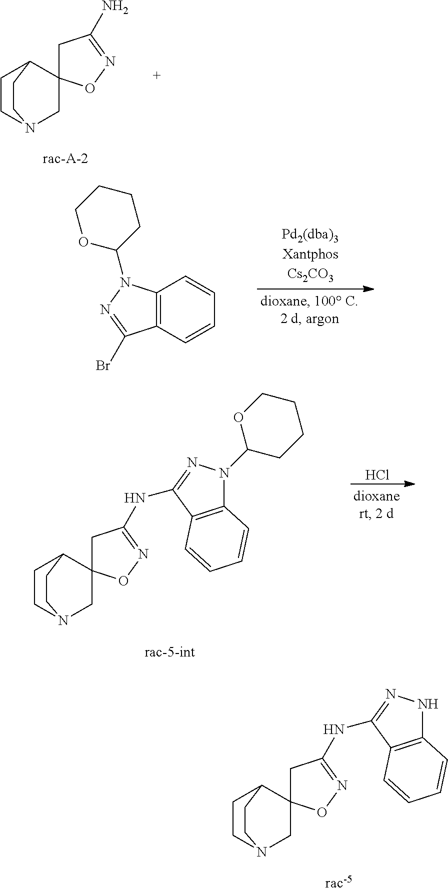 [SMILES: NC1=NOC2(C1)CN1CCC2CC1 ; Brc1nn(C2CCCCO2)c2ccccc12 ; c1ccc2c(c1)c(NC1=NOC3(C1)CN1CCC3CC1)nn2C1CCCCO1 ; Cl ; c1ccc2c(NC3=NOC4(C3)CN3CCC4CC3)n[nH]c2c1]

Compound rac-5-int (150 mg, 36% yield) was prepared from compound rac-A-2 (203 mg, 1.1 mmol) according to general procedure A using 6 mL of 1,4-dioxane, a reaction time of 2 days and cesium carbonate (2.8 eq) instead of sodium tert-butoxide as the base. The reaction mixture was filtered, washing with methanol, concentrated and further purified according to general procedure A. LCMS (1): tR=3.671 min., (ES⁺) m/z (M+H)⁺=382.2. ¹H NMR (300 MHz, Chloroform-d) δ 7.76-7.67 (m, 1H), 7.52-7.34 (m, 2H), 7.25-7.06 (m, 1H), 6.99-6.93 (m, 1H), 5.59 (dd, J=9.4, 2.5 Hz, 1H), 4.12-3.99 (m, 1H), 3.85-3.53 (m, 2H), 3.40-3.15 (m, 2H), 3.11-2.72 (m, 5H), 2.61-2.39 (m, 1H), 2.27-1.92 (m, 4H), 1.87-1.36 (m, 6H).

To a solution of compound rac-5-int (143 mg, 0.4 mmol) in 1,4-dioxane (6.0 mL) was added hydrochloric acid (1.25 M in ethanol, 6.0 mL, 7.5 mmol). The reaction mixture was stirred at room temperature for 24 hours. Additional hydrochloric acid (1.25 M in ethanol, 3.0 mL, 3.8 mmol) was added, and the mixture was stirred for an additional 24 hours. Then concentrated and purified by SCX chromatography. The resulting yellow oil was dissolved in chloroform (1.5 ml) and purified by column chromatography [chloroform: 7M NH₃ in methanol=1:0 to 9:1] to afford compound rac-5 (54 mg, 48% yield). LCMS (1): tR=2.666 min., (ES⁺) m/z (M+H)⁺=298.2.

Chiral Separation:

Compound rac-5 (54 mg, 0.2 mmol) in 1.3 mL of ethanol was separated by preparative chiral HPLC [Instrument: cHPLC1, Flow: 18 mL/min, isocratic, time: 30 min., Column temp: 25° C., Column: Chiralpak IC, 20×250 mm, Eluent: heptane+0.2% diethylamine/ethanol=8/2) to give:

N-(1H-indazol-3-yl)-4′H-4-azaspiro[bicyclo[2.2.2]octane-2,5′-isoxazol]-3′-amine enantiomer1 (compound-5-P1) (16 mg, 14% yield) as a white fluffy solid: cHPLC analytical (2): tR=18.474 min., purity: 100%; LCMS (1): tR=2.666 min., (ES) m/z (M+H)⁺=298.2; ¹H NMR (300 MHz, Chloroform-d) δ 9.46 (bs, 1H), 7.77 (d, J=8.2 Hz, 1H), 7.41-7.33 (m, 2H), 7.22-7.07 (m, 1H), 3.63 (d, J=16.7 Hz, 1H), 3.39-3.22 (m, 2H), 3.10-2.68 (m, 5H), 2.27-2.04 (m, 2H), 1.73-1.53 (m, 3H), 1.52-1.35 (m, 1H); and

N-(1H-indazol-3-yl)-4′H-4-azaspiro[bicyclo[2.2.2]octane-2,5¹-isoxazol]-3′-amine enantiomer2 (compound-5-P2) (14 mg, 12% yield) as a white fluffy solid: cHPLC analytical (2): tR=25.355 min., purity: 100%; LCMS (1): tR=2.650 min., (ES⁻) m/z (M+H)⁺=298.2; ¹H NMR (300 MHz, Chloroform-d) δ 9.42 (bs, 1H), 7.76 (d, J=8.2 Hz, 1H), 7.45-7.33 (m, 2H), 7.20-7.07 (m, 1H), 3.63 (d, J=16.7 Hz, 1H), 3.40-3.19 (m, 2H), 3.10-2.59 (m, 5H), 2.26-2.05 (m, 2H), 1.78-1.50 (m, 3H), 1.50-1.36 (m, 1H).

### Example 6

### N-(pyrrolo[2,1-f][1,2,4]triazin-4-yl)-4H-F-azaspiro[isoxazole-5,3¹- bicyclo[2.2.2]octan]-3-amine (rac-6)

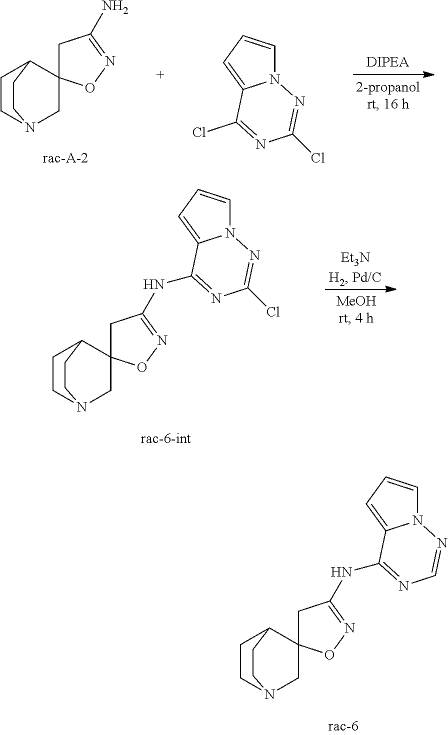 [SMILES: NC1=NOC2(C1)CN1CCC2CC1 ; Clc1nc(Cl)c2cccn2n1 ; Clc1nc(NC2=NOC3(C2)CN2CCC3CC2)c2cccn2n1 ; "Et3NH2, Pd/C" ; c1cc2c(NC3=NOC4(C3)CN3CCC4CC3)ncnn2c1]

Compound rac**-A-2 (50 mg, 0.3 mmol) and 2,4-dichloropyrrolo[2,1-f][1,2,4]triazine (52 mg, 0.3 mmol) were dissolved in 2-propanol (1 mL). After stirring at room temperature for 3 hours, N,N-diisopropylethylamine (80 μL, 0.5 mmol) was added, and the reaction mixture was stirred for 16 hours, concentrated onto silica and purified by column chromatography [chloroform: 7M NH₃ in methanol=1:0 to 9:1] to afford compound rac-6-int (59 mg, 64% yield). LCMS (1): tR=2.498 min., (ES⁻) m/z (M+H)⁺=333.0.

A suspension of compound rac-6-int (357 mg, 1.1 mmol) in methanol (30 mL) was flushed with nitrogen for 5 minutes. Then triethylamine (0.3 mL, 2.1 mmol) and palladium on carbon (10%, 114 mg, 0.1 mmol) were added, and the reaction mixture was stirred under an atmosphere of hydrogen at room temperature for 4 hours. On completion, the reaction mixture was flushed with nitrogen, filtered over Celite, concentrated onto silica and purified by column chromatography [chloroform: methanol=1:0 to 9:1] to afford compound rac-6 as a white solid (174 mg, 54%).

Chiral Separation:

Compound rac-6 (174 mg, 0.6 mmol) in 14 mL of N,N-dimethylformamide /methanol/chloroform (1:1:2) was separated by preparative chiral HPLC [Instrument: cHPLC1, Flow: 18 mL/min, isocratic, time: 30 min., Column temp: 25° C., Column: Chiralpak IC, 20×250 mm, 5 μ, Eluent: heptane+0.2% diethylamine/ethanol=8/2] to give:

N-(pyrrolo[2,1-f][1,2,3]triazin-4-yl)-4H-1′-azaspiro[isoxazole-5,3′-bicyclo[2.2.2]octan]-3-amine enantiomer1 (compound 6-P1) (70 mg, 21% yield) as a white solid: cHPLC analytical (2): tR=7.682 min., purity: 100%; LCMS (1): tR=2.898 min., (ES⁺) m/z (M+H)⁺=299.2; ¹H NMR (300 MHz, Chloroform-d) δ 8.37 (bs, 1H), 7.27 (s, 1H), 7.18 (dd, J=2.8, 1.7 Hz, 1H), 6.60 (dd, J=4.1, 1.7 Hz, 1H), 6.37 (dd, J=4.1, 2.8 Hz, 1H), 3.23 (d, J=15.2 Hz, 1H), 2.95 (s, 2H), 2.93-2.68 (m, 5H), 2.45-2.35 (m, 1H), 2.00-1.82 (m, 1H), 1.79-1.29 (m, 3H); and

N-(pyrrolo [2,1-f][1,2,4]triazin-4-yl)-4H-1′-azaspiro[isoxazole-5,3′-bicyclo[2.2.2]octan]-3-amine enantiomer2 (compound 6-P2) (73 mg, 22% yield) as a light yellow solid: cHPLC analytical (2): tR=10.926 min., purity: 100%; LCMS (1): tR=2.894 min., (ES⁺) m/z (M+H)⁺=299.1;¹H NMR (300 MHz, Chloroform-d) δ 8.45 (bs, 1H), 7.28 (s, 1H), 7.19 (dd, J=2.8, 1.7 Hz, 1H), 6.60 (dd, J=4.1, 1.6 Hz, 1H), 6.37 (dd, J=4.1, 2.8 Hz, 1H), 3.25 (d, J=13.3 Hz, 1H), 2.96 (s, 2H), 2.92-2.68 (m, 5H), 2.48-2.34 (m, 1H), 1.98-1.85 (m, 1H), 1.80-1.32 (m, 3H).

### Example 7

### N-(7-bromopyrrolo[1,2,4]triazin-4 -yl)-4H-1′-azaspiro[isoxazole-5,3′- bicyclo[2.2.2]octan]-3-amine enantiomer1 (7-P1)

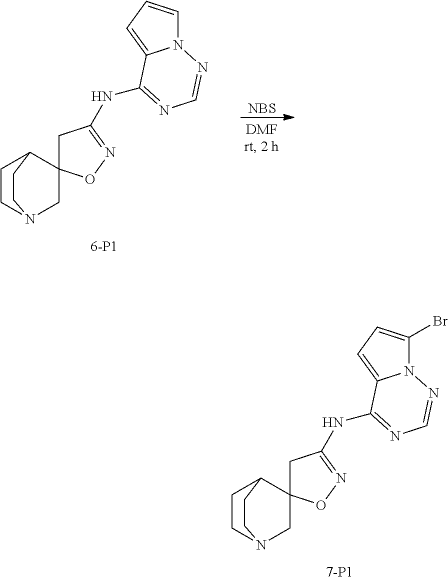

To a solution of compound-6-P1 (36 mg, 0.1 mmol) in anhydrous N,N-dimethylformamide (1.5 ml) was added N-bromosuccinimide (22 mg, 0.1 mmol). The mixture was stirred at room temperature for 1 hour. Additional N-bromosuccinimide (2 mg, 0.01 mmol) was added, and the mixture was stirred for another hour. The reaction mixture was purified by SCX chromatography and further purified by silica gel column chromatography [chloroform: 7M NH₃ in methanol=1:0 to 9:1], followed by preparative HPLC [Instrument: prep HPLC1; column: Phenomenex Gemini-NX(C18, 100 mm×21.2 mm, 10 μ); flow: 25 ml/min; column temp: RT; eluent A: 99% acetonitrile+1% 10 mM ammonium bicarbonate in water pH=9.0, eluent B: 10 mM ammonium bicarbonate in water pH=9.0; detection: DAD (220-320 nm); detection: MSD (ESI pos/neg) mass range: 100-800; fraction collection based on MS and DAD] and then by preparative chiral HPLC [Instrument: cHPLC1, Flow: 18 mL/min, isocratic, time: 30 min., Column temp: 25° C., Column: Chiralpak IC, 20×250 mm, 5 μ, Eluent: heptane+0.2% diethylamine/ethanol=8/2]. Finally, the product was purified by SCX chromatography and lyophilized from acetonitrile and water to afford compound 7-P1 as a white fluffy solid (8 mg, 17%). cHPLC analytical (2): tR=8.640 min., purity: 100%; LCMS (1): tR=3.191 min., (ES⁻) m/z (M+H)⁺=377.0/379.0; ¹H NMR (300 MHz, Chloroform-d) δ 8.56 (s, 1H), 7.38 (s, 1H), 6.63 (d, J=4.3 Hz, 1H), 6.41 (d, J=4.3 Hz, 1H), 3.24 (d, J=14.7 Hz, 1H), 3.00-2.68 (m, 7H), 2.46-2.30 (m, 1H), 1.97-1.32 (m, 4H).

N-(7-bromopyrrolo[2,1-f][1,2,4]triazin-4-yl)-4H-1′-azaspiro[isoxazole-5,3′-bicyclo[2.2.2]octan]-3-amine enantiomer2 (7-P2)

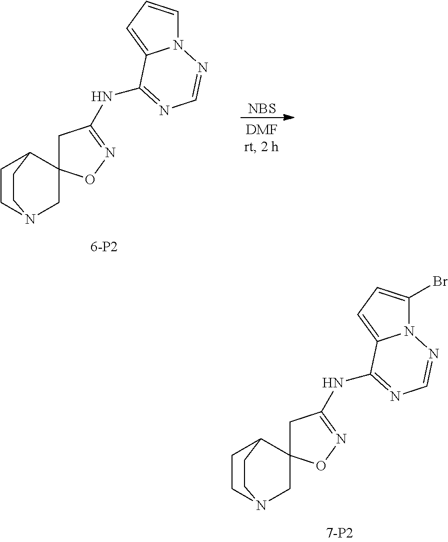

To a solution of compound-6-P2 (37 mg, 0.1 mmol) in anhydrous N,N-dimethylformamide (1.5 ml) was added N-bromosuccinimide (23 mg, 0.1 mmol). The mixture was stirred at room temperature for 1 hour. Additional N-bromosuccinimide (2 mg, 0.01 mmol) was added, and the mixture was stirred for another hour. The reaction mixture was purified by SCX chromatography and further purified by preparative HPLC [Instrument: prep HPLC1; column: Phenomenex Gemini-NX(C18, 100 mm×21.2 mm, 10 μ); flow: 25 ml/min; column temp: RT; eluent A: 99% acetonitrile+1% 10 mM ammonium bicarbonate in water pH=9.0, eluent B: 10 mM ammonium bicarbonate in water pH=9.0; detection: DAD (220-320 nm); detection: MSD (ESI pos/neg) mass range: 100-800; fraction collection based on MS and DAD.] and by preparative chiral HPLC [Instrument: cHPLC1, Flow: 18 mL/min, isocratic, time: 30 min., Column temp: 25° C., Column: Chiralpak IC, 20×250 mm, 5 μ, Eluent: heptane+0.2% diethylamine/ethanol=8/2]. Finally, the product was purified by SCX chromatography and lyophilized from acetonitrile and water overnight to afford compound 7-P2 as a white fluffy solid (11 mg, 23%). cHPLC analytical (2): tR=11.251 min., purity: 100%; LCMS (1): tR=3.189 min., (ES⁺) m/z (M+H)⁺=377.0/379.0; ¹H NMR (300 MHz, Chloroform-d) δ 8.50 (s, 1H), 7.38 (s, 1H), 6.63 (d, J=4.3 Hz, 1H), 6.41 (d, J=4.3 Hz, 1H), 3.23 (d, J=15.0 Hz, 1H), 3.02-2.69 (m, 7H), 2.45-2.32 (m, 1H), 1.96-1.32 (m, 4H).

### Example 8

### (S)—N-(4-chlorobenzo[d]thiazol-2-yl)-4H-1′-azaspiro[isoxazole-5,3′-bicyclo[2.2.2]octan]-3-amine ((S)-8)

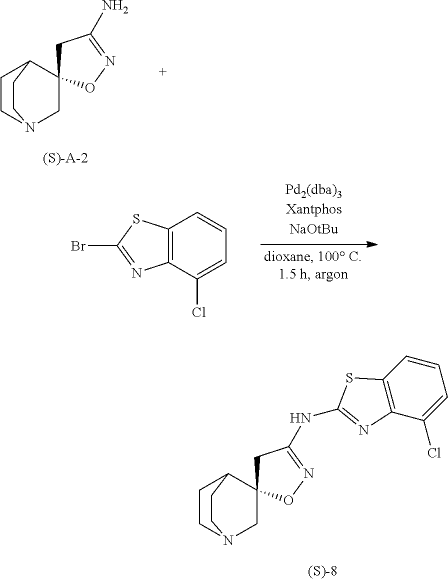 [SMILES: NC1=NO[C@]2(C1)CN1CCC2CC1 ; Clc1cccc2sc(Br)nc12 ; Clc1cccc2sc(NC3=NO[C@]4(C3)CN3CCC4CC3)nc12]

Compound (S)-8 (δ 9 mg, 75% yield) was prepared as a white solid from compound (S)-A-2 (50 mg, 0.3 mmol) according to general procedure A using 1.5 mL of 1,4-dioxane and a reaction time of 1.5 hoursThe reaction mixture was filtered, and the residue was further purified according to general procedure A. cHPLC analytical (3): tR=14.291 min., purity: 100%; LCMS (1): tR=3.100 min., (ES⁻) m/z (M+H)⁺=249.0; ¹H NMR (300 MHz, DMSO-d₆) δ 11.69 (s, 1H), 7.90 (dd, J=7.9, 1.0 Hz, 1H), 7.47 (dd, J=7.9, 1.1 Hz, 1H), 7.20 (t, J=7.9 Hz, 1H), 3.43-3.16 (m, 1H), 3.12-2.88 (m, 3H), 2.84-2.50 (m, 4H), 1.99-1.81 (m, 2H), 1.62-1.44 (m, 2H), 1.44-1.28 (m, 1H).

(R)—N-(4-chlorobenzo[d]thiazol-2-yl)-4H-1′-azaspiro[isoxazole-5,3′-bicyclo[2.2.2]octan]-3-amine ((R)-8)

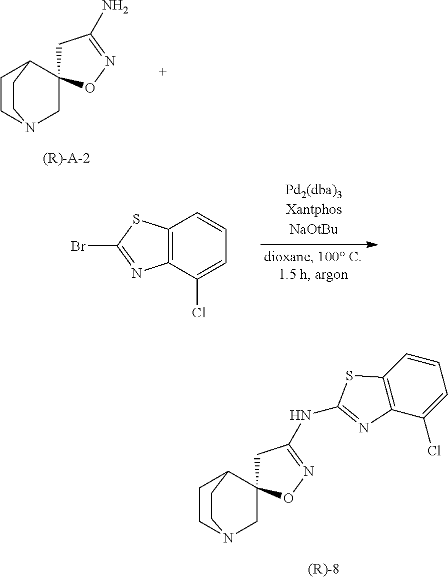 [SMILES: NC1=NO[C@@]2(C1)CN1CCC2CC1 ; Clc1cccc2sc(Br)nc12 ; Clc1cccc2sc(NC3=NO[C@@]4(C3)CN3CCC4CC3)nc12]

Compound (R)-8 (67 mg, 73% yield) was prepared as a white solid from compound (R)-A-2 (50 mg, 0.3 mmol) according to general procedure A using 1.5 mL of 1,4-dioxane and a reaction time of 1.5 hours. The reaction mixture was filtered, and the residue was further purified according to general procedure A. cHPLC analytical (3): tR=23.373 min., purity: 100%; LCMS (1): tR=3.122 min., (ES⁻) m/z (M+H)⁺=249.0; ¹H NMR (300 MHz, DMSO-d₆) δ 11.68 (bs, 1H), 7.90 (dd, J=7.9, 1.0 Hz, 1H), 7.47 (dd, J=7.8, 1.0 Hz, 1H), 7.20 (t, J=7.9 Hz, 1H), 3.41-3.16 (m, 1H), 3.15-2.94 (m, 3H), 2.79-2.60 (m, 4H), 1.98-1.82 (m, 2H), 1.65-1.46 (m, 2H), 1.46-1.29 (m, 1H).

### Example 9

### N -(5 -chlorobenzo[d]thiazol-2-yl)-4H-1′-azaspiro[isoxazole-5,3′-bicyclo[2.2.2]octan]-3-amine (rac-9)

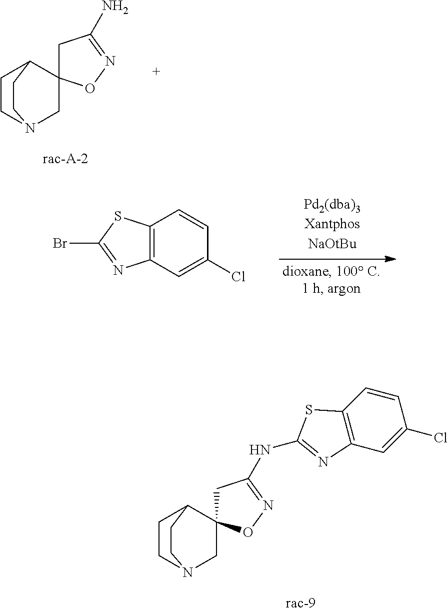 [SMILES: NC1=NOC2(C1)CN1CCC2CC1 ; Clc1ccc2sc(Br)nc2c1 ; Clc1ccc2sc(NC3=NO[C@@]4(C3)CN3CCC4CC3)nc2c1]

Compound rac-9 (150 mg, 82% yield) was prepared from compound rac-A-2 (100 mg, 0.60 mmol) according to general procedure A using 3 mL of 1,4-dioxane and a reaction time of 1 hours. The reaction mixture was filtered, and the residue was further purified according to general procedure A.

Chiral Separation:

Compound rac-9 (73 mg, 0.2 mmol) in 4.0 mL of ethanol/chloroform (1:1) was separated by preparative chiral HPLC (Column A, Eluent: heptane+0.2% diethylamine/ethanol=9/1) to give:

N-(5 -chlorobenzo[d]thiazol-2-yl)-4H-1′-azaspiro[isoxazole-5,3′-bicyclo[2.2.2]octan]-3-amine enantiomer1 (compound 9-P1) (35 mg, 19% yield) as a white fluffy solid: cHPLC analytical (3): tR=11.311 min., purity: 100%; LCMS (1): tR=3.456 min., (ES⁺) m/z (M+H)⁺=349.3; ¹H NMR (300 MHz, Chloroform-d) δ 7.75-7.62 (m, 2H), 7.23 (dd, J=8.4, 2.0 Hz, 1H), 3.48-3.23 (m, 2H), 3.16-2.63 (m, 6H), 2.27-2.01 (m, 2H), 1.83-1.66 (m, 1H), 1.66-1.37 (m, 2H); and

N-(5-chlorobenzo[d]thiazol-2-yl)-4H-1′-azaspiro[isoxazole-5,3′-bicyclo[2.2.2]octan]-3-amine enantiomer2 (compound 9-P2) (32 mg, 17% yield) as a white fluffy solid: cHPLC analytical (3): tR=16.901 min., purity: 100%; LCMS (1): tR=3.448 min., (ES⁺) m/z (M+H)⁺=349.0; ¹H NMR (300 MHz, Chloroform-d) δ 7.78-7.57 (m, 2H), 7.23 (dd, J=8.4, 2.0 Hz, 1H), 3.50-3.24 (m, 2H), 3.13-2.60 (m, 6H), 2.28-2.01 (m, 2H), 1.82-1.66 (m, 1H), 1.66-1.39 (m, 2H).

### Example 10

### N-(6-chlorobenzo[d]thiazol-2-yl)-4H-F-azaspiro[isoxazole-5,3′-bicyclo[2.2.2]octan]-3-amine (rac-10)

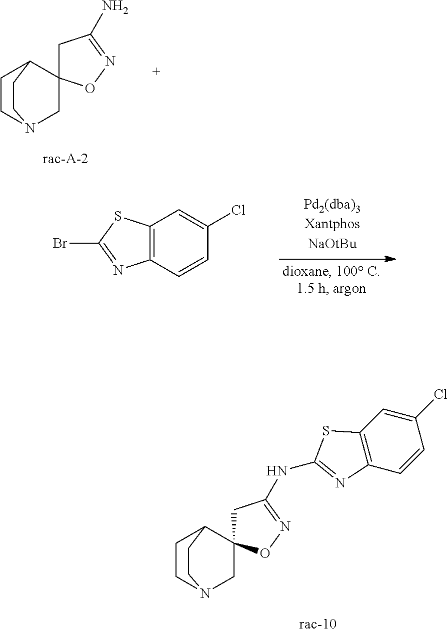 [SMILES: NC1=NOC2(C1)CN1CCC2CC1 ; Clc1ccc2nc(Br)sc2c1 ; Clc1ccc2nc(NC3=NO[C@@]4(C3)CN3CCC4CC3)sc2c1]

Compound rac-10 (118 mg, 64% yield) was prepared from compound rac-A-2 (100 mg, 0.60 mmol) according to general procedure A using 3 mL of 1,4-dioxane and a reaction time of 1.5 hours. The reaction mixture was concentrated and further purified according to general procedure A.

Chiral Separation:

Compound rac-10 (98 mg, 0.3 mmol) in 3.5 mL of ethanol/chloroform (1:1) was separated by preparative chiral HPLC (Column A, Eluent: heptane+0.2% diethylamine/ethanol=9/1) to give:

N-(6-chlorobenzo[d]thiazol-2-yl)-4H-1′-azaspiro[isoxazole-5,3′-bicyclo[2.2.2]octan]-3-amine enantiomer1 (compound 10-P1) (35 mg, 19% yield) as a white fluffy solid: cHPLC analytical (3): tR=11.347 min., purity: 100%; LCMS (1): tR=3.485 min., (ES⁺) m/z (M+H)⁺=349.0; ¹H NMR (300 MHz, Chloroform-d) δ 7.73 (s, 1H), 7.64-7.48 (m, 1H), 7.36 (dd, J=8.6, 2.1 Hz, 1H), 3.46-3.24 (m, 2H), 3.16-2.69 (m, 6H), 2.26-2.03 (m, 2H), 1.79-1.65 (m, 1H), 1.64-1.39 (m, 2H); and

N-(6-chlorobenzo[d]thiazol-2-yl)-4H-1′-azaspiro[isoxazole-5,3′-bicyclo[2.2.2]octan]-3-amine enantiomer2 (compound 10-P2) (31 mg, 17% yield) as a white fluffy solid: cHPLC analytical (3): tR=15.796 min., purity: 100%; LCMS (1): tR=3.467 min., (ES⁺) m/z (M+H)⁺=349.0; ¹H NMR (300 MHz, Chloroform-d) δ 7.73 (s, 1H), 7.62-7.51 (m, 1H), 7.36 (dd, J=8.6, 2.1 Hz, 1H), 3.50-3.22 (m, 2H), 3.15-2.68 (m, 6H), 2.24-2.04 (m, 2H), 1.81-1.65 (m, 1H), 1.63-1.39 (m, 2H).

### Example 11

### (S)—N-(7-chlorobenzo[d]thiazol-2-yl)-4H-1′-azaspiro[isoxazole-5,3′-bicyclo[2.2.2]octan]-3-amine ((S)-11)

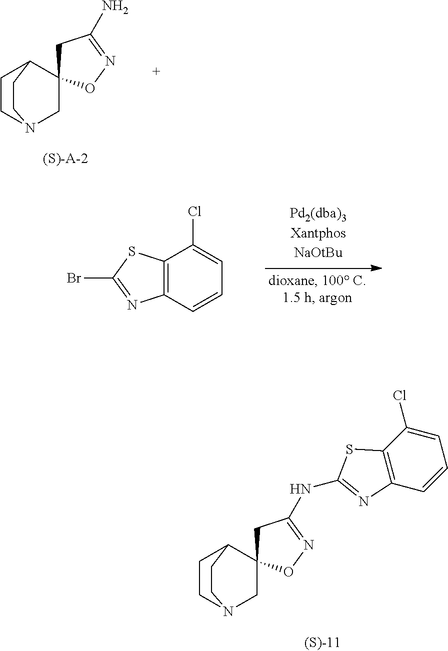 [SMILES: NC1=NO[C@]2(C1)CN1CCC2CC1 ; Clc1cccc2nc(Br)sc12 ; Clc1cccc2nc(NC3=NO[C@]4(C3)CN3CCC4CC3)sc12]

Compound (S)-11 (74 mg, 80% yield) was prepared as a white solid from compound (S)-A-2 (50 mg, 0.3 mmol) according to general procedure A using 1.5 mL of 1,4-dioxane and a reaction time of 1.5 hours. The reaction mixture was filtered, and the residue was further purified according to general procedure A. cHPLC analytical (3): tR=13.397 min., purity: 100%; LCMS (1): tR=3.146 min., (ES⁺) m/z (M+H)⁻=249.0; ¹H NMR (300 MHz, DMSO-d₆) δ 11.47 (bs, 1H), 7.74 - 7.51 (m, 1H), 7.41 (t, J=8.0 Hz, 1H), 7.35-7.23 (m, 1H), 3.34 (d, J=16.7 Hz, 1H), 3.16-2.83 (m, 3H), 2.80-2.58 (m, 4H), 2.06-1.83 (m, 2H), 1.69-1.29 (m, 3H).

(R)—N-(7-chlorobenzo[d]thiazol-2-yl)-4H-1′-azaspiro[isoxazole-5,3′-bicyclo[2.2.2]octan]-3-amine ((R)-11)

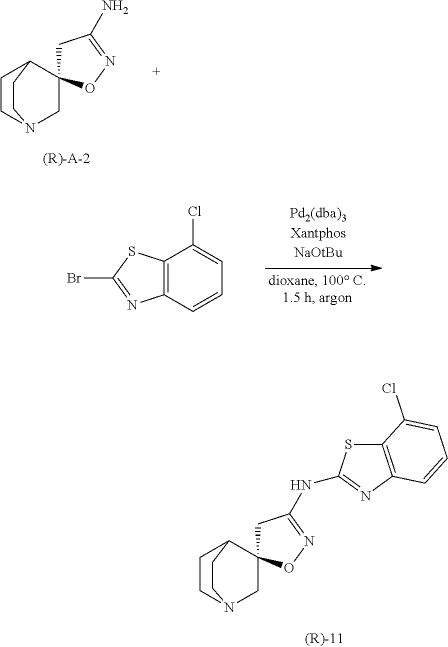 [SMILES: NC1=NO[C@@]2(C1)CN1CCC2CC1 ; Clc1cccc2nc(Br)sc12 ; Clc1cccc2nc(NC3=NO[C@@]4(C3)CN3CCC4CC3)sc12]

Compound (R)-11 (74 mg, 80% yield) was prepared as a yellow solid from compound (R)-A-2 (50 mg, 0.3 mmol) according to general procedure A using 1.5 mL of 1,4-dioxane and a reaction time of 1.5 hours. The reaction mixture was filtered, and the residue was further purified according to general procedure A. cHPLC analytical (3): tR=19.209 min., purity: 100%; LCMS (1): tR=3.171 min., (ES⁺) m/z (M+H)⁻=249.0; ¹H NMR (300 MHz, DMSO-d₆) δ 11.48 (bs, 1H), 7.60 (dd, J=8.0, 0.9 Hz, 1H), 7.42 (t, J=8.0 Hz, 1H), 7.30 (dd, J=7.8, 0.9 Hz, 1H), 3.34 (d, J=16.7 Hz, 1H), 3.17-2.87 (m, 3H), 2.85-2.58 (m, 4H), 2.07-1.82 (m, 2H), 1.68-1.30 (m, 3H).

### Example 12

### (+/−)-N-(benzo[b]thiophen-2-yl)-4H-1′-azaspiro[isoxazole-5,3′-bicyclo[2.2.2]octan]-3-amine (rac-12)

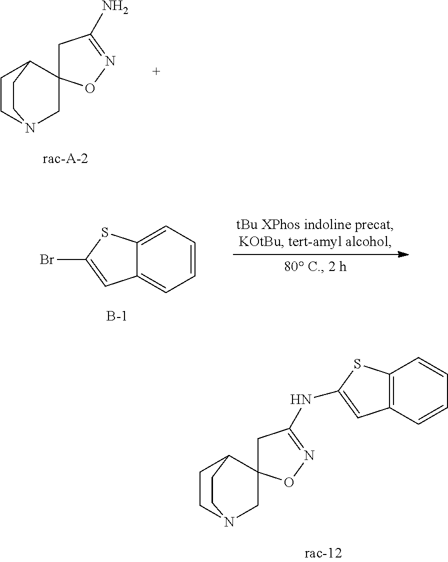

To a mixture of compound B-1 (0.10 g, 0.47 mmol) and compound rac-A-2 (0.13 g, 0.70 mmol) in tert-amyl alcohol (10 mL) under nitrogen at room temperature was added potassium tert-butoxide (0.16 g, 1.4 mmol) and chloro(2-ditbutylphosphino-2′, 4′, 6′-tri-i-propyl-1,1′-biphenyl)[2-(2-aminoethyl)phenyl] palladium(II) (26 mg, 0.037 mmol). The reaction mixture was stirred at 80° C. for 2 hours, then filtered and concentrated in vacuo. The residue was purified by prep-HPLC [Instrument: Shimadzu pump LC-20A; Column: GEMINI 200×50 mm, particle size: 10 μm; Mobile phase: 55-66.7% acetonitrile in H₂O (add 0.5% NH₃·H₂O, v/v)] to give compound rac-12 (10 mg, 6% yield, 80% purity) as a brown solid; LCMS (Y): tR=0.63 min., 314.1 m/z (M+1); ¹H-NMR (CD₃OD, 400 MHz): δ 7.68 (d, J=8.0 Hz, 1H), 7.57 (d, J=7.6 Hz, 1H), 7.28 (t, J=7.2 Hz, 1H), 7.18 (t, J=7.2 Hz, 1H), 3.78-3.61 (m, 4H), 3.48-3.42 (m, 2H), 3.39-3.36 (m, 3H), 2.45-2.40 (m, 2H), 2.15-2.10 (m, 1H), 2.01-1.92 (m, 2H).

### Example 13

### (+/−)-N-(benzo[d]oxazol-2-yl)-4H-1′-azaspiro[isoxazole-5,3′-bicyclo[2.2.2]octan]-3-amine (rac-13)

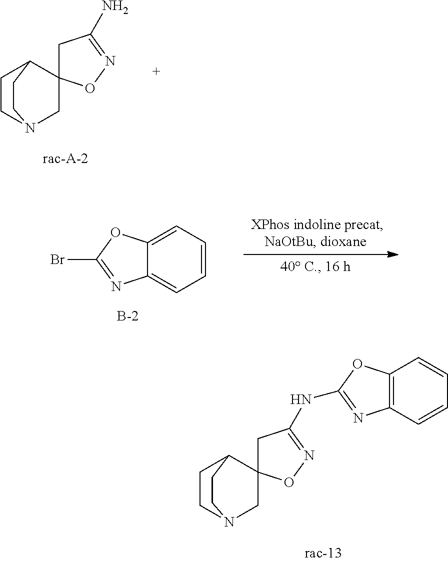

To a mixture of compound B-2 (0.50 g, 2.5 mmol) and compound rac-A-2 (0.46 g, 2.5 mmol) in dioxane (10 mL) under nitrogen at room temperature was added sodium tert-butoxide (0.48 g, 5.0 mmol) and chloro(2-dicyclohexyl phosphino-2′, 4′, 6′-tri-i-propyl-1,1′-biphenyl)[2-(2-aminoethyl)phenyl] palladium(II) methyl-t-butylether adduct (0.19 g, 0.25 mmol). The reaction mixture was stirred at 40° C. for 16 hours, then filtered and concentrated in vacuo. The residue was purified by prep-HPLC [Instrument: Shimadzu pump LC-20A; Column: GEMINI 200×50 mm, particle size: 10 μm; Mobile phase: 55-66.7% acetonitrile in H₂O (add 0.5% NH₃·H₂O, v/v)] to give compound rac-13 (0.10 g, 12% yield) as a brown solid.

Chiral Separation:

A solution of compound rac-13 (60 mg, 0.20 mmol) in 10 mL of ethanol was separated by SFC (Instrument: SFC 80; Column: Chiralpak OD-3 100×4.6 mm I.D., 3u,m; Mobile phase: ethanol (0.05% DEA) in CO₂) at room temperature. Each set of collected fractions was concentrated at room temperature and lyophilized to give:

N-(benzo[d]oxazol-2-yl)-4H-1′-azaspiro[isoxazole-5,3′-bicyclo[2.2.2]octan]-3-amine-enantiomer1 (free base) (compound 13-P1) (9.0 mg, 30% yield) as a white solid: cSFC analytical (D) tR=2.37 min., purity: 99.5%; LCMS (M): tR=0.77 min., 299.1 m/z (M+1); ¹H-NMR (CD₃OD, 400 MHz): δ 7.43 (t, J=8.4 Hz, 2H), 7.26 (t, J=6.8 Hz, 1H), 7.19 (t, J=7.2 Hz, 1H), 3.63 (d, J=17.2 Hz, 1H), 3.36 (m, 1H), 3.21-3.11 (m, 2H), 2.92-2.85 (m, 4H), 2.16-2.09 (m, 2H), 1.80-1.76 (m, 2H), 1.61-1.54 (m, 1H); and

N-(benzo[d]oxazol-2-yl)-4H-1′-azaspiro[isoxazole-5,3′-bicyclo[2.2.2]octan]-3-amine-enantiomer2 (free base) (compound 13-P2) (16 mg, 53% yield) as white solid: cSFC (D) analytical tR=2.93 min., purity: 99.4%; LCMS (M): tR=0.84 min., 299.1 m/z (M+1); ¹H-NMR (CD₃OD, 400 MHz): δ 7.43 (t, J=8.4 Hz, 2H), 7.26 (t, J=7.2 Hz, 1H), 7.19 (t, J=7.6 Hz, 1H), 3.63 (d, J=16.8 Hz, 1H), 3.37-3.35 (m, 1H), 3.21-3.11 (m, 2H), 2.92-2.81 (m, 4H), 2.16-2.09 (m, 2H), 1.80-1.78 (m, 2H), 1.61-1.54 (m, 1H).

### Example 14

### (+/−)-N-(6-methylbenzo[d]thiazol-2-yl)-4H-F-azaspiro[isoxazole-5,3′-bicyclo[2.2.2]octan]-3-amine (rac-14)

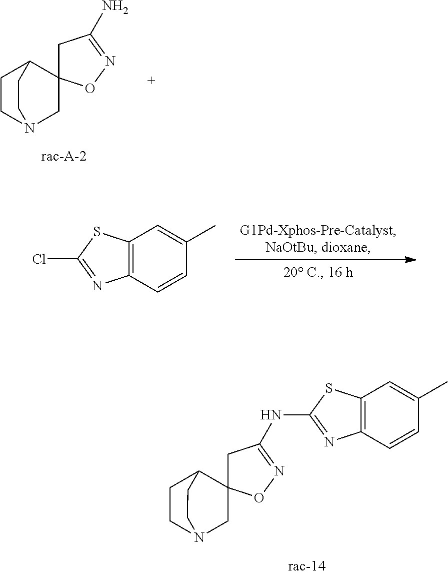 [SMILES: NC1=NOC2(C1)CN1CCC2CC1 ; Cc1ccc2nc(Cl)sc2c1 ; Cc1ccc2nc(NC3=NOC4(C3)CN3CCC4CC3)sc2c1]

To a mixture of 2-chloro-6-methylbenzo[d]thiazole (0.30 g, 1.6 mmol) and compound rac-A-2 (0.29 g, 1.6 mmol) in dioxane (10 mL) under nitrogen at room temperature was added sodium tert-butoxide (0.31 g, 3.2 mmol), dicyclohexyl42-(2,4,6-triisopropylphenyl)phenyll phosphane42-(2-aminoethyl)phenyll-chloro-palladium (0.12 g, 0.16 mmol). The reaction mixture was stirred at 20° C. for 16 hours, then filtered and concentrated in vacuo. The residue was purified by prep-HPLC [Instrument: Shimadzu pump LC-20A; Column: GEMINI 200×50 mm, particle size: 10 μm; Mobile phase: 55-66.7% acetonitrile in H₂O (add 0.5% NH₃·H₂O, v/v)] to give compound rac-14 (62 mg, 12% yield) as a white solid.

Chiral Separation:

A solution of compound rac-14 (60 mg, 0.18 mmol) in 10 mL of ethanol was separated by SFC (Instrument: SFC 80; Column: Chiralpak AY-3 100×4.6 mm I.D., 3 μm; Mobile phase: 40% ethanol (0.05% DEA) in CO₂) at room temperature. Each set of collected fractions was concentrated at room temperature and lyophilized. The resulting solids were dissolved in 0.2 M hydrochloric acid and again lyophilized to give:

N-(6-methylbenzo[d]thiazol-2-yl)-4H-1′-azaspiro[isoxazole-5,3′-bicyclo[2.2.2]octan]-3-amine-enantiomer1 hydrochloride (compound 14-P1) (27 mg, 90% yield) as a white solid : cSFC analytical (I) tR=2.97 min., purity: 100%; LCMS (J): tR=1.17 min., 329.1 m/z (M+1); ¹H-NMR (CD₃OD, 400 MHz): δ 7.78 (s, 1H), 7.68 (d, J=8.4 Hz, 1H), 7.43 (d, J=8.0 Hz, 1H), 7.08 (s, 1H), 3.80-3.62 (m, 3H), 3.54-3.45 (m, 2H), 3.41-3.38 (m, 3H), 2.50 (s, 3H), 2.44-2.41 (m, 2H), 2.19-1.94 (m, 3H); and

N-(6-methylbenzo[d]thiazol-2-yl)-4H-1′-azaspiro[isoxazole-5,3′-bicyclo[2.2.2]octan]-3-amine-enantiomer2 hydrochloride (compound 14-P2) (28 mg, 93% yield) as white solid : cSFC analytical (I) tR=3.75 min., purity: 98.7%; LCMS (M): tR=1.17 min., 329.1 m/z (M+1); ¹H-NMR (CD₃OD, 400 MHz): δ 7.77 (s, 1H), 7.68 (d, J=8.0 Hz, 1H), 7.42 (d, J=8.4 Hz, 1H), 7.08 (s, 1H), 3.80-3.62 (m, 3H), 3.54-3.45 (m, 2H), 3.41-3.37 (m, 3H), 2.50 (s, 3H), 2.47-2.37 (m, 2H), 2.19-1.90 (m, 3H).

### Example 15

### (+/−)-N-(6-cyclopropylbenzo[d]thiazol-2-yl)-4H-1′-azaspiro[isoxazole-5,3′-bicyclo[2.2.2]octan]-3-amine (rac-15)

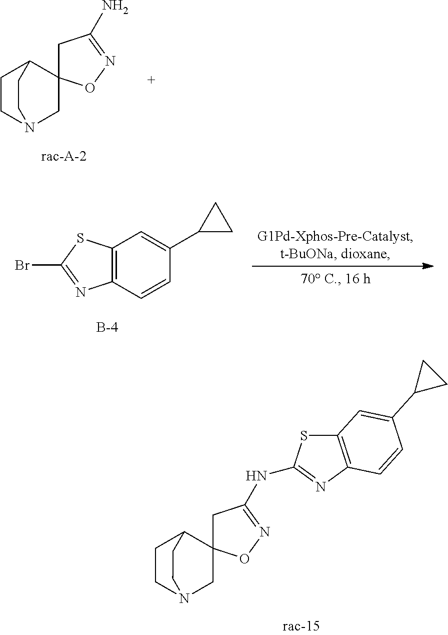

To a mixture of compound B-4 (0.42 g, 1.7 mmol) and compound rac- A-2 (0.30 g, 1.7 mmol) in dioxane (10 mL) under nitrogen at room temperature was added dicyclohexyl-[2-(2,4,6-triisopropylphenyl)phenyl]phosphane-[2-(2-aminoethyl)phenyl]-chloro-palladium (0.12 g, 0.17 mmol) and sodium tert-butoxide (0.48 g, 5.0 mmol). The reaction mixture was stirred at 70° C. for 16 hours, then filtered and concentrated in vacuo. The residue was purified by prep-HPLC [Instrument: GX-A; Column: GEMINI 250×50 mm, particle size: 10 μm; Mobile phase: 27-57% acetonitrile in H₂O (add 0.5% NH₃·H₂O, v/v)] to give compound rac-15 (0.17 g, 29% yield) as a white solid.

Chiral Separation:

A solution of compound rac-15 (0.17 g, 0.48 mmol) in 5 mL of methanol was separated by SFC (Instrument: SFC A; Column: Chiralpak AY-3 250×30 mm I.D., 10 μm; Mobile phase: 40% methanol (0.1% MEA) in CO₂) at room temperature. Each set of collected fractions was concentrated at room temperature and lyophilized. The resulting solids were dissolved in 0.2 M hydrochloric acid and again lyophilized to give:

N-(6-cyclopropylbenzo[d]thiazol-2-yl)-4H-1′-azaspiro[isoxazole-5,3′-bicyclo[2.2.2]octan]-3-amine-enantiomer1 hydrochloride (compound 15-P1) (70 mg, 75% yield) as a gray solid: cSFC analytical (I) tR=3.477 min., purity: 100%; LCMS (EE): tR=2.883 min., 355.1 m/z (M+1); ¹H-NMR (CD₃OD, 400 MHz): δ 7.66-7.64 (m, 2H), 7.32 (d, J=8.4 Hz, 1H), 3.79-3.61 (m, 3H), 3.53-3.33 (m, 5H), 2.46-2.41 (m, 2H), 2.15-1.96 (m, 4H), 1.09-1.04 (m, 2H), 0.80-0.76 (m, 2H); and

N-(6-cyclopropylbenzo[d]thiazol-2-yl)-4H-1′-azaspiro[isoxazole-5,3′-bicyclo[2.2.2]octan]-3- amine-enantiomer2 hydrochloride (compound 15-P2) (68 mg, 73% yield) as gray solid: cSFC analytical (I) tR=3.762 min., purity: 100%; LCMS (EE): tR=2.894 min., 355.1 m/z (M+1); ¹H-NMR (CD₃OD, 400 MHz): δ 7.67-7.65 (m, 2H), 7.32 (d, J=8.0 Hz, 1H), 3.79-3.60 (m, 3H), 3.53-3.33 (m, 5H), 2.46 (m, 2H), 2.15-1.93 (m, 4H), 1.07-1.05 (m, 2H), 0.78-0.77(m, 2H).

### Example 16

### (+/−)-N-(6-fluorobenzo[d]thiazol-2-yl)-4H-1′-azaspiro[isoxazole-5,3′-bicyclo[2.2.2]octan]-3-amine (rac-16)

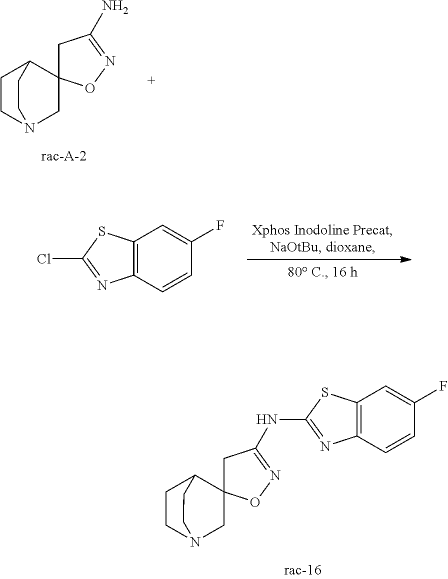 [SMILES: NC1=NOC2(C1)CN1CCC2CC1 ; Fc1ccc2nc(Cl)sc2c1 ; Fc1ccc2nc(NC3=NOC4(C3)CN3CCC4CC3)sc2c1]

To a mixture of 2-chloro-6-fluorobenzo[d]thiazole (0.30 g, 1.3 mmol) and compound rac-A-2 (0.23 g, 1.3 mmol) in dioxane (10 mL) under nitrogen at room temperature was added sodium tert-butoxide (0.25 g, 2.6 mmol), and chloro(2-dicyclohexyl phosphino-2′, 4′, 6′-tri-i-propyl-1,1′-biphenyl)[2-(2-aminoethyl)phenyl] palladium(II) methyl-t-butylether adduct (96 mg, 0.13 mmol). The reaction mixture was stirred at 80° C. for 16 hours, then filtered and concentrated in vacuo. The residue was purified by prep-HPLC [Instrument: Shimadzu pump LC-20A; Column: GEMINI 200 x 50 mm, particle size: 10 μm; Mobile phase: 55-66.7% acetonitrile in H₂O (add 0.5% NH₃·H₂O, v/v)] to give compound rac-16 (0.12 g, 28% yield) as a white solid.

Chiral Separation:

A solution of compound rac-16 (60 mg, 0.18 mmol) in 10 mL of ethanol was separated by SFC (Instrument: SFC 80; Column: Chiralpak AD-3 100×4.6 mm I.D., 3 μm; Mobile phase: 40% ethanol (0.05% DEA) in CO₂) at room temperature. Each set of collected fractions was concentrated at room temperature and lyophilized. The resulting solids were dissolved in 0.2 M hydrochloric acid and again lyophilized to give:

N-(6-fluorobenzo[d]thiazol-2-yl)-4H-1′-azaspiro[isoxazole-5,3′-bicyclo[2.2.2]octan]-3-amine-enantiomer1 hydrochloride (compound 16-P1) (10 mg, 30% yield) as a white solid: cSFC analytical (L) tR=2.64 min., purity: 99.8%; LCMS (M): tR=0.86 min., 333.1 m/z (M+1); ¹H-NMR (CD₃OD, 400 MHz): δ 7.75-7.71 (m, 2H), 7.33-7.28 (td, J₁=9.2 Hz, J₂=2.8 Hz, 1H), 3.78-3.68 (m, 2H), 3.64-3.60 (m, 1H), 3.52-3.37 (m, 5H), 2.45-2.41 (m, 2H), 2.18-1.93 (m, 3H); and

N-(6-fluorobenzo[d]thiazol-2-yl)-4H-1′-azaspiro[isoxazole-5,3′-bicyclo[2.2.2]octan]-3-amine -enantiomer2 hydrochloride (compound 16-P2) (12 mg, 36% yield) as white solid: cSFC analytical (L) tR=5.26 min., purity: 98.4%; LCMS (M): tR=0.86 min., 333.1 m/z (M+1); ¹H-NMR (CD₃OD, 400 MHz): δ 7.74-7.69 (m, 2H), 7.31-7.26 (td, J₁=9.2 Hz, J₂=2.8 Hz, 1H), 3.77-3.59 (m, 3H), 3.50-3.38 (m, 5H), 2.44 (m, 2H), 2.13-1.96 (m, 3H).

### Example 17

### (R)—N -(5-chloro-6-fluorobenzo[d]thiazol-2-yl)-4H-1′-azaspiro[isoxazole-5,3′-bicyclo[2.2.2]octan]-3-amine hydrochloride ((R)-17)

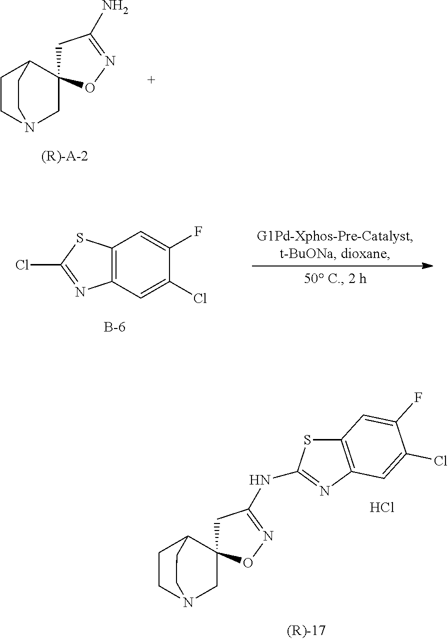

To a solution of compound B-6 (0.10 g, 0.45 mmol), compound (R)-A-2 (82 mg, 0.45 mmol) and dicyclohexyl[2-(2,4,6-triisopropylphenyl)phenyl]phosphane[2-(2-aminoethyl)phenyl]-chloro-palladium (17 mg, 0.023 mmol) in dioxane (4 mL) was added sodium tert-butoxide (2 mol/L in tetrahydrofuran, 0.68 mL). The mixture was stirred at 50° C. for 2 hours under nitrogen atmosphere, then filtered and concentrated in vacuo. The residue was purified by prep-HPLC [Instrument: GX-A; Column: GEMINI 250×50 mm, particle size: 10 μm; Mobile phase: 35-49% acetonitrile in H₂O (add 0.5% NH₃ H₂O, v/v)] and lyophilized. The resulting solids were dissolved in 0.2 M hydrochloric acid and again lyophilized to give:

Compound (R)-17 (50 mg, 26% yield) as a white solid: cSFC analytical (I) tR=3.243 min., purity: 100%; LCMS (FF): tR=2.848 min., (ES⁺) m/z (M+H)⁺=367.0; ¹H-NMR (CD₃OD, 400 MHz): δ 7.78-7.72 (m, 2H), 3.50 (d, J=16.4 Hz, 1H), 3.44-3.33 (m, 2H), 3.28 (s, 1H), 3.17-3.07 (m, 4H), 2.31-2.23 (m, 2H),1.98-1.90 (m, 2H), 1.87-1.74 (m, 1H).

### Example 18

### (R)—N-(6-chloroisoquinolin-3-yl)-4H-1′-azaspiroksoxazole-5,3¹-bicyclo[2.2.2]octan1-3-amine hydrochloride ((R)-18)

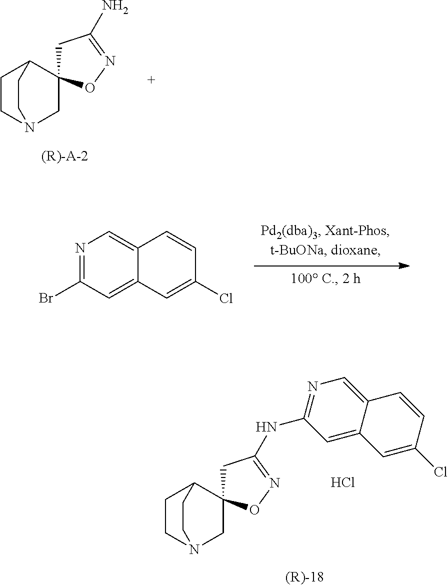 [SMILES: NC1=NO[C@@]2(C1)CN1CCC2CC1 ; Clc1ccc2cnc(Br)cc2c1 ; Clc1ccc2cnc(NC3=NO[C@@]4(C3)CN3CCC4CC3)cc2c1 ; Cl]

To a solution of 3-bromo-6-chloro-isoquinoline (0.2 mg, 0.83 mmo1) and compound (R)- A-2 (0.16 g, 0.87 mmol) in dioxane (2.0 mL) was added tris(dibenzylideneacetone)dipalladium(0) (30 mg, 0.033 mmol), 4,5-bis(diphenylphosphino)-9,9-dimethylxanthene (29 mg, 0.050 mmol) and sodium tert-butoxide (0.16 g, 1.65 mmol). The mixture was stirred at 100° C. for 2 hours under nitrogen, then filtered and concentrated in vacuo. The residue was purified by prep-HPLC [Instrument: GX-B; Column: Phenomenex Synergi-C18 150×30 mm, particle size: 4 μm; Mobile phase: 24-54% acetonitrile in H₂O (add 0.1% TFA, v/v)]. The solution was treated with 0.2 M hydrochloric acid and lyophilized to give:

Compound (R)-18 (30 mg, 9.6% yield) as a yellow solid: cSFC analytical (H) tR=3.34 min., purity: 99.7%; LCMS (FF): tR=2.339 min, 343.1, m/z (M+1); ¹H-NMR (DMSO-d6, 400 MHz): δ 10.60 (s 1H), 9.98 (s, 1H), 9.12 (s, 1H), 8.06 (d, J=8.8 Hz, 1H), 7.95 (d, J=6 Hz, 1H), 7.46 (dd, J₁=8.8 Hz, J₂=2 Hz , 1H), 3.65 (m, 1H), 3.46-3.11 (m, 7H), 2.27-2.17 (m, 2H), 1.94-1.90 (m, 1H), 1.79-1.77 (m, 2H).

### Example 19

### (+/−) N-(7-chloroisoquinolin-3-yl)-4H-1′-azaspiroksoxazole-5,3¹-bicyclo[2.2.2]octan1-3-amine (rac-19)

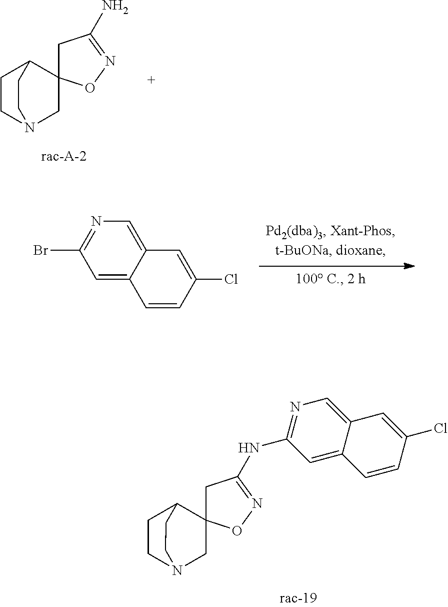 [SMILES: NC1=NOC2(C1)CN1CCC2CC1 ; Clc1ccc2cc(Br)ncc2c1 ; Clc1ccc2cc(NC3=NOC4(C3)CN3CCC4CC3)ncc2c1]

To a solution of 3-bromo-7-chloro-isoquinoline (50 mg, 0.21 mmol) and compound rac-A-2 (39 mg, 0.21 mmol) in dioxane (2.0 mL) under nitrogen was added tris(dibenzylideneacetone)dipalladium(0) (7.6 mg, 0.083 mmol), 4,5-bis(diphenylphosphino)-9,9-dimethylxanthene (7.2 mg, 0.012 mmol) and sodium tert-butoxide (39 mg, 0.41 mmol). The mixture was stirred at 100° C. for 2 hours, then filtered and concentrated in vacuo. The residue was purified by prep-HPLC [Instrument: GX-B; Column: Phenomenex Synergi-C18 150×30 mm, particle size: 4 μm; Mobile phase: 21-51% acetonitrile in H₂O (add 0.1% TFA, v/v)]. The solution was treated with 0.2 M hydrochloric acid and lyophilized to give compound rac-19 (50 mg, 13% yield) as a yellow solid.

Chiral Separation:

A solution of compound rac-19 (50 mg, 109.5 mmol) in 10 mL of ethanol was separated by SFC (Instrument: SFC 80; Column: Chiralpak AD-H 250×30 mm I.D., particle size: 10 μm; Mobile phase: 40% iso-propanol (0.05% DEA) in CO₂) at room temperature. Each set of collected fractions was concentrated at room temperature and lyophilized. The resulting solids were dissolved in 0.2 M hydrochloric acid and again lyophilized to give:

N-(7-chloroisoquinolin-3-yl)-4H-1′-azaspiro[isoxazole-5,3′-bicyclo[2.2.2]octan]-3-amine-enantiomer1 hydrochloride (compound 19-P1) (12.5 mg, 68% yield) as a yellow solid: cSFC analytical (G) tR=2.50 min., purity: 96.7%; LCMS (FF): tR=2.358 min, 343.1, m/z (M+1); ¹H-NMR (CD₃OD, 400 MHz): δ 9.16 (s, 1H), 8.18 (s, 1H), 8.01 (s, 1H), 7.93 (d, J=9.2 Hz, 1H), 7.79 (dd, J₁=8.4 Hz, J₂=5.2 Hz, 1H), 3.74-3.71 (m, 1H), 3.68-3.60 (m, 1H), 3.51-3.37 (m, 5H), 2.45-2.42 (m, 2H), 2.16-1.94 (m, 3H); and

N-(7-chloroisoquinolin-3-yl)-4H-1′-azaspiro[isoxazole-5,3′-bicyclo[2.2.2]octan]-3-amine-enantiomer2 hydrochloride (compound 19-P2) (12 mg, 58.6% yield) as a yellow solid: cSFC analytical (G) tR=3.84 min., purity: 95.5%; LCMS (FF): tR=2.354 min, 343.1, m/z (M+1); ¹H-NMR (CD₃OD, 400 MHz): δ 9.15 (m, 1H), 8.17 (m, 1H), 8.01 (s, 1H), 7.94-7.91 (m, 1H), 7.79-7.76 (m, 1H), 3.77-3.60 (m, 3H), 3.51-3.37 (m, 5H), 2.45-2.42 (m, 2H), 2.17-1.93 (m, 3H).

(R)—N-(7-chloroisoquinolin-3-yl)-4H-1′-azaspiro[isoxazole-5,3′-bicyclo[2.2.2]octan]-3-amine hydrochloride ((R)-19)

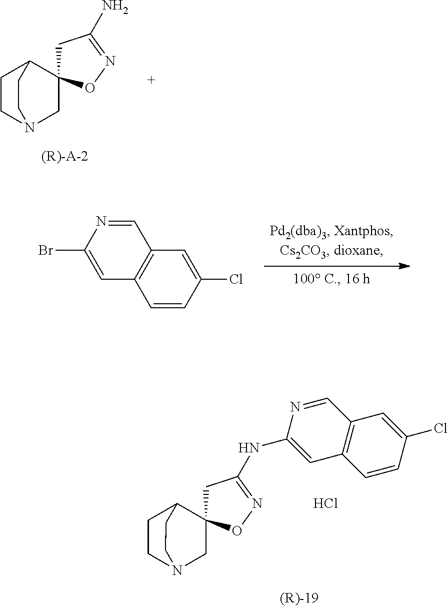 [SMILES: NC1=NO[C@@]2(C1)CN1CCC2CC1 ; Clc1ccc2cc(Br)ncc2c1 ; Clc1ccc2cc(NC3=NO[C@@]4(C3)CN3CCC4CC3)ncc2c1 ; Cl]

To a solution of 3-bromo-7-chloroisoquinoline (0.36 g, 1.5 mmol), compound (R)-A-2 (0.27 g, 1.5 mmol), tris(dibenzylideneacetone)dipalladium(0) (0.14 g, 0.15 mmol) and 4,5-bis(diphenylphosphino)-9,9-dimethylxanthene (0.17 g, 0.30 mmol) in dioxane (10 mL) was added cesium carbonate (0.97 g, 3.0 mmol). The mixture was stirred at 100° C. for 16 hours under nitrogen atmosphere, then filtered and concentrated in vacuo. The residue was purified by prep-HPLC [Instrument: GX-B; Column: Welch Ultimate AQ-C18 150×30 mm, particle size: 5 μm; Mobile phase: 18-48% acetonitrile in H₂O (add 0.1% TFA, v/v)]. The solution was treated with 0.2 M hydrochloric acid and lyophilized to give:

Compound (R)-19 (0.22 g, 39% yield) as a yellow solid: cSFC analytical (I) tR=3.896 min., purity: 100%; LCMS (GG): tR=2.032 min., (ES⁺) m/z (M+H)⁺=343.1; ¹H-NMR (CD₃OD, 400 MHz): δ 9.20 (s, 1H), 8.21 (s, 1H), 8.01 (s, 1H), 7.96 (d, J=9.2 Hz, 1H), 7.82 (d, J=8.8 Hz, 1H), 3.79-3.62 (m, 3H), 3.54-3.45 (m, 2H), 3.42-3.38 (m, 3H), 2.46-2.42 (m, 2H), 2.16-2.13 (m, 1H), 2.08-1.94 (m, 2 H).

### Example 20

### (+/−)-N-(4-chlorophenyl)-4H-1′-azaspiro[isoxazole-5,3¹-bicyclo[2.2.2]octan]-3-amine (rac-20)

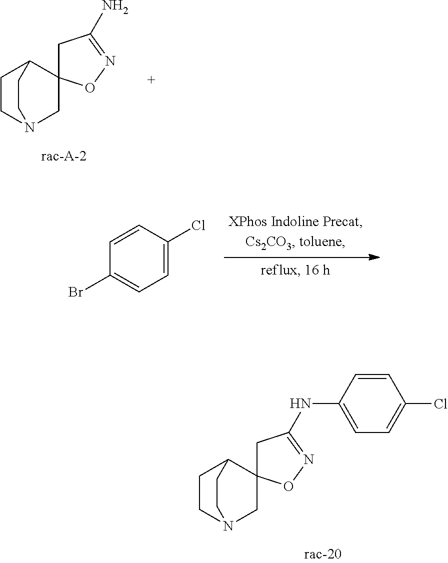 [SMILES: NC1=NOC2(C1)CN1CCC2CC1 ; Clc1ccc(Br)cc1 ; Clc1ccc(NC2=NOC3(C2)CN2CCC3CC2)cc1]

To a mixture of 1-bromo-4-chlorobenzene (0.40 g, 2.1 mmol) and compound rac-A-2 (0.38 g, 2.1 mmol) in toluene (10 mL) under nitrogen at room temperature was added cesium carbonate (2.0 g, 6.3 mmol), chloro(2-dicyclohexyl phosphino-2′, 4′, 6′-tri-i-propyl-1,1′-biphenyl)[2-(2-aminoethyl)phenyl] palladium(II) methyl-t-butylether adduct (0.15 g, 0.21 mmol). The reaction mixture was stirred at reflux for 16 hours, then filtered and concentrated in vacuo. The residue was purified by prep-HPLC [Instrument: Shimadzu pump LC-20A; Column: GEMINI 200×50 mm, particle size: 10 μm; Mobile phase: 55-66.7% acetonitrile in H₂O (add 0.5% NH₃·H₂O, v/v)] to give compound rac-20 (68 mg, 11% yield) as a white solid.

Chiral Separation:

A solution of compound rac-20 (48 mg, 0.16 mmol) in 10 mL of ethanol was separated by SFC (Instrument: SFC 80; Column: Chiralpak AD-3 100×4.6 mm I.D., 3 μm; Mobile phase: 40% methanol (0.1% MEA) in CO₂) at room temperature. Each set of collected fractions was concentrated at room temperature and lyophilized. The resulting solids were dissolved in 0.2 M hydrochloric acid and again lyophilized to give:

N-(4-chlorophenyl)-4H-1′-azaspiro[isoxazole-5,3′-bicyclo[2.2.2]octan]-3-amine-enantiomer1 hydrochloride (compound 20-P1) (12 mg, 44% yield) as a white solid : cSFC analytical tR=1.04 min., purity: 100%; LCMS (J): tR=1.18 min., 292.1 m/z (M+1); ¹H-NMR (CD₃OD, 400 MHz): δ 7.42 (d, J=8.0 Hz, 2H), 7.27 (d, J=9.2 Hz, 2H), 3.75-3.58 (m, 2H), 3.46-3.36 (m, 5H), 3.30 (s, 1H), 2.44-2.38 (m, 2H), 2.16-1.91 (m, 3H); and

N-(4-chlorophenyl)-4H-1′-azaspiro[isoxazole-5,3′-bicyclo[2.2.2]octan]-3-amine -enantiomer2 hydrochloride (compound 20-P2) (13 mg, 48% yield) as white solid : cSFC analytical(G) tR=2.09 min., purity: 99.6%; LCMS (M): tR=1.19 min., 292.1 m/z (M+1); ¹H-NMR (CD₃OD, 400 MHz): δ 7.42 (d, J=8.8 Hz, 2H), 7.27 (d, J=8.8 Hz, 2H), 3.70-3.58 (m, 2H), 3.45-3.36 (m, 5H), 3.30 (s, 1H), 2.43-2.37 (m, 2H), 2.12-1.89 (m, 3H).

(R)—N-(4-chlorophenyl)-4H-1′-azaspiro[isoxazole-5,3′-bicyclo[2.2.2]octan]-3-amine hydrochloride ((R)-20)

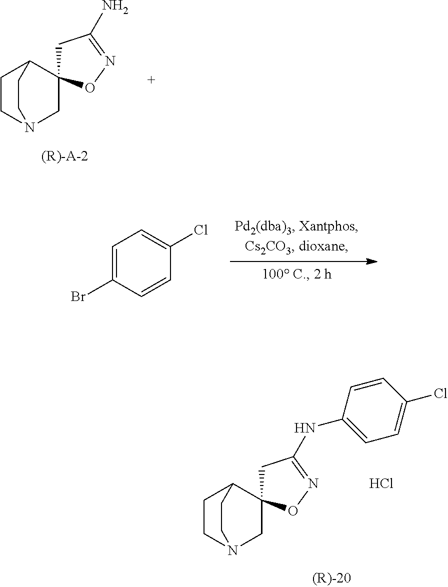 [SMILES: NC1=NO[C@@]2(C1)CN1CCC2CC1 ; Clc1ccc(Br)cc1 ; Clc1ccc(NC2=NO[C@@]3(C2)CN2CCC3CC2)cc1 ; Cl]

To a solution of 1-bromo-4-chlorobenzene (0.21 g, 1.1 mmol), compound (R)-A-2 (0.20 g, 1.1 mmol), tris(dibenzylideneacetone)dipalladium(0) (0.10 g, 0.11 mmol) and 4,5-bis(diphenylphosphino)-9,9-dimethylxanthene (0.13 g, 0.22 mmol) in dioxane (8 mL) under nitrogen was added cesium carbonate (0.72 g, 2.2 mmol). The reaction mixture was stirred at 100° C. for 2 hours, then filtered and concentrated in vacuo. The residue was purified by prep-HPLC [Instrument: GX-E; Column: Phenomenex Synergi C18 150×30 mm, particle size: 4 μm; Mobile phase: 22-47% acetonitrile in H₂O (add 0.05% HCl v/v)]. The resulting solution was lyophilized to give:

Compound (R)-20 (0.20 g, 55% yield) as a white solid : cSFC analytical (I) tR=3.010 min., purity: 98.24%; LCMS (GG): tR=1.656 min., (ES⁺) m/z (M+H)⁺=292.1; ¹H-NMR (CD₃OD, 400 MHz): δ 7.42 (d, J=8.8 Hz, 2H), 7.29 (d, J=8.8 Hz, 2H), 3.71 (d, J=14 Hz, 1H), 3.61 (d, J=14 Hz, 1H), 3.50-3.46 (m, 2H), 3.42-3.33 (m, 4H), 2.39-2.36 (m, 2H), 2.13-2.11 (m, 1H), 2.02-1.93 (m, 2H).

### Example 21

### (+/−)-N-(3, 4-dichlorophenyl)-4H-1′-azaspiro[isoxazole-5,3′-bicyclo[2.2.2]octan]-3-amine (rac-21)

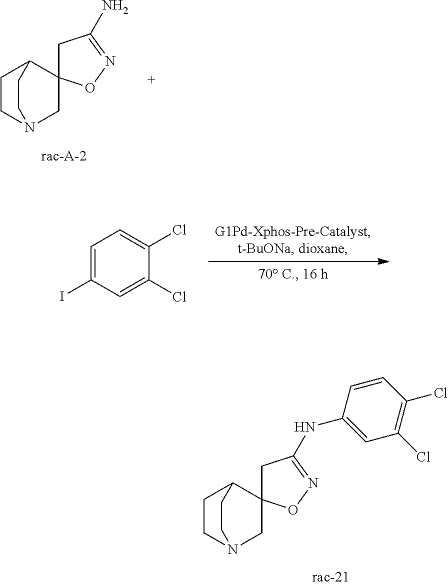 [SMILES: NC1=NOC2(C1)CN1CCC2CC1 ; Clc1ccc(I)cc1Cl ; Clc1ccc(NC2=NOC3(C2)CN2CCC3CC2)cc1Cl]

To a solution of 1,2-dichloro-4-iodo-benzene (0.90 g, 3.3 mmo1), compound rac- A-2 (0.40 mg, 2.2 mmol) and cesium carbonate (2.2 g, 6.6 mmol) in dioxane (10 mL) was added dicyclohexyl42-(2,4,6-triisopropylphenyl)phenyllphosphane42-(2-aminoethyl)phenyll-chloro-palladium (0.16 g, 0.22 mmol). The mixture was stirred at 70° C. for 16 hours under nitrogen, then filtered and concentrated in vacuo. The residue was purified by prep-HPLC [Instrument: GX-A; Column: GEMINI 250×50 mm, particle size: 10 μm; Mobile phase: 34-64% acetonitrile in H₂O (add 0.5% NH₃·H₂O, v/v)] to give compound rac-21 (0.25 g, 35% yield) as a white solid.

Chiral Separation:

A solution of compound rac-21 (0.12 g, 0.37 mmol) in 5 mL of ethanol was separated by SFC (Instrument: SFC A; Column: Chiralpak AD-3 250×30 mm I.D., lOμm; Mobile phase: 40% ethanol (0.1% MEA) in CO₂) at room temperature. Each set of collected fractions was concentrated at room temperature and lyophilized. The resulting solids were dissolved in 0.2 M hydrochloric acid and again lyophilized to give:

N-(3, 4-dichlorophenyl)-4H-1′-azaspiro[isoxazole-5,3′-bicyclo[2.2.2]octan]-3-amine-enantiomer1 hydrochloride (compound 21-P1) (35 mg, 52% yield) as a white solid: cSFC analytical(G) tR=3.573 min., purity: 96.32%; LCMS (J): tR=1.321 min., 326.1 m/z (M+1); ¹H-NMR (CD₃OD, 400 MHz): δ 7.76 (d, J=2.0 Hz, 1H), 7.40 (d, J=8.8 Hz, 1H), 7.27 (dd, J1=9.2 Hz, J2=2.8 Hz, 1H), 3.69-3.60 (m, 2H), 3.47-3.33 (m, 5H), 3.28 (m, 1H), 2.45-2.38 (m, 2H), 2.15-2.08 (m, 1H), 2.08-1.89(m, 2H); and

N-(3, 4-dichlorophenyl)-4H-1′-azaspiro[isoxazole-5,3′-bicyclo[2.2.2]octan]-3-amine-enantiomer2 hydrochloride (compound 21-P2) (35 mg, 52% yield) as white solid: cSFC analytical(G) tR=3.938 min., purity: 94.66%; LCMS (J): tR=1.320 min., 326.1 m/z (M+1); ¹H-NMR (CD₃OD, 400 MHz): δ 7.76 (d, J=2.4 Hz, 1H), 7.40 (d, J=8.8 Hz, 1H), 7.27 (dd, J1=8.8 Hz, J2=2.4 Hz, 1H), 3.70-3.59 (m, 2H), 3.47-3.33 (m, 5H), 3.29 (m, 1H), 2.45-2.37 (m, 2H), 2.16-2.09 (m, 1H), 2.02-1.89(m, 2H).

### Example 22

### (R)—N-(quinazolin-2-yl)-4H-1′-azaspiro[isoxazole-5,3′-bicyclo[2.2.2]octan]-3-amine hydrochloride ((R)-22)

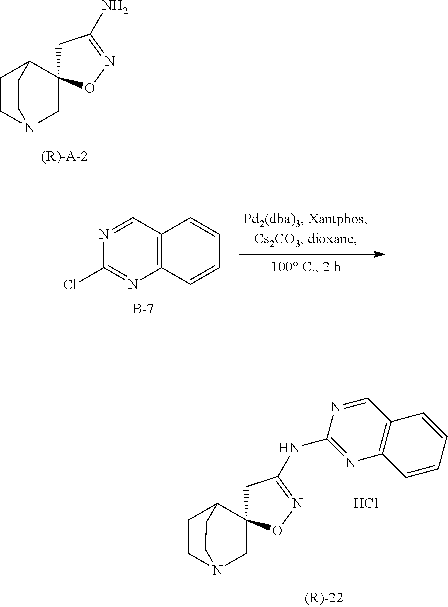

A mixture of compound B-7 (91 mg, 0.55 mmol), compound (R)-A-2 (100 mg, 0.55 mmol) and cesium carbonate (360 mg, 1.1 mmol) in dioxane (3 mL) was degassed and purged with nitrogen 3 times. Tris(dibenzylideneacetone)dipalladium(0) (50 mg, 0.055 mmol) and 4,5-bis(diphenylphosphino)-9,9-dimethylxanthene (32 mg, 0.055 mmol) were added, and the resulting mixture was stirred at 100° C. for 2 hours under nitrogen. The reaction mixture was diluted with methanol, filtered through a silica gel plug and concentrated under reduced pressure. The residue was purified by prep-HPLC [Instrument: GX-A; Column: Phenomenex Gemini C18 250*50 mm, particle size: 10 μm; Mobile phase: 22-52% acetonitrile in H₂O (add 0.05% ammonia, v/v)]. The solution was treated with 0.2 N hydrochloric acid and lyophilized to give:

Compound (R)-22 (30 mg, 17% yield) as a yellow solid: cSFC analytical (I) tR=3.082 min., purity: 100%; LCMS (FF): tR=1.913 min., (ES⁺) m/z (M+H)⁺=310.1; ¹H-NMR (CD₃OD, 400 MHz): δ 9.23 (s, 1H), 7.91 (d, J=8.4 Hz, 1H), 7.86-7.82 (m, 2H), 7.48-7.44 (m, 1H), 3.77 (d, J=17.2 Hz, 1H), 3.44 (d, J=16.8, 1H), 3.20-3.10 (m, 2H), 2.91-2.82 (m, 4H), 2.16-2.08 (m, 2H), 1.79-1.77 (m, 2H), 1.60-1.52 (m, 2H).

### Example 23

### (R)—N-(8 -chloroiso quin olin -3-yl)-4H-1′-azaspiroksoxazole-5,3¹-bicyclo[2.2.2]octan1-3-amine hydrochloride ((R)-23)

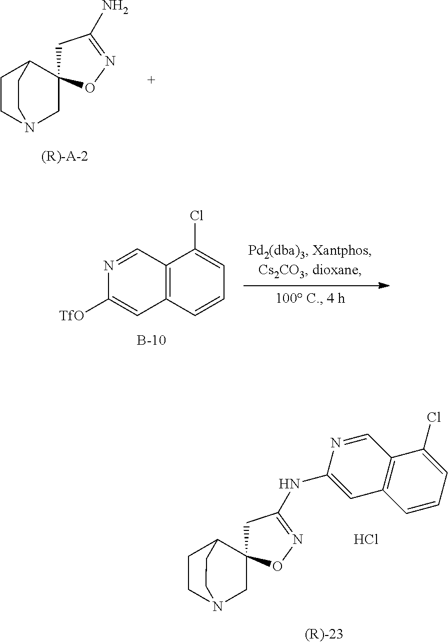

To a mixture of compound B-10 (0.16 g, 0.51 mmol), compound (R)-A-2 (0.77 mg, 0.43 mmol) in dioxane (8 mL) under nitrogen at room temperature was added tris(dibenzylideneacetone)dipalladium(0) (39 mg, 43 μmop, cesium carbonate (0.28 g, 0.86 mmol) and 4,5-bis(diphenylphosphino)-9,9-dimethylxanthene (50 mg, 86 μmol). The reaction mixture was stirred at 100° C. for 4 hours, then filtered and concentrated in vacuo. The residue was purified by prep-HPLC [Instrument: GX-E; Column: Phenomenex Synergi C18 150×30 mm, particle size: 4 μm; Mobile phase: 20-45% acetonitrile in H₂O (add 0.05% HCl v/v)] and lyophilized to give:

Compound (R)-23 (50 mg, 31% yield) as a yellow solid: cSFC analytical (I) tR=4.297 min., purity: 99.34%; LCMS (FF): tR=2.301 min., (ES⁺) m/z (M+H)⁺=343.1; ¹H-NMR (CD₃OD, 400 MHz): δ 9.50 (s, 1H), 8.03 (s, 1H), 7.94 (d, J=8.4 Hz, 1H), 7.83 (t, J=7.2 Hz, 1H), 7.73 (d, J=7.6 Hz, 1H), 3.82-23.66 (m, 3H), 3.58-3.33 (m, 5H), 2.48-2.41 (m, 2H), 2.17-1.98 (m, 3H).

### Example 24

### (R)—N -(5 -chloroisoquinolin-3-yl)-4H-1′-azaspiroksoxazole-5,3′-bicyclo[2.2.2]octan1-3-amine hydrochloride ((R)-24)

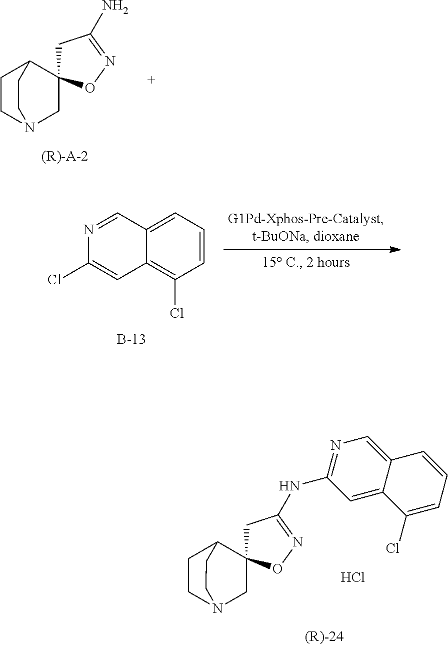

To a solution of compound B-13 (87 mg, 0.44 mmol), compound (R)-A-2 (80 mg, 0.44 mmol) and dicyclohexyl[2-(2,4,6-triisopropylphenyl)phenyl]phosphane[2-(2-aminoethyl)phenyl]-chloro-palladium (16 mg, 0.022 mmol) in dioxane (10 mL) was added sodium tert-butoxide (2 M in tetrahydrofuran, 0.44 mL). The mixture was stirred at 15° C. for 2 hours under nitrogen, then concentrated in vacuo. The residue was purified by prep-HPLC [Instrument: GX-I; Column: Xtimate C18 150×25 mm, particle size: 5 μm; Mobile phase: 14-44% acetonitrile in H₂O (add 0.1% TFA, v/v)]. The solution was treated with 0.2 M hydrochloric acid and lyophilized to give:

Compound (R)-24 (20 mg, 10% yield) as a white solid: cSFC analytical (I) tR=3.159 min., purity: 100%; LCMS (FF): tR=2.294 min., (ES⁺) m/z (M+H)⁺=343.1; ¹H-NMR (CD₃OD, 400 MHz): δ 9.23 (s, 1H), 8.30 (s, 1H), 8.09 (d, J=8.4 Hz, 1H), 7.94 (d, J=7.6 Hz, 1H), 7.54 (t, J1=16 Hz, J2=8 Hz 1H), 3.78-3.69 (m, 2H), 3.53-3.45 (m, 2H), 3.42-3.33 (m, 3H), 2.47 (m, 2H), 2.16-2.14 (m, 1H), 2.06-1.96 (m, 2H).

### Example 25

### (R)—N -(5 -methylbenzo[d]thiazol-2-yl)-4H-1′-azaspiro[isoxazole-5,3′-bicyclo[2.2.2]octan]-3-amine hydrochloride ((R)−25)

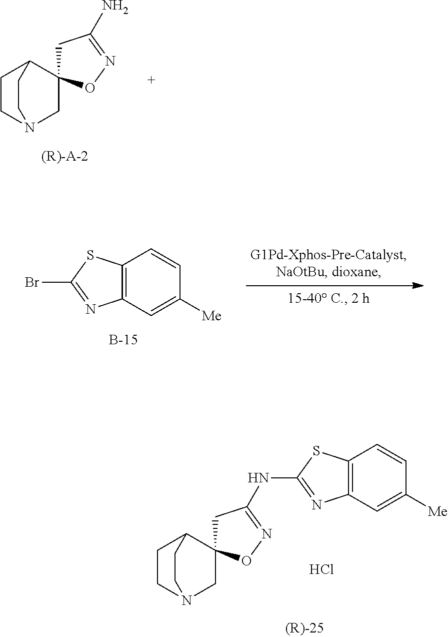

A mixture of compound B-15 (0.10 g, 0.44 mmol), compound (R)-A-2 (0.08 g, 0.44 mmol), dicyclohexyl42,-(2,4,6-triisopropylphenyl)phenyllphosphane-[2,-(2-aminoethyl)phenyl]-chloro-palladium (0.02 g, 0.02 mmol) and sodium tert-butoxide (0.08 g, 0.88 mmol) in dioxane (10 mL) at 15° C. was de-gassed and placed under nitrogen. The reaction was stirred at 40° C. for 2 hours, then filtered and concentrated in vacuo. The residue was purified by prep-HPLC [Instrument: GX-B; Column: Phenomenex Synergi C18 100×21.2 mm, particle size: 4 μm; Mobile phase: 20-50% acetonitrile in H₂O (add 0.1% TFA, v/v)]. The solution was treated with 0.2 M hydrochloric acid and lyophilized to give:

Compound (R)-25 (41 mg, 25% yield) as a yellow solid: cSFC analytical (I) tR=3.58 min., purity: 100%; LCMS (FF): tR=2.226 min., (ES⁺) m/z (M+H)⁺=329.1; ¹H-NMR (CD₃OD, 400 MHz): δ 7.80 (d, J=8.0 Hz, 1H), 7.58 (s, 1H), 7.29 (d, J=8.4 Hz, 1H), 3.76-3.68 (m, 2H), 3.64-3.58 (m, 1H), 3.51-3.43 (m, 2H), 3.36-3.31 (m, 3H), 2.47 (s, 3H), 2.42-2.35 (m, 2H), 2.13-2.07 (m, 1H), 2.00-1.92 (m, 2H).

### Example 26

### (R)—N -(5-cyclopropylbenzo[d]thiazol-2-yl)-4H-1′-azaspiro[isoxazole-5,3′-bicyclo[2.2.2]octan]-3-amine hydrochloride ((R)-26)

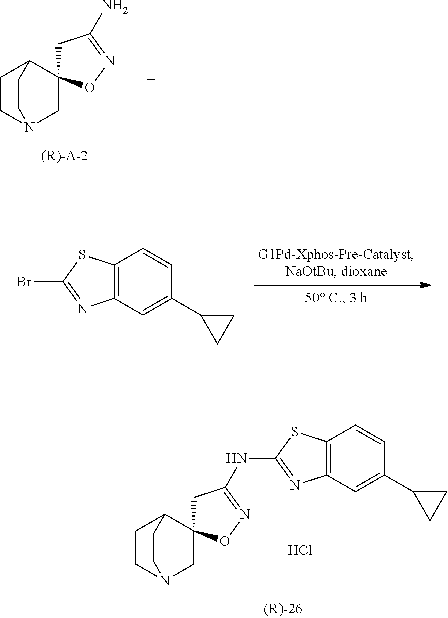 [SMILES: NC1=NO[C@@]2(C1)CN1CCC2CC1 ; Brc1nc2cc(C3CC3)ccc2s1 ; c1cc2sc(NC3=NO[C@@]4(C3)CN3CCC4CC3)nc2cc1C1CC1 ; Cl]

A mixture of compound B-17 (140 mg, 0.55 mmol), compound (R)-A-2 (100 mg, 0.55 mmol) and sodium tert-butoxide (1 M, 1.66 mL,) in dioxane (3.0 mL) was degassed and purged with nitrogen 3 times. Chloro-(2-dicyclohexylphosphino-2′, 4′, 6′-tri-i-propyl-1,1′-biphenyl)[2-(2-aminoethyl)phenyl]palladium(II) methyl-t-butyl ether adduct (20 mg, 0.028 mmol) was added, and the resulting mixture was stirred at 50° C. for 3 hours. On completion, the mixture was diluted with methanol, filtered through a silica-gel plug and concentrated in vacuo. The residue was purified by prep-HPLC [Instrument: GX-B; Column: YMC-Actus Pro C18 150×30 mm, particle size: 5 μm; Mobile phase: 27-57% acetonitrile in H₂O (add 0.1% TFA, v/v)] and lyophilized to give:

Compound (R)-26 (40 mg, 19% yield) as a yellow solid: cSFC analytical (I) tR=3.718 min., purity: 100%; LCMS (FF): tR=2.402 min., (ES⁺) m/z (M+H)⁺=355.1; ¹H-NMR (CD₃OD, 400 MHz): δ 7.78 (d, J=8.4 Hz, 1H), 7.46 (s, 1H), 7.17 (d, J=8.4 Hz, 1H), 3.77-3.71 (m, 2H), 3.59-3.51 (m, 1H), 3.46-3.37 (m, 2H), 3.35-3.32 (m, 3H), 2.44-2.42 (m, 2H), 2.13-1.97 (m, 4H), 1.10-1.05 (m, 2H), 0.79-0.76 (m, 2H).

### Example 27

### (R)—N-(5-fluorobenzo[d]thiazol-2-yl)-4H-1′-azaspiro[isoxazole-5,3′-bicyclo[2.2.2]octan]-3-amine hydrochloride ((R)-27)

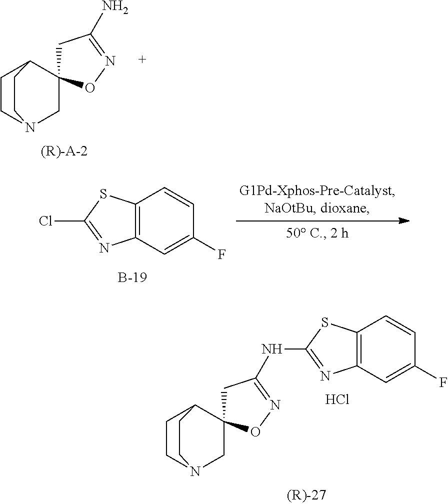

To a solution of compound B-19 (150 mg, 0.65 mmol) and compound (R)-A-2 (0.12 g, 0.68 mmol) in dioxane (2.0 mL) was added dicyclohexyl[2-(2,4,6-triisopropylphenyl)phenyl]phosphane-[2,-(2-aminoethyl)phenyl]-chloro-palladium (24 mg, 0.032 mmol) and sodium tert-butoxide (0.32 g, 3.31 mmol). The mixture was stirred at 50° C. for 2 hours under nitrogen, then concentrated in vacuo. The residue was purified by prep-HPLC [Instrument: GX-B; Column: Phenomenex Synergi C18 100×21.2 mm, particle size: 4 μm; Mobile phase: 15-25% acetonitrile in H₂O (add 0.1% TFA, v/v)]. The solution was treated with 0.2 M hydrochloric acid and lyophilized to give:

Compound (R)-27 (40 mg, 17% yield) as a white solid : cSFC analytical (G) tR=3.29 min., purity: 100.00%; LCMS (FF): tR=2.149 min, 333.1 m/z (M+1); ¹H-NMR (CD₃OD, 400 MHz): δ 7.89-7.86 (m, 1H), 7.46-7.44 (m, 1H), 7.16-7.12 (m, 1H), 3.77-3.71 (m, 2H), 3.63-3.59 (m, 1 H), 3.50-3.46 (m, 2H), 3.44-3.37 (m, 3H), 2.44-2.41 (m, 2H), 2.16-2.12 (m, 1H), 2.10-1.93 (m, 2H).

### Example 28

### (R)—N-(5 - (tr ifluo r omethyl)b en z o [d]thiazol-2-yl)-4H-1′-azaspiro[isoxazole-5,3′-bicyclo[2.2.2]octan]-3-amine hydrochloride ((R)-28)

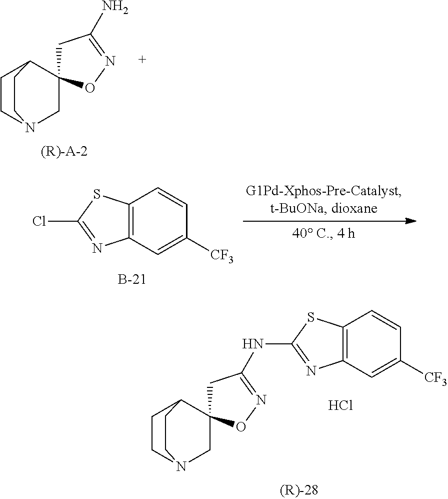

To a mixture of compound B-21 (66 mg, 0.28 mmol), compound (R)-A-2 (50 mg, 0.28 mmol) and dicyclohexyl[2-(2,4,6-triisopropylphenyl)phenyl]phosphane[2-(2-aminoethyl)phenyl]-chloro-palladium (10 mg, 0.014 mmol) in dioxane (2 mL) under nitrogen was added sodium tert-butoxide (2 M in tetrahydrofuran, 0.28 mL). The mixture was stirred at 40° C. for 4 hours, then filtered and concentrated in vacuo. The residue was purified by prep-HPLC [Instrument: GX-B; Column: Phenomenex Synergi C18 100×21.2 mm, particle size: 4μm; Mobile phase: 26-56% acetonitrile in H₂O (add 0.1% TFA, v/v)]. The solution was treated with 0.2 M hydrochloric acid and lyophilized to give:

Compound (R)-28 (44 mg, 42% yield) as a yellow solid : cSFC analytical (I) tR=2.58 min., purity: 100%; LCMS (FF): tR=2.413 min., 383.1 m/z (M+1); ¹H-NMR (CD₃OD, 400 MHz): δ 8.03 (d, J=8.4 Hz, 1H), 7.93 (s, 1H), 7.53 (d, J=8.4 Hz, 1H), 3.74-3.65 (m, 2H), 3.62-3.58 (m, 1H), 3.48-3.42 (m, 2H), 3.39-3.34 (m, 3H), 2.45-2.42 (m, 2H), 2.14-2.10 (m, 1H), 2.05-1.92 (m, 2H).

### Example 29

### (R)-2-44H-F-azaspiro[isoxazole-5,3¹-bicyclo[2.2.2]octan]-3-yl)amino)benzo[d]thiazole-5-carbonitrile hydrochloride ((R)-29)

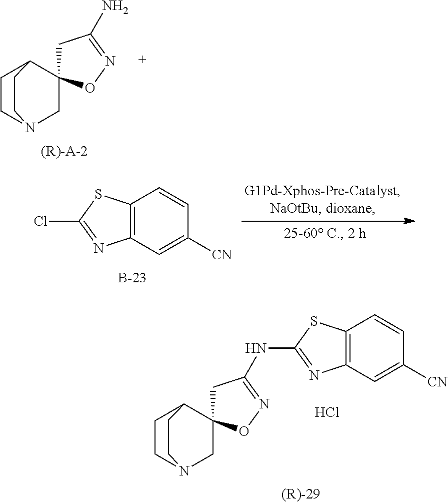

A mixture of compound B-23 (0.15 g, 0.77 mmol), compound (R)-A-2 (0.12 g, 0.64 mmol), dicyclohexyl[2-(2,4,6-triisopropylphenyl)phenyl]phosphane[2-(2-aminoethyl)phenyl]-chloro-palladium (0.02 g, 0.03 mmol) and sodium tert-butoxide (2 mol/L in tetrahydrofuran, 0.64 mL) in dioxane (10 mL) at 25° C. was degassed and placed under nitrogen. The mixture was stirred at 60° C. for 2 hours, then filtered and concentrated in vacuo. The residue was purified by prep-HPLC [Instrument: GX-A; Column: Phenomenex Gemini C18 250×50 mm, particle size: 10 μm; Mobile phase: 23-24% acetonitrile in H₂O (add 0.05% ammonia hydroxide, v/v)]. The resulting solids were dissolved in 0.2 M hydrochloric acid and again lyophilized to give:

Compound (R)-29 (99 mg, 40% yield) as a white solid: cSFC analytical (I) tR=3.26 min., purity: 100%; LCMS (H): tR=2.130 min., (ES⁺) m/z (M+H)⁺=340.1; ¹H-NMR (CD₃OD, 400 MHz): δ 8.00 (d, J=8.0 Hz, 1H), 7.98 (s, 1H), 7.52 (dd, J₁=8.0 Hz, J₂ =1.2 Hz, 1H), 3.68 (t, J=16 Hz, 2H), 3.58 (d, J=16.8 Hz, 1H), 3.46-3.33 (m, 5H), 2.44-2.41 (m, 2H), 2.13-2.10 (m, 1H), 2.01-1.92 (m, 2H).

### Example 30

### (R)—N-(5 ,6 - dichlor obenzo[d]thiazol-2-yl)-4H-1′-azaspiro[isoxazole-5,3′-bicyclo[2.2.2]octan]-3-amine hydrochloride ((R)−30)

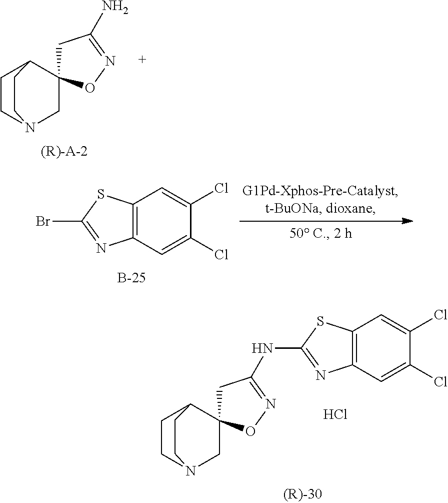

To a solution of compound B-25 (0.15 g, 0.53 mmol), compound (R)-A-2 (80 mg, 0.44 mmol) and dicyclohexyl[2-(2,4,6-triisopropylphenyl)phenyl]phosphane[2-(2-aminoethyl)phenyl]-chloro-palladium (16 mg, 0.022 mmol) in dioxane (5 mL) under nitrogen at room temperature was added sodium tert-butoxide (2 M in tetrahydrofuran, 0.66 mL). The reaction mixture was stirred at 50° C. for 2 hours, then filtered and concentrated in vacuo. The residue was purified by prep-HPLC [Instrument: GX-E; Column: Phenomenex Synergi C18 150×30 mm, particle size: 4 μm; Mobile phase: 20-45% acetonitrile in H₂O (add 0.5% HCl v/v)] and lyophilized to give:

Compound (R)-30 (60 mg, 33% yield) as a white solid: cSFC analytical (I) tR=3.968 min., purity: 100%; LCMS (FF): tR=2.476 min., (ES⁺) m/z (M+H)⁺=383.0; ¹H-NMR (CD₃OD, 400 MHz): δ 8.04 (s, 1H), 7.81 (s, 1H), 3.75-3.66 (m, 2H), 3.59 (d, J=16.8 Hz, 1H), 3.49-3.33 (m, 5H), 2.46-2.43 (m, 2H),2.16-2.12 (m, 1H), 2.03-1.94 (m, 2H).

### Example 31

### (R)—N-(6,7-dichlorobenzo[d]thiazol-2-yl)-4H-1′-azaspiro[isoxazole-5,3′-bicyclo[2.2.2]octan]-3-amine hydrochloride ((R)-31)

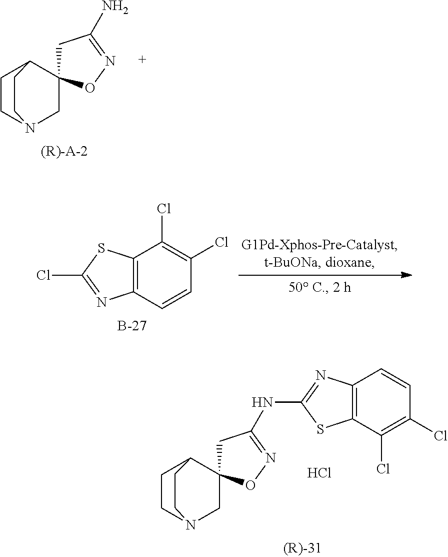

To a solution of compound (R)-A-2 (80 mg, 0.44 mmol), compound B-27 (0.13 g, 0.53 mmol) and [2-(2-aminoethyl)phenyl]-chloro-palladium;dicyclohexyl[2-(2,4,6-triisopropylphenyl)phenyl]phosphane (33 mg, 0.044 mmol) in dioxane (5 mL) under nitrogen at room temperature was added sodium tert-butoxide (2 M in tetrahydrofuran, 0.66 mL). The reaction mixture was stirred at 50° C. for 2 hours, then filtered and concentrated in vacuo. The residue was purified by prep-HPLC [Instrument: GX-A; Column: GEMINI 250×50 mm, particle size: 10 μm; Mobile phase: 35-44% acetonitrile in H₂O (add 0.5% NH₃·H₂O, v/v)]. The resulting product was lyophilized, dissolved 0.2 M hydrochloric acid and again lyophilized to give:

Compound (R)-31 (50 mg, 27% yield) as a white solid: cSFC analytical (I) tR=3.080 min., purity: 100%; LCMS (EE): tR=3.016 min., (ES⁻) m/z (M+H)⁺=383.0; ¹H-NMR (CD₃OD, 400 MHz): δ 7.58-7.53 (m, 2H), 3.48 (d, J=16.4 Hz, 1H), 3.38-3.33 (m, 2H), 3.29-3.24 (m, 1H), 3.09-3.00 (m, 4H), 2.29-2.19 (m, 2H),1.92-1.82 (m, 2H), 1.72-1.69 (m, 1H).

### Example 32

### (R)—N-(5, 7-dichlorobenzo[d]thiazol-2-yl)-4H-1′-azaspiro[isoxazole-5,3′-bicyclo[2.2.2]octan]-3-amine hydrochloride ((R)-32)

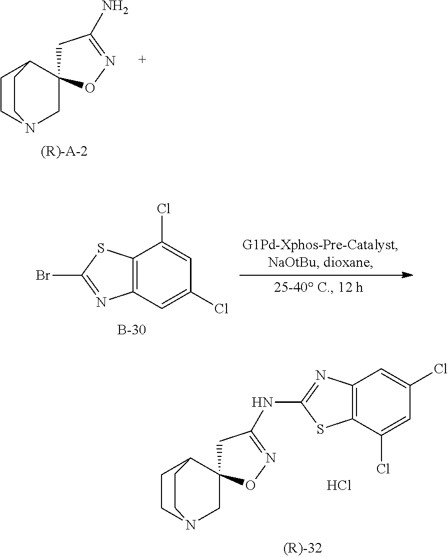

A mixture of compound B-30 (0.13 g, 0.46 mmol), compound (R)-A-2 (0.07 g, 0.39 mmol), dicyclohexyl[2-(2,4,6-triisopropylphenyl)phenyl]phosphane[2-(2-aminoethyl)phenyl]-chloro-palladium (0.01 g, 0.02 mmol) and sodium tert-butoxide (2 M in tetrahydrofuran, 0.39 mL) in dioxane (10 mL) under nitrogen was stirred at 40° C. for 12 hours. On completion, the mixture was filtered and concentrated in vacuo. The residue was purified by prep-HPLC [Instrument: GX-B; Column: Phenomenex Synergi C18 150×30 mm, particle size: 4μm; Mobile phase: 28-58% acetonitrile in H₂O (add 0.1% TFA, v/v)]. The combined fractions were treated with 0.2 M hydrochloric acid and lyophilized to give:

Compound (R)-32 (68 mg, 42% yield) as a white solid: cSFC analytical (I) tR=2.95 min., purity: 100%; LCMS (GG): tR=2.236 min., (ES⁺) m/z (M+H)⁺=383.0; ¹H-NMR (CD₃OD, 400 MHz): δ 7.57 (d, J=1.6 Hz, 1H), 7.29 (d, J=1.6 Hz, 1H), 3.67-3.62 (m, 2H), 3.54 (d, J=16.8 Hz, 1H), 3.42-3.31 (m, 5H), 2.43-2.39 (m, 2H), 2.10-2.08 (m, 1H), 1.99-1.89 (m, 2H).

### Example 33

### (R)—N-(6-chloro-5,7-difluorobenzo[d]thiazol-2-yl)-4H-1′-azaspiro[isoxazole-5,3′-bicyclo[2.2.2]octan]-3-amine hydrochloride ((R)-33)

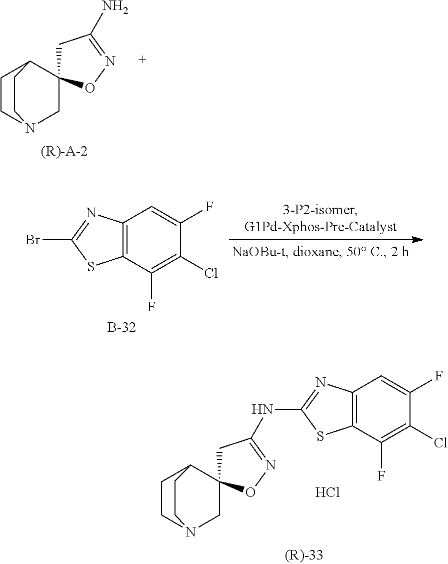

To a solution of compound (R)-A-2 (0.13 g, 0.74 mmol) and compound B-32 (0.2 g, 0.70 mmol) in dioxane (1.0 mL) at room temperature under nitrogen was added dicyclohexyl42-(2,4,6-triisopropylphenyl)phenyllphosphane42-(2-aminoethyl)phenyll-chloro-palladium (26 mg, 0.035 mmol) and sodium tert-butoxide (0.14 g, 1.4 mmol). The mixture was stirred at 50° C. for 2 hour, and then concentrated in vacuo. The residue was purified by prep-HPLC [Instrument: GX-B; Column: Phenomenex Synergi C18 150×30 mm, particle size: 4 μm; Mobile phase: 25-55% acetonitrile in H₂O (add 0.1% TFA, v/v)]. The combined fractions were treated with 0.2 M hydrochloric acid and lyophilized to give:

Compound (R)-33 (25 mg, 8.1% yield) as a white solid: cSFC analytical (I) tR=2.73 min., purity: 100.00%; LCMS (FF): tR=2.512 min, 385.0 m/z (M+1); ¹H-NMR (CD₃OD, 400 MHz): δ 7.41 (d, J=9.6 Hz, 1H), 3.76-3.67 (m, 2H), 3.60-3.56 (m, 1 H), 3.50-3.36 (m, 5H), 2.44 (m, 2H), 2.13-1.92 (m, 3H).

### Example 34

### (R)—N-(3-chlorophenyl)-4H-1′-azaspiro[isoxazole-5,3′-bicyclo[2.2.2]octan]-3-amine hydrochloride ((R)-34)

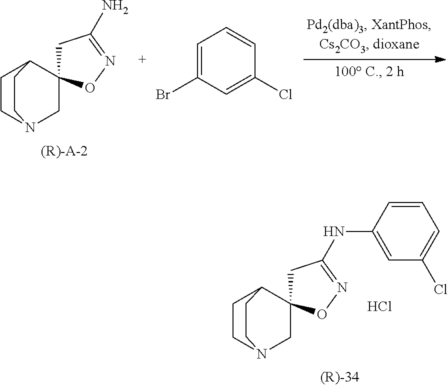 [SMILES: NC1=NO[C@@]2(C1)CN1CCC2CC1 ; Clc1cccc(Br)c1 ; Clc1cccc(NC2=NO[C@@]3(C2)CN2CCC3CC2)c1 ; Cl]

To a solution of 1-bromo-3-chlorobenzene (86 mg, 0.45 mmol), compound (R)-A-2 (90 mg, 0.50 mmol), tris(dibenzylideneacetone)dipalladium(0) (45 mg, 0.050 mmol) and 4,5-bis(diphenylphosphino)-9,9-dimethylxanthene (57 mg, 0.099 mmol) in dioxane (3 mL) under nitrogen at room temperature was added cesium carbonate (0.32 g, 0.99 mmol). The reaction mixture was stirred at 100° C. for 2 hours, then filtered and concentrated in vacuo. The residue was purified by prep-HPLC [Instrument: GX-B; Column: Phenomenex Synergi C18 150×30 mm, particle size: 4 μm; Mobile phase: 20-50% acetonitrile in H₂O (add 0.1% TFA, v/v)]. The combined fractions were treated with 0.2 M hydrochloric acid and lyophilized to give:

Compound (R)-34 (48 mg, 33% yield) as a yellow solid: cSFC analytical (Q) tR=3.45 min., purity: 100%; LCMS (FF): tR=2.153 min., 292.1 m/z (M+1); ¹H-NMR (CD₃OD, 400 MHz): δ 7.57 (s, 1H), 7.27-7.21 (m, 2H), 6.95-6.91 (m, 1H), 3.68-3.57 (m, 2H), 3.44-3.40 (m, 2H), 3.37-3.32 (m, 4H), 2.43-2.36 (m, 2H), 2.14-2.06 (m, 1H), 2.01-1.87 (m, 2H).

### Example 35

### (R)—N -(3-chlor o -4 -(trifluoromethyl)phenyl) - 4H - l′-azaspiro[isoxazole-5,3′-bicyclo[2.2.2]octan]-3-amine hydrochloride ((R)-35)

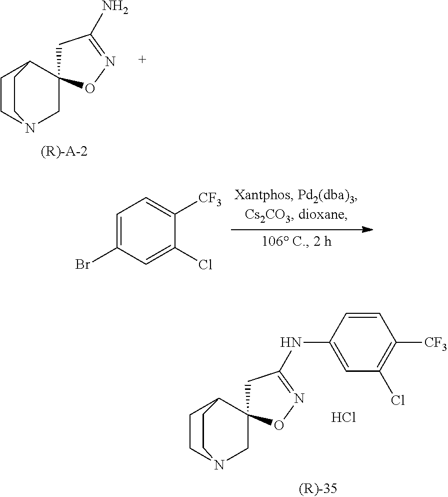 [SMILES: NC1=NO[C@@]2(C1)CN1CCC2CC1 ; FC(F)(F)c1ccc(Br)cc1Cl ; FC(F)(F)c1ccc(NC2=NO[C@@]3(C2)CN2CCC3CC2)cc1Cl ; Cl]

To a mixture of 4-bromo-2-chloro-1-(trifluoromethyl)benzene (0.11 g, 0.44 mmol) and compound (R)-A-2 (0.10 g, 0.55 mmol) in dioxane (10 mL) under nitrogen at room temperature was added cesium carbonate (0.36 g, 1.1 mmol), 4,5-bis(diphenylphosphino)-9,9-dimethylxanthene (32 mg, 0.050 mmol) and tris(dibenzylideneacetone)dipalladium(0) (25 mg, 0.028 mmol). The reaction mixture was stirred at 106° C. for 2 hours, then filtered and concentrated in vacuo. The residue was purified by prep-HPLC [Instrument: Shimadzu pump LC-20A; Column: GEMINI 200×50 mm, particle size: 10 μm; Mobile phase: 55-66.7% acetonitrile in H₂O (add 0.5% NH₃ H₂O, v/v)]. The combined fractions were lyophilized, treated with 0.2 M hydrochloric acid and again lyophilized to give:

Compound (R)-35 (65 mg, 37% yield) as a white solid: cSFC analytical tR=2.60 min., purity: 100%; LCMS (M): tR=2.42 min., 360.1 m/z (M+1); ¹H-NMR (CD₃OD, 400 MHz): δ 7.79 (s, 1H), 7.65 (d, J=8.8 Hz,1H), 7.43 (d, J=8.4 Hz,1H), 3.71-3.61 (m, 2H), 3.48-3.42 (m, 2H), 3.39-3.36 (m, 4H), 2.46-2.39 (m, 2H), 2.16-2.08 (m, 1H), 2.03-1.90 (m, 2H).

### Example 36

### (R)—N-(3-chloro-4-cyclopropylphenyl)-4H-1′-azaspiro[isoxazole-5,3′-bicyclo[2.2.2]octan]-3-amine hydrochloride ((R)-36)

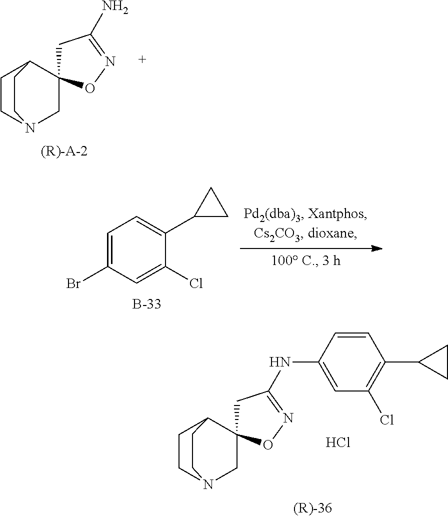

To a solution of compound B-33 (0.14 g, 0.60 mmol), compound (R)-A-2 (0.11 g, 0.60 mmol) , tris(dibenzylideneacetone)dipalladium(0) (28 mg, 0.030 mmol) and 4,5-bis(diphenylphosphino)-9,9-dimethylxanthene (35 mg, 0.060 mmol) in dioxane (10 mL) under nitrogen at room temperature was added cesium carbonate (0.39 g, 1.2 mmol). The reaction mixture was stirred at 100° C. for 3 hours, then filtered and concentrated in vacuo. The residue was purified by prep-HPLC [Instrument: GX-I; Column: Welch Ultimate AQ-C18 150×30 mm, particle size: 5 μm; Mobile phase: 25-55% acetonitrile in H₂O (add 0.1% TFA, v/v)]. The combined fractions were treated with 0.2 M hydrochloric acid and lyophilized to give:

Compound (R)-36 (25 mg, 11% yield) as a yellow solid: cSFC analytical (P) tR=3.073 min., purity: 100%; LCMS (FF): tR=2.396 min., (ES⁺) m/z (M+H)⁺=332.1; ¹H-NMR (CD₃OD, 400 MHz): δ 7.57 (d, J=2.4 Hz, 1H), 7.18 (dd, J1=8.4 Hz, J2=2.4 Hz, 1H), 6.93 (d, J=8.4 Hz, 1H), 3.69-3.62 (m, 2H), 3.44-3.28 (m, 6H), 2.45-2.37 (m, 2H), 2.12-2.08 (m, 2H), 1.97-1.93 (m, 2H), 0.99-0.95 (m, 2H), 0.65-0.61 (m, 2H).

### Example 37

### (R)—N-(4-chloro-3-(trifluoromethyl)phenyl)-4H-1′-azaspiro[isoxazole-5,3′-bicyclo[2.2.2]octan]-3-amine hydrochloride ((R)-37)

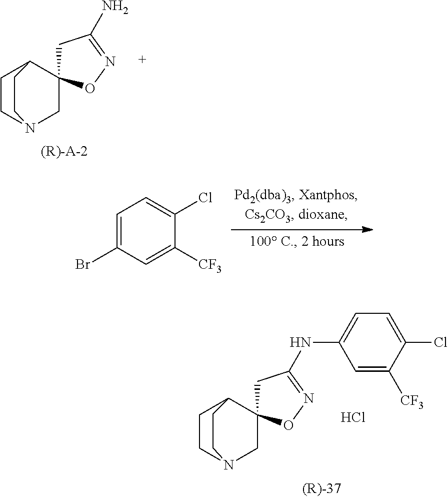 [SMILES: NC1=NO[C@@]2(C1)CN1CCC2CC1 ; FC(F)(F)c1cc(Br)ccc1Cl ; FC(F)(F)c1cc(NC2=NO[C@@]3(C2)CN2CCC3CC2)ccc1Cl ; Cl]

To a solution of compound (R)-A-2 (80 mg, 0.44 mmol), 4-bromo-l-chloro-2-(trifluoromethyl)benzene (0.11 g, 0.44 mmol), tris(dibenzylideneacetone)dipalladium(0) (40 mg, 0.044 mmol) and 4,5-bis(diphenylphosphino)-9,9-dimethylxanthene (51 mg, 0.088 mmol) in dioxane (8 mL) under nitrogen at room temperature was added cesium carbonate (0.29 g, 0.88 mmol). The mixture was stirred at 100° C. for 2 hours, then filtered and concentrated in vacuo. The residue was purified by prep-HPLC [Instrument: GX-H; Column: Waters Xbridge 150×25 mm, particle size: 5 μm; Mobile phase: 26-56% acetonitrile in H₂O (add 0.5% NH₃·H₂O, v/v)]. The combined fractions were lyophilized, treated with 0.2 M hydrochloric acid and again lyophilized to give:

Compound (R)-37 (70 mg, 40% yield) as a white solid: cSFC analytical (I) tR=1.933 min., purity: 100%; LCMS (FF): tR=2.412 min., (ES⁺) m/z (M+H)⁺=360.1; ¹H-NMR (CD₃OD, 400 MHz): δ 7.97 (d, J=2.4 Hz, 1H), 7.61 (dd, J₁=8.8 Hz, J₂=2.4 Hz, 1H), 7.49 (d, J=8.8 Hz, 1H), 3.70 (d, J=13.6 Hz, 1H), 3.61 (d, J=13.6 Hz. 1H), 3.48-3.33 (m, 6H), 2.45-2.38 (m, 2H), 2.13-2.10 (m, 1H), 2.02-1.90 (m, 2 H).

### Example 38

### (R)—N-(4-chloro-3-cyclopropylphenyl)-4H-1′-azaspiro[isoxazole-5,3′-bicyclo[2.2.2]octan]-3-amine hydrochloride ((R)-38)

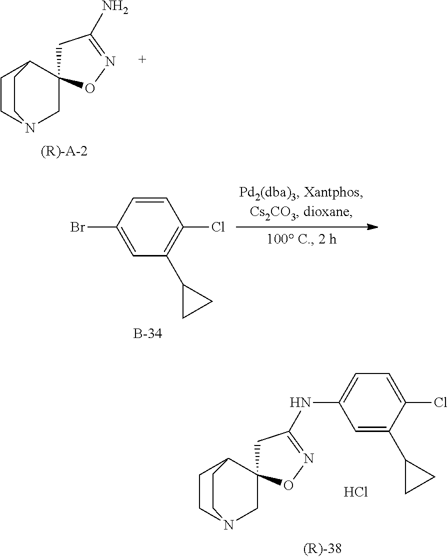

To a solution of compound B-34 (0.13 g, 0.55 mmol), compound (R)-A-2 (90 mg, 0.50 mmol), tris(dibenzylideneacetone)dipalladium(0) (23 mg, 0.025 mmol) and 4,5-bis(diphenylphosphino)-9,9-dimethylxanthene (29 mg, 0.050 mmol) in dioxane (3 mL) under nitrogen at room temperature was added cesium carbonate (0.32 g, 0.99 mmol). The reaction mixture was stirred at 100° C. for 2 hours, then filtered and concentrated in vacuo. The residue was purified by prep-HPLC [Instrument: GX-C; Column: Phenomenex Synergi C18 150×50 mm, particle size: 10 μm; Mobile phase: 40-70% acetonitrile in H₂O (add 0.05% NH₃·H₂O, v/v)]. The combined fractions were lyophilized, treated with 0.2 N HCl (2 mL) and lyophilized to give:

Compound (R)-38 (25 mg, 14% yield) as a white solid: cSFC analytical (Q) tR=2.527 min., purity: 100%; LCMS (FF): tR=2.423 min., (ES⁺) m/z (M+H)⁺=332.1; ¹H-NMR (CD₃OD, 400 MHz): δ 7.20 (d, J=8.8 Hz, 1H), 6.91 (dd, J₁=8.4 Hz, J₂=2.0 Hz, 1H), 6.81 (s, 1H), 3.54 (s, 2H), 3.51-3.17 (m, 6H), 2.26-1.98 (m, 4H), 1.83-1.81 (m, 2H), 0.93-0.91 (m, 2H), 0.56-0.55 (m, 2H).

### Example 39

### (R)—N-(4-chloro-3-methoxyphenyl)-4H-1′-azaspiro[isoxazole-5,3′-bicyclo[2.2.2]octan]-3-amine hydrochloride ((R)-39)

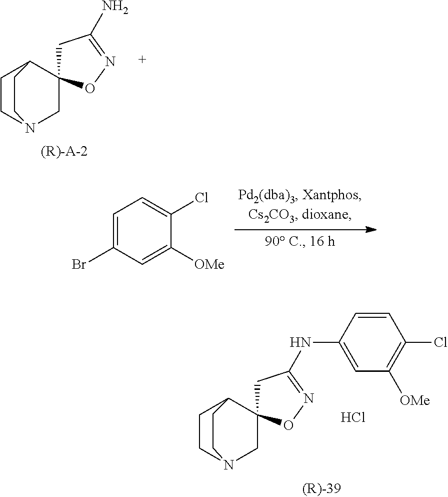 [SMILES: NC1=NO[C@@]2(C1)CN1CCC2CC1 ; COc1cc(Br)ccc1Cl ; COc1cc(NC2=NO[C@@]3(C2)CN2CCC3CC2)ccc1Cl ; Cl]

To a solution of 4-bromo-l-chloro-2-methoxybenzene (61 mg, 0.28 mmol), compound (R)-A-2 (50 mg, 0.28 mmol), tris(dibenzylideneacetone)dipalladium(0) (13 mg, 0.014 mmol) and 4,5-bis(diphenylphosphino)-9,9-dimethylxanthene (16 mg, 0.028 mmol) in dioxane (5 mL) under nitrogen at room temperature was added cesium carbonate (0.18 g, 0.55 mmol). The reaction mixture was stirred at 90° C. for 16 hours, then filtered and concentrated in vacuo. The residue was purified by prep-HPLC [Instrument: GX-E; Column: Phenomenex Synergi C18 150×30 mm, particle size: 4 μm; Mobile phase: 22-47% acetonitrile in H₂O (add 0.05% HCl v/v)] and lyophilized to give:

Compound (R)-39 (40 mg, 40% yield) as a yellow solid: cSFC analytical (I) tR=3.031 min., purity: 100.00%; LCMS (FF): tR=2.092 min., (ES⁺) m/z (M+H)⁺=322.1; ¹H-NMR (CD₃OD, 400 MHz): δ 7.33 (d, J=2.0 Hz, 1H), 7.23 (d, J=8.8 Hz, 1H), 6.89 (dd, J₁=8.4 Hz, J₂=2.4 Hz , 1H), 3.88 (s, 3H), 3.69 (dd, J₁=14 Hz, J₂=2.0 Hz , 1H), 3.61 (d, J=14 Hz, 1H), 3.47-3.44 (m, 2H), 3.43-3.31 (m, 4H), 2.44-2.38 (m, 2H), 2.15-2.10 (m, 1H), 2.03-1.90 (m, 2H).

### Example 40

### (R)—N-(2-methylbenzofuran-6-yl)-4H-1′-azaspiro[isoxazole-5,3′-bicyclo[2.2.2]octan]-3-amine hydrochloride ((R)−40)

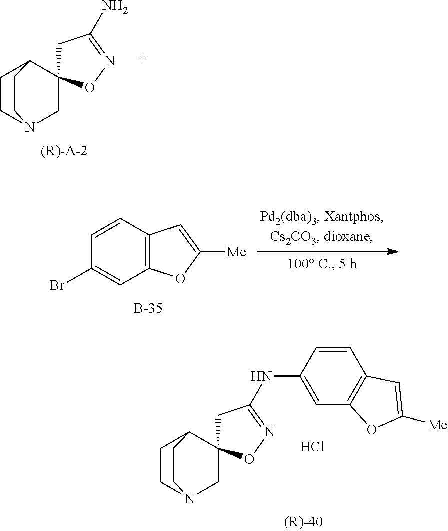

To a solution of compound B-35 (0.35 g, 1.65 mmol), compound (R)-A-2 (0.20 g, 1.10 mmol), tris(dibenzylideneacetone)dipalladium(0) (50 mg, 0.055 mmol) and 4,5-bis(diphenylphosphino)-9,9-dimethylxanthene (64 mg, 0.11 mmol) in dioxane (10 mL) under nitrogen at room temperature was added cesium carbonate (0.72 g, 2.0 mmol). The reaction was stirred at 100° C. for 5 hours, then filtered and concentrated in vacuo. The residue was purified by prep-HPLC [Instrument: GX-A; Column: Phenomenex Gemini C18 150×25 mm, particle size: 10 μm; Mobile phase: 30-60% acetonitrile in H₂O (add 0.5% NH₃·H₂O, v/v)]. The combined fractions were lyophilized, treated with in 0.2 M hydrochloric acid and again lyophilized to give:

Compound (R)-40 (60 mg, 17% yield) as an off-white solid: cSFC analytical (A) tR=2.262 min., purity: 98.79%; LCMS (GG): tR=1.930 min., (ES⁺) m/z (M+H)⁺=312.1; ¹H-NMR (CD₃OD, 400 MHz): δ 7.68 (s, 1H), 7.33 (d, J=8.8 Hz, 1H), 7.049 (dd, J=2.0 Hz, J=8.8 Hz, 1H), 6.35 (s, 1H), 3.64-3.58 (m, 2H), 3.45-3.40 (m, 2H), 3.36-3.35 (m, 2H), 3.28 (m, 1H), 2.40 (s, 3H), 2.38 (m, 2H), 2.12-2.10 (m, 1H), 1.99-1.90 (m, 2H).

### Example 41

### (R)—N-(2 -methylbenzofuran-5 -yl)-4H-1′-azaspiro[isoxazole-5,3′-bicyclo[2.2.2.]octan]-3-amine hydrochloride ((R)-41)

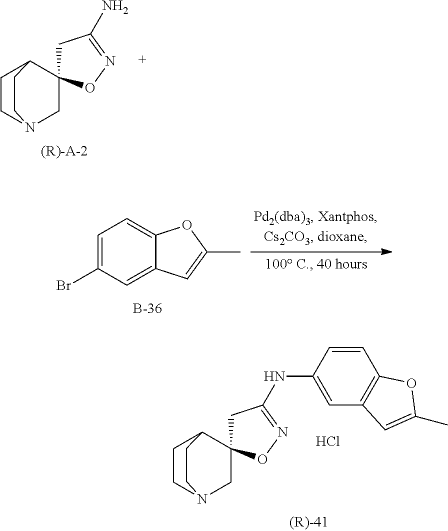

To a solution of compound (R)-A-2 (0.15 g, 0.83 mmol), compound B-36 (0.21 g, 0.99 mmol), tris(dibenzylideneacetone)dipalladium(0) (76 mg, 0.083 mmol) and 4,5-bis(diphenylphosph-ino)-9,9-dimethylxanthene (96 mg, 0.17 mmol) in dioxane (10 mL) under nitrogen at room temperature was added cesium carbonate (0.81 g, 2.5 mmol). The mixture was stirred at 100° C. for 40 hours, then filtered and concentrated in vacuo. The residue was purified by prep-HPLC [Instrument: GX-A; Column: Phenomenex Gemini 150×25 mm, particle size: 5 μm; Mobile phase: 28-58% acetonitrile in H₂O (add 0.5% NH₃·H₂O, v/v)]. The combined fractions were lyophilized, treated with 0.2 M hydrochloric acid and again lyophilized to give:

Compound (R)-41 (28 mg, 10% yield) as a white solid: cSFC analytical (I) tR=4.440 min., purity: 100%; LCMS (FF): tR=2.275 min., (ES⁺) m/z (M+H)⁺=312.1; ¹H-NMR (CD₃OD, 400 MHz): δ 7.61 (d, J=2 Hz, 1H), 7.32 (d, J=8.8 Hz, 1H), 7.14 (dd, J₁=8.8 Hz, J₂=2.4 Hz, 1H), 6.41 (s, 1H), 3.71-3.65 (m, 2H), 3.51-3.33 (m, 6H), 2.44-2.42 (m, 5H), 2.16-2.12 (m, 1H), 2.03-1.93 (m, 2H).

### Example 42

### (R)—N-(2-methylbenzo[b]thiophen-6-yl)-4H-1′-azaspiro[isoxazole-5,3′-bicyclo[2.2.2]octan]-3-amine hydrochloride ((R)-42)

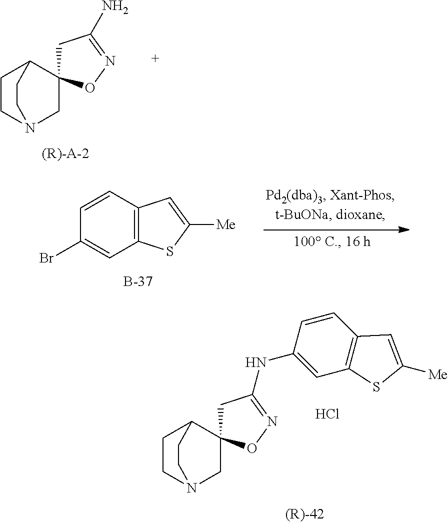

To a solution of compound B-37 (95 mg, 0.42 mmol) and compound (R)-A-2 (80 g, 0.44 mmol) in dioxane (2.0 mL) under nitrogen at room temperature was added tris(dibenzylideneacetone)dipalladium(0) (38 mg, 0.042 mmol) and 4,5-bis(diphenylphosphino)-9,9-dimethylxanthene (37 mg, 0.063 mmol) and sodium tert-butoxide (121 mg, 1.3 mmol). The mixture was stirred at 100° C. for 16 hours, then concentrated in vacuo. The residue was purified by prep-HPLC [Instrument: GX-B; Column: Welch Ultimate AQ-C18 150×30 mm, particle size: 5 μm; Mobile phase: 32-32% acetonitrile in H₂O (add 0.5% TFA, v/v)]. The combined fractions were treated with 0.2 M hydrochloric acid and lyophilized to give:

Compound (R)-42 (26 mg, 16% yield) as a white solid: cSFC analytical (H) tR=3.61 min., purity: 100.00%; LCMS (FF): tR=2.334 min, 328.1 m/z (M+1); (CD₃OD, 400 MHz): δ 8.00 (s, 1H), 7.56 (d, J=8.5 Hz, 1H), 7.20 (m, 1H), 6.94 (s, 1H), 3.70-3.60 (m, 2H), 3.48-3.37 (m, 6H), 2.55 (s, 3H), 2.40 (m, 2H), 2.15-2.12 (m, 1H), 2.03-1.90 (m, 2H).

### Example 43

### (R)—N-(2 -methylbenzo[b]thiophen-5-yl)-4H-1′-azaspiro[isoxazole-5,3′-bicyclo[2.2.2.]octan]-3-amine hydrochloride ((R)-43)

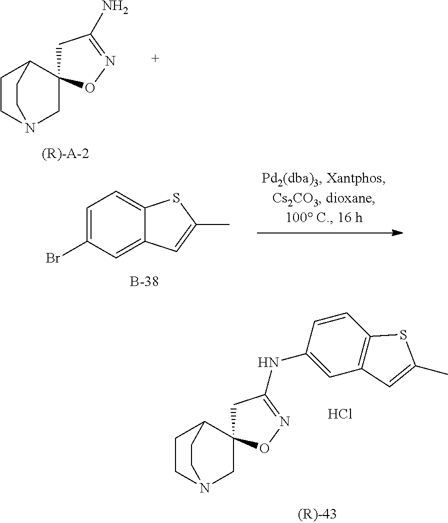

To a solution of compound B-38 (0.12 g, 0.53 mmol), compound (R)-A-2 (80 mg, 0.44 mmol), tris(dibenzylideneacetone)dipalladium(0) (40 mg, 44 μmol) and 4,5-bis(diphenylphosph-ino)-9,9-dimethylxanthene (51 mg, 88 μmol) in dioxane (2 mL) under nitrogen at room temperature was added cesium carbonate (0.43 g, 1.3 mmol). The mixture was stirred at 100° C. for 16 hours, then filtered and concentrated in vacuo. The residue was purified by prep-HPLC [Instrument: GX-B; Column: Welch Ultimate AQ-C18 150×30 mm, particle size: 5 μm; Mobile phase: 25-55% acetonitrile in H₂O (add 0.1% TFA, v/v)]. The combined fractions were treated with 0.2 M hydrochloric acid and lyophilized to give:

Compound (R)-43 (26 mg, 16% yield) as a white solid: cSFC analytical (I) tR=3.765 min., purity: 100%; LCMS (FF): tR=2.405 min., (ES⁺) m/z (M+H)⁺=328.1; ^{I}I-I-NMR (CD₃OD, 400 MHz): δ 7.84 (d, J=2 Hz, 1H), 7.66 (d, J=8.8 Hz, 1H), 7.20 (dd, J₁=8.8 Hz, J₂=2 Hz, 1H), 6.97 (s, 1H), 3.73-3.62 (m, 2H), 3.53-3.33 (m, 6H), 2.58 (s, 3H), 2.42-2.39 (m, 2H), 2.17-2.09 (m, 1H), 2.04-1.91 (m, 2H).

### Example 44

### (R)—N-(7-fluoroisoquinolin-3-yl)-4H-F-azaspiro[isoxazole-5,3¹-bicyclo[2.2.2]octan]-3-amine hydrochloride ((R)-44)

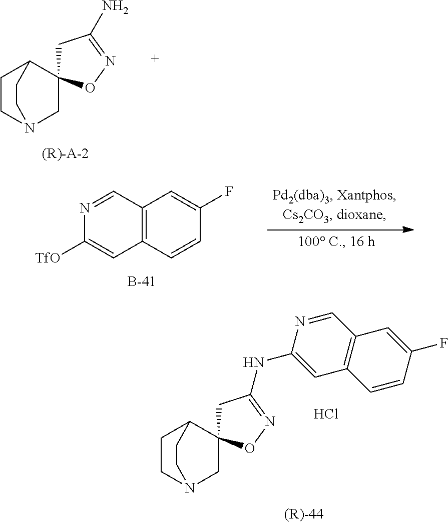

A mixture of compound B-41 (0.16 g, 0.53 mmol), compound (R)-A-2 (80 mg, 0.44 mmol), tris(dibenzylideneacetone)dipalladium(0) (40 mg, 44 μmol), cesium carbonate (0.29 g, 0.88 mmol) and 4,5-bis(diphenylphosphino)-9,9-dimethylxanthene (51 mg, 88 μmol ) in dioxane (5 mL) was stirred at 100° C. for 16 hours under nitrogen, then filtered and concentrated in vacuo. The residue was purified by prep-HPLC [Instrument: GX-H; Column: Phenomenex Gemini C18 250×50 mm, particle size: 10 μm; Mobile phase: 28-58% acetonitrile in H₂O (add 0.05% ammonia hydroxide, v/v)] The combined fractions were lyophilized, treated with 0.2 M hydrochloric acid and again lyophilized to give:

Compound (R)-44 (26 mg, 16% yield) as a yellow solid: cSFC analytical (I) tR=3.670 min., purity: 98.63%; LCMS (GG): tR=1.874 min., (ES⁺) m/z (M+H)⁺=327.1; ¹H-NMR (CD₃OD, 400 MHz): δ 9.24 (s, 1H), 8.07-8.04 (m, 2H), 7.87 (dd, J₁=8.8 Hz, J₂ =2 Hz, 1H), 7.78-7.73 (m, 1H), 3.80-3.63 (m, 3H), 3.55-3.33 (m, 5H), 2.47-2.42 (m, 2H), 2.18-2.13 (m, 1H), 2.09-1.97 (m, 2H).

### Example 45

### (R)—N-(6-fluoroisoquinolin-3-yl)-4H-1′-azaspiro[isoxazole-5,3′-bicyclo[2.2.2]octan]-3-amine hydrochloride ((R)-45)

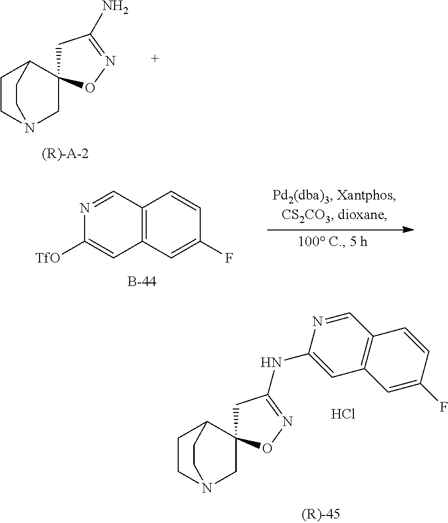

To a solution of compound B-44 (0.16 g, 0.53 mmol), compound (R)-A-2 (80 mg, 0.44 mmol), tris(dibenzylideneacetone)dipalladium(0) (40 mg, 0.044 mmol) and 4,5-bis(diphenylphosphino)-9,9-dimethylxanthene (51 mg, 0.088 mmol) in dioxane (3.0 mL) under nitrogen at room temperature was added cesium carbonate (0.29 g, 0.88 mmol). The reaction was stirred at 100° C. for 5 hours, then filtered and concentrated in vacuo. The residue was purified by prep-HPLC [Instrument: GX-B; Column: Welch Ultimate AQ-C18 150×30 mm, particle size: 5 μm; Mobile phase: 18-48% acetonitrile in H₂O (add 0.1% TFA, v/v)]. The combined fractions were treated with 0.2 M hydrochloric acid and lyophilized to give:

Compound (R)-45 (40 mg, 25% yield) as a yellow solid: cSFC analytical (A) tR=1.027 min., purity: 100%; LCMS (GG): tR=1.885 min., (ES⁺) m/z (M+H)⁺=327.1; ¹H-NMR (CD₃OD, 400 MHz): δ 9.26 (s, 1H), 8.28 (dd, J=5.6 Hz, J=9.2 Hz, 1H), 7.95 (s, 1H), 7.66 (dd, J=7.0 Hz, J=9.6 Hz, 1H), 7.48 (td, J=2.4 Hz, J=8.8 Hz, 1H), 3.78-3.77 (m, 1H), 3.74-3.73 (m, 1H), 3.70 (m, 1H), 3.59-3.52 (m, 2H), 3.40-3.36 (m, 3H), 2.45-2.40 (s, 2H), 2.18-2.11 (m, 1H), 2.04-1.95 (m, 2H).

### Example 46

### (R)—N-(7-methoxyisoquinolin-3-yl)-4H-1′-azaspiro[isoxazole-5,3′-bicyclo[2.2.2]octan]-3-amine hydrochloride ((R)-46)

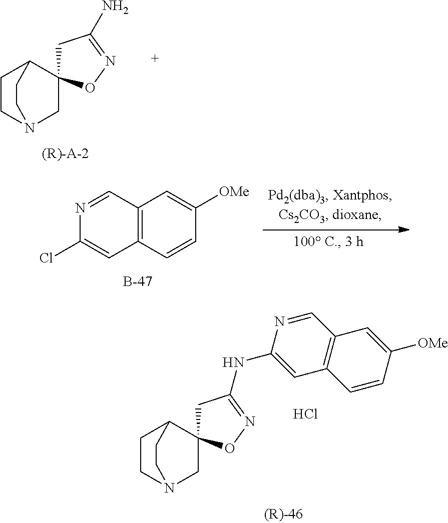

To a solution of compound B-47 (0.11g, 0.57 mmo1), compound (R)-A-2 (0.10 g, 0.57 mmol) , tris(dibenzylideneacetone)dipalladium(0) (52 mg, 0.057 mmol) and 4,5-bis(diphenylphosphino)-9,9-dimethylxanthene (49 mg, 0.058 mmol) in dioxane (20 mL) under nitrogen at room temperature was added cesium carbonate (0.37 g, 1.1 mmol). The reaction mixture was stirred at 100° C. for 3 hours, then filtered and concentrated in vacuo. The residue was purified by prep-HPLC [Instrument: GX-C; Column: Phenomenex Gemini C18 250×50 mm, particle size: 10 μm; Mobile phase: 32-62% acetonitrile in H₂O (add 0.05% ammonia hydroxide, v/v)]. The combined fractions were lyophilized, treated with 0.2 M hydrochloric acid and again lyophilized to give:

Compound (R)-46 (45 mg, 21% yield) as a yellow solid: cSFC analytical (0) tR=6.133 min., purity: 100%; LCMS (GG): tR=1.827 min., (ES⁺) m/z (M+H)⁺=339.1; ¹H-NMR (CD₃OD, 400 MHz): δ 9.20 (s, 1H), 7.99-7.94 (m, 2H), 7.66-7.64 (m, 1H), 7.58 (s, 1H), 4.01 (s, 3H), 3.81-3.63 (m, 3H), 3.56-3.39 (m, 5H) , 2.48-2.45 (m, 2H), 2.16-1.95 (m, 3H).

### Example 47

### (R)—N-(6-methoxyisoquinolin-3-yl)-4H-1′-azaspiro[isoxazole-5,3′-bicyclo[2.2.2]octan]-3-amine hydrochloride ((R)-47)

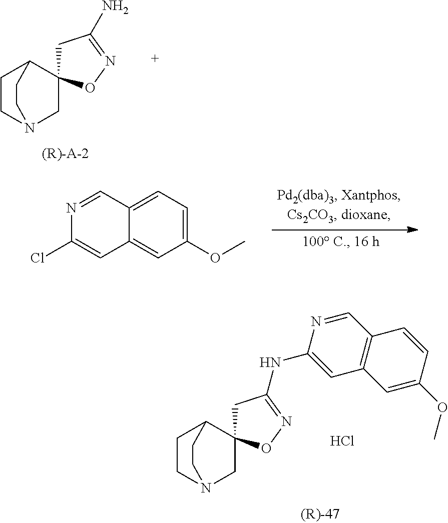 [SMILES: NC1=NO[C@@]2(C1)CN1CCC2CC1 ; COc1ccc2cnc(Cl)cc2c1 ; COc1ccc2cnc(NC3=NO[C@@]4(C3)CN3CCC4CC3)cc2c1 ; Cl]

To a solution of 3-chloro-6-methoxyisoquinoline (85 mg, 0.44 mmol), compound (R)-A-2 (80 mg, 0.44 mmol) and tris(dibenzylideneacetone)dipalladium(0) (40 mg, 0.044 mmol) and 4,5-bis(diphenylphosphino)-9,9-dimethylxanthene (51 mg, 0.088 mmol) in dioxane (3 mL) under nitrogen at room temperature was added cesium carbonate (0.29 g, 0.88 mmol). The reaction mixture was stirred at 100° C. for 16 hours, then filtered and concentrated in vacuo. The residue was purified by prep-HPLC [Instrument: GX-A; Column: Phenomenex Gemini C18 250×50 mm, particle size: 10 μm; Mobile phase: 30-60% acetonitrile in H₂O (add 0.5% NH₃·H₂O, v/v)]. The combined fractions were lyophilized, treated with 0.2 M hydrochloric acid and again lyophilized to give:

Compound (R)-47 (30 mg, 18% yield) as a white solid: cSFC analytical (I) tR=1.447 min., purity: 98.81%; LCMS (FF): tR=2.075 min., (ES⁺) m/z (M+H)⁺=339.1; ¹H-NMR (CD₃OD, 400 MHz): δ 9.11 (s, 1H), 8.13 (d, J=9.6Hz, 1H), 7.80 (s, 1H), 7.37 (s, 1H), 7.33 (d, J=9.2Hz, 1H), 4.07 (s, 3H), 3.80-3.70 (m, 2H), 3.65-3.61 (m, 1H), 3.53-3.33 (m, 5H), 2.47-2.42 (m, 2H), 2.16-2.14 (m, 1H), 2.08-1.96 (m, 2H).

### Example 48

### (R)—N-(6-chloroquinazolin-2-yl)-4H-1′-azaspiro[isoxazole-5,3′-bicyclo[2.2.2]octan]-3-amine hydrochloride ((R)-48)

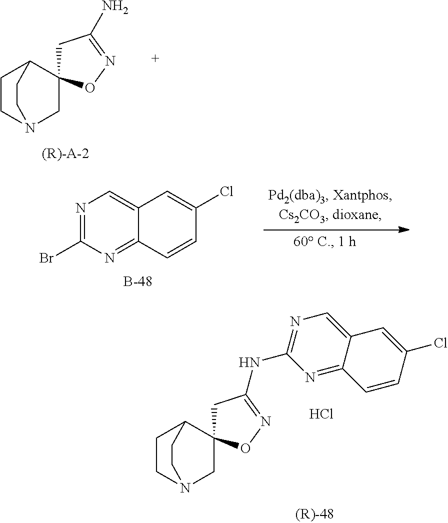

To a solution of compound B-48 (0.16 mg, 0.61 mmol), compound (R)-A-2 (0.10 g, 0.44 mmol), tris(dibenzylideneacetone)dipalladium(0) (50 mg, 55 μmol) and 4,5bis(diphenylphosphino)-9,9-dimethylxanthene (64 mg, 0.11 mmol) in dioxane (2 mL) under nitrogen at room temperature was added cesium carbonate (0.54 g, 1.7 mmol). The reaction mixture was stirred at 60° C. for 1 hour, then filtered and concentrated in vacuo. The residue was purified by silica gel chromatography [ethyl acetate: methanol=50:1˜1:1] and prep-HPLC [Instrument: GX-B; Column: Welch Ultimate AQ-C18 150×30 mm, particle size: 5 μm; Mobile phase: 17-47% acetonitrile in H₂O (add 0.1% TFA, v/v)]. The combined fractions were treated with 0.2 M hydrochloric acid and lyophilized to give:

Compound (R)-48 (10 mg, 6% yield) as a yellow solid: cSFC analytical (I) tR=3.413 min., purity: 100%; LCMS (GG): tR=1.790 min., (ES⁺) m/z (M+H)⁺=344.1; ¹H-NMR (DMSO-d₆, 400 MHz): δ 10.60 (s, 1H), 9.34 (s, 1H), 8.12 (d, J=2.4 Hz, 1H), 7.88 (dd, J₁=9.2 Hz, J₂=2.4 Hz, 1H), 7.70 (d, J=9.2Hz, 1H), 3.88 (d, J=17.6 Hz, 1H), 3.74 (d, J=18 Hz, 1H), 3.66 (d, J=13.6 Hz, 1H), 3.48-3.75 (m, 1H), 3.31-3.18 (m, 4H), 2.28 (s, 1H), 2.19-2.16 (m, 1H), 1.93-1.75 (m, 3H).

### Example 49

### (R)—N-(7-chloroquinazolin-2-yl)-4H-1′-azaspiro[isoxazole-5,3′-bicyclo[2.2.2]octan]-3-amine ((R)-49)

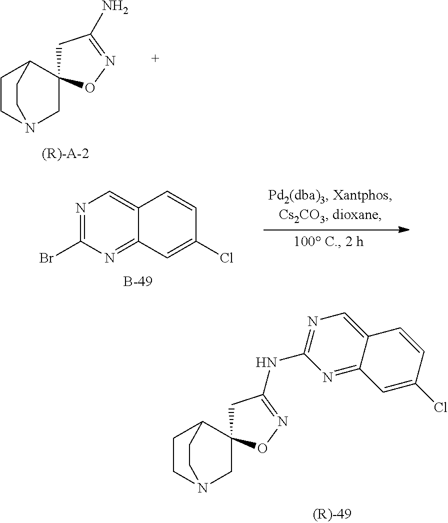

To a solution of compound B-49 (0.20 mg, 0.83 mmol), compound (R)-A-2 (0.15 g, 0.83 mmol), tris(dibenzylideneacetone)dipalladium(0) (76 mg, 83 μmol) and 4,5bis(diphenylphosphino)-9,9-dimethylxanthene (96 mg, 0.17 mmol) in dioxane (5 mL) under nitrogen at room temperature was added cesium carbonate (0.54 g, 1.7 mmol). The reaction mixture was stirred at 100° C. for 2 hours, then filtered and concentrated in vacuo. The residue was purified flash silica gel chromatography [ethyl acetate: methanol=50:1 to1:11 ]and prep-HPLC [Instrument: GX-I; Column: Phenomenex Gemini 150×25 mm, particle size: 10 μm; Mobile phase: 27-57% acetonitrile in H₂O (add 0.05% ammonia hydroxide, v/v)] and lyophilized to give:

Compound (R)-49 (6 mg, 2% yield) as a yellow solid: cSFC analytical (I) tR=3.525 min., purity: 100%; LCMS (GG): tR=1.826 min., (ES⁺) m/z (M+H)⁺=344.1; ¹H-NMR (CD₃OD, 400 MHz): δ 9.24 (s, 1H), 7.92 (d, J=8.8 Hz, 1H), 7.88 (s, 1H), 7.45 (dd, J₁=8.4 Hz, J₂=1.6 Hz, 1H), 3.77 (d, J=17.2 Hz, 1H), 3.45 (d, J=17.2 Hz, 1H), 3.21-3.12 (m, 2H), 2.93-2.83 (m, 4H), 2.18 (m, 1H), 2.09 (m, 1H), 1.80 (m, 2H), 1.62-1.55 (m, 1H).

### Example 50

### (R)—N-(2-methylbenzo[d]thiazol-6-yl)-4H-1′-azaspiro[isoxazole-5,3′-bicyclo[2.2.2]octan]-3-amine hydrochloride ((R)-50)

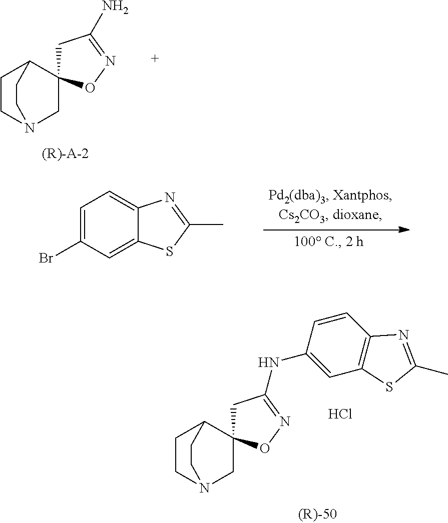 [SMILES: NC1=NO[C@@]2(C1)CN1CCC2CC1 ; Cc1nc2ccc(Br)cc2s1 ; Cc1nc2ccc(NC3=NO[C@@]4(C3)CN3CCC4CC3)cc2s1 ; Cl]

To a solution of 6-bromo-2-methylbenzo[d]thiazole (0.11 g, 0.50 mmol), compound (R)-A-2 (0.10 g, 0.55 mmol), tris(dibenzylideneacetone)dipalladium(0) (51 mg, 0.055 mmol) and 4,5-bis(diphenylphosphino)-9,9-dimethylxanthene (64 mg, 0.11 mmol) in dioxane (1 mL) under nitrogen at room temperature was added cesium carbonate (0.36 g, 1.1 mmol). The reaction mixture was stirred at 100° C. for 2 hours, then filtered and concentrated in vacuo. The residue was purified by prep-HPLC [Instrument: GX-E; Column: Phenomenex Synergi C18 150×30 mm, particle size: 4 μm; Mobile phase: 10-30% acetonitrile in H₂O (add 0.1% HCl v/v)] and lyophilized to give:

Compound (R)-50 (26 mg, 14% yield) as a yellow solid: cSFC analytical (O) tR=1.851 min., purity: 100%; LCMS (GG): tR=1.766 min., (ES⁺) m/z (M+H)⁺=329.1; ¹H-NMR (CD₃OD, 400 MHz): δ 8.38 (d, J=1.6 Hz, 1H), 7.85 (d, J=9.2 Hz, 1H), 7.56 (dd, J₁=8.8 Hz, J₂=2.0 Hz , 1H), 3.69 (d, J=14.0 Hz , 1H), 3.61 (d, J=14.0 Hz , 1H), 3.50-3.34 (m, 6H), 2.99 (m, 3H), 2.44-2.38 (m, 2H), 2.12-2.07 (m, 1H), 2.03-1.89 (m, 2H).

### Example 51

### (R)—N-(2-methylbenzo[d]oxazol-6-yl)-4H-1′-azaspiro[isoxazole-5,3′-bicyclo[2.2.2]octan]-3-amine ((R)-51)

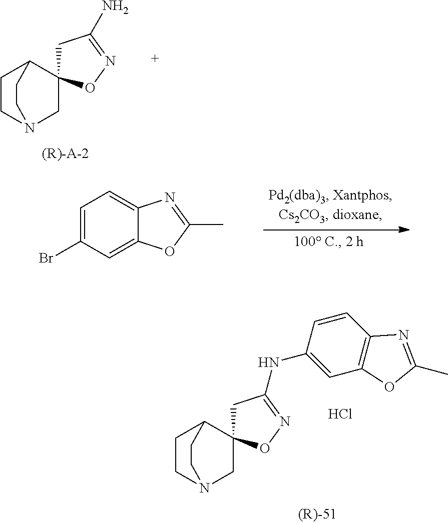 [SMILES: NC1=NO[C@@]2(C1)CN1CCC2CC1 ; Cc1nc2ccc(Br)cc2o1 ; Cc1nc2ccc(NC3=NO[C@@]4(C3)CN3CCC4CC3)cc2o1 ; Cl]

To a solution of 6-bromo-2-methylbenzo[d]oxazole (0.12 g, 0.55 mmol), compound (R)-A-2 (0.10 g, 0.55 mmol), tris(dibenzylideneacetone)dipalladium(0) (51 mg, 0.055 mmol) and 4,5-bis(diphenylphosphino)-9,9-dimethylxanthene (64 mg, 0.11 mmol) in dioxane (2 mL) under nitrogen at room temperature was added cesium carbonate (0.36 g, 1.1 mmol). The reaction mixture was stirred at 100° C. for 2 hours, then filtered and concentrated in vacuo. The residue was purified by prep-HPLC [Instrument: GX-A; Column: Phenomenex Gemini C18 250×50 mm, particle size: 10 μm; Mobile phase: 23-53% acetonitrile in H₂O (add 0.05% NH₃ H₂O, v/v)] and lyophilized to give:

Compound (R)-51 (27 mg, 16% yield) as a yellow solid: cSFC analytical (R) tR=6.239 min., purity: 97.35%; LCMS (GG): tR=1.664 min., (ES⁺) m/z (M+H)⁺=313.2; ¹H-NMR (CD₃OD, 400 MHz): δ 7.89 (d, J=1.6 Hz, 1H), 7.45 (d, J=8.8 Hz, 1H), 7.16 (dd, J₁=8.4 Hz, J₂=2.0 Hz , 1H), 3.34 (s, 1H), 3.16-3.06 (m, 3H), 2.91-2.76 (m, 4H), 2.59 (s, 3H), 2.19-2.14 (m, 1H), 2.04 (s, 1H), 1.79-1.70 (m, 2H), 1.57-1.53 (m, 1H).

### Example 52

### (R)—N-(6-methylthieno[2,3-d]pyrimidin-2-yl)-4H-1′-azaspiro[isoxazole-5,3′-bicyclo[2.2.2]octan]-3-amine hydrochloride ((R)-52)

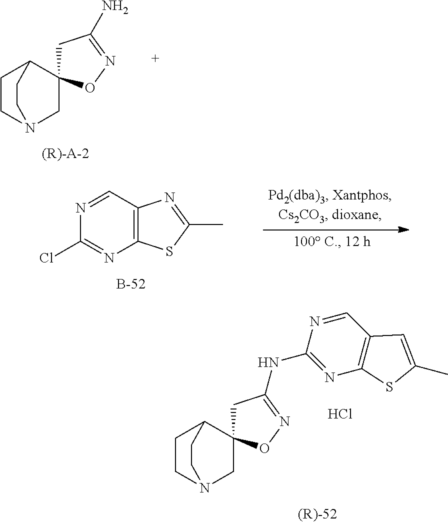

To a solution of compound B-52 (0.16 g, 0.87 mmol), compound (R)-A-2 (0.16 g, 0.87 mmol) , tris(dibenzylideneacetone)dipalladium(0) (79 mg, 0.087 mmol) and 4,5-bis(diphenylphosphino)-9,9-dimethylxanthene (75 mg, 0.13 mmol) in dioxane (20 mL) under nitrogen at room temperature was added cesium carbonate (0.56 g, 1.7 mmol). The reaction mixture was stirred at 100° C. for 12 hours, then filtered and concentrated in vacuo. The residue was purified by prep-HPLC [Instrument: GX-h; Column: Phenomenex Gemini C18 250×50 mm, particle size: 10 μm; Mobile phase: 23-53% acetonitrile in H₂O (add 0.05% ammonia hydroxide, v/v)]. The combined fractions were lyophilized, treated with 0.2 M hydrochloric acid and again lyophilized to give:

Compound (R)-52 (20 mg, 6.3% yield) as a yellow solid: cSFC analytical (S) tR=1.730 min., purity: 99.25%; LCMS (GG): tR=1.686 min., (ES⁺) m/z (M+H)⁺=330.1; ¹H-NMR (CD₃OD, 400 MHz): δ 8.93 (s, 1H), 7.19 (s, 1H), 3.81-3.61 (m, 4H), 3.48-3.33 (m, 4H), 2.65 (s, 3H),2.47-2.42 (m, 2H), 2.15-1.97 (m, 3H).

### Example 53

### (R)—N-(2-methylthieno[2,3-b]pyridin-6-yl)-4H-1′-azaspiro[isoxazole-5,3′-bicyclo[2.2.2]octan]-3-amine hydrochloride ((R)-53)

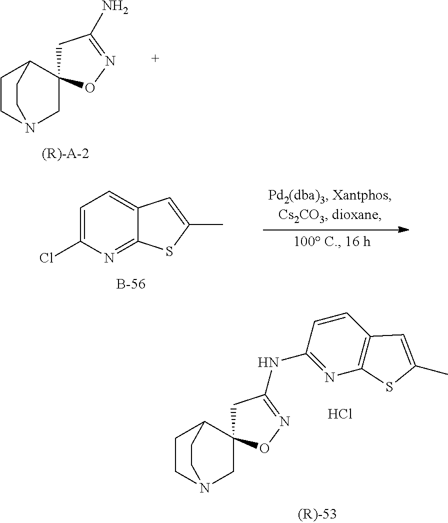

To a solution of compound B-56 (90 mg, 0.49 mmol), compound (R)-A-2 (0.89 g, 0.49 mmol), tris(dibenzylideneacetone)dipalladium(0) (45 mg, 0.049 mmol) and 4,5-bis(diphenylphosphino)-9,9-dimethylxanthene (57 mg, 0.098 mmol) in dioxane (6 mL) under nitrogen at room temperature was added cesium carbonate (0.32 g, 0.98 mmol). The reaction mixture was stirred at 100° C. for 16 hours, then filtered and concentrated in vacuo. The residue was purified by prep-HPLC [Instrument: GX-B; Column: Welch Ultimate AQ-C18 150×30 mm, particle size: 5 μm; Mobile phase: 19-49% acetonitrile in H₂O (add 0.1% TFA, v/v)]. The combined fractions were treated with 0.2 M hydrochloric acid and lyophilized to give:

Compound (R)-53 (30 mg, 17% yield) as a yellow solid: cSFC analytical (I) tR=1.846 min., purity: 97.55%; LCMS (FF): tR=2.256 min., (ES+) m/z (M+H)+=329.1; 1H-NMR (CD₃OD, 400 MHz): δ 8.12 (d, J=8.8 Hz, 1H), 7.37 (d, J=8.4 Hz, 1H), 7.03 (s, 1H), 3.77-3.67 (m, 3H), 3.58-3.37 (m, 5H), 2.60 (s, 3H), 2.46-2.41 (m, 2H), 2.15-2.11 (m, 1H), 2.05-1.96 (m, 2H).

### Example 54

### (R)—N-(2-chlorobenzo[b]thiophen-6-yl)-4H-F-azaspiroksoxazole-5,3¹-bicyclo[2.2.2]octan1-3-amine hydrochloride ((R)-54)

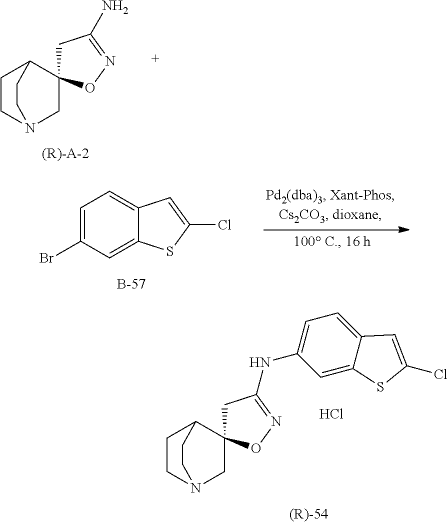

To a solution of compound B-57 (0.25 g, 1.0 mmol) and compound (R)-A-2 (0.18 g, 1.0 mmol) in dioxane (2.0 mL) under nitrogen at room temperature was added tris(dibenzylideneacetone)dipalladium(0) (92 mg, 0.10 mmol), 4,5-bis(diphenylphosphino)-9,9-dimethylxanthene (58 mg, 0.10 mmol) and cesium carbonate (0.66 g, 2.0 mmol). The mixture was stirred at 100° C. for 16 hours, then filtered and concentrated in vacuo. The residue was purified by prep-HPLC [Instrument: GX-H; Column: Phenomenex Gemini C18 250×50, particle size: 10 μm; Mobile phase: 40-70% acetonitrile in H₂O (add 0.05% ammonia hydroxide, v/v)]. The combined fractions were lyophilized, treated with 0.2 M hydrochloric acid and again lyophilized to give:

Compound (R)-54 (72 mg, 19% yield) as a brown solid: cSFC analytical (I) tR=3.552 min., purity: 98.50%; LCMS (GG): tR=2.142 min., (ES+) m/z (M+H)+=348.1; 1H-NMR (CD₃OD, 400 MHz): δ 8.05 (d, J=1.6 Hz, 1H), 7.62 (d, J=8.8 Hz , 1H), 7.27 (dd, J=8.8 Hz, 1H), 7.20 (s, 1H), 3.71-3.60 (m, 2H), 3.48-3.42 (m, 2H), 3.39-3.34 (m, 4H), 2.50-2.40 (m, 2H), 2.16-2.08 (m, 1H), 2.04-1.90 (m, 2H).

### Example 55

### (R)—N-(2,3-difluorobenzo[b]thiophen-6-yl)-3H-1′-azaspiro[furan-2,3¹-bicyclo[2.2.2]octan]-4-amine hydrochloride ((R)-55)

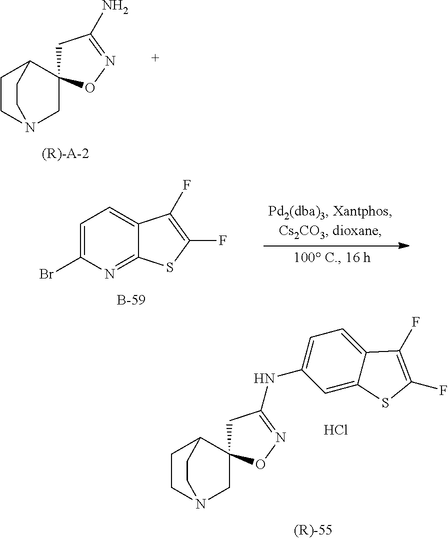

To a solution of compound (R)-A-2 (0.15 g, 0.83 mmol), compound B-59 (0.29 g, 0.83 mmol), tris(dibenzylideneacetone)dipalladium(0) (76 mg, 83 μmop and 4,5-bis(diphenylphosph-ino)-9,9-dimethylxanthene (96 mg, 0.17 mmol) in dioxane (8 mL) under nitrogen at room temperature was added cesium carbonate (0.54 g, 1.7 mmol). The mixture was stirred at 100° C. for 16 hours, then filtered and concentrated in vacuo. The residue was purified by prep-HPLC [Instrument: GX-B; Column: YMC-Actus ODS-AQ 100×30 mm, particle size: 5 μm; Mobile phase: 25-55% acetonitrile in H₂O (add 0.1% TFA, v/v)]. The combined fractions were treated with 0.2 N hydrochloric acid and lyophilized to give:

Compound (R)-55 (25 mg, 8% yield) as a white solid: cSFC analytical (I) tR=2.869 min., purity: 97.80%; LCMS (GG): tR=2.131 min., (ES⁺) m/z (M+H)⁺=350.1; ¹H-NMR (CD₃OD, 400 MHz): δ 8.05 (s, 1H), 7.61 (d, J=8.4 Hz, 1H), 7.39-7.36 (m, 1H), 3.70-3.61 (m, 2H), 3.48-3.35 (m, 6H), 2.46-2.40 (m, 2H), 2.16-2.13 (m, 1H), 2.06-1.90 (m, 2H).

### Example 56

### (R)—N -(3-methylbenzo[b]thiophen-6-yl)-4H-1′-azaspiro[isoxazole-5,3′-bicyclo[2.2.2]octan]-3-amine hydrochloride ((R)-56)

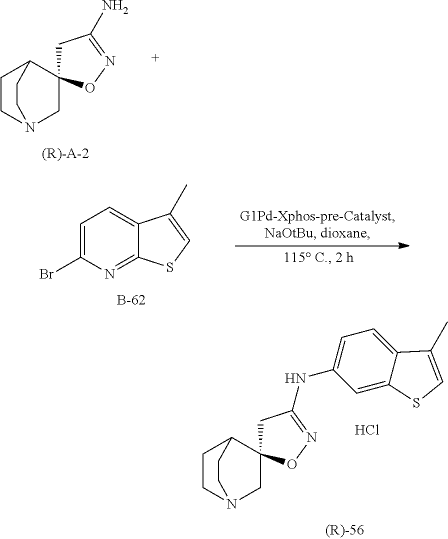

To a mixture of compound B-62 (0.15 g, 0.82 mmol) and compound (R)-A-2 (0.12 g, 0.68 mmol) in dioxane (10 mL) under nitrogen at room temperature was added sodium tert-butoxide (0.20 g, 2.1 mmol) and dicyclohexyl[2-(2,4,6-triisopropylphenyl)pheny]phosphane[2-(2-aminoethyl)phenyl]-chloro-palladium (0.06 g, 0.07 mmol). The reaction mixture was stirred at 115° C. for 2 hours, then filtered and concentrated in vacuo. The residue was purified by prep-HPLC [Instrument: Column: Phenomenex Gemini 250×50 mm, particle size: 10 μm; Mobile phase: 35-65% acetonitrile in H₂O (add 0.05% ammonia hydroxide v/v)]. The combined fractions were lyophilized, treated with 0.2 M hydrochloric acid and lyophilized again to give:

Compound (R)-56 (80 mg, 36% yield) as a white solid: cSFC analytical (I) tR=3.37 min., purity: 96.9%; LCMS (J): tR=2.04 min., 328.1 m/z (M+1); ¹H-NMR (CD₃OD, 400 MHz): δ 8.10 (d, J=1.6 Hz, 1H), 7.65 (d, J=8.8 Hz, 1H), 7.30 (dd, J₁=8.4 Hz, J₂=1.6 Hz 1H), 7.04 (s, 1H), 3.71-3.61 (m, 2H), 3.49-3.51 (m, 6H), 2.47-2.41 (m, 5H), 2.17-2.11 (m, 1H), 2.05-1.93 (m, 2H).

### Example 57

### (R)—N -(1-methyl-1H-benzo[d]imidazol-6-yl)-4H-1′-azaspiro[isoxazole-5,3′-bicyclo[2.2.2]octan]-3-amine hydrochloride ((R)-57)

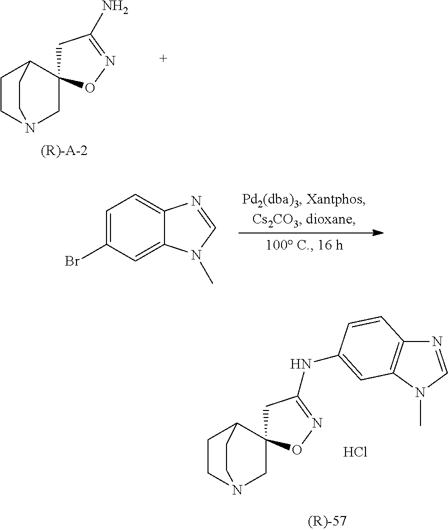 [SMILES: NC1=NO[C@@]2(C1)CN1CCC2CC1 ; Cn1cnc2ccc(Br)cc21 ; Cn1cnc2ccc(NC3=NO[C@@]4(C3)CN3CCC4CC3)cc21 ; Cl]

To a solution of 6-bromo-l-methyl-1H-benzo[d]imidazole (0.18 g, 0.83 mmol), compound (R)-A-2 (0.15 g, 0.83 mmol), tris(dibenzylideneacetone)dipalladium(0) (76 mg, 0.083 mmol) and 4,5-bis(diphenylphosphino)-9,9-dimethylxanthene (96 mg, 0.17 mmol) in dioxane (10 mL) under nitrogen at room temperature was added cesium carbonate (0.54 g, 1.7 mmol). The reaction mixture was stirred at 100° C. for 16 hours, then filtered and concentrated in vacuo. The residue was purified by prep-HPLC [Instrument: GX-E; Column: Phenomenex Gemini C18 250×50 mm, particle size: 10 μm; Mobile phase: 15-45% acetonitrile in H₂O (add 0.05% NH₃·H₂O, v/v)]. The combined fractions were lyophilized, treated with 0.2 M hydrochloric acid and again lyophilized to give:

Compound (R)-57 (50 mg, 17% yield) as a yellow solid: cSFC analytical (I) tR=3.545 min., purity: 100.00%; LCMS (FF): tR=1.630 min., (ES+) m/z (M+H)+=312.1; 1H-NMR (CD₃OD, 400 MHz): δ 9.28 (s, 1H), 8.22 (d, J=1.6 Hz, 1H), 7.75 (d, J=8.8 Hz, 1H), 7.49 (dd, J₁=9.2 Hz, J₂=2.0 Hz, 1H), 4.11 (s, 3H), 3.74 (dd, J1=14 Hz, J2=2.0 Hz , 1H), 3.64 (d, J=14 Hz, 1H), 3.55-3.37 (m, 6H), 2.46-2.41 (m, 2H), 2.16-2.12 (m, 1H), 2.05-1.95 (m, 2H).

### Example 58

### (R)—N-(1-methyl-1H-benzo[d]imidazol-5-yl)-4H-1′-azaspiro[isoxazole-5,3′-bicyclo[2.2.2 ]octan]-3-amine hydrochloride ((R)-58)

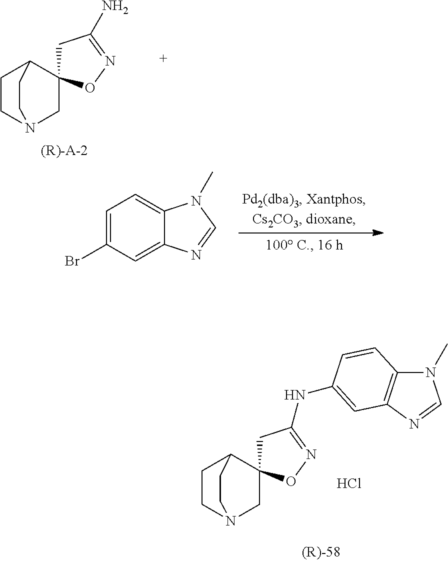 [SMILES: NC1=NO[C@@]2(C1)CN1CCC2CC1 ; Cn1cnc2cc(Br)ccc21 ; Cn1cnc2cc(NC3=NO[C@@]4(C3)CN3CCC4CC3)ccc21 ; Cl]

To a solution of 5-bromo-l-methyl-1H-benzo[d]imidazole (0.18 g, 0.83 mmol), compound (R)-A-2 (0.15 g, 0.83 mmol), tris(dibenzylideneacetone)dipalladium(0) (76 mg, 0.083 mmol) and 4,5-bis(diphenylphosphino)-9,9-dimethylxanthene (96 mg, 0.17 mmol) in dioxane (10 mL) under nitrogen at room temperature was added cesium carbonate (0.54 g, 1.7 mmol). The reaction mixture was stirred at 100° C. for 16 hours, then filtered and concentrated in vacuo. The residue was purified by prep-HPLC [Instrument: GX-E; Column: Phenomenex Gemini C18 250×50 mm, particle size: 10 μm; Mobile phase: 15-45% acetonitrile in H₂O (add 0.05% NH₃′ H₂O, v/v)]. The combined fractions were lyophilized, diluted with 0.2 M hydrochloric acid and again lyophilized to give:

Compound (R)-58 (70 mg, 27% yield) as a yellow solid: cSFC analytical (I) tR=3.076 min., purity: 100.00%; LCMS (FF): tR=1.637 min., (ES+) m/z (M+H)+=312.1; 1H-NMR (CD₃OD, 400 MHz): δ 9.20 (s, 1H), 8.22 (d, J=1.6 Hz, 1H), 7.81 (d, J=9.2 Hz, 1H), 7.50 (dd, J₁=8.8 Hz, J₂=2.0 Hz, 1H), 4.12 (s, 3H), 3.72 (dd, J1=13.6 Hz, J2=2.0 Hz , 1H), 3.63 (d, J=14.4 Hz, 1H), 3.53-3.37 (m, 6H), 2.46-2.40 (m, 2H), 2.14-2.11 (m, 1H), 2.06-1.94 (m, 2H).

### Example 59

### (R)—N-(7-methylisoquinolin-3-yl)-4H-1′-azaspiro[isoxazole-5,3′-bicyclo[2.2.2]octan]-3-amine hydrochloride ((R)-59)

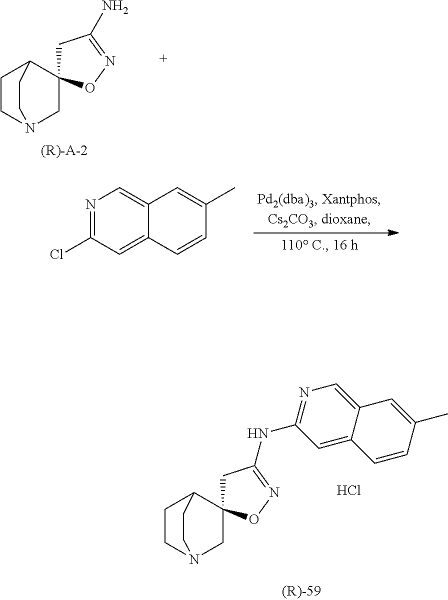 [SMILES: NC1=NO[C@@]2(C1)CN1CCC2CC1 ; Cc1ccc2cc(Cl)ncc2c1 ; Cc1ccc2cc(NC3=NO[C@@]4(C3)CN3CCC4CC3)ncc2c1 ; Cl]

To a solution of 3-chloro-7-methylisoquinoline (0.20 g, 1.1 mmol), compound (R)-A-2 (0.20 g, 1.1 mmol), tris(dibenzylideneacetone)dipalladium(0) (0.10 g, 0.11 mmol) and 4,5bis(diphenylphosphino)-9,9-dimethylxanthene (0.13 g, 0.22 mmol) in dioxane (10 mL) under nitrogen at room temperature was added cesium carbonate (1.1 g, 3.3 mmol). The reaction mixture was stirred at 110° C. for 16 hours, then filtered and concentrated in vacuo. The residue was purified by prep-HPLC [Instrument: GX-B; Column: Welch Ultimate AQ-C18 150×30 mm, particle size: 5 μm; Mobile phase: 20-50% acetonitrile in H₂O (add 0.1% TFA, v/v)]. The combined fractions were treated with 0.2 M hydrochloric acid and lyophilized to give:

Compound (R)-59 (22 mg, 6% yield) as a yellow solid: cSFC analytical (I) tR=3.179 min., purity: 100%; LCMS (GG): tR=1.881 min., (ES⁺) m/z (M+H)⁺=323.2; ¹H-NMR (CD₃OD, 400 MHz): δ 9.25 (s, 1H), 8.03 (s, 1H), 7.98-7.95 (m, 2H), 7.87 (d, J=8.8 Hz, 1H), 3.81-3.63 (m, 3H), 3.56-3.38 (m, 5H), 2.59 (s, 3H), 2.48-2.42 (m, 2H), 2.16-1.95 (m, 2H).

### Example 60

### (R)—N-(6-methylisoquinolin-3-yl)-4H-1′-azaspiro[isoxazole-5,3′-bicyclo[2.2.2]octan]-3-amine hydrochloride ((R)−60)

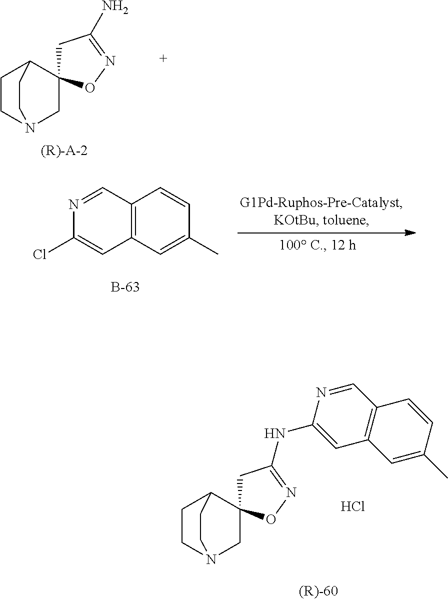

To a mixture of compound B-63 (0.35 g, 2.0 mmol) and compound (R)-A-2 (0.54 g, 3.0 mmol) in toluene (5 mL) under nitrogen at room temperature was added potassium tert-butoxide (0.66 g, 5.9 mmol), chloro-(2-dicyclohexylphosphino-2′, 6′-diisopropoxy-1,1′-biphenyl)[2-(2-aminoethyl)phenyl]palladium(II)-methyl-t-butyl ether adduct (0.16 g, 0.20 mmol). The reaction mixture was stirred at 100° C. for 12 hours, then filtered and concentrated in vacuo. The residue was purified by prep-HPLC [Instrument: GX-B; Column: Welch Ultimate AQ-C18 150×30 mm, particle size: 5 μm; Mobile phase: 28-58% acetonitrile in H₂O (add 0.1% TFA, v/v)]. The combined fractions were treated with 0.2 M hydrochloric acid and lyophilized to give:

Compound (R)-60 (49 mg, 8% yield) as a yellow solid: cSFC analytical (I) tR=3.152 min., purity: 99.77%; LCMS (GG): tR=1.837 min., (ES⁺) m/z (M+H)⁺=323.2; ¹H-NMR (CD₃OD, 400 MHz): δ 9.30 (s, 1H), 8.17 (d, J=8.8 Hz, 1H), 7.87 (s, 1H), 7.84 (s, 1H), 7.61 (d, J=8.0 Hz, 1H), 3.81-3.72 (m, 2H), 3.68-3.37 (m, 6H), 2.62 (s, 3H), 2.46-2.39 (m, 2H), 2.18-1.93 (m, 3H).

### Example 61

### (R)—N-(6,7-difluoroisoquinolin-3-yl)-4H-1′-azaspiro[isoxazole-5,3′-bicyclo[2.2.2]octan]-3-amine hydrochloride ((R)-61)

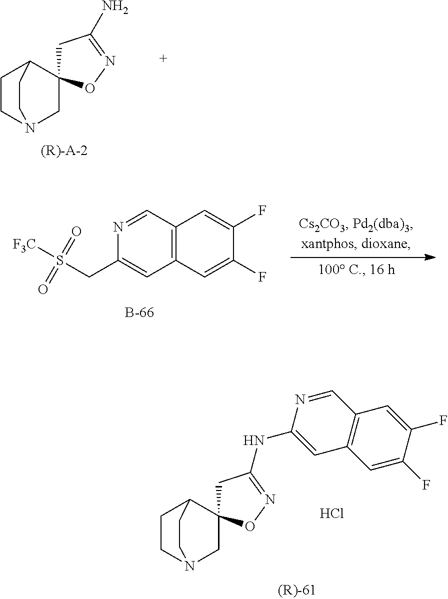

To a solution of compound B-66 (0.22 g, 0.70 mmol) and compound (R)-A-2 (0.13 g, 0.70 mmol) in dioxane (10 mL) under nitrogen at room temperature was added tris(dibenzylideneacetone)dipalladium(0) (64 mg, 0.070 mmol), cesium carbonate (0.69 g, 2.1 mmol) and 4,5-bis(diphenylphosphino)-9,9-dimethylxanthene (81 mg, 0.14 mmol). The mixture was stirred at 100° C. for 16 hours, then filtered and concentrated in vacuo. The residue was purified by prep-HPLC [Instrument: GX-B; Column: Welch Ultimate AQ- C18 150×30 mm, particle size: 5 μm; Mobile phase: 25-55% acetonitrile in H₂O (add 0.1% TFA, v/v)]. The combined fractions were treated with 0.2 M hydrochloric acid (1.5 mL) and lyophilized to give:

Compound (R)-61 (52 mg, 20% yield) as a yellow solid: cSFC analytical (G) tR=4.304 min., purity: 97.7%; LCMS (GG): tR=1.969 min., (ES⁺) m/z (M+H)⁺=345.1; ¹H-NMR (CD₃OD, 400 MHz): δ 9.04 (s, 1H), 7.97 (s, 1H), 7.94 (dd, J₁=10.3 Hz, J₂=8.2 Hz, 1H), 7.72 (dd, J₁=11.1 Hz, J₂=7.6 Hz, 1H), 3.74-3.55 (m, 3H), 3.46-3.35 (m, 5H), 2.46-2.42 (m, 2H), 2.12-2.10 (m, 1H), 2.03-1.93 (m, 2H).

### Example 62

### (R)—N-(6,7-dichloroisoquinolin-3-yl)-4H-1′-azaspiro[isoxazole-5,3′-bicyclo[2.2.2]octan]-3-amine trifluoroacetate ((R)-62)

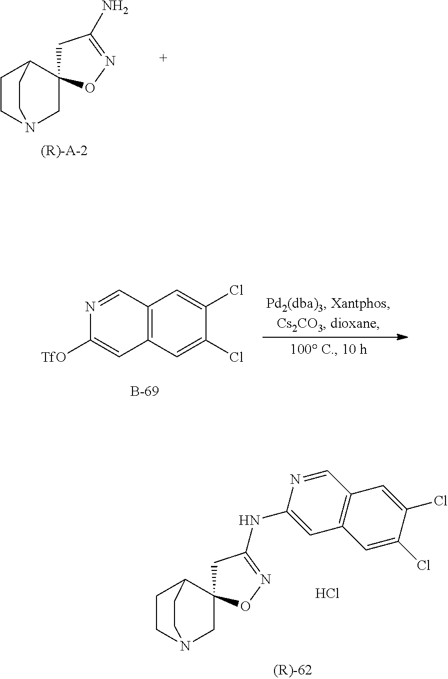

To a solution of compound B-δ 9 (0.20 g, 0.58 mmol), compound (R)-A-2 (0.10 g, 0.58 mmol), tris(dibenzylideneacetone)dipalladium(0) (0.05 g, 0.06 mmol) and 4,5-bis(diphenylphosphino)-9,9-dimethylxanthene (0.07 g, 0.12 mmol) in dioxane (5 mL) under nitrogen at room temperature was added cesium carbonate (0.38 g, 1.2 mmol). The reaction mixture was stirred at 100° C. for 10 hours, then filtered and concentrated in vacuo. The residue was purified by prep-HPLC [Instrument: GX-B; Column: Welch Ultimate AQ-C18 150×30 mm, particle size: 5 μm; Mobile phase: 30-60% acetonitrile in H2O (add 0.1% TFA, v/v)] and lyophilized to give:

Compound (R)-62 (13 mg, 5% yield) as a green solid: cSFC analytical (P) tR=3.58 min., purity: 98.27%; LCMS (GG): tR=2.171 min., (ES⁺) m/z (M+H)⁺=377.0; ¹H-NMR (CD₃OD, 400 MHz): δ 8.95 (s, 1H), 8.15 (s, 1H), 7.95 (s, 1H), 7.91 (s, 1H), 3.70-3.62 (m, 2H), 3.53 (d, J=16.8 Hz, 1H), 3.47-3.34 (m, 5H), 2.46-2.40 (m, 2H), 2.14-2.09 (m, 1H), 2.03-1.92 (m, 2H).

### Example 63

### (R)—N-(2-chlorobenzofuran-6-yl)-4H-1′-azaspiro[isoxazole-5,3′-bicyclo[2.2.2]octan]-3-amine hydrochloride ((R)-63)

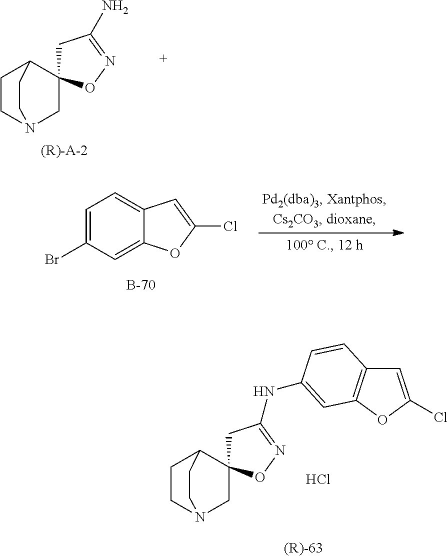

To a solution of compound B-70 (0.15 g, 0.65 mmol), compound (R)-A-2 (0.18 g, 0.97 mmol), tris(dibenzylideneacetone)dipalladium(0) (59 mg, 0.065 mmol) and 4,5-bis(diphenylphosphino)-9,9-dimethylxanthene (75 mg, 0.13 mmol) in dioxane (5 mL) under nitrogen was added cesium carbonate (0.42 g, 1.3 mmol). The reaction mixture was stirred at 100° C. for 12 hours, then filtered and concentrated in vacuo. The residue was purified by prep-HPLC [Instrument: GX-I; Column: YMC-Actus ODS-AQ 150×30 mm, particle size: 5 μm; Mobile phase: 25-55% acetonitrile in H₂O (add 0.1% TFA, v/v)]. The combined fractions were treated with 0.2 M hydrochloric acid and lyophilized to give:

Compound (R)-63 (24 mg, 11% yield) as a yellow solid: cSFC analytical (I) tR=3.162 min., purity: 100%; LCMS (GG): tR=2.008 min., (ES⁺) m/z (M+H)⁺=332.1; ¹H-NMR (CD₃OD, 400 MHz): δ 7.80 (s, 1H), 7.41 (d, J=8.4 Hz, 1H), 7.13 (dd, J₁=8.4 Hz, J₂=2.0 Hz , 1H), 6.65 (s, 1H), 3.63 (dd, J₁=24.0 Hz, J₂=15.2 Hz, 2H), 3.46-3.33 (m, 6H), 2.44-2.38 (m, 2H), 2.15-2.07 (m, 1H), 2.02-1.88 (m, 2H).

### Example 64

### (R)—N-(1-methyl-1H-indazol-6-yl)-4H-1′-azaspiro[isoxazole-5,3′-bicyclo[2.2.2]octan]-3-amine hydrochloride ((R)-64)

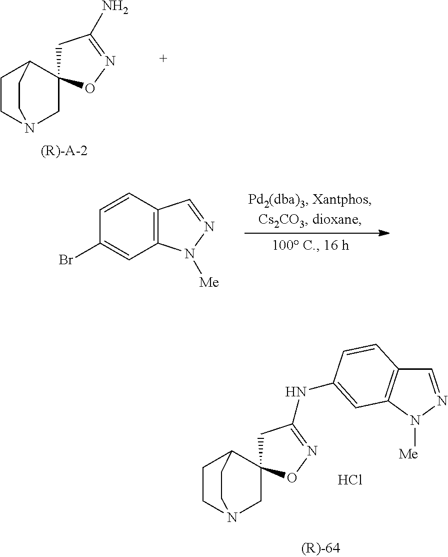 [SMILES: NC1=NO[C@@]2(C1)CN1CCC2CC1 ; Cn1ncc2ccc(Br)cc21 ; Cn1ncc2ccc(NC3=NO[C@@]4(C3)CN3CCC4CC3)cc21 ; Cl]

To a solution of 6-bromo-l-methyl-1H-indazole (0.15 g, 0.72 mmol), compound (R)-A-2 (0.13 g, 0.72 mmol), tris(dibenzylideneacetone)dipalladium(0) (66 mg, 0.072 mmol) and 4,5-bis(diphenylphosphino)-9,9-dimethylxanthene (83 mg, 0.14 mmol) in dioxane (2 mL) under nitrogen at room temperature was added cesium carbonate (0.70 g, 2.2 mmol). The reaction mixture was stirred at 100° C. for 16 hours, then filtered and concentrated in vacuo. The residue was purified by prep-HPLC [Instrument: GX-H; Column: Phenomenex Gemini C18 250×50 mm, particle size: 10 μm; Mobile phase: 25-46% acetonitrile in H₂O (add 0.5% NH₃·H₂O, v/v)]. The combined fractions were lyophilized, treated with 0.2 M hydrochloric acid solution and again lyophilized to give:

Compound (R)-64 (75 mg, 30% yield) as a brown solid: cSFC analytical (I) tR=2.902 min., purity: 100.00%; LCMS (GG): tR=2.078 min., (ES⁺) m/z (M+H)⁺=312.1; ¹H-NMR (CD₃OD, 400 MHz): δ 8.24 (s, 1H), 7.93 (s, 1H), 7.75 (d, J=8.8 Hz, 1H), 7.10 (d, J=8.8 Hz, 1H), 4.09 (s, 3H), 3.74 (d, J=14 Hz, 1H), 3.64 (d, J=14 Hz, 1H), 3.55-3.37 (m, 6H), 2.47-2.41 (m, 2H), 2.16-2.12 (m, 1H), 2.04-1.94 (m, 2H).

### Example 65

### (R)—N-(1-methyl-1H-indazol-5-yl)-4H-1′-azaspiro[isoxazole-5,3′-bicyclo[2.2.2]octan]-3-amine hydrochloride ((R)-65)

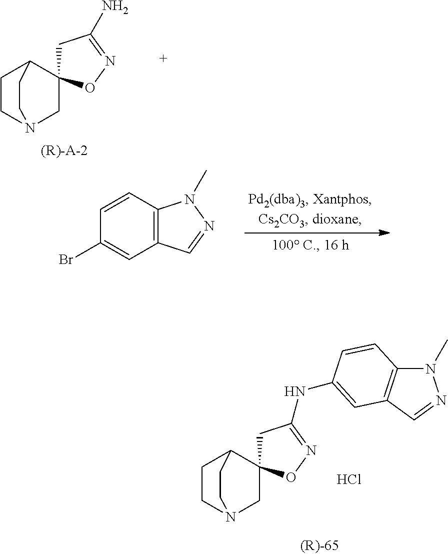 [SMILES: NC1=NO[C@@]2(C1)CN1CCC2CC1 ; Cn1ncc2cc(Br)ccc21 ; Cn1ncc2cc(NC3=NO[C@@]4(C3)CN3CCC4CC3)ccc21 ; Cl]

To a solution of 5-bromo-l-methyl-1H-indazole (0.15 g, 0.72 mmol), compound (R)-A-2 (0.13 g, 0.72 mmol), tris(dibenzylideneacetone)dipalladium(0) (66 mg, 0.072 mmol) and 4,5-bis(diphenylphosphino)-9,9-dimethylxanthene (83 mg, 0.14 mmol) in dioxane (2 mL) under nitrogen at room temperature was added cesium carbonate (0.70 g, 2.2 mmol). The reaction mixture was stirred at 100° C. for 16 hours, then filtered and concentrated in vacuo. The residue was purified by prep-HPLC [Instrument: GX-H; Column: Phenomenex Gemini C18 250×50 mm, particle size: 10 μm; Mobile phase: 25-45% acetonitrile in H₂O (add 0.5% NH₃·H₂O, v/v)]. The combined fractions were lyophilized, treated with 0.2 M hydrochloric acid and again lyophilized to give:

Compound (R)-65 (75 mg, 30% yield) as a white solid: cSFC analytical (I) tR=2.864 min., purity: 98.23%; LCMS (GG): tR=2.041 min., (ES⁺) m/z (M+H)⁺=312.2; ¹H-NMR (CD₃OD, 400 MHz): δ 8.03 (s, 1H), 7.98 (s, 1H), 7.55 (d, J=8.8 Hz, 1H), 7.40 (d, J=8.8 Hz, 1H), 4.08 (s, 3H), 3.73 (d, J=14 Hz, 1H), 3.63 (d, J=14 Hz, 1H), 3.53-3.36 (m, 6H), 2.44-2.42 (m, 2H), 2.16-2.11 (m, 1H), 2.03-1.93 (m, 2H).

### Example 66

### (R)—N -(3-methylbenzo[d]isoxazol-6-yl)-4H-1′-azaspiro[isoxazole-5,3′-bicyclo[2.2.2]octan]-3-amine hydrochloride ((R)-66)

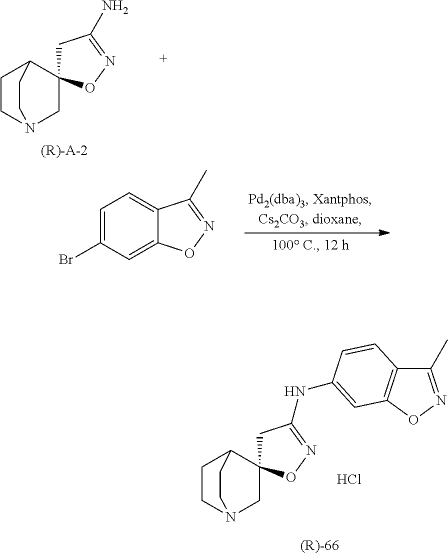 [SMILES: NC1=NO[C@@]2(C1)CN1CCC2CC1 ; Cc1noc2cc(Br)ccc12 ; Cc1noc2cc(NC3=NO[C@@]4(C3)CN3CCC4CC3)ccc12 ; Cl]

To a solution of 6-bromo-3-methylbenzo[d]isoxazole (0.1 g, 0.47 mmol), compound (R)-A-2 (85 mg, 0.47 mmol), tris(dibenzylideneacetone)dipalladium(0) (43 mg, 0.047 mmol) and 4,5-bis(diphenylphosphino)-9,9-dimethylxanthene (55 mg, 0.094 mmol) in dioxane (2 mL) under nitrogen at room temperature was added cesium carbonate (0.31 g, 0.94 mmol). The reaction mixture was stirred at 100° C. for 12 hours, then filtered and concentrated in vacuo. The residue was purified by prep-HPLC [Instrument: GX-H; Column: Phenomenex Gemini C18 250×50 mm, particle size: 10 μm; Mobile phase: 25-55% acetonitrile in H₂O (add 0.05% ammonia hydroxide, v/v)]. The combined fractions were lyophilized, treated with 0.2 M hydrochloric acid and again lyophilized to give:

Compound (R)-66 (26 mg, 15% yield) as a yellow solid: cSFC analytical (P) tR=2.884 min., purity: 100%; LCMS (FF): tR=2.118 min., (ES⁺) m/z (M+H)⁺=313.1; ¹H-NMR (CD₃OD, 400 MHz): δ 7.90(d, J=1.2 Hz, 1H), 7.62 (d, J=8.4 Hz, 1H), 7.18 (dd, J₁=1.6 Hz, J₂=8.4 Hz, 1H), 3.65 (dd, J₁=8.4 Hz, J₂=14 Hz, 2H), 3.49-3.33 (m, 6H), 2.51 (s, 3H), 2.46-2.38 (m, 2H), 2.16-2.07 (m, 1H), 2.02-1.89 (m, 2H).

### Example 67

### (R)—N-(2-(trifluoromethyl)benzo[b]thiophen-6-yl)-4H-1′-azaspiro[isoxazole-5,3′-bicyclo[2.2.2]octan]-3-amine hydrochloride ((R)-67)

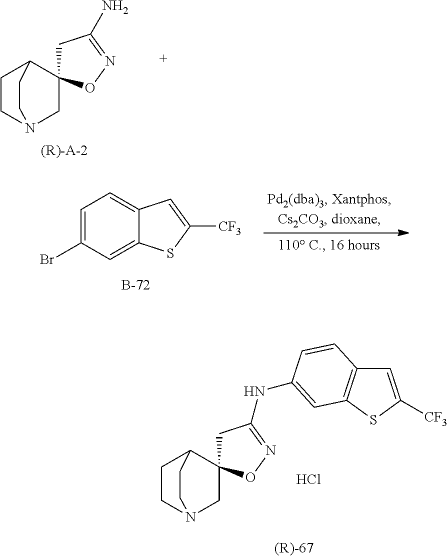

To a solution of compound (R)-A-2 (0.15 mg, 0.83 mmol), compound B-72 (0.23 g, 0.83 mmol), tris(dibenzylideneacetone)dipalladium(0) (76 mg, 83 μmol) and 4,5-bis(diphenylphosph-ino)-9,9-dimethylxanthene (96 mg, 0.17 mmol) in dioxane (2 mL) under nitrogen at room temperature was added cesium carbonate (0.81 g, 2.5 mmol). The mixture was stirred at 100° C. for 16 hours, then filtered and concentrated in vacuo. The residuw was purified by prep-HPLC [Instrument: GX-E; Column: YMC-Actus Triart C18 150×30 mm, particle size: 5 μm; Mobile phase: 25-55% acetonitrile in H₂O (add 0.1% TFA, v/v)]. The combined fractions were treated with 0.2 N hydrochloride and lyophilized to give:

Compound (R)-67 (60 mg, 48% yield) as a white solid: cSFC analytical (I) tR=2.320 min., purity: 100%; LCMS (EE): tR=2.555 min., (ES⁻) m/z (M+H)⁺=382.1; ¹H-NMR (CD₃OD, 400 MHz): δ 8.27 (s, 1H), 7.85 (d, J=8.8 Hz, 1H), 7.77 (s, 1H), 7.36 (d, J=8.8 Hz, 1H), 3.72-3.62 (m, 2H), 3.51- 3.36 (m, 6H), 2.47-2.41 (m, 2H), 2.14-2.11 (m, 1H), 2.05-1.93 (m, 2H).

### Example 68

### (R)—N-(1,2-dimethyl -1H-benzo[d]imidazol-6-yl)-4H-1′-azaspiro[isoxazole-5,3′-bicyclo[2.2.2]octan]-3-amine hydrochloride ((R)-68)

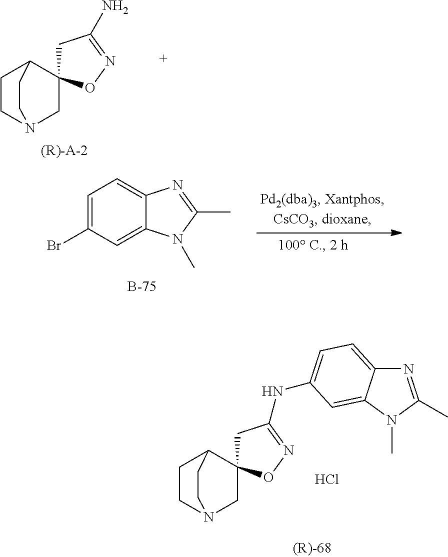

To a solution of compound B-75 (0.20 g, 0.89 mmol), compound (R)-A-2 (0.16 g, 0.89 mmol), tris(dibenzylideneacetone)dipalladium(0) (0.081 g, 0.089 mmol) and 4,5-bis(diphenylphosphino)-9,9-dimethylxanthene (0.10 g, 0.18 mmol) in dioxane (8 mL) under nitrogen at room temperature was added cesium carbonate (0.58 g, 1.8 mmol). The mixture was stirred at 100° C. for 2 hours, then filtered and concentrated in vacuo. The residue was purified by prep-HPLC [Instrument: GX-d; Column: Phenomenex Gemini C18 250×50 mm, particle size: 10 μm; Mobile phase: 20-38% acetonitrile in H₂O (add 0.05% NH₃·H₂O, v/v)]. The solution was lyophilized, diluted with 0.2 M hydrochloric acid and again lyophilized to give:

Compound (R)-68 (32 mg, 9.8% yield) as a white solid: cSFC analytical (I) tR=4.087 min., purity: 100%; LCMS (EE): tR=1.670 min., (ES⁻) m/z (M+H)⁺=326.2; ¹H-NMR (CD₃OD, 400 MHz): δ 7.73 (d, J=1.6 Hz, 1H), 7.40 (d, J=8.8 Hz, 1H), 6..99 (dd, J₁=2.0 Hz, J₂=1.6 Hz, 1H), 3.72 (s, 3H), 3.23 (dd, J₁=14.2 Hz, J₂=11.2 Hz, 1H), 2.93-2.86 (m, 3H), 2.84-2.81 (m, 4H), 2.55 (s, 3H), 1.79-1.77 (m, 1H), 1.75 (s, 1H), 1.75-1.72 (m, 2H), 1.58-1.55 (m, 1H).

### Example 69

### (R)—N -(1-methyl-1H-benzo[d]imidazol-2-yl)-4H-1′-azaspiro[isoxazole-5,3′-bicyclo[2.2.2]octan]-3-amine hydrochloride ((R)-69)

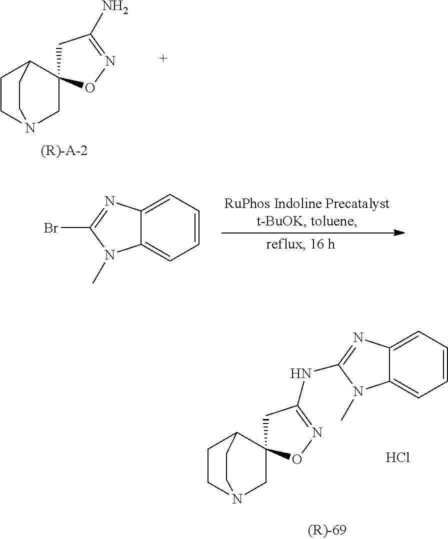 [SMILES: NC1=NO[C@@]2(C1)CN1CCC2CC1 ; Cn1c(Br)nc2ccccc21 ; Cn1c(NC2=NO[C@@]3(C2)CN2CCC3CC2)nc2ccccc21 ; Cl]

To a solution of 2-bromo-l-methyl-1H-benzo[d]imidazole (0.23 g, 1.1 mmol), compound (R)-A-2 (0.20 mg, 1.1 mmol), chloro-(2-dicyclohexylphosphino-2′, 6′-diisopropoxy-1,1′-bipheny)[2-(2- aminoethyl)phenyl]palladium(II)-methyl-t-butyl ether adduct (45 mg, 55 μmol) in toluene (5 mL) under nitrogen at room temperature was added potassium tert-butoxide (0.25 g, 2.2 mmol). The mixture was stirred at reflux for 16 hours, then filtered and concentrated in vacuo. The residue was purified by silica gel chromatography Al₂O₃,[ ethyl acetate : methanol=100:1˜0:1] and prep-HPLC [Instrument: GX-J; Column: Venusil XBP C18 150×25 mm, particle size: 10 μm; Mobile phase: 2-32% acetonitrile in H₂O (add 0.1% TFA, v/v)]. The solution was treated with 0.2 N hydrochloride acid to give:

Compound (R)-δ 9 (20 mg, 5% yield) as a white solid. : cSFC analytical (I) tR=2.824 min., purity: 100%; LCMS (FF): tR=1.793 min., (ES⁺) m/z (M+H)⁺=312.2; ¹H-NMR (CD₃OD, 400 MHz): δ 7.72-7.69 (m, 2H), 7.55-7.47 (m, 2H), 3.94 (s, 3H), 3.84-3.67 (m, 3H), 3.60-3.33 (m, 5H), 2.49-2.42 (m, 2H), 2.17-1.96 (m, 3H).

### Example 70

### (R)—N-(6-methylquinazolin-2-yl)spiro[4H-isoxazole-5,3¹-quinuclidine]-3-amine ((R)-70)

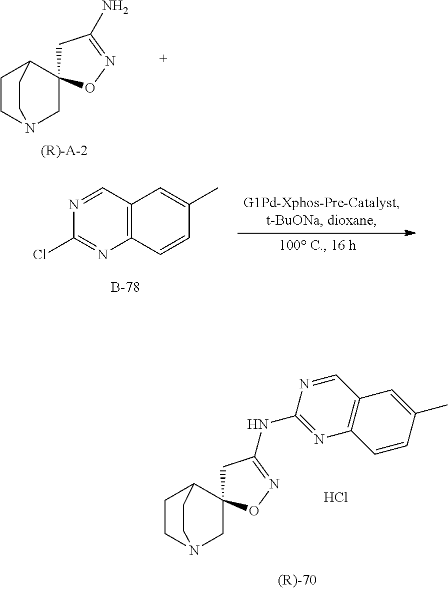

To a mixture of compound B-78 (170 mg, 0.95 mmol) and compound (R)-A-2 (190 mg, 1.05 mmol) in dioxane (5 mL) under nitrogen at room temperature was added sodium tert-butoxide (2 M in THF, 0.95 mL) and dicyclohexyl-[2-(2,4,6-triisopropylphenyl)phenyl]phosphane-[2-(2-aminoethyl)phenyl]-chloro-palladium (35 mg, 0.48 mmol). The mixture was stirred at 100° C. for 16 hours, then filtered and concentrated in vacuo. The residue was purified twice by prep-HPLC [Instrument: GX-A; Column: GEMINI 250×50 mm, particle size: 10 μm; Mobile phase: 35-44% acetonitrile in H₂O (add 0.5% NH₃·H₂O, v/v)] and lyophilized to give:

Compound (R)-70 (15 mg, 5% yield) as a white solid: cSFC analytical (I) tR=2.941 min., purity: 100%; LCMS (FF): tR=1.736 min., (ES⁺) m/z (M+H)⁺=324.2; ¹H-NMR (CD₃OD, 400 MHz): δ 9.15 (s, 1H), 7.75-7.70 (m, 3H), 3.77 (d, J=16.8 Hz, 1H), 3.44 (d, J=17.2 Hz, 1H), 3.33-3.16 (m, 2H), 2.93-2.84 (m, 4H), 2.51 (s, 3H), 2.20-2.10 (m, 2H), 1.81-1.79 (m, 2H), 1.60-1.57 (m, 1H).

### Example 71

### (R)—N-(7-methylquinazolin-2-yl)-4H-1′-azaspiro[isoxazole-5,3′-bicyclo[2.2.2]octan]-3-amine hydrochloride ((R)-71)

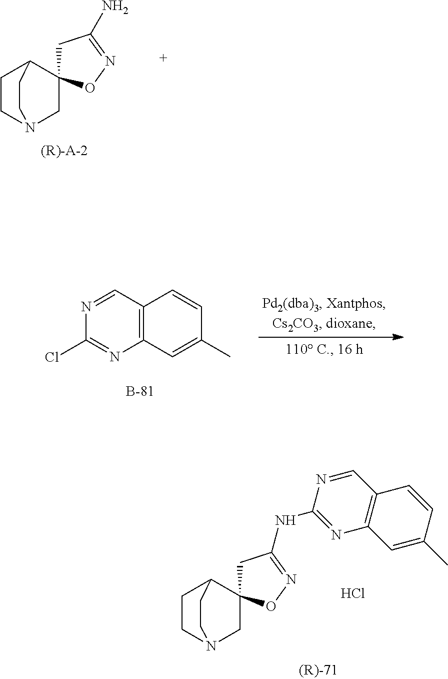

To a mixture of compound B-81 (150 mg, 0.84 mmol) and compound (R)-A-2 (167 mg, 0.92 mmol) in dioxane (1.00 mL) under nitrogen at room temperature was added 4,5-bis(diphenylphosphino)-9,9-dimethylxanthene (97 mg, 0.17 mmol), cesium carbonate (547 mg, 1.68 mmol) and tris(dibenzylideneacetone)dipalladium(0) (77 mg, 0.084 mmol). The mixture was stirred at 110° C. for 16 hours, then filtered and concentrated in vacuo. The resulting solids were purified by prep-HPLC [GX-A; Welch Ultimate AQ-C18 150×20 mm, particle size: 10 μm; Mobile phase: 20-48% water (0.05% ammonia hydroxide v/v)-ACN]. The combined fractions were lyophilized, treated with 2 M hydrochloric acid solution and again lyophilized to give:

Compound (R)-71 (15 mg, 5% yield) as yellow solid: cSFC analytical (I) tR=2.980 min., purity: 100%; LCMS (FF): tR=1.608 min., (ES+) m/z (M+H)+=324.2; 1H-NMR (CD3OD, 400 MHz): δ 9.50 (s, 1H), 8.36 (s, 1H), 8.29 (d, J=8.0 Hz, 1H), 7.74 (d, J=8.0 Hz, 1H), 3.68 (d, J=13.6 Hz, 1H), 3.56 (d, J=14.8 Hz, 1H), 3.45-3.35 (m, 6H), 2.75 (s, 3H), 2.36-2.28 (m, 3H), 2.16-2.10 (m, 1H), 1.91-1.83 (m, 1H).

### Example 72

### (R)—N-(3-methylbenzo[d]isothiazol-6-yl)-4H-1′-azaspiro[isoxazole-5,3′-bicyclo[2.2.2]octan]-3-amine hydrochloride ((R)-72)

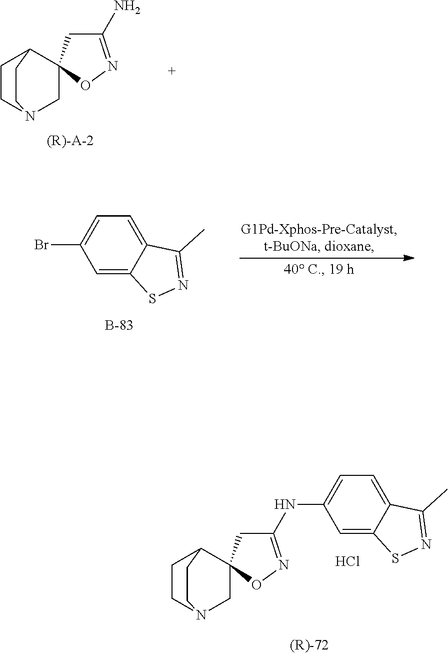

To a mixture of compound B-83 (0.36 g, 1.6 mmol) and compound (R)-A-2 (0.29 g, 1.6 mmol) in dioxane (10 mL) under nitrogen at room temperature was added sodium tert-butoxide (0.30 g, 3.2 mmol) and dicyclohexyl[2,-(2,4,6-triisopropylphenyl)phenyl]phosphane[2-(2-aminoethyl)phenyl]-chloro-palladium (0.12 g, 0.16 mmol). The reaction mixture was stirred at 40° C. for 19 hours, then filtered and concentrated in vacuo. The residue was purified by prep-HPLC [Instrument: GX-J; Column: Venusil XBP C8 150×25 mm, particle size: 10 μm; Mobile phase: 22-52% acetonitrile in H₂O (add 0.1% TFA, v/v)]. The combined fractions were treated with 0.2 M hydrochloric acid and lyophilized to give:

Compound (R)-72 (44 mg, 7.5% yield) as a yellow solid. cSFC analytical (I) tR=3.599 min., purity: 98.15%; LCMS (FF): tR=2.200 min., (ES⁺) m/z (M+H)⁺=292.1; ¹H-NMR (CD₃OD, 400 MHz): δ 8.29 (s, 1H), 7.96 (d, J=8.8 Hz, 1H), 7.36 (dd, J₁=1.2 Hz, J₂=1.2 Hz, 1H), 3.71-3.64 (m, 2H), 3.48-3.31 (m, 6H), 2.69 (s, 3H), 2.45-2.39 (m, 2H), 2.12-2.09 (m, 1H), 1.99-1.92 (m, 2H).

### Example 73

### (R)—N-(3-chlorobenzofuran-6-yl)-4H-1′-azaspiro[isoxazole-5,3′-bicyclo[2.2.2]octan]-3-amine hydrochloride ((R)-73-1) and (R)—N -(3 ,5-dichlorobenzofuran-6-yl)-4H-1′-azaspiro[isoxazole-5,3′-bicyclo[2.2.2.]octan]-3-amine hydrochloride ((R)-73-2)

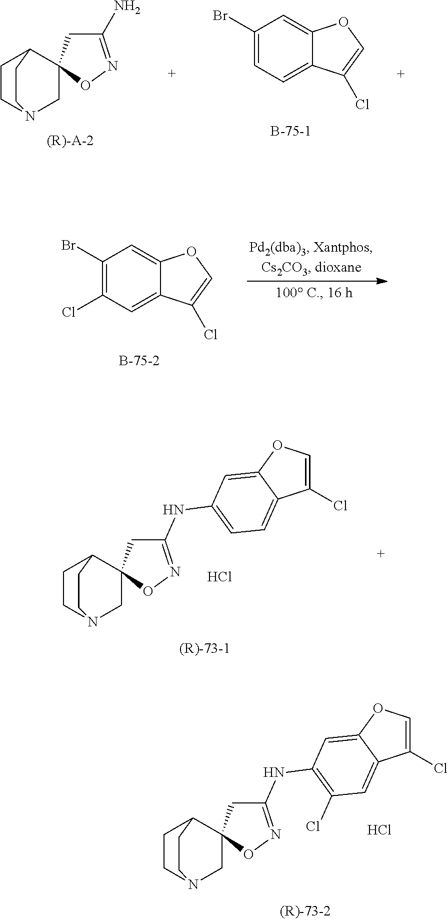

To a mixture of compound B-84-1 and B-84-2 (300 mg, crude), compound (R)-A-2 (155 mg, 0.86 mmol), tris(dibenzylideneacetone)dipalladium(0) (78 mg, 0.086 mmol) and 4,5- bis(diphenylphosphino)-9,9-dimethylxanthene (50 mg, 0.086 mmol) in dioxane (3 mL) under nitrogen at room temperature was added cesium carbonate (0.56 g, 1.7 mmol). The reaction mixture was stirred at 100° C. for 16 hours, then filtered and concentrated in vacuo. The components were separated by prep-HPLC [Instrument: GX-I; Column: YMC-Actus ODS-AQ C18 150×30 mm, particle size: 5 μm; Mobile phase: 30-60% acetonitrile in H₂O (add 0.05% HCl v/v)], and each set of combined fractions was lyophilized to give:

Compound (R)-73-1 (45 mg) as a white solid.: cSFC analytical (I) tR=2.965 min., purity: 93.58%; LCMS (GG): tR=1.656 min., (ES⁺) m/z (M+H)⁺=2.373; ¹H-NMR (CD₃OD, 400 MHz): δ 7.88 (d, J=1.6 Hz, 1H), 7.78 (s, 1H), 7.46 (d, J=8.4 Hz, 1H), 7.26-7.24 (m, 1H), 3.72-3.38 (m, 8H), 2.45-2.39 (m, 2H), 2.12-2.11 (m, 1H), 2.03-1.90 (m, 2H), and

Compound (R)-73-2 (15 mg) as a white solid.: cSFC analytical (I) tR=2.648 min., purity: 98.13%; LCMS (GG): tR=2.172 min., (ES⁺) m/z (M+H)⁺=366.1; ¹H-NMR (CD₃OD, 400 MHz): δ 8.37 (s, 1H), 7.89 (s, 1H), 7.65 (s, 1H), 3.72-3.66 (m, 2H), 3.63-3.58 (m, 1H), 3.50-3.36 (m, 5H), 2.41 (m, 2H), 2.17-2.10 (m, 1H), 2.05-1.91 (m, 2H).

### Example 74

### (R)—N-(3-methylbenzofuran-6-yl)-4H-1′-azaspiro[isoxazole-5,3′-bicyclo[2.2.2]octan]-3-amine hydrochloride ((R)-74)

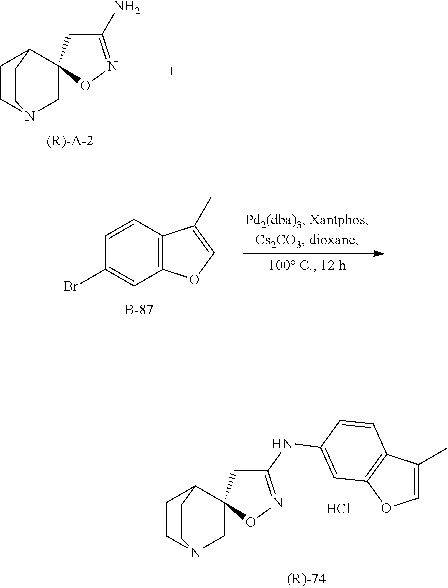

A mixture of compound B-87 (0.23 g, 1.1 mmol), compound (R)-A-2 (0.20 g, 1.1 mmol), tris(dibenzylideneacetone)dipalladium(0) (0.10 g, 0.11 mmol) and 4,5-bis(diphenylphosphino)-9,9-dimethylxanthene (0.13 g, 0.22 mmol) and cesium carbonate (0.72 g, 2.2 mmol) in dioxane (10 mL) under nitrogen was stirred at 100° C. for 12 hours, then filtered and concentrated in vacuo. The residue was purified by prep-HPLC [Instrument: GX-B; Column: Welch Ultimate AQ-C18 150×30 mm, particle size: 5 μm; Mobile phase: 18-48% acetonitrile in H₂O (add 0.1% TFA, v/v)]. The combined fractions were treated with 0.2 M hydrochloric acid and lyophilized to give:

Compound (R)-74 (88 mg, 23% yield) as a white solid.: cSFC analytical (P) tR=2.80 min., purity: 100%; LCMS (GG): tR=1.948 min., (ES⁺) m/z (M+H)⁺=312.2; ¹H-NMR (CD₃OD, 400 MHz): δ 7.71 (d, J=1.6 Hz, 1H), 7.44-7.32 (m, 2H), 7.14 (dd, J=8.4 Hz, J=1.6 Hz, 1H), 3.68 (d, J=12.0 Hz, 1H), 3.61 (d, J=12.0 Hz, 1H), 3.49-3.43 (m, 2H), 3.44-3.40 (m, 4H), 2.42-2.39 (m, 2H), 2.21 (s, 3H), 2.11-2.09 (m, 1H), 2.00-1.90 (m, 2H).

### Example 75

### (R)—N-(6-methylfuro[2,3-d]pyrimidin-2-yl)spiro[4H-isoxazole-5,3′-quinuclidine]-3-amine hydrochloride ((R)-75)

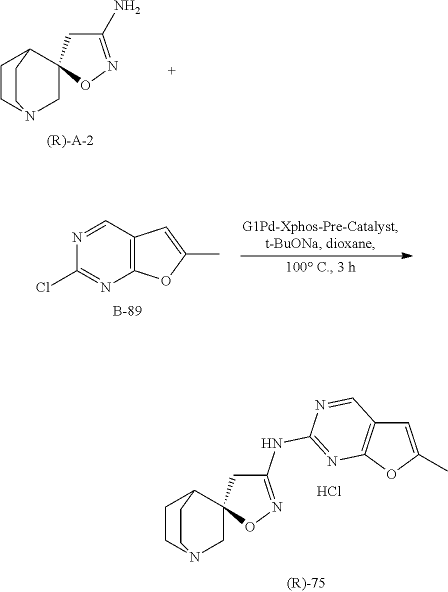

To a mixture of compound B-89 (100 mg, 0.59 mmol) and compound (R)-A-2 (118 mg, 0.65 mmol) in dioxane (2 mL) under nitrogen at room temperature was added sodium tert-butoxide (2 M in THF, 0.6 mL) and dicyclohexyl[2-(2,4,6-triisopropylphenyl)phenyl]phosphane[2-(2-aminoethyl)phenyl]-chloro-palladium (22 mg, 0.030 mmol). The mixture was stirred at 100° C. for 3 hours, then filtered and concentrated in vacuo. The residue was purified by prep-HPLC [Instrument: GX-J; Column: Venusil XBP C8 150×25 mm, particle size: 10 μm; Mobile phase: 22-52% acetonitrile in H₂O (add 0.1% TFA, v/v)]. The combined fractions were treated with 0.2 M hydrochloric acid and lyophilized to give:

Compound (R)-75 (14 mg, 6% yield) as a yellow solid: cSFC analytical (I) tR=2.857 min., purity: 100%; LCMS (FF): tR=1.970 min., (ES⁺) m/z (M+H)⁺=314.1; ¹H-NMR (CD₃OD, 400 MHz): δ 8.81 (s, 1H), 6.76 (s, 1H), 3.77-3.69 (m, 3H),3.60 (d, J=17.2 Hz, 1H), 3.42-3.38 (m, 4H), 2.55 (s, 3H), 2.47-2.40 (m, 2H),2.18-1.92 (m, 3H).

### Example 76

### (R)—N -(3-chlorobenzo[b]thiophen-6-yl)-4H-1′-azaspiro[isoxazole-5,3′-bicyclo[2.2.2]octan]-3-amine hydrochloride ((R)-76)

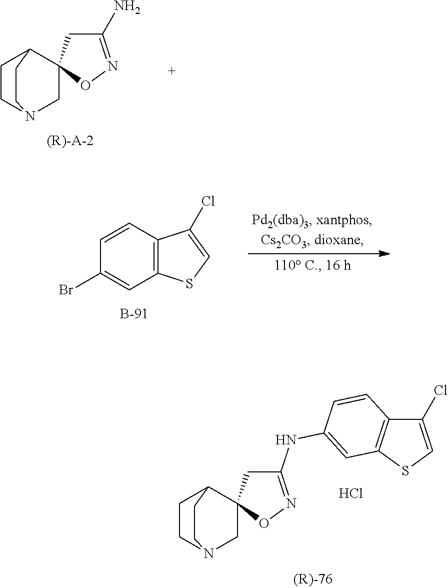

To a mixture of compound B-91 (68 mg, 0.28 mmol), compound (R)-A-2 (50 mg, 0.28 mmol) and cesium carbonate (180 mg, 0.56 mmol) in dioxane (10 mL) under nitrogen at room temperature was added tris(dibenzylideneacetone)dipalladium(0) (25 mg, 0.028 mmol) and 4,5-bis(diphenylphosphino)-9,9-dimethylxanthene (32 mg, 0.055 mmol). The mixture was stirred at 110° C. for 16 hours, then filtered and concentrated in vacuo. The residue was purified by prep-HPLC [Instrument: GX-B; Column: Welch Ultimate AQ-C18 150×30 mm, particle size: 5 μm; Mobile phase: 27-57%]. The solution was treated with 0.2 M hydrochloric acid and lyophilized to give:

Compound (R)-76 (21 mg, 20% yield) as a white solid: cSFC analytical (I) tR=3.350 min., purity: 100%; LCMS (FF): tR=2.441 min., (ES⁺) m/z (M+H)⁺=348.1; ¹H-NMR (DMSO, 400 MHz): δ 10.37 (s, 1H), 9.56 (s, 1H), 8.24 (d, J=1.2 Hz, 1H), 7.71-7.68 (m, 1H), 7.46 (dd, J₁=8.8 Hz, J₂=2.0 Hz, 1H) 3.66-3.62 (m, 1H), 3.15-3.15 (m, 9H), 2.18-2.16 (m, 1H), 1.94-1.93 (m, 1H), 1.82-1.74(m, 2H).

### Example 77

### (R)—N-(3-(trifluoromethyl)benzo[b]thiophen-6-yl)-4H-1′-azaspiro[isoxazole-5,3′-bicyclo[2.2.2]octan]-3-amine hydrochloride ((R)-77)

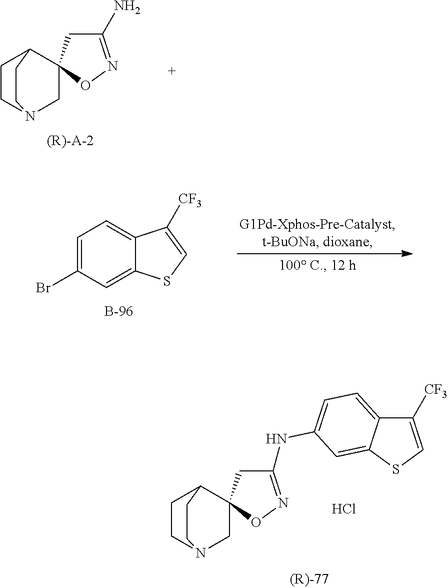

To a solution of compound B-96 (0.47 g, 1.7 mmol), compound (R)-A-2 (0.30 g, 1.7 mmol) and dicyclohexyl[2-(2,4,6-triisopropylphenyl)phenyl]phosphane[2-(2-aminoethyl)phenyl]-chloro-palladium (0.12 g, 0.17 mmol) in dioxane (3 mL) under nitrogen at room temperature was added sodium tert-butoxide (2 M in tetrahydrofuran, 1.7 mL). The mixture was stirred at 100° C. for 12 hours, then poured into water (10 mL) and extracted with ethyl acetate (2×20 mL). The combined organic layers were concentrated in vacuo, and the residue was purified by prep-HPLC [Instrument: GX-J; Column: Welch Ultimate AQ-C18 150×30 mm, particle size: 5 μm; Mobile phase: 25-55% acetonitrile in H₂O (add 0.1% TFA, v/v)]. The combined fractions were treated with 0.2 M hydrochloric acid and lyophilized to give:

Compound (R)-77 (0.10 g, 16% yield) as a brown solid.: cSFC analytical (I) tR=2.541 min., purity: 100%; LCMS (FF): tR=2.516 min., (ES⁺) m/z (M+H)⁺=382.1; ¹H-NMR (CD₃OD, 400 MHz): δ 8.26 (d, J=2.0 Hz, 1H), 8.02 (d, J=0.8 Hz, 1H), 7.78 (d, J=8.8 Hz, 1H), 7.38 (dd, J₁=8.8 Hz, J₂=2.0 Hz, 1H), 3.69 (dd, J₁=14.0 Hz, J₂=2.0 Hz, 1H), 3.61 (d, J=13.2 Hz, 1H), 3.50-3.34 (m, 6H), 2.44-2.38 (m, 2H), 2.12-2.09 (m, 1H), 2.01-1.91 (m, 2H).

### Example 78

### (R)—N-(2-chloro-3-fluorobenzo[b]thiophen-6-yl)-4H-1′-azaspiro[isoxazole-5,3′-bicyclo[2.2.2]octan]-3-amine hydrochloride ((R)−78)

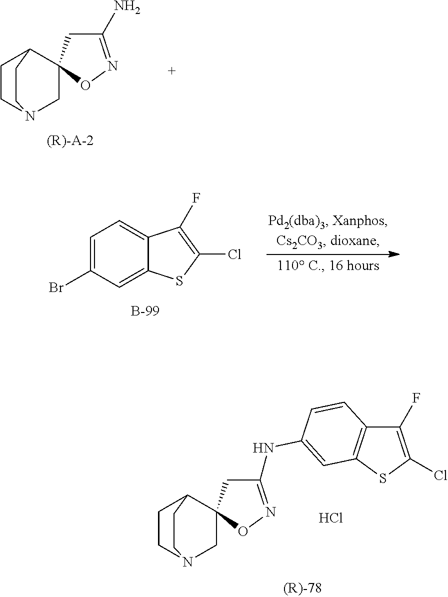

To a solution of compound B-99 (0.20 g, 0.75 mmol), compound (R)-A-2 (0.14 g, 0.75 mmol), tris(dibenzylideneacetone)dipalladium(0) (δ 9 mg, 0.075 mmol) and 4,5-bis(diphenylphosph-ino)-9,9-dimethylxanthene (87 mg, 0.15 mmol) in dioxane (2 mL) under nitrogen at room temperature was added cesium carbonate (0.74 g, 2.3 mmol). The mixture was stirred at 110° C. for 16 hours, then filtered and concentrated in vacuo. The residue was purified by flash column chramotagraphy (Al₂O₃, ethyl acetate: methanol=100:1˜1:1) and prep-HPLC [Instrument: GX-B; Column: Welch Ultimate AQ-C18 150×30 mm, particle size: 5 μm; Mobile phase: 33-63% acetonitrile in H₂O (add 0.1% TFA, v/v)]. The combined fractions were treated with 0.2 N hydrochloric acid and lyophilized to give:

Compound (R)-78 (28 mg, 9% yield) as a white solid: cSFC analytical (I) tR=2.867 min., purity: 99.53%; LCMS (GG): tR=2.211 min., (ES⁺) m/z (M+H)⁺=366.1; ¹H-NMR (CD₃OD, 400 MHz): δ 8.09 (s, 1H), 7.61 (d, J=8.8 Hz, 1H), 7.36 (dd, J₁=8.8 Hz, J₂=2 Hz, 1H), 3.71-3.61 (m, 2H), 3.49-3.33 (m, 6H), 2.47-2.40 (m, 2H), 2.14-2.11 (m, 1H), 2.06-1.93 (m, 2H).

### Example 79

### (R)—N-(quinolin-7-yl)-4H-1′-azaspiro[isoxazole-5,3′-bicyclo[2.2.2]octan]-3-amine hydrochloride ((R)-79)

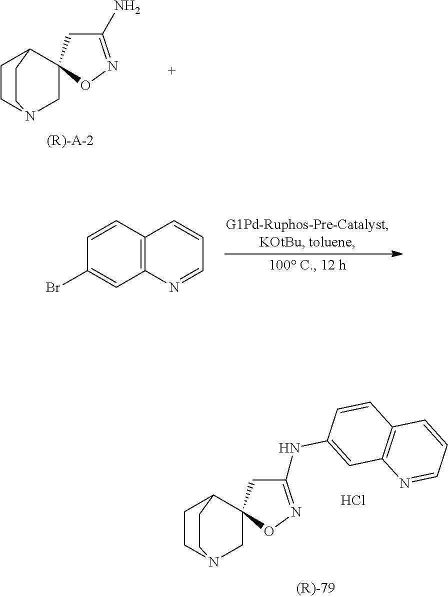 [SMILES: NC1=NO[C@@]2(C1)CN1CCC2CC1 ; Brc1ccc2cccnc2c1 ; c1cnc2cc(NC3=NO[C@@]4(C3)CN3CCC4CC3)ccc2c1 ; Cl]

To a mixture of 7-bromoquinoline (0.20 g, 0.96 mmol) and compound (R)-A-2 (0.21 g, 1.2 mmol) in toluene (2 mL) under nitrogen at room temperature was added potassium tert-butoxide (0.22 g, 1.9 mmol) and chloro-(2-dicyclohexylphosphino-2′, 6′-diisopropoxy-1,1′-biphenyl)[2-(2-aminoethyl)phenyl]palladium(II)-methyl-t-butyl ether adduct (39 mg, 0.048 mmol). The reaction mixture was stirred at 100° C. for 12 hours, then filtered and concentrated in vacuo. The residue was purified by prep-HPLC [Instrument: GX-J; Column: Agela Venusil XBP-C18 150×30 mm, particle size: 5 μm; Mobile phase: 1-30% acetonitrile in H₂O (add 0.1% TFA, v/v)]. The combined fractions were treated with 0.2 M hydrochloric acid and lyophilized to give:

Compound (R)-79 (20 mg, 6% yield) as a yellow solid: cSFC analytical (I) tR=3.066 min., purity: 100.00%; LCMS (GG): tR=1.733 min., (ES⁺) m/z (M+H)⁺=309.1; ¹H-NMR (CD₃OD, 400 MHz): δ 9.01 (d, J=8.0 Hz, 1H), 8.97 (d, J=5.2 Hz, 1H), 8.64 (s, 1H), 8.22 (d, J=8.8 Hz, 1H), 7.85 (t, J=6.8 Hz, 1H), 7.75 (d, J=8.8 Hz, 1H), 3.79-3.75 (m, 1H), 3.69-3.65 (m, 1H), 3.61-3.57 (s, 1H), 3.50-3.38 (m, 5H), 2.48-2.43 (m, 2H), 2.17-1.96 (m, 3H).

### Example 80

### (R)—N-(quinolin-6-yl)-41-1-1¹-azaspiro[isoxazole-5,3′-bicyclo[2.2.2]octan]-3-amine hydrochloride ((R)-80)

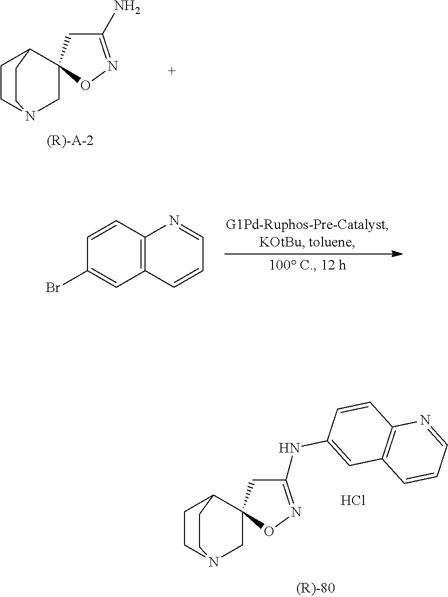 [SMILES: NC1=NO[C@@]2(C1)CN1CCC2CC1 ; Brc1ccc2ncccc2c1 ; c1cnc2ccc(NC3=NO[C@@]4(C3)CN3CCC4CC3)cc2c1 ; Cl]

To a mixture of 6-bromoquinoline (0.12 g, 0.55 mmol) and compound (R)-A-2 (0.12 g, 0.66 mmol) in toluene (2 mL) under nitrogen at room temperature was added potassium tert-butoxide (0.12 g, 1.1 mmol) and chloro-(2-dicyclohexylphosphino-2′, 6′-diisopropoxy-1,1′-biphenyl)[2-(2-aminoethyl)phenyl]palladium(II)-methyl-t-butyl ether adduct (22 mg, 0.027 mmol). The reaction mixture was stirred at 100° C. for 12 hours, then filtered and concentrated in vacuo. The residue was purified by prep-HPLC [Instrument: GX-J; Column: Agela Venusil XBP-C18 150×25 mm, particle size: 5 μm; Mobile phase: 1-30% acetonitrile in H₂O (add 0.1% TFA, v/v)]. The combined fractions were treated with 0.2 M hydrochloric acid and lyophilized to give:

Compound (R)-80 (15 mg, 8% yield) as a yellow solid: cSFC analytical (I) tR=3.412 min., purity: 100.00%; LCMS (GG): tR=1.721 min., (ES⁺) m/z (M+H)⁺=309.1; ¹H-NMR (CD₃OD, 400 MHz): δ 9.04 (d, J=8.8 Hz, 1H), 9.00 (d, J=5.2 Hz, 1H), 8.58 (s, 1H), 8.18 (d, J=9.2 Hz, 1H), 8.07-8.01 (m, 2H), 3.77-3.65 (m, 2H), 3.58-3.54 (m, 1H), 3.50-3.37 (m, 5H), 2.48-2.42 (m, 2H), 2.17-1.12 (m, 1H), 2.08-1.93 (m, 2H).

### Example 81

### (R)—N-(6-chloro-7-fluoroisoquinolin-3-yl)-4H-1′-azaspiro[isoxazole-5,3¹-bicyclo[2.2.2]octan]-3-amine hydrochloride ((R)-81)

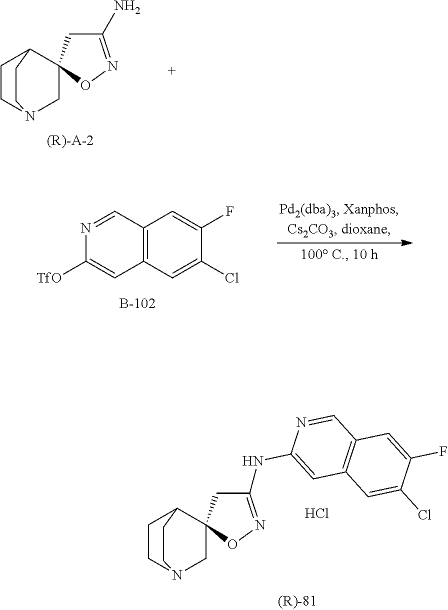 [SMILES: NC1=NO[C@@]2(C1)CN1CCC2CC1 ; O=S(=O)(Oc1cc2cc(Cl)c(F)cc2cn1)C(F)(F)F ; Fc1cc2cnc(NC3=NO[C@@]4(C3)CN3CCC4CC3)cc2cc1Cl ; Cl]

A mixture of compound B-102 (0.36 g, 0.83 mmol), compound (R)-A-2 (0.15 g, 0.83 mmol), tris(dibenzylideneacetone)dipalladium(0) (0.08 g, 0.08 mmol), cesium carbonate (0.54 g, 1.7 mmol) and 4,5-bis(diphenylphosphino)-9,9-dimethylxanthene (0.10 g, 0.17 mmol) in dioxane (5 mL) under nitrogen was stirred at 100° C. for 10 hours. On completion, the mixture was filtered and concentrated in vacuo. The residue was purified by prep-HPLC [Instrument: GX-B; Column: Welch Ultimate AQ-C18 150×30 mm, particle size: 5 μm; Mobile phase: 24-54% acetonitrile in H₂O (add 0.1% TFA, v/v)]. The combined fractions were treated with 0.2N hydrochloric acid and lyophilized to give:

Compound (R)-81 (46 mg, 15% yield) as a yellow solid.: cSFC analytical (P) tR=3.29 min., purity: 100%; LCMS (GG): tR=2.084 min., (ES⁺) m/z (M+H)⁺=361.1; ¹H-NMR (CD₃OD, 400 MHz): δ 9.21 (s, 1H), 8.16 (d, J=7.2 Hz, 1H), 8.00 (d, J=4.8 Hz, 1H), 7.94 (s, 1H), 3.78 (d, J=12.4 Hz, 1H), 3.76-3.62 (m, 2H), 3.55-3.37 (m, 5H), 2.45-2.42 (m, 2H), 2.14-2.05 (m, 1H), 2.03-1.96 (m, 2H).

### Example 82

### (R)—N-(7-chloro-6-fluoroisoquinolin-3-yl)-4H-1′-azaspiro[isoxazole-5,3′-bicyclo[2.2.2]octan]-3-amine hydrochloride ((R)-82)

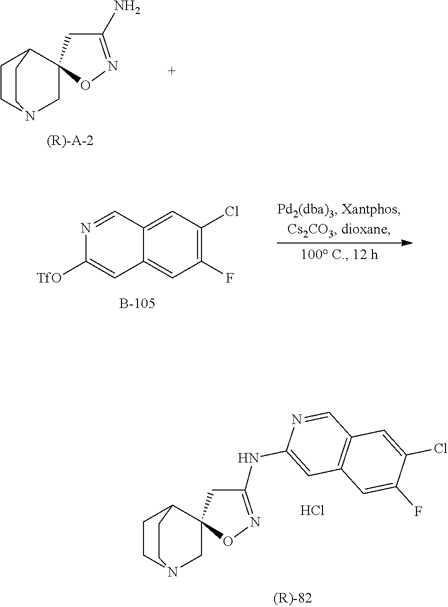 [SMILES: NC1=NO[C@@]2(C1)CN1CCC2CC1 ; O=S(=O)(Oc1cc2cc(F)c(Cl)cc2cn1)C(F)(F)F ; Fc1cc2cc(NC3=NO[C@@]4(C3)CN3CCC4CC3)ncc2cc1Cl ; Cl]

To a solution of compound B-105 (0.36 g, 1.1 mmol), compound (R)-A-2 (0.20 g, 1.1 mmol), tris(dibenzylideneacetone)dipalladium(0) (0.10 g, 0.11 mmol), and 4,5-bis(diphenylphosphino)-9,9-dimethylxanthene (0.13 g, 0.22 mmol) in dioxane (3 mL) under nitrogen at room temperature was added cesium carbonate (0.72 g, 2.2 mmol). The reaction mixture was stirred at 100° C. for 12 hours, then poured into water (10 mL) and extracted with ethyl acetate (2×20 mL). The combined organic layers were concentrated in vacuo. The residue was purified by prep-HPLC [Instrument: GX-J; Column: Welch Ultimate AQ-C18 150×30 mm, particle size: 5 μm; Mobile phase: 28-58% acetonitrile in H₂O (add 0.1% TFA, v/v)]. The combined fractions were treated with 0.2 M hydrochloric acid and lyophilized to give:

Compound (R)-82 (64 mg, 16% yield) as a yellow solid.: cSFC analytical (I) tR=3.253 min., purity: 100%; LCMS (FF): tR=2.378 min., (ES⁻) m/z (M+H)⁺=361.1; ¹H-NMR (CD₃OD, 400 MHz): δ 9.15 (s, 1H), 8.34 (d, J=7.2 Hz, 1H), 7.96 (s, 1H), 7.76 (d, J=10.0 Hz, 1H), 3.76-3.66 (m, 2H), 3.63-3.59 (m, 1H), 3.50-3.38 (m, 5H), 2.43-2.40 (m, 2H), 2.17-2.11 (m, 1H), 2.04-1.92 (m, 2H).

### Example 83

### (R)—N-(6-(trifluoromethypisoquinolin-3-yl)-4H-1′-azaspiro[isoxazole-5,3′-bicyclo[2.2.2]octan]-3-amine hydrochloride ((R)-83)

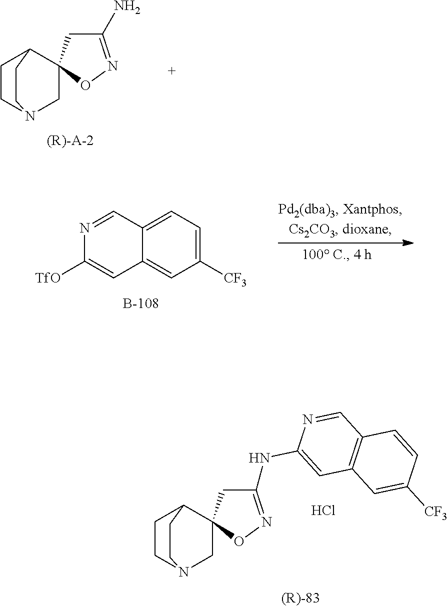 [SMILES: NC1=NO[C@@]2(C1)CN1CCC2CC1 ; O=S(=O)(Oc1cc2cc(C(F)(F)F)ccc2cn1)C(F)(F)F ; FC(F)(F)c1ccc2cnc(NC3=NO[C@@]4(C3)CN3CCC4CC3)cc2c1 ; Cl]

To a solution of compound B-108 (0.38 g, 1.1 mmol), compound (R)-A-2 (0.20 g, 1.1 mmol), tris(dibenzylideneacetone)dipalladium(0) (0.10 g, 0.11 mmol) and 4,5-bis(diphenylphosphino)-9,9-dimethylxanthene (0.13 g, 0.22 mmol) in dioxane (4 mL) under nitrogen at room temperature was added cesium carbonate (0.72 g, 2.2 mmol). The reaction mixture was stirred at 100° C. for 4 hours, then poured into water (10 mL) and extracted with ethyl acetate (2×30 mL). The combined organic layers were concentrated in vacuo and purified by prep-HPLC [Instrument: GX-B; Column: Welch Ultimate AQ-C18 150×30 mm, particle size: 5 μm; Mobile phase: 24-54% acetonitrile in H₂O (add 0.1% TFA, v/v)]. The combined fractions were treated with 0.2 M hydrochloric acid and lyophilized to give:

Compound (R)-83 (52 mg, 13% yield) as a yellow solid.: cSFC analytical (I) tR=2.516 min., purity: 100%; LCMS (FF): tR=2.477 min., (ES⁻) m/z (M+H)⁺=377.1; ¹H-NMR (CD₃OD, 400 MHz): δ 9.35 (s, 1H), 8.33 (d, J=8.0 Hz, 1H), 8.32 (s, 1H), 8.13 (s, 1H), 7.76 (d, J=9.6 Hz, 1H), 3.79- 3.64 (m, 3H), 3.56-3.34 (m, 5H), 2.46-2.40 (m, 2H), 2.16-1.96 (m, 3H).

### Example 84

### (R)—N-(2,2-difluorobenzo[d] [1,3]dioxo1-5-yl)-4H-1′-azaspiro[isoxazole-5,3′-bicyclo[2.2.2]octan]-3-amine hydrochloride ((R)-84)

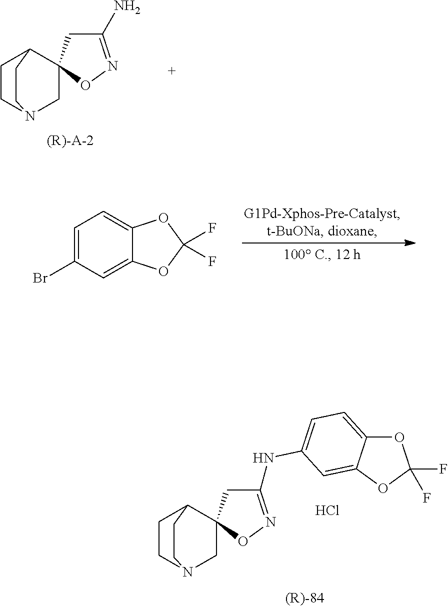 [SMILES: NC1=NO[C@@]2(C1)CN1CCC2CC1 ; FC1(F)Oc2ccc(Br)cc2O1 ; FC1(F)Oc2ccc(NC3=NO[C@@]4(C3)CN3CCC4CC3)cc2O1 ; Cl]

To a solution of 5-bromo-2,2-difluorobenzo[d][1,3]dioxole (0.39 g, 1.7 mmol), compound (R)-A-2 (0.30 g, 1.7 mmol) and dicyclohexyl[2-(2,4,6-triisopropylphenyl)phenyl]phosphane8 2-(2-aminoethyl)phenyl]-chloro-palladium (0.12 g, 0.17 mmol) in dioxane (4 mL) under nitrogen at room temperature was added sodium tert-butoxide (2 M in tetrahydrofuran, 1.7 mL). The mixture was stirred at 100° C. for 12 hours, then poured into water (5 mL) and extracted with ethyl acetate (2×10 mL). The combined organic layers were concentrated and purified by prep-HPLC [Instrument: GX-I; Column: YMC-Actus ODS-AQ 150×30 mm, particle size: 5 μm; Mobile phase: 15-45% acetonitrile in H₂O (add 0.1% TFA, v/v)]. The combined fractions were treated with 0.2 M hydrochloric acid and lyophilized to give:

Compound (R)-84 (0.11 g, 20% yield) as a yellow solid. : cSFC analytical (I) tR=2.377 min., purity: 100%; LCMS (GG): tR=2.008 min., (ES⁺) m/z (M+H)⁺=338.1; ¹H-NMR (CD₃OD, 400 MHz): δ 7.47 (s, 1H), 7.09 (s, 2H), 3.68 (dd, J₁=14.0 Hz, J₂=2.0 Hz, 1H), 3.59 (d, J=14.0 Hz, 1H), 3.45-3.33 (m, 6H), 2.42-2.36 (m, 2H), 2.14-2.07 (m, 1H), 2.00-1.89 (m, 2H).

### Example 85

### The compounds in Table 2 Were Made According to the Following General Procedure

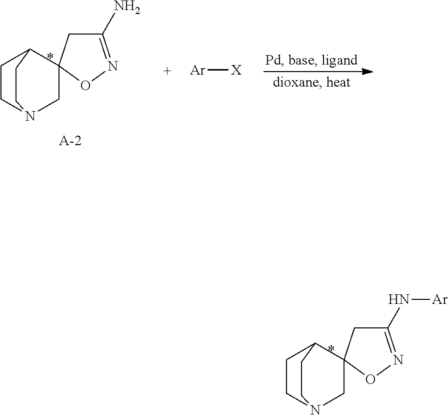

A mixture of aryl halide (1-2 eq.), compound A-2 (racemic or a single enantiomer, 1 eq.), palladium complex (0.05-0.10 eq.), ligand (0.05-0.10 eq), and base (1-2 eq.) in dioxane (5-10 mL/mmol) under nitrogen was heated with stirring at the indicated temperature for the indicated time. On completion, the mixture was filtered and concentrated under vacuum. The residue was purified by prep HPLC or silica gel chromatography, and mixtures of enantiomers were separated by chiral SFC. Most compounds were treated with 0.2 M hydrochloric acid and lyophilized to give hydrochloride salts.

TABLE 2

| | | | | | | MS |
|---|---|---|---|---|---|---|
| | | | | Analytical | tR | (M + |
| Compund | Name | X | Conditions | conditions | (min) | H)⁺ |
| (R)-85 | (R)-N-(5,6,7,8-tetrahydroisoquinolin-3-yl)-4H-1′-azaspiro[isoxazole- | Br | Pd₂(dba)₃, Xantphos, | cHPLC (1) | 25.73 | 313.2 |
| | 5,3′-bicyclo[2.2.2]octan]-3-amine | | NaOtBu, 100° C., 1.5 h | | | |
| (S)-85 | (S)-N-(5,6,7,8-tetrahydroisoquinolin-3-yl)-4H-1′-azaspiro[isoxazole- | Br | Pd₂(dba)₃, Xantphos, | cHPLC (1) | 17.78 | 313.2 |
| | 5,3′-bicyclo[2.2.2]octan]-3-amine | | NaOtBu, 100° C., 1.5 h | | | |
| 86-P1 | N-(5-methoxypyrimidin-2-yl)-4H-1′-azaspiro[isoxazole-5,3′- | Cl | Pd₂(dba)₃, X-phos, | cSFC | 2.48 | 290.1 |
| | bicyclo[2.2.2]octan]-3-amine-enantiomer1 hydrochloride | | NaOtBu, 106° C., 12 h | analytical (D) | | |
| 86-P2 | N-(5-methoxypyrimidin-2-yl)-4H-1′-azaspiro[isoxazole-5,3′- | Cl | Pd₂(dba)₃, X-phos, | cSFC | 3.16 | 290.1 |
| | bicyclo[2.2.2]octan]-3-amine-enantiomer1 hydrochloride | | NaOtBu, 106° C., 12 h | analytical (D) | | |
| 87-P1 | N-(furo[3,2-b]pyridin-5-yl)-4H-1′-azaspiro[isoxazole-5,3′- | Cl | Pd₂(dba)₃, X-phos, | cSFC | 3.05 | 299.1 |
| | bicyclo[2.2.2]octan]-3-amine-enantiomer1 hydrochloride | | NaOtBu, 106° C., 12 h | analytical (H) | | |
| 87-P2 | N-(furo[3,2-b]pyridin-5-yl)-4H-1′-azaspiro[isoxazole-5,3′- | Cl | Pd₂(dba)₃, X-phos, | cSFC | 3.33 | 299.1 |
| | bicyclo[2.2.2]octan]-3-amine-enantiomer2 hydrochloride | | NaOtBu, 106° C., 12 h | analytical (H) | | |
| 88-P1 | 2-((4H-1′-azaspiro[isoxazole-5,3′-bicyclo[2.2.2]octan]-3- | Br | G1Pd-Xphos-Pre-Catalyst, | cSFC | 2.46 | 340.1 |
| | yl)amino)benzo[d]thiazole-6-carbonitrile hydrochloride | | NaOtBu, 110° C., 16 h | analytical (L) | | |
| 88-P2 | 2-((4H-1′-azaspiro [isoxazole-5,3′-bicyclo[2.2.2]octan]-3- | Br | G1Pd-Xphos-Pre-Catalyst, | cSFC | 5.05 | 340.1 |
| | yl)amino)benzo[d]thiazole-6-carbonitrile hydrochloride | | NaOtBu, 110° C., 16 h | analytical (L) | | |
| 89-P1 | N-(6-(trifluoromethyl) benzo[d]thiazol-2-yl)-4H-1′-azaspiro[isoxazole- | Cl | G1Pd-Xphos-Pre-Catalyst, | cSFC | 7.49 | 383.0 |
| | 5,3′-bicyclo[2.2.2]octan]-3-amine-enantiomer1 hydrochloride | | NaOtBu, 100° C., 2 h | analytical (J) | | |
| 89-P2 | N-(6-(trifluoromethyl benzo[d]thiazol-2-yl)-4H-1′-azaspiro[isoxazole- | Cl | G1Pd-Xphos-Pre-Catalyst, | cSFC | 10.64 | 383.0 |
| | 5,3′-bicyclo[2.2.2]octan]-3-amine-enantiomer2 hydrochloride | | NaOtBu, 100° C., 2 h | analytical (J) | | |
| 90-P1 | N-(quinolin-2-yl)-4H-1′-azaspiro[isoxazole-5,3′-bicyclo[2.2.2]octan]-3- | Cl | G1Pd-Xphos-Pre-Catalyst, | cSFC | 1.26 | 309.2 |
| | amine-enantiomer1 hydrochloride | | Cs₂CO₃, 80° C., 16 h | analytical (H) | | |
| 90-P2 | N-(quinolin-2-yl)-4H-1′-azaspiro[isoxazole-5,3′-bicyclo[2.2.2]octan]-3- | Cl | G1Pd-Xphos-Pre-Catalyst, | cSFC | 1.98 | 309.2 |
| | amine-enantiomer2 hydrochloride | | Cs₂CO₃, 80° C., 16 h | analytical (H) | | |
| 91-P1 | N-(pyridin-2-yl)-4H-1′-azaspiro[isoxazole-5,3′-bicyclo[2.2.2]octan]-3- | Br | G1Pd-Xphos-Pre-Catalyst, | cSFC | 2.80 | 259.1 |
| | amine-enantiomer1 hydrochloride | | NaOtBu, 80° C., 16 h | analytical (D) | | |
| 91-P2 | N-(pyridin-2-yl)-4H-1′-azaspiro[isoxazole-5,3′-bicyclo[2.2.2]octan]-3- | Br | G1Pd-Xphos-Pre-Catalyst, | cSFC | 3.08 | 259.1 |
| | amine-enantiomer2 hydrochloride | | NaOtBu, 80° C., 16 h | analytical (D) | | |
| (R)-92 | (R)-N-(4,5,6-trifluorobenzo[d]thiazol-2-yl)-4H-1′-azaspiro[isoxazole- | Br | G1Pd-Xphos-Pre-Catalyst, | cSFC | 2.66 | 369.0 |
| | 5,3′-bicyclo[2.2.2]octan]-3-amine hydrochloride | | NaOtBu, 50° C., 2 h | analytical (H) | | |
| (R)-93 | (R)-N-(3,4,5-trichlorophenyl)-4H-1′-azaspiro[isoxazole-5,3′- | Br | Pd₂(dba)₃, Xantphos, | cSFC | 2.80 | 360.0 |
| | bicyclo[2.2.2]octan]-3-amine hydrochloride | | Cs₂CO₃, 100° C., 3 h | analytical (I) | | |
| (R)-94 | (R)-N-(4-cyclopropyl-3-methoxyphenyl)-4H-1′-azaspiro[isoxazole-5,3′- | Cl | G1Pd-Xphos-Pre-Catalyst, | cSFC | 3.05 | 328.2 |
| | bicyclo[2.2.2]octan]-3-amine hydrochloride | | NaOtBu, 80° C., 3 h | analytical (P) | | |
| (R)-95 | (R)-N-(quinolin-3-yl)-4H-1′-azaspiro[isoxazole-5,3′- | Br | Pd₂(dba)₃, Xantphos, | cSFC | 2.59 | 309.1 |
| | bicyclo[2.2.2]octan]-3-amine hydrochloride | | Cs₂CO₃, 110° C., 2 h | analytical (I) | | |
| (R)-96 | (R)-N-(2-methylimidazo[1,2-a]pyridin-7-yl)-4H-1′-azaspiro[isoxazole- | Br | Pd₂(dba)₃, Xantphos, | cSFC | 3.09 | 312.1 |
| | 5,3′-bicyclo[2.2.2]octan]-3-amine hydrochloride | | Cs₂CO₃, 100° C., 16 h | analytical (P) | | |
| (R)-97 | (R)-N-(1,2-dimethyl-1H-benzo[d]imidazol-5-yl)-4H-1′-azaspiro[isoxazole- | Br | G1Pd-Xphos-Pre-Catalyst, | cSFC | 2.90 | 326.2 |
| | 5,3′-bicyclo[2.2.2]octan]-3-amine hydrochloride | | NaOtBu, 100° C., 12 h | analytical (I) | | |
| 98-P1 | N-(furo[3,2-c]pyridin-5-yl)-4H-1′-azaspiro[isoxazole-5,3′- | Cl | Pd₂(dba)₃, X-phos, | cSFC | 5.92 | 299.1 |
| | bicyclo[2.2.2]octan]-3-amine-enantiomer1 hydrochloride | | NaOtBu, 106° C., 12 h | analytical (D) | | |
| 98-P2 | N-(furo[3,2-c]pyridin-5-yl)-4H-1′-azaspiro[isoxazole-5,3′- | Cl | Pd₂(dba)₃, X-phos, | cSFC | 7.22 | 299.1 |
| | bicyclo[2.2.2]octan]-3-amine-enantiomer2 hydrochloride | | NaOtBu, 106° C., 12 h | analytical (D) | | |
| (R)-99 | (R)-N-(2-methylthieno[3,2-c]pyridin-6-yl)spiro[4H-isoxazole-5,3′- | Cl | G1Pd-Xphos-Pre-Catalyst, | cSFC | 3.19 | 329.1 |
| | quinuclidine]-3-amine hydrochloride | | NaOtBu, 120° C., 2 h | analytical (I) | | |
| (R)-100 | (R)-N-(2-methylfuro[3,2-c]pyridin-6-yl)spiro[4H-isoxazole-5,3′- | Cl | G1Pd-Xphos-Pre-Catalyst, | cSFC | 0.81 | 313.1 |
| | quinuclidine]-3-amine hydrochloride | | NaOtBu, 100° C., 2 h | analytical (I) | | |

Crystallization experiments

### Example 86

### (S)-4H-1′-azaspiro[isoxazole-5,3′-bicyclo[2.2.2]octan]-3-amine (2R,3R)-2,3-dihydroxysuccinate ((S)-A-2 L tartaric acid salt)

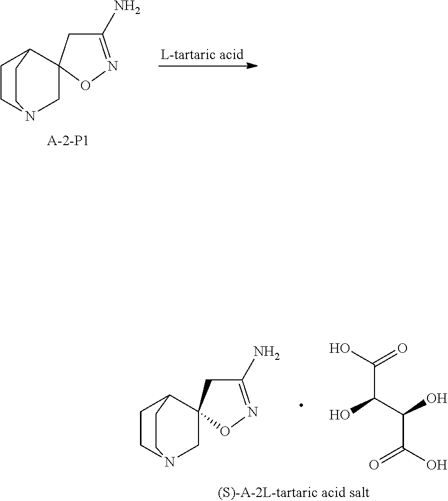 [SMILES: NC1=NOC2(C1)CN1CCC2CC1 ; NC1=NO[C@]2(C1)CN1CCC2CC1 ; O=C(O)[C@H](O)[C@@H](O)C(=O)O]

A solution of (2R,3R)-2,3-dihydroxysuccinic acid (86 mg, 0.6 mmol) in methanol (2 mL) was added to a solution of compound A-2-P1 (104 mg, 0.6 mmol) in methanol (2 mL) and water (2 mL). After standing overnight, the crystals were collected by filtration, washed with MeOH and air dried to afford compound (S)-A-2 L-tartaric acid salt (113 mg, 59% yield) as white crystals. ¹H NMR (300 MHz, D₂O) δ 4.28-4.17 (m, 2H), 3.28-3.11 (m, 3H), 3.07-2.81 (m, 5H), 2.14-2.08 (m, 1H), 2.05-1.90 (m, 1H), 1.90-1.75 (m, 1H), 1.75-1.55 (m, 2H); mp: 205° C. (decomp.). X-ray analysis showed the absolute configuration of compound A-2-P1 to be S.

Single-crystal diffraction was performed on a Nonius KappaCCD single-crystal diffractometer using graphite monochromated Mo Ka radiation. During the measurement the crystal was cooled to −65° C. Diffraction images were integrated using Eval14. Intensity data were corrected for Lorentz and polarization effects. A semi empirical multi scan absorption correction was applied (SADABS).

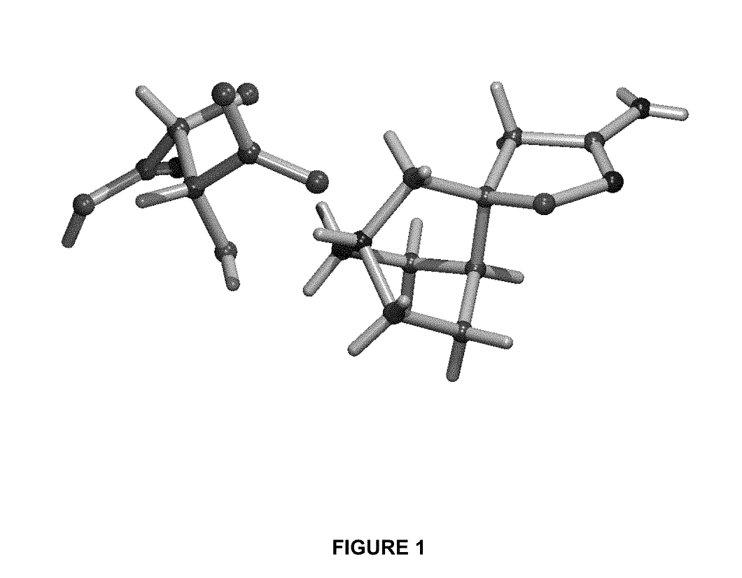

Refinement was performed with standard methods (refinement against F2 of all reflections with SHELXL97) with anisotropic displacement parameters for the non-hydrogen atoms. All hydrogen atoms were placed at calculated positions and refined riding on the parent atoms. The right enantiomer was determined by careful examination of the Bijvoet pairs. Coordinate data from the X-ray analysis of the formed crystal of compound (S)-A-2 L-tartaric acid salt are shown in Table 3, and its 3-D representation is shown in FIG. 1.

TABLE 3
X-ray Data:
Unit cell: 9.165 10.474 14.889 90.000 90.000 90.000
Space group: P 21 21 21

| | | | |
|---|---|---|---|
| O003 | 0.74942(10) | 0.58306(7) | 0.46092(5) |
| N008 | 0.74271(10) | 0.67478(9) | 0.24342(6) |
| N009 | 0.79099(11) | 0.61198(9) | 0.55225(6) |
| N00A | 0.81260(11) | 0.79690(11) | 0.63895(6) |
| C00C | 0.79191(11) | 0.73540(10) | 0.56061(6) |
| C00F | 0.77836(10) | 0.69584(9) | 0.40702(6) |
| C00H | 0.93193(10) | 0.68742(10) | 0.36639(6) |
| C00I | 0.95311(11) | 0.79701(11) | 0.29946(7) |
| C00J | 0.76188(12) | 0.80471(9) | 0.47423(7) |
| C00K | 0.94802(12) | 0.56083(11) | 0.31590(7) |
| C00L | 0.84122(13) | 0.78563(11) | 0.22345(8) |
| C00M | 0.66691(11) | 0.69589(11) | 0.33054(7) |
| C00N | 0.82770(14) | 0.55234(11) | 0.24472(9) |
| O001 | 0.59787(9) | 0.62815(8) | 0.08693(6) |
| O002 | 0.22327(9) | 0.57772(9) | 0.15078(6) |
| O004 | 0.53472(10) | 0.83463(9) | 0.04852(5) |
| O005 | 0.43260(11) | 0.82455(8) | 0.18356(5) |
| O006 | 0.43285(11) | 0.51536(9) | 0.21135(6) |
| O007 | 0.61980(11) | 0.93711(9) | 0.07305(6) |
| C00B | 0.36138(11) | 0.57389(9) | 0.15384(6) |
| C00D | 0.53610(12) | 0.86280(9) | 0.03676(6) |
| C00E | 0.44515(11) | 0.64758(9) | 0.08062(6) |
| C00G | 0.42062(11) | 0.79173(9) | 0.09132(6) |
| H1 | 1.005(2) | 0.6973(19) | 0.4145(12) |
| H2 | 1.049(2) | 0.7902(18) | 0.2743(12) |
| H3 | 0.659(2) | 0.8380(19) | 0.4757(13) |
| H5 | 0.602(2) | 0.625(2) | 0.3398(14) |
| H6 | 1.042(2) | 0.5589(19) | 0.2869(13) |
| H8 | 0.620(2) | 0.7769(19) | 0.3258(13) |
| H9 | 0.826(2) | 0.873(2) | 0.4658(14) |
| H10 | 0.782(3) | 0.853(2) | 0.2211(15) |
| H11 | 0.939(2) | 0.8797(19) | 0.3292(13) |
| H12 | 0.945(3) | 0.487(2) | 0.3566(15) |
| H13 | 0.831(3) | 0.880(3) | 0.6336(18) |
| H14 | 0.864(2) | 0.5420(19) | 0.1844(13) |
| H15 | 0.890(2) | 0.768(2) | 0.1660(15) |
| H16 | 0.678(2) | 0.665(2) | 0.1992(14) |
| H17 | 0.836(3) | 0.761(2) | 0.6821(16) |
| H19 | 0.752(3) | 0.490(3) | 0.2568(15) |
| H4 | 0.328(2) | 0.8138(19) | 0.0683(12) |
| H7 | 0.409(2) | 0.6222(19) | 0.0205(13) |
| H18 | 0.618(3) | 0.562(3) | 0.0799(16) |
| H20 | 0.492(2) | 0.887(2) | 0.1898(14) |
| H21 | 0.622(4) | 0.859(3) | 0.085(2) |

### Example 87

Human α7 nAChR Binding Assay

The ability of compounds to displace binding of radioactive ligands from human α7 nAChR was determined, as a measure of the affinity of the compounds for these ligand-gated ion channels. The [¹²⁵I]-αBungarotoxin competition binding assay was performed under contract by Cerep Poitiers, France following published the methods (Sharples et al., J Neurosci. 2000; 20(8):2783-91). “SH-SY5Y cells stably expressing human α7 nicotinic acetylcholine receptors, grown to confluency in 175 cm² flasks, were washed briefly with warm PBS containing (in mm): (150 NaCl, 8 K₂HPO₄, 2 KH₂PO₄, pH 7.4, 37° C.) and scraped into cold phosphate buffer. Cells were washed by centrifugation for 3 min at 500× g and resuspended in 10 mL of ice-cold phosphate buffer. The suspension was homogenized for 10 sec using an Ultraturax and centrifuged for 30 min at 45,000×g. The pellet was resuspended in phosphate buffer (0.5 mL per original flask). SH-SY5Y membranes (30 μg protein) were incubated in a total volume of 2 mL in 50 mM phosphate buffer with 0.05 nM [¹²⁵I]-αBgt and serial dilutions of test compound. Nonspecific binding was determined in the presence of a-bungarotoxin (1 μM). Samples were incubated for 120 min at 37° C. The reaction was terminated by filtration through Whatman GFA/E filter paper (presoaked overnight in 0.3% polyethyleneimine in PBS), using a Brandel Cell Harvester. Each condition was measured in duplicate. Filters were counted for radioactivity using a scintillation counter. The results were expressed as a percent inhibition of control specific binding obtained in the presence of the test compounds where Inhibition (%)=100−[(measured specific binding/control specific binding)×100].

The IC₅₀ values (concentration causing a half-maximal inhibition of control specific binding) and Hill coefficients (nH) were determined by non-linear regression analysis of the competition curves generated with mean replicate values using Hill equation:

$$Y = D + [ \frac{A - D}{1 + {( C / {C}_{50} )}^{nH}} ]$$

where Y=specific binding, A=left asymptote of the curve, D=right asymptote of the curve, C=compound concentration, C₅₀=IC₅₀, and nH=slope factor.

This analysis was performed using software developed at Cerep (Hill software) and validated by comparison with data generated by the commercial software SigmaPlot® 4.0 for Windows® (0 1997 by SPSS Inc.). The inhibition constants (K_{;}) were calculated using the Cheng Prusoff equation:

$${K}_{i} = \frac{{IC}_{50}}{( 1 + L / {K}_{D} )}$$

where L=concentration of radioligand in the assay, and K_{D}=affinity of the radioligand for the receptor.

A scatchard plot is used to determine the K_{D}. Results are provided in Table 4 (reported as h-α7 Ki (μM)).

[³H]BRL 43694 competition binding (h-5HT₃ Ki (μM))

[³H]BRL 43694competition binding assay was performed under contract by Cerep Poitiers, France following the methods described in Hope, A.G et al., “Characterization of a human 5-hydroxytryptamine3 receptor type A (h5-HT3R-AS) subunit stably expressed in HEK 293 cells,” Brit. J. Pharmacol., (1996) 118: 1237-1245.

In brief, Chinese Hamster Ovary (CHO) cells stably expressing human 5-HT₃ serotonin receptors, grown to confluence in 175 cm² flasks. Following aspiration of the culture medium, cells were harvested by mechanical agitation in ice cold PBS containing (in mM): (150 NaCl, 8 K₂HPO₄, 2 KH₂PO₄, pH 7.4, 37° C.), centrifuged at 4,000 g for 10 min and subsequently stored as a cell pellet at −80 C. When required, the pellet was thawed and resuspended in ice cold homogenization buffer (Tris 50 mM, EGTA 5.0 mM, phenylmethylsulphonylfluoride 0.1 mM, pH 7.6) and homogenized. The homogenate was centrifuged at 48,000 g for 10 minutes at 40° C. The resulting pellet was resuspended in ice cold binding buffer comprising (in mM): NaCl 140, KCl2.8, CaCl₂ 1.0; MgCl₂, 2.0; HEPES 10 (pH 7.4) and centrifuged as above. The pellet was resuspended in ice cold binding buffer and the protein concentration was determined by the method of Lowry et al., “Protein measurement with the Folin phenol reagent,” J. Biol. Chem., (1953) 193, 265-275). The membrane homogenate was adjusted to a protein concentration of approximately 600 mg/mL in binding buffer. Assay tubes were loaded with equal volumes of binding buffer containing [³H]BRL 43694 and test compound and 0.5 mL of membrane homogenate in a total reaction volume of 1 ml. Binding was initiated by the addition of the membrane homogenate and allowed to proceed for 120 min. at room temperature. Bound and free radioligand were separated by the addition of 3 ml of ice-cold binding buffer and immediate vacuum filtration through pre-soaked (0.1% (v/v) polyethyleneimine) Whatman GF/B filters. Filters were washed with a further 2×3 mL applications of binding buffer and counted for radioactivity using a scintillation counter.

The results were expressed as a percent inhibition of control specific binding obtained in the presence of the test compounds where Inhibition (%)=100−[(measured specific binding/control specific binding)×100].

The IC₅₀ values (concentration causing a half-maximal inhibition of control specific binding) and Hill coefficients (nH) were determined by non-linear regression analysis of the competition curves generated with mean replicate values using Hill equation

$$Y = D + [ \frac{A - D}{1 + {( C / {C}_{50} )}^{nH}} ]$$

where Y=specific binding, A=left asymptote of the curve, D=right asymptote of the curve, C=compound concentration, C₅₀=IC₅₀, and nH=slope factor. This analysis was performed using software developed at Cerep (Hill software) and validated by comparison with data generated by the commercial software SigmaPlot® 4.0 for Windows® (© 1997 by SPSS Inc.).

The inhibition constants (Kᵢ) were calculated using the Cheng Prusoff equation

$${K}_{i} = \frac{{IC}_{50}}{( 1 + L / {K}_{D} )}$$

where L=concentration of radioligand in the assay, and K_{D}=affinity of the radioligand for the receptor.

A scatchard plot is used to determine the K_{D}. Results are provided in Table 4 (reported as h-5HT₃ Ki (μM)).

TABLE 4

| | h- α7 | h-5HT₃ |
|---|---|---|
| Compound | Kᵢ (μM) | Kᵢ (μM) |
| 1-P1 | 1 | 0.0084 |
| 1-P2 | >30 | |
| 2-P1 | 6.7 | |
| 2-P2 | 0.12 | 0.81 |
| 3-P1 | 6.1 | |
| 3-P2 | 0.077 | 0.35 |
| 4-P1 | 3.2 | |
| 4-P2 | 0.13 | >10 |
| 5-P1 | 1 | 0.75 |
| 5-P2 | >30 | |
| 6-P1 | >30 | |
| 6-P2 | 18 | |
| 7-P1 | >30 | |
| 7-P2 | 10 | |
| (S)-8 | 17 | |
| (R)-8 | 0.62 | |
| 9-P1 | 6.3 | |
| 9-P2 | 0.046 | >10 |
| 10-P1 | 1.6 | |
| 10-P2 | 0.071 | >10 |
| (S)-11 | 5.4 | 0.13 |
| (R)-11 | 0.21 | 0.305 |
| rac-12 | 0.062 | 1.8 |
| 13-P1 | 0.23 | 2.4 |
| 13-P2 | 9.3 | |
| 14-P1 | 0.0675 | |
| 14-P2 | 2.4 | |
| 15-P1 | 0.16 | >10 |
| 15-P2 | 3.3 | |
| 16-P1 | 0.63 | |
| 16-P2 | 9.2 | |
| (R)-17 | 0.34 | |
| (R)-18 | 0.175 | >10 |
| 19-P1 | 3 | |
| 19-P2 | 0.19 | >10 |
| (R)-19 | 0.143 | >10 |
| 20-P1 | 0.200 | 0.069 |
| 20-P2 | 8.95 | |
| (R)-20 | 0.195 | 0.11 |
| 21-P1 | 0.165 | 0.0415 |
| 21-P2 | 2.3 | |
| (R)-22 | 0.25 | 5.2 |
| (R)-23 | 0.4 | 1.2 |
| (R)-24 | 0.89 | 0.26 |
| (R)-25 | 0.068 | 4.2 |
| (R)-26 | 0.086 | 3.1 |
| (R)-27 | 0.23 | 5.7 |
| (R)-28 | 0.56 | |
| (R)-29 | 0.66 | |
| (R)-30 | 0.08 | >10 |
| (R)-31 | 1.5 | |
| (R)-32 | 1.3 | |
| (R)-33 | 0.44 | |
| (R)-34 | 0.64 | 0.012 |
| (R)-35 | 1.7 | |
| (R)-36 | 0.63 | |
| (R)-37 | 0.75 | |
| (R)-38 | 1 | |
| (R)-39 | 1.2 | |
| (R)-40 | 0.047 | >10 |
| (R)-41 | 0.47 | |
| (R)-42 | 0.066 | >10 |
| (R)-43 | 0.21 | 5.9 |
| (R)-44 | 0.39 | >10 |
| (R)-45 | 0.225 | >10 |
| (R)-46 | 1.8 | >10 |
| (R)-47 | 0.335 | >10 |
| (R)-48 | 0.0425 | >10 |
| (R)-49 | 0.043 | >10 |
| (R)-50 | 0.205 | 6.2 |
| (R)-51 | 0.77 | |
| (R)-52 | 0.0375 | >10 |
| (R)-53 | 0.025 | >10 |
| (R)-54 | 0.0827 | >10 |
| (R)-55 | 0.17 | 2.2 |
| (R)-56 | 0.051 | >10 |
| (R)-57 | 1.7 | |
| (R)-58 | 1.6 | |
| (R)-59 | 0.145 | >10 |
| (R)-60 | 0.1025 | >10 |
| (R)-61 | 0.4 | >10 |
| (R)-62 | 0.047 | >10 |
| (R)-63 | 0.0545 | >10 |
| (R)-64 | 0.205 | >10 |
| (R)-65 | 0.41 | >10 |
| (R)-66 | 0.0945 | 4.5 |
| (R)-67 | 0.425 | >10 |
| (R)-68 | 1.4 | |
| (R)-69 | >30 | |
| (R)-70 | 0.03 | >10 |
| (R)-71 | | 6.4 |
| (R)-72 | 0.135 | >10 |
| (R)-73-1 | 0.033 | 4.9 |
| (R)-73-2 | 4.2 | |
| (R)-74 | 0.0225 | >10 |
| (R)-75 | 0.57 | >10 |
| (R)-76 | 0.073 | 2.8 |
| (R)-77 | 0.77 | |
| (R)-78 | 0.27 | >10 |
| (R)-79 | 0.18 | 5.6 |
| (R)-80 | 0.18 | >10 |
| (R)-81 | 0.18 | >10 |
| (R)-82 | 0.087 | >10 |
| (R)-83 | 0.23 | >10 |
| (R)-84 | 0.95 | |
| (S)-85 | >30 | |
| (R)-85 | >30 | |
| 86-P1 | >30 | |
| 86-P2 | 2.5 | |
| 87-P1 | >30 | |
| 87-P2 | >30 | |
| 88-P1 | 4.2 | |
| 88-P2 | >30 | |
| 89-P1 | 3.3 | |
| 89-P2 | 7.1 | |
| 90-P1 | >30 | |
| 90-P2 | >30 | |
| 91-P1 | >30 | |
| 91-P2 | >30 | |
| (R)-92 | 2.1 | |
| (R)-93 | 2.9 | |
| (R)-94 | 4.5 | |
| (R)-95 | 2.4 | |
| (R)-96 | 20 | >10 |
| (R)-97 | 12 | |
| 98-P1 | >30 | |
| 98-P2 | >30 | |
| (R)-99 | >30 | >10 |
| (R)-100 | >30 | >10 |

For reference, the literature reported α7 nAChR agonist AQW051 has a Kᵢ of 255 nM in the above described human α7 nAChR binding assay provided by Cerep (lit: Kᵢ =28 nM; radioligand binding assay using recombinantly expressed human α7-nAChR and [¹²⁵I] α-BTX radioligand; Feuerbach et al., Br. J. Pharmacol., 2014, doi: 10.1111/bph.13001).

All publications and patent applications mentioned in this specification are herein incorporated by reference to the same extent as if each individual publication or patent application was specifically and individually indicated to be incorporated by reference.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

1.-187. (canceled)

188. A compound represented by Formula (I): 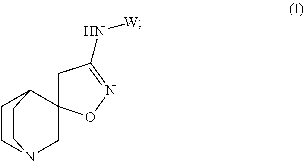
wherein:
W represents a moiety represented by ring system M-I, M-II, M-III, or M-IV: 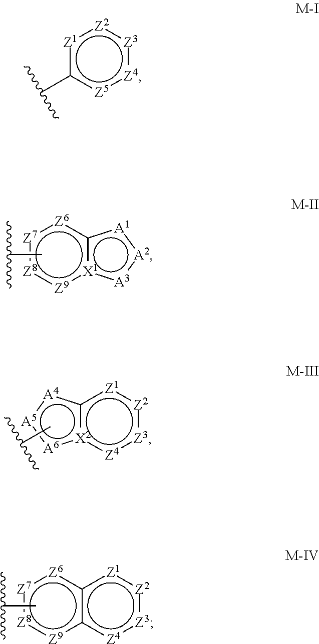
wherein:
Z¹, Z², Z³, Z⁴, and Z⁵independently represent N or CR¹; with the proviso that no more than two of Z¹, Z², Z³, Z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein:
i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂ₘN(R²(R₃), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the N(R²)(R³) moiety forms a cycle, wherein R² and R³ taken together represent a C₂-C₅-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁ C₄ alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O;
wherein the C₂-C₅-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁶, and Z⁹ are N;
R⁴ independently represents —H, —D, halogen radical, —CN, an unbranched C₁-C₄-alkyl radical, a branched C₃-C₄-alkyl radical, a C₃⁻ C₆-cycloalkyl radical, a (3-6 membered)-heterocycloalkyl radical, an unbranched —OC₁-C₄-alkyl, a branched or cyclic —OC₃-C₄-alkyl, —N(R⁵)(R⁶), —(CO)C₁-C₄-alkyl, —(CO)N(R⁵)(R⁶), —NR⁵(CO)(R⁶), —SO₂C₁-C₄-alkyl, —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, an aryl radical, a heteroaryl radical, or the bond directly attaching the W moiety with the aminoisoxazoline moiety; wherein:
i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁵)SO₂C₁-C₄-alkyl, may be independently substituted with up to 5 radical substituents comprising: —D, halogen radical, ═O, —OR⁵, —(CH₂)ₘOR⁵, —N(R⁵)(R⁶), —NR⁵(CO)(R⁶), —(CH₂)ₘN(R⁵)(R⁶), —SO₂C₁-C₄-alkyl, —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, halogen radical, —CN, —OR⁵, —(CH₂)ₘOR⁵, —N(R⁵)(R⁶), —NR⁵(CO)(R⁶), —(CH₂)ₘN(R⁵)(R⁶), —SO₂C₁-C₄-alkyl, —SO₂N(R⁵)(R⁶), —(CH₂)ₘSO₂C₁-C₄-alkyl, —(CH₂)ₘSO₂N(R⁵)(R⁶), —N(R⁵)SO₂C₁-C₄-alkyl, —(CO)(CH₂)ₘR⁵, —(CO)N(R⁵)(R⁶), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁵ and R⁶ independently represent —H, —D, an unbranched C₁-C₄-alkyl radical, a branched C₃-C₄-alkyl radical, a C₃-C₄-cycloalkyl radical, or the N(R⁵)(R⁶) moiety forms a cycle, wherein R⁵ and R⁶ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X¹ independently represents N or C;
A¹, A², and A³ independently represent N, —NR⁷, N(CH₂)ₘR⁷, O, S, or CR⁸; with the proviso that only one A¹, A², and A³ is NR⁷, N(CH₂)ₘR⁷, O, or S; with the further proviso that when X¹ is N, then A¹, A², and A³ independently represent N or CR⁸;
R⁷ independently represents —H, —D, —SO₂(CH₂)ₘR⁹, —(CO)(CH₂)ₘR⁹, —(CO)N(R⁹)(R¹⁰), an unbranched C₁-C₄-alkyl radical, a branched C₃-C₄-alkyl radical, a C₃-C₆-cycloalkyl radical, a (3-6 membered)-heterocycloalkyl radical, an aryl radical, or a heteroaryl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR⁹, —(CH₂)ₘOR⁹, —N(R⁹)(R¹⁰), —NR⁹(CO)(R¹⁹), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR⁹, —(CH₂)ₘOR⁹, —N(R⁹)(R¹⁰), —NR⁹(CO)(R¹⁹), —(CH₂)ₘN(R⁹)(R¹⁰), —SO₂C₁-C₄-alkyl, —SO₂N(R⁹)(R¹⁹), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁸ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁹)(R¹⁰), —(CO)C₁-C₄-alkyl, —(CO)N(R⁹)(R¹⁰), —NR⁹(CO)(R¹⁰) —SO₂C₁-C₄-alkyl, —SO₂N(R⁹)(R¹⁰), —(CH₂)ᵣₙSO₂C₁-C₄-alkyl, —(CH₂)SO₂N(R⁹)(R¹⁰), —N(R⁹)SO₂C₁-C₄-alkyl, an aryl radical, or a heteroaryl radical; wherein:
i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C4-alkyl, or the —N(R⁹)SO₂C₁-C₄-alkyl, may be independently substituted with up to 5 radical substituents comprising: —D, halogen radical, ═O, —OR⁹, —(CH₂)ₘOR⁹, —N(13⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, halogen radical, —CN, —(CH₂)ₘOR⁹, —N(R⁹)(R¹⁰), —NR⁹(CO)(R¹⁰), —(CH₂)ₘN(R⁹)(R¹⁰), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁰), —N(R⁹)SO₂C₁-C₄-alkyl, —(CO)(CH₂)ₘR⁹, —(CO)N(R⁹)(R¹⁰), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R⁹ and R¹° independently represent —H, —D, an unbranched C₁-C₄-alkyl radical, a branched C₃-C₄-alkyl radical, a C₃-C₄-cycloalkyl radical, or the N(R⁹)(R¹⁰) moiety forms a cycle, wherein R⁹ and R¹⁰ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, —NR¹¹, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²;
R¹¹ independently represents —H, —D, —SO₂(CH₂)ₘR¹³, —(CO)(CH₂)ₘR¹³, —(CO)N(R¹³)(R¹⁴), an unbranched C₁-C₄-alkyl radical, a branched C₃-C₄-alkyl radical, a C₃-C₆-cycloalkyl radical, a (3-6 membered)-heterocycloalkyl radical, an aryl radical, a heteroaryl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂),R¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and wherein the aryl radical or the heteroaryl radical, may be independently substituted with up to 5 radical substituents comprising: —D, halogen radical, —CN, —OR¹³ —(CH₂)ₘOR¹³, —N(R¹³)(R¹⁴), —NR¹³(CO)(R¹⁴), —(CH₂)ₘN(R¹³)(R¹⁴) —SO₂C₁-C₄-alkyl, —SO₂N(R¹³)(R¹⁴), —(CH₂)ₘSO₂C₁-C₄⁻alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹² independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R¹³)(R¹⁴), —(CO)C₁-C₄-alkyl, —(CO)N(R¹³)(R¹⁴), —NR¹³(CO)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, an aryl radical, a heteroaryl radical, or the bond directly attaching the W moiety with the aminoisoxazoline moiety; wherein:
i) the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R¹³)SO₂C₁-C₄-alkyl, may be independently substituted with up to 5 radical substituents comprising: —D, halogen radical, ═O, —OR¹³, —(CH₂)ₘOR¹³, —N(R¹³)(R¹⁴), —NR¹³(CO)(R¹⁴), —(CH₂)ₘN(R¹³)(R¹⁴), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁ C₄ alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, halogen radical, —CN, —OR¹³, —(CH₂),OR¹³, —N(R¹³)(R¹⁴), —NR¹³(CO)(R¹⁴), —(CH₂),N(R¹³)(R^{14.,}), —SO₂C₁-C₄-alkyl, —SO₂N(R¹³)(R¹⁴), —(CH₂)ₘSO₂C₁-C₄-alkyl, —(CH₂)ₘSO₂N(R¹³)(R¹⁴), —N(R¹³)SO₂C₁-C₄-alkyl, —(CO)(CH₂)ₘR¹³, —(CO)N(R¹³)(R¹⁴), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R¹³ and R¹⁴ independently represent —H, —D, an unbranched C₁-C₄-alkyl radical, a branched C₃-C₄-alkyl radical, a C₃-C₄-cycloalkyl radical, or the N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof;
with the proviso that the aminoisoxazoline compound represented by Formula (I) is exclusive of:
N-phenyl-4H-1′-azaspiro[isoxazole-5,3′-bicyclo[2.2.2]octan]-3-amine;
N-(isoquinolin-3-yl)-4H-1′-azaspiro[isoxazole-5,3′-bicyclo[2.2.2]octan]-3-amine;
N-(benzo[d]thiazol-2-yl)-4H-1′-azaspiro[isoxazole-5,3′-bicyclo[2.2.2]octan]-3-amine;
N-(6-methoxybenzo[d]thiazol-2-yl)-4H-1′-azaspiro[isoxazole-5,3′-bicyclo[2.2.2]octan]-3-amine;
N-(1H-indazol-3-yl)-4′H-4-azaspiro[bicyclo[2.2.2]octane-2,5′-isoxazol]-3′-amine;
N-(7-bromopyrrolo[2,1-f][1,2,4]triazin-4-yl)-4H-1′-azaspiro[isoxazole-5,3′-bicyclo[2.2.2]octan]-3-amine;
N-(4-chlorobenzo[d]thiazol-2-yl)-4H-1′-azaspiro[isoxazole-5,3′-bicyclo[2.2.2]octan]-3-amine;
N-(5-chlorobenzo[d]thiazol-2-yl)-4H-1′-azaspiro[isoxazole-5,3¹-bicyclo[2.2.2]octan]-3-amine;
N-(6-chlorobenzo[d]thiazol-2-yl)-4H-1′-azaspiro[isoxazole-5,3′-bicyclo[2.2.2]octan]-3-amine;
N-(7-chlorobenzo[d]thiazol-2-yl)-4H-1′-azaspiro[isoxazole-5,3¹-bicyclo[2.2.2]octan]-3-amine;
N-(benzo[b]thiophen-2-yl)-4H-1-azaspiro[isoxazole-5,3′-bicyclo[2.2.2]octan]-3-amine;
N-(benzo[d]oxazol-2-yl)-4H-1′-azaspiro[isoxazole-5,3′-bicyclo[2.2.2]octan]-3-amine;
N-(5,6,7,8-tetrahydroisoquinolin-3-y0-4H-1′-azaspiro[isoxazole-5,3′-bicyclo[2.2.2]octan]-3-amine;
N-(5-methoxypyrimidin-2-yl)-4H-1-azaspiro[isoxazole-5,3′-bicyclo[2.2.2]octan]-3-amine;
N-(furo[3,2-b]pyridin-5-yl)-4H-1-azaspiro[isoxazole-5,3′-bicyclo[2.2.2]octan]-3-amine;
and
N-(furo[2,3-c]pyridin-5-yl)-4H-1-azaspiro[isoxazole-5,3′-bicyclo[2.2.2]octan]-3-amine.

189. The compound of claim 188, wherein the W is represented by the moiety represented by the ring system M-I: 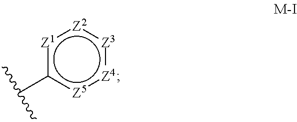
wherein:
Z¹, Z⁴, and Z⁵independently represent N or CR¹; with the proviso that no more than two of Z¹, Z², Z³, Z⁴, and Z⁵ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, an aryl radical, or a heteroaryl radical; or when adjacent members of Z¹, Z², Z³, Z⁴, and Z⁵, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁ C₄ alkyl, —(CO)-branched C₃-C₄-alkyl, —(SO₂)-unbranched C₁-C₄-alkyl, or —(SO₂)-branched C₃ C₄-alkyl, and with the further proviso that when the at least one ring atom is sulfur, the sulfur may be independently substituted with 0 to 2 ═O; wherein:
i) the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)SO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂),—OR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; and
ii) the aryl radical or the heteroaryl radical may be independently substituted with up to 5 radical substituents comprising: —D, halogen radical, —CN, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or 0C₁-C₂-haloalkyl radical;
with the proviso that:
i) when each member of Z¹, Z², Z³, Z⁴, and Z⁵ independently represent CR¹, then no more than four of the R¹ is —H;
ii) when both of Z¹ and Z⁵ are N and Z², Z³, and Z⁴ independently represent CR¹ with the R¹ of Z³ representing —OCH₃, then no more than one of the R¹ of the Z² and Z⁴ is —H; or
iii) when Z¹ is N, each of Z², Z³, Z⁴, and Z⁵ independently represent CR¹, and adjacent members Z³ and Z⁴ form a cycle such that the adjacent R¹ substitutents taken together represents a C₄-alkyl di-radical; then:
a) no more than one of the R¹ of the Z² and Z⁵ is —H; or
b) the C₄-alkyl di-radical is independently substituted with at least one radical substituent;
R² and R³ independently represent —H, —D, an unbranched C₁-C₄-alkyl radical, a branched C₃-C₄-alkyl radical, a C₃-C₄-cycloalkyl radical, or the N(R²)(R³) moiety forms a cycle, wherein R² and R³ taken together represent a C₂-C₅-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₅-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof.

190. The compound of claim 188, wherein the W is represented by the moiety represented by the ring system M-II: 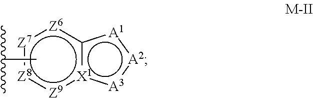
wherein:
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, or the bond directly attaching the W moiety with the aminoisoxazoline moiety; wherein the alkyl portion of the unbranched Cₗ-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, or ═O;
X¹ independently represents N or C;
A¹, A², and A³ independently represent N, —NR⁷, N(CH₂)ₘR⁷, N(CH₂)ₘR⁷, O, S, or CR⁸; with the proviso that only one A¹, A², and A³ is NR⁷, O, or S; with the further proviso that when X¹ is N, then A¹, A², and A³ independently represent N or CR⁸;
R⁷ independently represents —H, —D, —SO₂(CH₂)ₘR⁹, —(CO)(CH₂)ₘR⁹, —(CO)N(R⁹)(R¹⁰), an unbranched C₁-C₄-alkyl radical, a branched C₃-C₄-alkyl radical, a C₃-C₆-cycloalkyl radical, or a (3-6 membered)-heterocycloalkyl radical; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, or ═O;
R⁸ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, a branched or cyclic —OC₃-C₄-alkyl, —N(R⁹)(R¹⁰), —(CO)C₁-C₄-alkyl, —(CO)N(R⁹)(R¹⁰), —NR⁹(CO)(R¹⁹), —SO₂C₁-C₄-alkyl, —SO₂N(R⁹)(R¹⁰), —(CH₂)ₘSO₂C₁-C₄-alkyl, —(CH₂)ₘSO₂N(R⁹)(R¹⁹), or —N(R⁹)SO₂C₁-C₄-alkyl; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, the branched or cyclic —OC₃-C₄-alkyl, the —(CO)C₁-C₄-alkyl, the —SO₂C₁-C₄-alkyl, the —(CH₂)ₘSO₂C₁-C₄-alkyl, or the —N(R⁹)SO₂C₁-C₄-alkyl, may be independently substituted with up to 5 radical substituents comprising: —D, halogen radical, or ═O; with the proviso that:
a) when Z⁶ represents CR⁴ and the R⁴ is the bond directly attaching the W moiety with the aminoisoxazoline moiety, each of X¹, Z⁷, and Z⁹ represent N, Z⁸ represents CR⁴, both A¹ and A² independently represent CR⁸, A³ represents CR⁸ and the R⁸ of the A³ represents —Br, then no more than two of the following is —H: the R⁴ of the Z⁸, the R⁸ of the A¹, and the R⁸ of the A²;
b) when Z⁷ represents CR⁴ and the R⁴ is the bond directly attaching the W moiety with the aminoisoxazoline moiety, X¹ represents C, both Z⁸ and Z⁹ independently represent CR⁴, Z⁶ represents N, A³ represents O, and both A¹ and A² independently represent CR⁸, then no more than three of the following is —H: the R⁴ of the Z⁸, the R⁴ of the Z⁹, the R⁸ of the A¹, and the R⁸ of the A²; or
c) when Z⁷ represents CR⁴ and the R⁴ is the bond directly attaching the W moiety with the aminoisoxazoline moiety, X¹ represents C, both Z⁶ and Z⁹ independently represent CR⁴, Z⁸ represents N, A³ represents O, and both A¹ and A² independently represent CR⁸, then no more than three of the following is H: the R⁴ of the Z⁶, the R⁴ of the Z⁹, the R⁸ of the A¹, and the R⁸ of the A²;
R⁹ and R¹⁹ independently represent —H, —D, an unbranched C₁-C₄-alkyl radical, a branched C₃-C₄-alkyl radical, a C₃-C₄-cycloalkyl radical, or the N(R⁹)(R¹⁰) moiety forms a cycle, wherein R⁹ and R¹¹ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄ alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof.

191. The compound of claim 190, wherein M-II represents a moiety represented by ring system M-II-2:

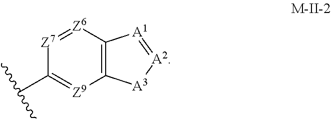

192. The compound of claim 191, wherein each of Z⁶, Z⁷, and Z⁹, independently represent CR⁴; and wherein R⁴ independently represents —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, or a branched or cyclic —OC₃-C₄-alkyl; wherein the alkyl portion of the unbranched C₁-C₄-alkyl radical, the branched C₃-C₄-alkyl radical, the C₃-C₆-cycloalkyl radical, the unbranched —OC₁-C₄-alkyl, or the branched or cyclic —OC₃-C₄-alkyl, may be independently substituted with up to 5 radical substituents comprising: —D, halogen radical or ═O.

193. The compound of claim 192, wherein A¹ and A² independently represent CR⁸;
and wherein R⁸ independently represents —H, —D, halogen radical, —CN, an unbranched C₁-C₄-alkyl radical, a branched C₃-C₄-alkyl radical, a C₃-C₄-cycloalkyl radical, an unbranched —OC₁-C₄-alkyl, a branched or cyclic —OC₃-C₄-alkyl; wherein the alkyl portion of the unbranched C₁-C₄-alkyl radical, the branched C₃-C₄-alkyl radical, the C₃-C₄-cycloalkyl radical, the unbranched —OC₁-C₄-alkyl, or the branched or cyclic —OC₃-C₄-alkyl, may be independently substituted with up to 5 radical substituents comprising: —D, halogen radical, or ═O.

194. The compound of claim 191, wherein:
Z⁶, Z⁷, and Z⁹ independently represent CR⁴; wherein R⁴ independently represents —H —D, —F, —Cl;
A¹ represents CR⁸, wherein the R⁸ independently represents —H or —D;
A² represents CR⁸, wherein R⁸ independently represents —F, —Cl, —CH₃, —CH₂F, —CHF₂, or —CF₃; and
A³ represents O or S.

195. The compound of claim 188, wherein the W is represented by the moiety represented by the ring system M-III: 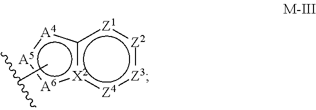
wherein:
Z², Z³, and Z⁴ independently represent N or CR¹; with the proviso that no more than two of Z¹, Z², Z³, and Z⁴ are N;
independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), or —N(R²)SO₂C₁-C₄-alkyl; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, or the —N(R²)SO₂C₁-C₄-alkyl, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
X² independently represents N or C;
A⁴, A⁵, and A⁶ independently represent N, —NR¹¹, N(CH₂)ₘR¹¹, O, S, or CR¹²; with the proviso that only one A⁴, A⁵, and A⁶ is NR¹¹, N(CH₂)ₘR¹¹, O, or S; with the further proviso that when X² is N, then A⁴, A⁵, and A⁶ independently represent N or CR¹²;
R¹¹ independently represents —H, —D, —SO₂(CH₂)ₘR¹³, —(CO)(CH₂)ₘR¹³, —(CO)N(R¹³)(R¹⁴), an unbranched C₁-C₄-alkyl radical, a branched C₃-C₄-alkyl radical, a C₃-C₆-cycloalkyl radical, or a (3-6 membered)-heterocycloalkyl radical, or the bond directly attaching the W moiety with the aminoisoxazoline moiety; wherein the unbranched C₁-C₄-alkyl radical, the branched C₃-C₄-alkyl radical, the C₃-C₆-cycloalkyl radical, or the (3-6 membered)-heterocycloalkyl radical, may be independently substituted with up to 5 radical substituents comprising: —D, halogen radical, or ═O,
R¹² independently represents —H, —D, hₐlo_{g}en radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, or a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, ═O, an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical; with the proviso that:
a) when A⁵ represents CR¹² and the R¹² is the bond directly attaching the W moiety with the aminoisoxazoline moiety, X² represents C, A⁴ represents N, and A⁶ represents S, and each of Z¹, Z², Z³, and Z⁴ independently represent CR¹, then:
i) no more than three of the R¹ is —H;
ii) if one R¹ is Cl, then no more than two of the R¹ is —H; or
iii) if one of the R¹ of either Z² or Z³ is —OMe, then no more than two of the R¹ is —H;
b) when A⁴ represents CR¹² and the R¹² is the bond directly attaching the W moiety with the aminoisoxazoline moiety, X² represents C, A⁵ represents N, and A⁶ represents NR¹¹, and each of Z¹, Z², Z³, and Z⁴ independently represent CR¹, then no more than four of the following is —H: the R¹¹ of the A⁶ and the R¹ of the Z¹ to Z⁴;
c) when A⁵ represents CR¹² and the R¹² is the bond directly attaching the W moiety with the aminoisoxazoline moiety, X² represents C, A⁴ represents CR¹², A⁶ represents S, and each of Z¹, Z², Z³, and Z⁴ independently represent CR¹, then no more than four of the following is H: the R¹² of the A⁴ and the R¹ of the Z¹ to Z⁴; or
d) when A⁵ represents CR¹² and the R¹² is the bond directly attaching the W moiety with the aminoisoxazoline moiety, X² represents C, A⁴ represents N, A⁶ represents O, and each of Z¹, L Z³, and Z⁴ independently represent CR¹, then no more than three of the R¹ is —H;
R¹³ and R¹⁴ independently represent —H, —D, an unbranched C₁-C₄-alkyl radical, a branched C₃-C₄-alkyl radical, a C₃-C₄-cycloalkyl radical, or the N(R¹³)(R¹⁴) moiety forms a cycle, wherein R¹³ and R¹⁴ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical, may be independently substituted with up to 5 radical substituents comprising: —D, halogen radical, ═O, an unbranched C₁-C₄-alkyl radical, or a branched C₃-C₄-alkyl radical; and
m independently represents an integer from 1 to 6;
or a pharmaceutically acceptable salt thereof.

196. The compound of claim 188, wherein the W is represented by the moiety represented by the ring system M-IV: 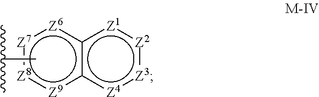
wherein:
Z¹, Z², Z³, and Z⁴ independently represent N or CR¹; with the proviso that no more than two of Z¹, Z², Z³, and Z⁴ are N;
R¹ independently represent —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), or —N(R²)SO₂C₁-C₄-alkyl; or when adjacent members of Z¹, Z², Z³, and Z⁴, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁ C₄ alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²)(R³), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a C₁-C₂-haloalkyl radical, or —OC₁-C₂-haloalkyl radical;
R² and R³ independently represent —H, —D, an unbranched C₁-C₄-alkyl radical, a branched C₃-C₄-alkyl radical, a C₃-C₄-cycloalkyl radical, or the N(R²)(R³) moiety forms a cycle, wherein R² and R³ taken together represent a C₂-C₆-alkyl di-radical or a (3-6 membered)-heteroalkyl di-radical; wherein the (3-6 membered)-heteroalkyl di-radical comprises at least one ring atom selected from the group consisting of oxygen, nitrogen, and sulfur, with the proviso that when the at least one ring atom is nitrogen, the nitrogen is independently substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the C₂-C₆-alkyl di-radical or the alkyl portion of said (3-6 membered)-heteroalkyl di-radical may be independently substituted with up to 5 radical substituents comprising: —D, halogen radical, ═O, an unbranched C₁-C₄-alkyl radical, or a branched C₃-C₄-alkyl radical;
Z⁶, Z⁷, Z⁸, and Z⁹ independently represent N or CR⁴; with the proviso that no more than two of Z⁶, Z⁷, Z⁸, and Z⁹ are N;
R⁴ independently represents —H, —D, halogen radical, —CN, an unbranched C₁-C₄-alkyl radical, a branched C₃-C₄-alkyl radical, a C₃-C₆-cycloalkyl radical, a (3-6 membered)-heterocycloalkyl radical, an unbranched —OC₁-C₄-alkyl, or a branched or cyclic —OC₃-C₄-alkyl, or the bond directly attaching the W moiety with the aminoisoxazoline moiety; wherein the alkyl portion of the unbranched C₁-C₄-alkyl radical, the branched C₃-C₄-alkyl radical, the C₃-C₆-cycloalkyl radical, the (3-6 membered)-heterocycloalkyl radical, the unbranched —OC₁-C₄-alkyl, or the branched or cyclic —OC₃-C₄-alkyl, may be independently substituted with up to 5 radical substituents comprising: —D, halogen radical, or ═O; with the proviso that when Z⁸ represents CR⁴ and the R⁴ is the bond directly attaching the W moiety with the aminoisoxazoline moiety, each of Z¹, Z², Z³, and Z⁴ independently represent CR¹, both Z⁶ and Z⁹ independently represent CR⁴, and Z⁷ represents N, then no more than five of the following is H: the R¹ of the Z¹ to Z⁴, the R⁴ of the Z⁶, and the R⁴ of the Z⁹; and
m independently represents an integer from 1 to 6;
or a pharmaceutically acceptable salt thereof.

197. The compound of claim 196, wherein M-IV represents a moiety represented by ring system M-IV-2: 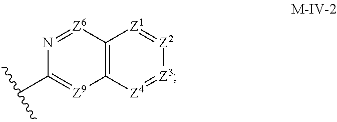
wherein Z¹, Z², Z³, and Z⁴ may independently represent N or CR¹; with the proviso that no more than two of Z¹, Z², Z³, and Z⁴ are N, and wherein Z⁶ and Z⁹ may independently represent N or CR⁴; with the proviso that no more than one of Z⁶ and Z⁹ are N; with the further proviso that when each of Z¹, Z², Z³, and Z⁴ represents CR¹, and both Z⁶ and Z⁹ represent CR⁴, then no more than five of the following is —H: the R¹ of the Z¹ to Z⁴, the R⁴ of the Z⁶, and the R⁴ of the Z⁹.

198. The compound of claim 197, wherein R¹ independently represents —H, —D, halogen radical, —CN, an unbranched C₁-C₄-alkyl radical, a branched C₃-C₄-alkyl radical, a C₃-C₅-cycloalkyl radical, an unbranched —OC₁-C₄-alkyl, a branched or cyclic —OC₃-C₄-alkyl, —N(R²)(R³), —(CO)N(R²)(R³), —NR²(CO)(R³), —SO₂C₁-C₄-alkyl, —SO₂N(R²)(R³), —(CH₂)ₘSO₂C₁-C₄-alkyl, —(CH₂)ₘSO₂N(R²)(R³), or —N(R²)SO₂C₁-C₄-alkyl; or when adjacent members of Z¹, Z², Z³, and Z⁴, is (CR¹)(CR¹), the (CR¹)(CR¹) may form a cycle such that the adjacent R¹ substitutents taken together represents a C₃-C₄-alkyl di-radical or a (3-4 membered)-heteroalkyl di-radical with at least one ring atom of the (3-4 membered)-heteroalkyl di-radical selected from the group consisting of oxygen, nitrogen, and sulfur, with the proviso that when the at least one ring atom is nitrogen, the nitrogen is substituted with —H, —D, an unbranched C₁-C₄-alkyl radical, a branched C₃-C₄-alkyl radical, a C₃-C₄-cycloalkyl radical, —(CO)-unbranched C₁-C₄-alkyl, —(CO)-branched C₃-C₄-alkyl, —(SO₂)-unbranched C₁-C₄-alkyl, or —(SO₂)-branched C₃-C₄-alkyl, and with the further proviso that when the at least one ring atom is sulfur, the sulfur may be independently substituted with 0 to 2 ═O; wherein the alkyl portion of the unbranched C₁-C₄-alkyl radical, the branched C₃-C₄-alkyl radical, the C₃-C₆-cycloalkyl radical, the unbranched —OC₁-C₄-alkyl, the branched or cyclic —OC₃-C₄-alkyl, the —SO₂C₁-C₄-alkyl, the —(CH₂)ₘSO₂C₁-C₄-alkyl, the —N(R²)SO₂C₁-C₄-alkyl, the C₃-C₄-alkyl di-radical, or the (3-4 membered)-heteroalkyl di-radical, may be independently substituted with up to 5 radical substituents comprising: —D, halogen radical, ═O, —OR², —(CH₂)ₘOR², —N(R²)(R³), —NR²(CO)(R³), —(CH₂)ₘN(R²(R₃), —SO₂C₁-C₄-alkyl, —SO₂N(R²)(R³), —(CH₂)ₘSO₂C₁-C₄-alkyl, —(CH₂)ₘSO₂N(R²)(R³), —N(R²)SO₂C₁-C₄-alkyl, —(CO)(CH₂)ₘR², —(CO)N(R²)(R³), an unbranched C₁-C₄-alkyl radical, a branched C₃-C₄-alkyl radical, a C₃-C₄-cycloalkyl radical, a C₁-C₄-hydroxyalkyl radical, a Cₗ-C₂-haloalkyl radical, or —OC₁-C₂-haloalkyl radical.

199. The compound of claim 198, wherein each of Z⁶ and Z⁹, independently represent CR⁴; and wherein R⁴ independently represents —H, —D, halogen radical, —CN, an unbranched C₁-C₄-alkyl radical, a branched C₃-C₄-alkyl radical, a C₃-C₆-cycloalkyl radical, an unbranched —OC₁-C₄-alkyl, a branched or cyclic —OC₃-C₄-alkyl; wherein the alkyl portion of the unbranched Cₗ-C₄-alkyl radical, the branched C₃-C₄-alkyl radical, the C₃-C₆-cycloalkyl radical, the unbranched —OC₁-C₄-alkyl, or the branched or cyclic —OC₃-C₄-alkyl, may be independently substituted with up to 5 radical substituents comprising: —D, halogen radical, or ═O.

200. The compound of claim 197, wherein R¹ independently represents —H, —D, —F, —Cl, or —CH₃; and wherein R⁴ independently represents —H, —D, —F, —Cl, —CH₃, —CH₂F, —CHF₂, or —CF₃.

201. The compound of claim 188, wherein the compound is selected from the group consisting of:
N-(pyrrolo[2,1-f][1,2,4]triazin-4-yl)-4H-1′-azaspiro[isoxazole-5,3′-bicyclo[2.2.2]octan]-3-amine;
N-(6-methylbenzo[d]thiazol-2-yl)-4H-1′-azaspiro[isoxazole-5,3′-bicyclo[2.2.2]octan]-3-amine;
N-(6-cyclopropylbenzo[d]thiazol-2-yl)-4H-1′-azaspiro[isoxazole-5,3′-bicyclo[2.2.2]octan]-3-amine;
N-(6-fluorobenzo[d]thiazol-2-y0-4H-1′-azaspiro[isoxazole-5,3′-bicyclo[2.2.2]octan]-3-amine;
N-(5-chloro-6-fluorobenzo[d]thiazol-2-yl)-4H-1′-azaspiro[isoxazole-5,3′-bicyclo[2.2.2]octan]-3-amine;
N-(6-chloroisoquinolin-3-yl)-4H-1′-azaspiro[isoxazole-5,3′-bicyclo[2.2.2]octan]-3-amine;
N-(7-chloroisoquinolin-3-yl)-4H-1-azaspiro[isoxazole-5,3′-bicyclo[2.2.2]octan]-3-amine;
N-(4-chlorophenyl)-4H-1′-azaspiro[isoxazole-5,3′-bicyclo[2.2.2]octan]-3-amine;
N-(3,4-dichlorophenyI)-4H-1′-azaspiro[isoxazole-5,3′-bicyclo[2.2.2]octan]-3-amine;
N-(quinazolin-2-yl)-4H-1′-azaspiro[isoxazole-5,3′-bicyclo[2.2.2]octan]-3-amine;
N-(8-chloroisoquinolin-3-yl)-4H-1′-azaspiro[isoxazole-5,3¹-bicyclo[2.2.2]octan]-3-amine;
N-(5-chloroisoquinolin-3-yl)-4H-1′-azaspiro[isoxazole-5,3′-bicyclo[2.2.2]octan]-3-amine;
N-(5-methylbenzo[d]thiazol-2-yl)-4H-1′-azaspiro[isoxazole-5,3′-bicyclo[2.2.2]octan]-3-amine;
N-(5-cyclopropylbenzo[d]thiazol-2-yl)-4H-1′-azaspiro[isoxazole-5,3′-bicyclo[2.2.2]octan]-3-amine;
N-(5-fluorobenzo[d]thiazol-2-yl)-4H-1′-azaspiro[isoxazole-5,3′-bicyclo[2.2.2]octan]-3-amine;
N-(5-(trifluoromethyl)benzo[d]thiazol-2-yl)-4H-1′-azaspiro[isoxazole-5,3′-bicyclo[2.2.2]octan]-3-amine;
2-((4H-1′-azaspiro[isoxazole-5,3′-bicyclo[2.2.2]octan]-3-yDamino)benzo[d]thiazole-5-carbonitrile;
N-(5,6-dichlorobenzo[d]thiazol-2-yl)-4H-1′-azaspiro[isoxazole-5,3′-bicyclo[2.2.2]octan]-3-amine;
N-(6,7-dichlorobenzo[d]thiazol-2-yl)-4H-1′-azaspiro[isoxazole-5,3′-bicyclo[2.2.2]octan]-3-amine;
N-(5,7-dichlorobenzo[d]thiazol-2-yl)-4H-1′-azaspiro[isoxazole-5,3′-bicyclo[2.2.2]octan]-3-amine;
N-(6-chloro-5,7-difluorobenzo[d]thiazol-2-yl)-4H-l-azaspiro[isoxazole-5,3′-bicyclo[2.2.2]octan]-3-amine;
N-(3-chlorophenyl)-4H-1′-azaspiro[isoxazole-5,3′-bicyclo[2.2.2]octan]-3-amine;
N-(3-chloro-4-(trifluoromethyl)phenyI)-4H-1′-azaspiro[isoxazole-5,3′-bicyclo[2.2.2]octan]-3-amine;
N-(3-chloro-4-cyclopropylphenyI)-4H-1′-azaspiro[isoxazole-5,3′-bicyclo[2.2.2]octan]-3-amine;
N-(4-chloro-3-(trifluoromethyl)phenyl)-4H-1′-azaspiro[isoxazole-5,3′-bicyclo[2.2.2]octan]-3-amine;
N-(4-chloro-3-cyclopropylphenyl)-4H-1′-azaspiro[isoxazole-5,3′-bicyclo[2.2.2]octan]-3-amine;
N-(4-chloro-3-methoxyphenyl)-4H-1-azaspiro[isoxazole-5,3′-bicyclo[2.2.2]octan]-3-amine;
N-(2-methylbenzofuran-6-yI)-4H-1′-azaspiro[isoxazole-5,3′-bicyclo[2.2.2]octan]-3-amine;
N-(2-methylbenzofuran-5-yl)-4H-1′-azaspiro[isoxazole-5,3′-bicyclo[2.2.2]octan]-3-amine;
N-(2-methylbenzo[b]thiophen-6-yl)-4H-1′-azaspiro[isoxazole-5,3′-bicyclo[2.2.2]octan]-3-amine;
N-(2-methylbenzo[b]thiophen-5-yl)-4H-1′-azaspiro[isoxazole-5,3′-bicyclo[2.2.2]octan]-3-amine;
N-(7-fluoroisoquinolin-3-yI)-4H-1′-azaspiro[isoxazole-5,3′-bicyclo[2.2.2]octan]-3-amine;
N-(6-fluoroisoquinolin-3-yl)-4H-1′-azaspiro[isoxazole-5,3′-bicyclo[2.2.2]octan]-3-amine;
N-(7-methoxyisoquinolin-3-yl)-4H-1′-azaspiro[isoxazole-5,3′-bicyclo[2.2.2]octan]-3-amine;
N-(6-methoxyisoquinolin-3-yI)-4H-1′-azaspiro[isoxazole-5,3′-bicyclo[2.2.2]octan]-3-amine;
N-(6-chloroquinazolin-2-yl)-4H-1′-azaspiro[isoxazole-5,3′-bicyclo[2.2.2]octan]-3-amine;
N-(7-chloroquinazolin-2-yl)-4H-1′-azaspiro[isoxazole-5,3′-bicyclo[2.2.2]octan]-3-amine N-(2-methylbenzo[d]thiazol-6-yl)-4H-1′-azaspiro[isoxazole-5,3′-bicyclo[2.2.2]octan]-3-amine;
N-(2-methylbenzo[d]oxazol-6-yl)-4H-1′-azaspiro[isoxazole-5,3′-bicyclo[2.2.2]octan]-3-amine;
N-(6-methylthieno[2,3-d]pyrimidin-2-yI)-4H-1′-azaspiro[isoxazole-5, 3′-bicyclo[2.2.2]octan]-3-amine;
N-(2-methylthieno[2,3-b]pyridin-6-yI)-4H-1′-azaspiro[isoxazole-5,3′-bicyclo[2.2.2]octan]-3-amine;
N-(2-chlorobenzo[b]thiophen-611)-4H-1′-azaspiro[isoxazole-5,3′-bicyclo[2.2.2]octan]-3-amine;
N-(2,3-difluorobenzo[b]thiophen-6-yl)-3H-1¹-azaspiro[fura n-2, 3′-bicyclo[2.2.2]octan]-4-amine;
N-(3-rriethylbenzo[b]thiophen-6-yl)-4H-1′-azaspiro[isoxazole-5,3′-bicyclo[2.2.2]octan]-3-amine;
N-(1-methyl-1 H-benzo[d]imidazol-6-yl)-4H-1′-azaspiro[isoxazole-5,3′-bicyclo[2.2.2]octan]-3-a mine;
N-(1-methyl-1 H-benzo[d]imidazol-5-yl)-4H-1-azaspiro[isoxazole-5,3′-bicyclo[2.2.2]octan]-3-amine;
N-(7-methylisoquinolin-3-yl)-4H-1′-azaspiro[isoxazole-5,3′-bicyclo[2.2.2]octan]-3-amine;
N-(6-methylisoquinolin-3-yl)-4H-1′-azaspiro[isoxazole-5,3′-bicyclo[2.2.2]octan]-3-amine;
N-(6,7-difluoroisoquinolin-3-yl)-4H-1′-azaspiro[isoxazole-5,3′-bicyclo[2.2.2]octan]-3-amine;
N-(6,7-dichloroisoq uinolin-3-yI)-4H-1′-azaspiro[isoxazole-5,3′-bicyclo[2.2.2]octan]-3-amine;
N-(2-chlorobenzofuran-6-yl)-4H-1′-azaspiro[isoxazole-5,3′-bicyclo[2.2.2]octan]-3-amine;
N-(1 -methyl-1 H-indazol-6-yl)-4H-1′-azaspiro[isoxazole-5,3′-bicyclo[2.2.2]octan]-3-amine;
N-(1 -methyl-1 H-indazol-5-yl)-4H-1′-azaspiro[isoxazole-5,3′-bicyclo[2.2.2]octan]-3-amine;
N-(3-methylbenzo[d]isoxazol-6-yl)-4H-1′-azaspiro[isoxazole-5,3′-bicyclo[2.2.2]octan]-3-amine;
N-(2-(trifluoromethyl)benzo[b]thiophen-6-yI)-4H-1′-azaspiro[isoxazole-5,3′-bicyclo[2.2.2]octan]-3-amine;
N-(1,2-dimethyl-1 H-benzo[d]imidazol-6-yl)-4H-1′-azaspiro[isoxazole-5,3′-bicyclo[2.2.2]octan]-3-amine;
N-(1-methyl-1 H-benzo[d]imidazol-2-yl)-4H-1′-azaspiro[isoxazole-5,3′-bicyclo[2.2.2]octan]-3-a mine;
N-(6-methylquinazolin-2-yl)spiro[4H-isoxazole-5,3′-quinuclidine]-3-amine;
N-(7-methylquinazolin-2-yI)-4H-1′-azaspiro[isoxazole-5,3′-bicyclo[2.2.2]octan]-3-amine;
N-(3-methylbenzo[d]isothiazol-6-yl)-4H-1′-azaspiro[isoxazole-5,3′-bicyclo[2.2.2]octan]-3-a mine;
N-(3-chlorobenzofuran-6-yI)-4H-1′-azaspiro[isoxazole-5,3′-bicyclo[2.2.2]octan]-3-amine;
N-(3,5-dichlorobenzofuran-6-yl)-4H-1-azaspiro[isoxazole-5,3′-bicyclo[2.2.2]octan]-3-amine;
N-(3-methylbenzofuran-6-yI)-4H-1′-azaspiro[isoxazole-5,3′-bicyclo[2.2.2]octan]-3-amine;
N-(6-methylfuro[2,3-d]pyrimidin-2-yl)spiro[4H-isoxazole-5,3′-quinuclidine]-3-amine;
N-(3-chlorobenzo[b]thiophen-6-yl)-4H-1′-azaspiro[isoxazole-5,3′-bicyclo[2.2.2]octan]-3-amine;
N-(3-(trifluoromethyl)benzo[b]thiophen-6-yI)-4H-1′-azaspiro[isoxazole-5,3′-bicyclo[2.2.2]octan]-3-amine;
N-(2-chloro-3-fluorobenzo[b]thiophen-6-yI)-4H-1′-azaspiro[isoxazole-5,3′-bicyclo[2.2.2]octan]-3-amine;
N-(quinolin-7-yl)-4H-1′-azaspiro[isoxazole-5,3¹-bicyclo[2.2.2]octan]-3-amine;
N-(quinolin-6-yI)-4H-1′-azaspiro[isoxazole-5,3′-bicyclo[2.2.2]octan]-3-amine;
N-(6-chloro-7-fluoroisoquinolin-3-yI)-4H-1′-azaspiro[isoxazole-5,3′-bicyclo[2.2.2]octan]-3-amine;
N-(7-chloro-6-fluoroisoquinolin-3-yI)-4H-1′-azaspiro[isoxazole-5,3′-bicyclo[2.2.2]octan]-3-amine;
N-(6-(trifluoromethypisoquinolin-3-yl)-4H-1′-azaspiro[isoxazole-5,3′-bicyclo[2.2.2]octan]-3-amine;
N-(2,2-difluorobenzo[d][1,3]dioxol-5-yl)-4H-1′-azaspiro[isoxazole-5,3′-bicyclo[2.2.2]octan]-3-amine;
2-((4H-1′-azaspiro[isoxazole-5,3′-bicyclo[2.2.2]octan]-3-yl)amino)benzo[d]thiazole-6-carbonitrile;
N-(6-(trifluoromethyl) benzo[d]thiazol-2-yl)-4H-l-azaspiro[isoxazole-5,3′-bicyclo[2.2.2]octan]-3-amine;
N-(quinolin-2-yl)-4H-V-azaspiro[isoxazole-5,3′-bicyclo[2.2.2]octan]-3-amine;
N-(pyridin-2-yl)-4H-1′-azaspiro[isoxazole-5,3¹-bicyclo[2.2,2]octan]-3-amine;
N-(4,5,6-trifluorobenzo[d]thiazol-2-yl)-4H-1′-azaspiro[isoxazole-5,3¹-bicyclo[2.2.2]octan]-3-amine;
N-(3,4,5-trichlorophenyl)-4H-1′-azaspiro[isoxazole-5,3¹-bicyclo[2.2.2]octan]-3-amine;
N-(4-cyclopropyl-3-methoxyphenyl)-4H-1′-azaspiro[isoxazole-5,3′-bicyclo[2.2.2]octan]-3-amine;
N-(quinolin-3-yI)-4H-1′-azaspiro[isoxazole-5,3′-bicyclo[2.2.2]octan]-3-amine;
N-(2-methylimidazo[1,2-a]pyridin-7-yl)-4H-1′-azaspiro[isoxazole-5,3′-bicyclo[2.2.2]octan]-3-amine;
N-(1,2-dimethyl-1H-benzo[d]imidazol-5-yl)-4H-1′-azaspiro[isoxazole-5,3′-bicyclo[2.2.2]octan]-3-amine;
N-(2-methylthieno[3,2-c]pyridin-6-yl)spiro[4H-isoxazole-5,3′-quinuclidine]-3-amine;
and
N-(2-methylfuro[3,2-c]pyridin-6-yl)spiro[4H-isoxazole-5,3′-quinuclidine]-3-amine;
and single (R)- or (S)- enantiomers and pharmaceutically acceptable salts thereof.

202. The compound of claim 188, wherein the compound is selected from the group consisting of:
N-(2-fluorobenzofuran-6-yl)-4H-1′-azaspiro[isoxazole-5,3′-bicyclo[2.2.2]octan]-3-amine;
N-(6,7-dichloroquinazolin-2-yl)-4H-1′-azaspiro[isoxazole-5,3′-bicyclo[2.2.2]octan]-3-amine;
N-(6-chloro-7-fluoroquinazolin-2-yI)-4H-1′-azaspiro[isoxazole-5,3′-bicyclo[2.2.2]octan]-3-amine;
N-(6-chlorothieno[2,3-d]pyrimidin-2-yl)-4H-1′-azaspiro[isoxazole-5,3′-bicyclo[2.2.2]octan]-3-amine;
N-(5-methylthieno[2,3-d]pyrimidin-2-yl)-4H-1′-azaspiro[isoxazole-5,3′-bicyclo[2.2.2]octan]-3-amine;
N-(6-(trifluoromethyl)thieno[2,3-d]pyrimidin-2-yl)-4H-1′-azaspiro[isoxazole-5,3′-bicyclo[2.2.2]octan]-3-amine;
N-(7-(trifluoromethyl)isoquinolin-3-yl)-4H-1′-azaspiro[isoxazole-5,3′-bicyclo[2.2.2]octan]-3-amine;
N-(6-(trifluoromethyl)quinazolin-2-yl)-4H-1′-azaspiro[isoxazole-5,3′-bicyclo[2.2.2]octan]-3-amine;
N-(7-(trifluoromethyl)quinazolin-2-yl)-4H-1′-azaspiro[isoxazole-5,3′-bicyclo[2.2.2]octan]-3-amine;
N-(6,7-bis(trifluoromethyl)quinazolin-2-yl)-4H-1′-azaspiro[isoxazole-5,3′-bicyclo[2.2.2]octan]-3-amine;
N-(2-methylfuro[2,3-b]pyridin-6-yl)-4H-1-azaspiro[isoxazole-5,3′-bicyclo[2.2.2]octan]-3-amine;
N-(2-chlorofuro[2,3-b]pyridin-6-yl)-4H-1′-azaspiro[isoxazole-5,3′-bicyclo[2.2.2]octan]-3-amine;
N-(3-methylthieno[2,3-b]pyridin-6-yl)-4H-1′-azaspiro[isoxazole-5,3′-bicyclo[2.2.2]octan]-3-amine;
N-(3-methylthieno[3,2-c]pyridin-6-yl)-4H-1′-azaspiro[isoxazole-5,3′-bicyclo[2.2.2]octan]-3-amine;
N-(3-(difluoromethyl)benzo[b]thiophen-6-yl)-4H-1-azaspiro[isoxazole-5,3′-bicyclo[2.2.2]octan]-3-amine;
N-(2-chlorothieno[2,3-b]pyridin-6-yl)-4H-1′-azaspiro[isoxazole-5,3′-bicyclo[2.2.2]octan]-3-amine;
N-(3-methylisothiazolo[5,4-b]pyridin-6-yl)-4H-1′-azaspiro[isoxazole-5,3′-bicyclo[2.2.2]octan]-3-amine;
N-(3-methylisothiazolo[4,5-c]pyridin-6-yl)-4H-l-azaspiro[isoxazole-5,3¹-bicyclo[2.2.2]octan]-3-amine;
N-(2-cyclopropylbenzo[b]thiophen-6-yl)-4H-1-azaspiro[isoxazole-5,3′-bicyclo[2.2.2]octan]-3-amine;
2,2-difluoro-N-(4H-1′-azaspiro[isoxazole-5,3′-bicyclo[2.2.2]octan]-3-yl)-[1,3]dioxolo[4,5-g]isoquinolin-7-amine;
N-(5-chlorothieno[2,3-d]pyrimidin-2-yI)-4H-1′-azaspiro[isoxazole-5,3′-bicyclo[2.2.2]octan]-3-amine;
N-(5-fluoro-6-methylthieno[2,3-d]pyrimidin-2-yl)-4H-1′-azaspiro[isoxazole-5,3′-bicyclo[2.2.2]octan]-3-amine;
N-(6-chloro-5-fluorothieno[2,3-d]pyrimidin-2-yl)-4H-1′-azaspiro[isoxazole-5,3′-bicyclo[2.2.2]octan]-3-amine;
N-(6-cyclopropylquinazolin-2-yI)-4H-1′-azaspiro[isoxazole-5,3′-bicyclo[2.2.2]octan]-3-amine;
N-(7-cyclopropylquinazolin-2-yl)-4H-1′-azaspiro[isoxazole-5,3′-bicyclo[2.2.2]octan]-3-amine;
N-(2-chloro-3-fluorothieno[2,3-b]pyridin-6-yl)-4H-1′-azaspiro[isoxazole-5,3′-bicyclo[2.2.2]octan]-3-amine;
N-(3-fluoro-2-methylthieno[2,3-b]pyridin-6-yl)-4H-l-azaspiro[isoxazole-5,3′-bicyclo[2.2.2]octan]-3-amine;
N-(3-chlorofuro[2,3-b]pyridin-6-yl)-4H-1′-azaspiro[isoxazole-5,3′-bicyclo[2.2.2]octan]-3-amine;
N-(3-chlorothieno[2,3-b]pyridin-6-yl)-4H-1′-azaspiro[isoxazole-5,3′-bicyclo[2.2.2]octan]-3-amine; and
N-(6-(1H-imidazol-1-yl)pyrimidin-4-yl)-4H-1′-azaspiro[isoxazole-5,3′-bicyclo[2.2.2]octan]-3-amine;
and single (R)- or (S)- enantiomers and pharmaceutically acceptable salts thereof.

203. A pharmaceutical composition, comprising:
i) the compound, or pharmaceutically acceptable salt thereof, of claim 188; and
ii) at least one pharmaceutically acceptable carrier, excipient or diluent.

204. A method of improving cognition of a patient in need thereof, comprising:
administering to the patient the compound, or pharmaceutically acceptable salt thereof, of claim 188.

205. The method of claim 204, wherein the patient suffers from a cognitive impairment, suffers from a cognitive loss associated with a cognitive impairment, or suffers from one or more symptoms associated with a cognitive impairment.

206. The method of claim 205, wherein the cognitive impairment comprises Limited Cognitive Impairment (LCI), Mild Cognitive Impairment (MCI), Alzheimer's disease, dementia of an Alzheimer's-type, schizophrenia, schizophreniform disorder, schizoaffective disorder, delusional disorder, positive symptoms of schizophrenia, negative symptoms of schizophrenia, schizophrenia with dementia, or major depressive disorder.

207. A method of treating or improving one or more symptoms associated with a cognitive disease and/or a cognitive impairment in a patient in need thereof, comprising:
administering to the patient the compound, or pharmaceutically acceptable salt thereof, of claim 188.
